(19) United States
(12) Patent Application Publication
(10) Pub. No.: US 2011/0189183 A1
(43) Pub. Date: Aug. 4, 2011

(54) Antibodies against candida, collections thereof and methods of use

(76) Inventors: Robert Anthony Williamson, La Jolla, CA (US); Jehangir Wadia, San Diego, CA (US); Michelle Wagner, San Diego, CA (US)

(21) Appl. No.: 12/807,955

(22) Filed: Sep. 17, 2010

Related U.S. Application Data
(60) Provisional application No. 61/277,091, filed on Sep. 18, 2009.
(60) Provisional application No. 61/341,175, filed on Mar. 25, 2010.

(51) Int. Cl. A61K 39/395; C12N 15/63; C07K 16/14; C07H 21/00; C12N 1/00; C12N 5/10; A61P 31/10

(52) U.S. Cl. 424/135.1; 435/320.1; 530/389.1; 530/387.3; 530/391.1; 424/172.1; 424/133.1; 536/23.53; 435/243; 435/419; 435/325

(57) ABSTRACT

Provided herein are antibodies that immunospecifically bind to species of the genus Candida. Also provided are methods for of prevention, treatment and diagnosis of Candida infection and/or the treatment of one more symptoms of Candida infection. Methods of generating antibodies that immunospecifically bind Candida also are provided.

### RELATED APPLICATIONS

Benefit of priority is claimed to U.S. Provisional Application Ser. No. 61/277,091, entitled “ANTIBODIES AGAINST CANDIDA AND METHODS OF USE,” filed on Sep. 18, 2009, and to U.S. Provisional Application Ser. No. 61/341,175, entitled “ANTIBODIES AGAINST CANDIDA, COLLECTIONS THEREOF AND METHODS OF USE,” filed on Mar. 25, 2010.

This application is related to corresponding International Application No. [Attorney Docket No. 3800013-00029/1157PC], filed the same day herewith, entitled “ANTIBODIES AGAINST CANDIDA, COLLECTIONS THEREOF AND METHODS OF USE,” which also claims priority to U.S. Provisional Application Ser. Nos. 61/277,091 and 61/341,175.

This application is related to U.S. application Ser. No. 12/586,273, entitled “METHODS FOR CREATING DIVERSITY IN LIBRARIES AND LIBRARIES, DISPLAY VECTORS AND METHODS, AND DISPLAYED MOLECULES,” filed Sep. 18, 2009, and to U.S. application Ser. No. 12/586,307, entitled “METHODS AND VECTORS FOR DISPLAY OF MOLECULES AND DISPLAYED MOLECULES AND COLLECTIONS,” filed Sep. 18, 2009.

This application is related to U.S. Provisional Application Ser. No. 61/192,916, entitled “METHODS FOR CREATING DIVERSITY IN LIBRARIES AND LIBRARIES, DISPLAY VECTORS AND METHODS, AND DISPLAYED MOLECULES,” filed Sep. 22, 2008, and to U.S. Provisional Application Ser. No. 61/192,960, entitled “VECTORS FOR EXPRESSION OF DISPLAYED PROTEINS,” filed Sep. 22, 2008, and to U.S. Application Ser. No. 61/192,982, entitled “METHODS AND VECTORS FOR DISPLAY OF MOLECULES AND DISPLAYED MOLECULES AND COLLECTIONS,” filed Sep. 22, 2008.

The subject matter of each of the above-referenced applications is incorporated by reference in its entirety.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy # 1 and Copy # 2), the contents of which are incorporated by reference in their entirety. The computer-readable file on each of the aforementioned compact discs, created on Sep. 17, 2010, is identical, 991 kilobytes in size, and titled 1157seq.001.txt.

### FIELD OF INVENTION

Provided are domain-exchanged antibodies that immunospecifically bind to Candida species, including Candida albicans. Also provided are diagnostic and therapeutic methods that employ anti-Candida antibodies. The therapeutic methods include administering the anti-Candida antibodies provided for the prevention or treatment of a Candida infection and/or amelioration of one or more symptoms of a Candida infection, such as infections in immunocompromised patients or patients receiving large quantities of antibiotics. Combinations of a plurality of different anti-Candida antibodies provided herein and/or with other anti-fungal antibodies or anti-fungal agents can be used for combination therapy. Compositions containing the mixtures of anti-Candida antibodies also are provided.

### BACKGROUND

Members of the genus Candida are responsible for a wide variety of opportunistic infections in humans. In normal hosts, Candida infections, such as those caused by Candida albicans, can range from mild (e.g. thrush in newborn infants and paronychia) to more severe, chronic infections (e.g. Candida vaginitis). Vaginitis is particularly frequent in otherwise normal females with diabetes or a history of prolonged antibiotic or oral contraceptive use. While short-term topical therapy is effective in treating individual episodes of vaginitis, such agents do not prevent recurrences. Thus, even in the normal host, infection with C. albicans can occur at epithelial surfaces, and recurrences are not prevented by presently available therapies. In immunocompromised hosts such as cancer patients, transplant patients, post-operative surgical patients and premature newborns, C. albicans is the leading fungal pathogen. In T cell compromised patients, Candida infection can cause disease ranging from aggressive local infections such as periodontitis, oral ulceration, or esophagitis to complex and potentially lethal infections of the bloodstream with subsequent dissemination to brain, eye, heart, liver, spleen, kidneys, or bone. Such grave prognoses require more toxic therapy, with attendant consequences from both the underlying infection and the treatment. The infection typically begins at an epithelial site, evades local defenses, and invades the bloodstream in the face of immunosuppression. Hence, there exists a need for the development of effective therapies to prevent and treat infections caused by Candida species.

### SUMMARY

Provided herein are anti-Candida antibodies for the prophylaxis or treatment of Candida infection and Candida-mediated diseases or conditions. Also provided herein are anti-Candida antibodies for the diagnosis and/or monitoring of Candida infection. Provided herein are anti-Candida antibodies that immunospecifically bind to Candida.

Provided herein are anti-Candida antibodies that are domain-exchanged antibodies. The anti-Candida antibodies provided herein are domain-exchanged antibodies that bind to an epitope presented on a Candida cell wall or surface with an affinity of equal to or less than or about 100 nM, where the antibody is not 2G12. For example, the antibodies provided herein do not have a sequence of amino acids for a variable heavy chain set forth as amino acids 4-120 of SEQ ID NO:154 and a variable light chain comprising a sequence of amino acids set forth as amino acids 4-105 of SEQ ID NO:155. Exemplary anti-Candida domain-exchanged antibodies provided herein are variant or modified 2G12 antibodies.

In some examples, the anti-Candida domain-exchanged antibody contains a variable heavy chain that includes one or more amino acid residues selected from amino acid residues isoleucine (Ile) at position 19, arginine (Arg) at position 57, phenylalanine (Phe) at position 77 and proline (Pro) or serine (Ser) at position 113, based on Kabat numbering. In other examples, the anti-Candida domain-exchanged antibody contains a variable heavy chain that includes an isoleucine (Ile) at position 19, an arginine (Arg) at position 57, a phenylalanine (Phe) at position 77 and a proline (Pro) or serine (Ser) at position 113, based on Kabat numbering. In some instances, the variable heavy chain of the anti-Candida domain-exchanged antibody further contains an arginine (Arg) at position 39, a serine (Ser) at position 70, an aspartic acid (Asp) at position 72, a tyrosine (Tyr) at position 79, a glutamine (Gln) at position 81, or a valine (Val) at position 84, based on Kabat numbering. In further examples, the domain-exchanged antibody has a variable heavy chain that comprises one or more of amino acid residues isoleucine (Ile) at position 19; arginine (Arg) at position 39; arginine (Arg) at position 57; phenylalanine (Phe) at position 77; valine (Val) at position 84; and proline (Pro) or serine at position 113, based on kabat numbering. The variable heavy chain can also contain one or both of an alanine (Ala) at position 14 or a glutamic acid (Glu) at position 75, based on kabat numbering.

In some examples, the anti-Candida antibodies provided herein have greater affinity for Candida compared to the affinity of the corresponding form of the domain-exchanged antibody 2G12. A 2G12 includes the full-length 2G12 that has a light chain having the amino acid sequence set forth in SEQ ID NO: 162 and a heavy chain having the amino acid sequence set forth in SEQ ID NO: 160, or fragments thereof.

Provided herein are anti-Candida antibodies that are full-length domain-exchanged antibodies or antibody fragments of domain-exchanged antibodies. In some examples, the domain-exchanged antibody is a domain-exchanged Fab fragment, a domain-exchanged scFv fragment, a domain-exchanged Fab hinge fragment, domain-exchanged scFv tandem fragment, domain-exchanged scFv tandem fragment, or a domain-exchanged single chain Fab fragment. In some examples, where the domain-exchanged antibody is a domain-exchanged single chain Fab fragment, the antibody contains a peptide linker located between the heavy chain and light chain of the domain-exchanged single chain Fab fragment. Exemplary linkers include, but are not limited to linkers that are about 1-50 amino acids in length.

Provided herein are anti-Candida antibodies that are modified 2G12 IgG domain-exchanged antibodies. The modified 2G12 anti-Candida domain-exchanged antibodies provided herein contain at least one amino acid replacement, addition or deletion in an unmodified 2G12 that contains a variable heavy chain including at least the sequence of amino acids set forth as amino acids 4-120 of SEQ ID NO: 154 and a variable light chain including at least the sequence of amino acids set forth as amino acids 4-105 of SEQ ID NO: 155. Thus, provided herein are anti-Candida antibodies that are modified 2G 12 IgG domain-exchanged antibodies where the unmodified 2G12 domain-exchanged antibody contains a variable heavy chain having a sequence of amino acids set forth in SEQ ID NO: 154 and a variable light chain having a sequence of amino acids set forth in SEQ ID NO: 155, SEQ ID NO: 176 or SEQ ID NO: 677. In some examples, the one or more modified amino acid residues are in a 2G 12 complementarity determining region (CDR), provided that heavy chain amino acid residue 57, based on Kabat numbering, is not modified.

Provided herein are anti-Candida antibodies that are modified full-length 2G12 IgG domain-exchanged antibodies. In some examples, where the anti-Candida antibody is a modified 2G12 IgG domain-exchanged antibody, the anti-Candida antibody has greater affinity for Candida compared to the unmodified 2G12 IgG domain-exchanged antibody, where the unmodified 2G12 IgG domain-exchanged antibody comprises a light chain having the amino acid sequence set forth in SEQ ID NO: 162, SEQ ID NO:11 or SEQ ID NO:553 and a heavy chain having the amino acid sequence set forth in SEQ ID NO: 160 or SEQ ID NO:210.

Also provided herein are anti-Candida antibodies that are modified 2G12 domain-exchanged Fab antibodies. In some examples, where the anti-Candida antibody is a modified 2G12 domain-exchanged Fab antibody, the anti-Candida antibody has greater affinity for Candida compared to the unmodified 2G12 domain-exchanged Fab antibody, the unmodified 2G12 domain-exchanged Fab antibody comprises a light chain having the amino acid sequence set forth in SEQ ID NO: 162, SEQ ID NO: 11 or SEQ ID NO: 553 and a heavy chain having the amino acid sequence set forth in SEQ ID NO: 161. In other examples, where the anti-Candida antibody is a modified 2G12 domain-exchanged Fab antibody, the anti-Candida antibody has greater affinity for Candida compared to the unmodified 2G12 domain-exchanged Fab antibody, the unmodified 2G12 domain-exchanged Fab antibody comprises a light chain having the amino acid sequence set forth in SEQ ID NO: 162, SEQ ID NO: 11 or SEQ ID NO: 553 and a heavy chain having the amino acid sequence set forth in SEQ ID NO: 10.

Provided herein are anti-Candida antibodies that contain one or more modifications in the variable light chain (V_{L}) complementary determining region 3 (CDR3) compared to the V_{L} CDR3 of 2G12 set forth in SEQ ID NO: 2. Exemplary modifications included, but are not limited to one or more amino acid additions, substitutions or deletions in the V_{L} CDR3 of 2G12. In some examples, the one or more modifications are at one or more positions selected from among L89, L90, L91, L92, L93, L94, and L95 of the V_{L} CDR3 of 2G12, based on Kabat numbering. In other examples, the one or more modifications are amino acid replacements at one or more positions selected from among L89, L90, L91, L92, L93, L94, and L95 of the V_{L} CDR3 of 2G12, based on Kabat numbering. In further examples, the one or more modifications are amino acid additions immediately before or immediately following one or more positions selected from among L89, L90, L91, L92, L93, L94, and L95 of the V_{L} CDR3 of 2G12, based on Kabat numbering.

For example, modified anti-Candida antibodies provided herein contain one or more modifications at one or more positions selected from among L92, L93, L94, and L95, based on Kabat numbering. In some instances, the modified anti-Candida antibodies provided herein further contain one or more amino acid additions after amino acid residue L95, based on Kabat numbering. In another example, modified anti-Candida antibodies provided herein contain one or more modifications at one or more positions selected from among L89, L90, L91, and L92, based on Kabat numbering.

Provided herein are anti-Candida antibodies that contain a V_{L} CDR3 having an amino acid sequence set forth in any of SEQ ID NOS:30-90, 218-248, 280-281 or 678-686, or a sequence having 70%, 75%, 80%, 85%, 90%, 91%, 92%, 93%, 94%, 95%, 96%, 97%, 98%, or 99% sequence identity therewith. In some examples, the anti-Candida antibody contains a V_{L} CDR3 selected from among QHYMPYRAS (SEQ ID NO:71), QHYLPFNAT (SEQ ID NO:41), QHYKEWRAT (SEQ ID NO:30), QHYTDHKGAT (SEQ ID NO:88), QHYTDHRGAT (SEQ ID NO:89), QHYRAHTGAT (SEQ ID NO:85), QHYTAHTGAT (SEQ ID NO:86), QHYTDHHGAT (SEQ ID NO:87), QHYTDHYGAT (SEQ ID NO:90), QHYTAHRGAT (SEQ ID NO:84), and QHYRPHTGAT (SEQ ID NO:82), or a sequence having 70%, 75%, 80%, 85%, 90%, 91%, 92%, 93%, 94%, 95%, 96%, 97%, 98%, or 99% sequence identity to any of SEQ ID NOS: 30, 41, 71, 82 or 84-90.

Provided herein are anti-Candida antibodies that contain a variable light chain containing a sequence of amino acids set forth as amino acids 4-105 of any of SEQ ID NO: 91-151, 249-279, 282-283, 457-550, 687-704, or a sequence having at least 70% sequence identity therewith, and a variable heavy chain containing a sequence of amino acids set forth as amino acids 4-120 of SEQ ID NO: 154. In some examples, the anti-Candida antibody has a variable light chain having a sequence of amino acids set forth as amino acids 1-107 of SEQ ID NOS: 457-508, 518-550 or 696-701, amino acids 1-108 of SEQ ID NOS: 91-142, 249-279, 282-283, 509-517, 687-692 or 702-704 or amino acids 1-109 of SEQ ID NOS: 143-151 or 693-695, or a sequence having at least 70% sequence identity therewith, and a variable heavy chain having a sequence of amino acids set forth in SEQ ID NO:154.

Provided herein are anti-Candida antibodies that are Fab antibodies that contain a light chain having a sequence of amino acids set forth as amino acids 91-151, 249-279, 282-283, 457-550 or 687-704, or a sequence having at least 70% sequence identity therewith, and a heavy chain containing a sequence of amino acids set forth in SEQ ID NO:161. Also provided herein are full-length anti-Candida antibodies that contain a heavy chain set forth in SEQ ID NO: 160 or SEQ ID NO:210 and a light chain set forth in any of SEQ ID NO: 91-151, 249-279, 282-283, 457-550 or 687-704, or a sequence having 70%, 75%, 80%, 85%, 90%, 91%, 92%, 93%, 94%, 95%, 96%, 97%, 98%, or 99% sequence identity therewith.

Provided herein are anti-Candida antibodies containing a light chain having a sequence of amino acids set forth in SEQ ID NO:91, 102, 132, 143, or 145-151 and a heavy chain having a sequence of amino acids set forth in SEQ ID NO:10. Also provided herein are anti-Candida antibodies containing a light chain having a sequence of amino acids set forth in SEQ ID NO:91, 102, 132, 143, or 145-151 and a heavy chain having a sequence of amino acids set forth in SEQ ID NO: 209 or 210. In some examples, the anti-Candida antibody provided herein is designated 1H12, 1F8, 4F8, A1E8, A1G7, P1F9, A2A12, P2H12, A4F10, P4H12, or A5G10.

Provided herein are anti-Candida antibodies that contain a heavy chain that contains a 2G12 variable heavy chain (V_{H}) CDR1 set forth in SEQ ID NO: 163, a 2G12 V_{H} CDR2 set forth in SEQ ID NO:164, and a 2G12 V_{H} CDR3 set forth in SEQ ID NO:152, and a light chain that contains a 2G12 V_{L} CDR1 set forth in SEQ ID NO:165, a 2G12 V_{L} CDR2 set forth in SEQ ID NO:166, and a modified 2G12 V_{L} CDR3. Exemplary modified 2G12 V_{L} CDR3 are set forth in any of SEQ ID NOS: 30-90, 218-248, 280-281 or 678-686.

Provided herein are anti-Candida antibodies that bind to members of the genus Candida including, but not limited to, C. albicans, C. tropicalis, C. parapsilosis, C. krusei, C. glabrata, C. lusitaniae, C. dubliniensis and C. guilliermondii. In some examples, the anti-Candida antibody provided herein binds to a Candida cell wall mannoprotein.

The domain-exchanged antibodies provided herein exhibit an affinity for binding to an epitope on Candida of less than or about 100 nM to less than or about 0.1 nM; less than or about 50 nM to less than or about 0.1 nM; less than or about 20 nM to less than or about 0.1 nM; less than or about 10 nM to less than or about 0.1 nM; less than or about 20 nM to less than or about 0.5 nM; less than or about 20 nM to less than or about 1 nM; or less than or about 20 nM to less than or about 5 nM. In some examples, the antibody has an affinity of less than 100 nM for binding to Candida, such as Candida albicans, in an in vitro ELISA binding assay. In some examples, the anti-Candida antibody has an affinity for binding to Candida albicans of less than or about 100 nM to less than or about 0.1 nM; less than or about 50 nM to less than or about 0.1 nM; less than or about 20 nM to less than or about 0.1 nM; less than or about 10 nM to less than or about 0.1 nM; less than or about 20 nM to less than or about 0.5 nM; less than or about 20 nM to less than or about 1 nM; or less than or about 20 nM to less than or about 5 nM. In some examples, the affinity of the anti-Candida antibody provided herein is assessed using an in vitro ELISA binding assay.

In some examples, the anti-Candida antibodies provided herein are further modified to improve one or more properties of the antibody, such as, but not limited to improved binding or decreased degradation. In some examples, the anti-Candida antibody provided herein is conjugated to polyethylene glycol (PEG). In some examples, the anti-Candida antibodies provided herein are multimerized antibodies, such as a dimer.

Provided herein are domain-exchanged anti-Candida antibodies that bind to the same epitope as an anti-Candida antibody provided herein, whereby the antibody has greater affinity for Candida compared to the affinity of the corresponding form of the domain-exchanged antibody 2G12.

Provided herein are anti-Candida antibodies that are conjugates. In some examples, the anti-Candida antibodies can be conjugated directly or indirectly via a linker to a therapeutic agent. In other examples, the anti-Candida antibodies can be conjugated directly or indirectly via a linker to a diagnostic agent. Provided herein are anti-Candida antibodies that contain a therapeutic or a diagnostic agent. Exemplary diagnostic agents include, but are not limited to, an enzyme that induces a detectable signal, a dye label, a fluorescent compound, an electron transfer agent, or a chemiluminescent label. Conjugates of anti-Candida antibodies provided herein are chemically conjugated or are a fusion protein.

Provided herein are combinations that contain an anti-Candida antibody provided herein and an antifungal agent. In some examples, the antifungal agent is a triazole or an imidazole. Exemplary antifungal agents include, but are not limited to fluconazole, itraconazole, voriconazole, ketoconazole, miconazole, terconazole, clotrimazole, econazole, fenticonazole, sulconazole, tioconazole, isoconazole, omoconazole, oxiconazole, flutrimazole, butoconazole, amphotericin, nystatin, flucytosine, caspofungin, terbinafine, and gentian violet. The antibody and the antifungal agent can be formulated as a single composition or separate compositions.

Provided herein are combinations that contain a first antibody that is an anti-Candida antibody provided herein and one or more additional antifungal antibodies that differ from the first antibody. In some examples, the combination contains two or more different anti-Candida antibodies. In some example, the combination contains two or more different anti-Candida antibodies selected from among the anti-Candida antibodies provided herein. In some examples, the combination contains one or more additional anti-Candida antibodies where the one or more additional anti-Candida antibodies is selected from among anti-glucan antibodies, anti-mannoprotein antibodies, anti-integrin-like protein antibodies, and antibodies that bind to Candida secretory aspartic proteases. In some examples, the anti-glucan antibody is an anti-β-1,3-glucan antibody or an anti-β-1,6-glucan antibody. In some examples, the combination contains one or more additional antifungal antibodies, where the one or more additional antifungal antibodies is a single-chain Fv (scFv), Fab, Fab′, F(ab′)₂, Fv, dsFv, diabody, Fd, Fd′ fragment, or a domain-exchanged antibody.

Provided herein are pharmaceutical compositions containing an anti-Candida antibody provided herein or a combination containing an antibody provided herein and a pharmaceutically acceptable carrier or excipient. In some examples, the pharmaceutical composition further contains one or more additional antifungal antibodies that differ from the first antibody. In some examples, the one or more additional antifungal antibodies is an anti-Candida antibody, including, but not limited to, an anti-Candida antibody provided herein. In some examples, the one or more additional antifungal antibodies is selected from among anti-glucan antibodies, anti-mannoprotein antibodies, anti-integrin-like protein antibodies, and antibodies that bind to Candida secretory aspartic proteases. Exemplary anti-glucan antibodies include an anti-β-1,3-glucan antibody or an anti-β-1,6-glucan antibody. In some examples, the pharmaceutical composition further contains one or more additional antifungal antibodies, where the one or more additional antifungal antibodies is a single-chain Fv (scFv), Fab, Fab′, F(ab′)₂, Fv, dsFv, diabody, Fd, Fd′ fragment, or a domain-exchanged antibody.

Provided herein are pharmaceutical compositions containing an anti-Candida antibody provided herein formulated as a gel, ointment, cream, paste, suppository, flush, liquid, suspension, aerosol, tablet, pill or powder. In some examples, the pharmaceutical compositions provided herein are formulated for systemic, parenteral, topical, oral, mucosal, intranasal, subcutaneous, aerosolized, intravenous, bronchial, pulmonary, vaginal, vulvovaginal, esophageal, or oesophageal administration. In some examples, the pharmaceutical compositions provided herein are formulated for single dosage administration. In some examples, the pharmaceutical compositions provided herein are formulated for sustained release.

Provided herein are methods of treating a fungal infection in a subject, which involve administering to the subject a therapeutically effective amount of a pharmaceutical composition containing an anti-Candida antibody provided herein. Provided herein are methods of treating or inhibiting one or more symptoms of a fungal infection in a subject, which involve administering to the subject a therapeutically effective amount of a pharmaceutical composition containing an anti-Candida antibody provided herein. Provided herein are methods of preventing a fungal infection in a subject, which involve administering to the subject a therapeutically effective amount of a pharmaceutical composition containing an anti-Candida antibody provided herein. In some examples of the methods, the fungal infection is a Candida infection, such as, but not limited to Candida vaginitis, mucocutaneous candidiasis, or disseminated candidiasis. In some examples of the methods, the pharmaceutical composition provided herein is administered to a mammal. In some examples of the methods, the pharmaceutical composition provided herein is administered to a human subject. In some examples of the methods, the pharmaceutical composition provided herein is administered to a human infant, a human infant born prematurely or at risk of hospitalization for a fungal infection, an elderly human, a human subject who has congenital immunodeficiency, acquired immunodeficiency, leukemia, or non-Hodgkin lymphoma, is receiving or has received high dosage antibiotic therapy, or a human subject having an organ or tissue transplant or a blood transfusion. In some examples, an organ or tissue transplant subject is a patient that has received a bone marrow transplant or a liver transplant.

In some examples of the methods, the pharmaceutical composition containing an anti-Candida antibody provided herein is administered topically, parenterally, locally, or systemically. For examples, the pharmaceutical composition provided herein can be administered intranasally, intramuscularly, intradermally, intraperitoneally, intravenously, subcutaneously, orally, vaginally, vulvovaginally, esophageally, oroesophageally, bronchially, or by pulmonary administration. In some examples, the pharmaceutical composition is administered one time, two times, three times, four times, five times, six times, seven times, eight times, nine times, or ten times for the treatment of a fungal infection.

Provided herein are methods of preventing or treating a fungal infection in a subject, involving the administration of a pharmaceutical composition provided herein and one or more antifungal agents. In some examples, the antifungal agent that is administered is a triazole or an imidazole. Exemplary antifungal agents include, but are not limited to fluconazole, itraconazole, voriconazole, ketoconazole, miconazole, terconazole, clotrimazole, econazole, fenticonazole, sulconazole, tioconazole, isoconazole, omoconazole, oxiconazole, flutrimazole, butoconazole, amphotericin, nystatin, flucytosine, caspofungin, terbinafine, and gentian violet. The antibody and the antifungal agent can be formulated as a single composition or separate compositions. The pharmaceutical composition and the antifungal agent can be administered sequentially, simultaneously or intermittently.

Provided herein are methods of preventing or treating a fungal infection in a subject, involving the administration of a pharmaceutical composition provided herein and one or more additional antifungal antibodies. In some examples, the one or more additional antifungal antibodies are anti-Candida antibodies. In some examples, the one or more additional anti-Candida antibodies are selected from among anti-glucan antibodies, anti-mannoprotein antibodies, anti-integrin-like protein antibodies, and antibodies that bind to Candida secretory aspartic proteases. In some examples, the anti-glucan antibody is an anti-β-1,3-glucan antibody or an anti-β-1,6-glucan antibody. In some examples, the pharmaceutical composition and the one or more additional antifungal antibodies are formulated as a single composition or separate compositions. The pharmaceutical composition and the one or more additional antifungal antibodies can be administered sequentially, simultaneously or intermittently.

In some examples, the pharmaceutical composition provided herein can be used to formulate a medicament for treating a fungal infection in a subject. In other examples, the pharmaceutical composition provided herein can be used to treat or inhibit one or more symptoms of a fungal infection in a subject. In further examples, the pharmaceutical composition provided herein can be used to prevent a fungal infection in a subject. Also provided herein are pharmaceutical compositions containing an anti-Candida antibody provided herein, for treating a fungal infection in a subject; for treating or inhibiting one or more symptoms of a fungal infection in a subject; or for preventing a fungal infection in a subject.

Provided herein are methods detecting a fungal infection, involving the steps of (a) contacting an anti-Candida antibody provided herein with a sample; and (b) comparing the assayed level of Candida antigen with a control level, whereby an increase in the assayed level of Candida antigen compared to the control level of the Candida antigen is indicative of a Candida infection. In some examples of the methods, the sample is a fluid, cell, or tissue sample, such as, but not limited to, a blood, urine, saliva, vaginal mucous, lung sputum, lavage or lymph sample. In some examples, the fluid, cell, or tissue sample is obtained from a human subject.

Provided herein are nucleic acids that encode an anti-Candida antibody provided herein. Provided herein are vectors that contain a nucleic acid that encodes an anti-Candida antibody provided herein. Provided herein are cells that contain nucleic acid encoding the anti-Candida antibody provided herein. Provided herein are nucleic acids that encode a light chain of an anti-Candida antibody provided herein. Provided herein are vectors that contain a nucleic acid that encodes a light chain of an anti-Candida antibody provided herein. Provided herein are cells that contain nucleic acid encoding a light chain of an anti-Candida antibody provided herein. Also provided herein are cell that contains an anti-Candida antibody provided herein, a nucleic acid provided herein, or a vector provided herein. In some examples, the cell is a prokaryotic or eukaryotic cell. Also provided herein are transgenic animals that contain a nucleic acid encoding the anti-Candida antibody provided herein.

Provided herein are methods of expression an anti-Candida antibody provided herein, which involve culturing a cell that contains nucleic acid encoding an anti-Candida antibody provided herein under conditions which express the encoded antibody and isolating the antibody from the cell culture. Provided herein are methods of expression an anti-Candida antibody provided herein, which involve introducing a vector provided herein containing a light chain of the antibody and a vector provided herein containing a heavy chain of the antibody into a host cell, and culturing the host cell under conditions which express the encoded antibody and recovering the antibody. Provided herein are methods of expression an anti-Candida antibody provided herein, which involve isolating the antibody from the transgenic animal that expresses the anti-Candida antibody provided herein. In some examples, the antibody is isolated from the serum or milk of the transgenic animal.

Provided herein are kits that contain an anti-Candida antibody provided herein, in one or more containers, and instructions for use for prophylactic, therapeutic, or diagnostic use.

### BRIEF DESCRIPTION OF THE DRAWINGS

FIG. 1: Comparison of Conventional and Domain-Exchanged Antibodies

FIG. 1 is an illustrative comparison of a full-length conventional IgG antibody (left) and an exemplary full-length domain-exchanged IgG antibody. As shown, the conventional full-length antibody contains two heavy (H and H′) and two light (L and L′) chains, and two antibody combining sites, each formed by residues of one heavy and one light chain. By contrast, the heavy chains in the exemplary domain-exchanged antibody are interlocked, resulting in pairing of the heavy chain variable regions (V_{H} and V_{H}′) with the opposite light chain variable regions (V_{L}′ and V_{L}, respectively), forming a pair of conventional antibody combining sites, locked in space. As described herein, the V_{H}-V_{H}′ interface can form a non-conventional antibody combining site, containing residues of the two adjacent heavy chain variable regions (V_{H} and V_{H}′). The number (35 Å (angstroms)) represents the distance between the two conventional antibody combining sites in this exemplary domain-exchanged antibody. For each antibody, the two heavy chains, H and H′ are illustrated in grey and black, respectively; the two light chains, L and L′, are illustrated with open and hatched boxes, respectively. The specific domains (e.g. V_{H}, V_{L}, C_{H}1, C_{L}) are indicated.

FIG. 2. 2G12 pCAL IT* Vector

FIG. 2 depicts the 2G12 pCAL IT* vector. The 2G12 pCAL IT* vector can be used to express, with reduced toxicity, Fab fragments of the domain-exchanged 2G12 antibody, which recognize the HIV gp120 antigen. Expression as both soluble 2G12 Fab fragments and 2G12-gIII coat protein fusion proteins for display on phage particles can be effected in partial amber suppressor cells by virtue of the amber stop codon between the nucleotides encoding the 2G12 heavy chain and nucleotides encoding the truncated gIII coat protein. The polynucleotide encoding the 2G12 light chain is linked to the PelB leader sequence, and the 2G12 heavy chain is linked to the OmpA leader sequence. The inclusion of an amber stop codon in each of the leader sequences results in reduced expression of the 2G12 heavy and light chains in partial amber suppressor strains following induction with, for example IPTG. The reduced expression can lead to reduced toxicity of the 2G12 Fab to the host cells.

FIG. 3: Randomization of 3-ALA LC 2G12 Fragment Target Polypeptide Using mFAL-SPA

FIG. 3 illustrates the mFAL-SPA process that was used to randomize the CDR3 of the light chain of 2G12 domain-exchanged Fab fragment target polypeptide, as described in Example 2, below. FIG. 3A: Six pools of randomized oligonucleotides (AGYS, SYGA, AGYS+1, SYGA+1, AGYS+2, and SYGA+2); illustrated as open boxes with hatched portions representing randomized portions) were designed and hybridized to form three pools of randomized duplexes (DO, DO+1, and DO+2), containing overhangs. FIG. 3B: Two pools of reference sequence duplexes (1, and 2) were generated using PCR with two pools of forward oligonucleotide primers (2G12LCF and L2F) and two pools of reverse oligonucleotide primers (L1R and 2G12LCR). Two of the primers, 2G12LCF and 2G12LCR, contained a recognition site for the Sap I restriction endonuclease (indicated by a portion with vertical lines). FIG. 3C: Reference sequence duplexes were cut with the Sap I restriction endonuclease, generating reference sequence duplexes with Sap I overhangs compatible to those in the randomized duplexes. FIG. 3D: The reference sequence and randomized pools of duplexes with overhangs then were combined under conditions whereby they hybridized through complementary overhangs and nicks (indicated with arrows) were sealed with a ligase, forming a pool of intermediate duplexes, which then was used in an SPA reaction (not shown) with a CALX24 single primer pool to generate a collection of variant assembled duplexes. One forward primer pool (F1), and one reverse primer pool (R3) contained a non gene-specific nucleotide sequence (Region X; depicted in black), which was identical to the nucleotide sequence of the CALX24 primer, such that reference sequence duplexes 1 and 2 contained a sequence of nucleotides including Region X, and a complementary Region Y, which served as template sequences for the primers in the SPA. The assembled duplexes can be digested to form assembled duplex cassettes with restriction enzymes recognizing restriction sites within the portion illustrated in black.

FIG. 4: Sequence of Domain-Exchanged Antibody 2G12. FIG. 4 depicts the sequence of 2G12 (SEQ ID NOS:154 and 155). FIG. 4A depicts the sequence of the variable heavy chain. FIG. 4B depicts the sequence of the variable light chain. The sequences are numbered according to Kabat and complementarity determining regions (CDRs) are identified in boldface type. For purposes herein, reference to residues in a light chain of a domain-exchanged antibody, for example, 2G12, recites “L” for light followed by the amino acid position based on Kabat numbering. Reference to residues in a heavy chain of a domain-exchanged antibody, for example 2G12, recites “H” for heavy followed by the amino acid position based on Kabat numbering. For example, L96 refers to residue 96, based on Kabat numbering, which is in the CDRL3 of the light chain. Residue L96 also can be referenced as Ala^{L96}, which means that the amino acid residue at position 96, based on Kabat numbering, in the light chain is Ala.

### DETAILED DESCRIPTION

### A. DEFINITIONS

### B. DOMAIN-EXCHANGED ANTIBODIES

2G12 Domain-exchanged Antibody

### C. ANTI-Candida ANTIBODIES

1. Variant 2G12 Anti-Candida Antibodies

2. Other anti-Candida domain-exchanged antibodies

### D. ADDITIONAL MODIFICATIONS OF ANTI-Candida ANTIBODIES

1. Modifications to reduce immunogenicity

2. Fc Modifications

3. Pegylation

4. Modification with other heterologous peptides

### E. METHODS OF IDENTIFYING ANTI-CANDIDA DOMAIN-EXCHANGED ANTIBODIES

1. Domain-Exchanged Antibody Libraries
a. Variant libraries
   i. Selecting Residues
   ii. Randomization and diversification methods
      (1) Design, Synthesis and Assembly of Randomized Oligonucleotides
b. Quick Libraries and Hybrid Libraries

2. Display Libraries and Screening
a. Vectors
   i. pCAL Phagemid Vectors
b. Display Methods
   i. Phage Display
   ii. Other Display Methods
      (1) Cell surface display
      (2) Other display systems
c. Screening
   Functional Screening

### F. METHODS OF PRODUCTION OF ANTIBODIES

1. Methods of Expression of Domain-Exchanged Antibodies
a. Domain-Exchanged Fab Fragment
b. Domain-exchanged scFv fragment
c. Domain-exchanged Fab hinge fragment
d. Domain-exchanged scFv tandem fragment
e. Domain-exchanged single chain Fab fragments
f. Domain-exchanged Fab Cys 19
g. Domain-exchanged scFv hinge

2. Vectors

3. Cells and Expression Systems
a. Prokaryotic Expression
b. Yeast
c. Insects
d. Mammalian Cells
e. Plants

4. Purification

### G. ASSESSING ANTI-CANDIDA ANTIBODY PROPERTIES AND ACTIVITIES

1. Binding Assays

2. In vitro assays for analyzing Candida inhibitory effects of antibodies

3. In vivo animal models for assessing antibody efficacy

### H. DIAGNOSTIC USES

1. In vitro detection of pathogenic infection

2. In vivo detection of pathogenic infection

3. Monitoring Infection

### I. PROPHYLACTIC AND THERAPEUTIC USES

1. Subjects for therapy

2. Dosages

3. Routes of Administration

4. Combination therapies

5. Gene Therapy

J. Pharmaceutical Compositions, Combinations and Articles of manufacture/Kits

1. Pharmaceutical Compositions

2. Articles of Manufacture/Kits

3. Combinations

### K. Examples

### A. DEFINITIONS

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, “antibody” refers to immunoglobulins and immunoglobulin fragments, whether natural or partially or wholly synthetically, such as recombinantly, produced, including any fragment thereof containing at least a portion of the variable region of the immunoglobulin molecule that retains the binding specificity ability of the full-length immunoglobulin. Hence, an antibody includes any protein having a binding domain that is homologous or substantially homologous to an immunoglobulin antigen-binding domain (antibody combining site). For example, an antibody refers to an antibody that contains two heavy chains (which can be denoted H and H′) and two light chains (which can be denoted L and L′), where each heavy chain can be a full-length immunoglobulin heavy chain or a portion thereof sufficient to form an antigen binding site (e.g. heavy chains include, but are not limited to, V_{H}, chains V_{H}-C_{H}1 chains and V_{H}-C_{H}1-C_{H}2-C_{H}3 chains), and each light chain can be a full-length light chain or a portion thereof sufficient to form an antigen binding site (e.g. light chains include, but are not limited to, V_{L} chains and V_{L}-C_{L} chains). Each heavy chain (H and H′) pairs with one light chain (L and L′, respectively). Typically, antibodies minimally include all or at least a portion of the variable heavy (V_{H}) chain and/or the variable light (V_{L}) chain. The antibody also can include all or a portion of the constant region. Antibodies include antibody fragments, such as anti-Candida antibody fragments. As used herein, the term antibody, thus, includes synthetic antibodies, recombinantly produced antibodies, multispecific antibodies (e.g., bispecific antibodies), human antibodies, non-human antibodies, humanized antibodies, chimeric antibodies, intrabodies, and antibody fragments, such as, but not limited to, Fab fragments, Fab′ fragments, F(ab′)₂ fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the above. Antibodies provided herein include members of any immunoglobulin type (e.g., IgG, IgM, IgD, IgE, IgA and IgY), any class (e.g. IgG1, IgG2, IgG3, IgG4, IgA1 and IgA2) or subclass (e.g., IgG2a and IgG2b).

As used herein, a full-length antibody is an antibody having two full-length heavy chains (e.g. V_{H}-C_{H}1-C_{H}2-C_{H}3 or V_{H}-C_{H}1-C_{H}2-C_{H}3-C_{H}4) and two full-length light chains (V_{L}-C_{L}) and hinge regions, such as human antibodies produced by antibody secreting B cells and antibodies with the same domains that are produced synthetically.

As used herein, an “antibody fragment” or antibody portion with reference to a “portion thereof” or “fragment thereof” of an antibody refers to any portion of a full-length antibody that is less than full length but contains at least a portion of the variable region of the antibody sufficient to form an antigen binding site (e.g. one or more CDRs and/or one or more antibody combining sites) and thus retains the binding specificity, and at least a portion of the specific binding ability of the full-length antibody. Antibody fragments include antibody derivatives produced by enzymatic treatment of full-length antibodies, as well as synthetically, e.g. recombinantly produced derivatives. An antibody fragment is included among antibodies. Examples of antibody fragments include, but are not limited to, Fab, Fab′, F(ab′)₂, single-chain Fvs (scFv), Fv, dsFv, diabody, Fd and Fd′ fragments, domain-exchanged fragments, such as domain-exchanged scFv fragments, domain-exchanged scFv tandem fragments, domain-exchanged scFv hinge fragments, domain-exchanged Fab fragments, domain-exchanged single chain Fab fragments (scFab), domain-exchanged Fab hinge fragments, and other modified domain-exchanged fragments and other fragments, including modified fragments (see, for example, Methods in Molecular Biology, Vol 207: Recombinant Antibodies for Cancer Therapy Methods and Protocols (2003); Chapter 1; p 3-25, Kipriyanov). The fragment can include multiple chains linked together, such as by disulfide bridges and/or by peptide linkers. An antibody fragment generally contains at least about 50 amino acids and typically at least 200 amino acids.

Hence, reference to an “antibody or portion thereof that is sufficient to form an antigen binding site” means that the antibody or portion thereof contains at least 1 or 2, typically 3, 4, 5 or all 6 CDRs of the V_{H} and V_{L} sufficient to retain at least a portion of the binding specificity of the corresponding full-length antibody containing all 6 CDRs. Generally, a sufficient antigen binding site at least requires CDR3 of the heavy chain (CDRH3). It typically further requires the CDR3 of the light chain (CDRL3). As described herein, one of skill in the art knows and can identify the CDRs based on Kabat or Chothia numbering (see e.g., Kabat, E. A. et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917).

As used herein, an antigen-binding fragment refers to an antibody fragment that contains an antigen-binding portion that binds to the same antigen as the antibody from which the antibody fragment is derived. An antigen-binding fragment, as used herein, includes any antibody fragment that when inserted into an antibody framework (such as by replacing a corresponding region) results in an antibody that immunospecifically binds (i.e. exhibits Ka of at least or at least about 10⁷-10⁸ M⁻¹) to the antigen. Antigen-binding fragments include, antibody fragments, such as Fab fragments, Fab′ fragments, F(ab′)₂ fragments, Fv fragments, disulfide-linked Fvs (dsFv), Fd fragments, Fd′ fragments, single-chain Fvs (scFv), single-chain Fabs (scFab), and also includes other fragments, such as CDR-containing fragments, and polypeptides that immunospecifically bind to an antigen or that when inserted into an antibody framework results in an antibody that immunospecifically binds to the antigen.

As used herein, an Fv antibody fragment is composed of one variable heavy domain (V_{H}) and one variable light (V_{L}) domain linked by noncovalent interactions.

As used herein, a dsFv refers to an Fv with an engineered intermolecular disulfide bond, which stabilizes the V_{H}-V_{L} pair.

As used herein, an Fd fragment is a fragment of an antibody containing a variable domain (V_{H}) and one constant region domain (C_{H}1) of an antibody heavy chain.

As used herein, a Fab fragment is an antibody fragment that results from digestion of a full-length immunoglobulin with papain, or a fragment having the same structure that is produced synthetically, e.g. by recombinant methods. A Fab fragment contains a light chain (containing a V_{L} and C_{L}) and another chain containing a variable domain of a heavy chain (V_{H}) and one constant region domain of the heavy chain (C_{H}1).

As used herein, a F(ab′)₂ fragment is an antibody fragment that results from digestion of an immunoglobulin with pepsin at pH 4.0-4.5, or a fragment having the same structure that is produced synthetically, e.g. by recombinant methods. The F(ab′)₂ fragment essentially contains two Fab fragments where each heavy chain portion contains an additional few amino acids, including cysteine residues that form disulfide linkages joining the two fragments.

As used herein, a Fab′ fragment is a fragment containing one half (one heavy chain and one light chain) of the F(ab′)₂ fragment.

As used herein, an Fd′ fragment is a fragment of an antibody containing one heavy chain portion of a F(ab′)₂ fragment.

As used herein, an Fv′ fragment is a fragment containing only the V_{H} and V_{L} domains of an antibody molecule.

As used herein, hsFv refers to antibody fragments in which the constant domains normally present in a Fab fragment have been substituted with a heterodimeric coiled-coil domain (see, e.g., Arndt et al. (2001) J Mol Biol. 7(312):221-228).

As used herein, an scFv fragment refers to an antibody fragment that contains a variable light chain (V_{L}) and variable heavy chain (V_{H}), covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

As used herein, an “antibody hinge region” or “hinge region” refers to a polypeptide region that exists naturally in the heavy chain of the gamma, delta and alpha antibody isotypes, between the C_{H}1 and C_{H}2 domains that has no homology with the other antibody domains. This region is rich in proline residues and gives the IgG, IgD and IgA antibodies flexibility, allowing the two “arms” (each containing one antibody combining site) of the Fab portion to be mobile, assuming various angles with respect to one another as they bind antigen. This flexibility allows the Fab arms to move in order to align the antibody combining sites to interact with epitopes on cell surfaces or other antigens. Two interchain disulfide bonds within the hinge region stabilize the interaction between the two heavy chains. In some embodiments provided herein, the synthetically produced antibody fragments contain one or more hinge region, for example, to promote stability via interactions between two antibody chains. Hinge regions are exemplary of dimerization domains.

As used herein, diabodies are dimeric scFv; diabodies typically have shorter peptide linkers than scFvs, and preferentially dimerize.

As used herein, “monoclonal antibody” refers to a population of identical antibodies, meaning that each individual antibody molecule in a population of monoclonal antibodies is identical to the others. This property is in contrast to that of a polyclonal population of antibodies, which contains antibodies having a plurality of different sequences. Monoclonal antibodies can be produced by a number of well-known methods (Smith et al. (2004) J. Clin. Pathol. 57, 912-917; and Nelson et al., (2000) J Clin Pathol 53, 111-117). For example, monoclonal antibodies can be produced by immortalization of a B cell, for example through fusion with a myeloma cell to generate a hybridoma cell line or by infection of B cells with virus such as EBV. Recombinant technology also can be used to produce antibodies in vitro from clonal populations of host cells by transforming the host cells with plasmids carrying artificial sequences of nucleotides encoding the antibodies.

As used herein, a “conventional antibody” refers to an antibody that contains two heavy chains (which can be denoted H and H′) and two light chains (which can be denoted L and L′) and two antibody combining sites, where each heavy chain can be a full-length immunoglobulin heavy chain or any functional region thereof that retains antigen-binding capability (e.g. heavy chains include, but are not limited to, V_{H}, chains V_{H}-C_{H}1 chains and V_{H}-C_{H}1-C_{H}2-C_{H}3 chains), and each light chain can be a full-length light chain or any functional region of (e.g. light chains include, but are not limited to, V_{L} chains and V_{L}-C_{L} chains). Each heavy chain (H and H′) pairs with one light chain (L and L′, respectively).

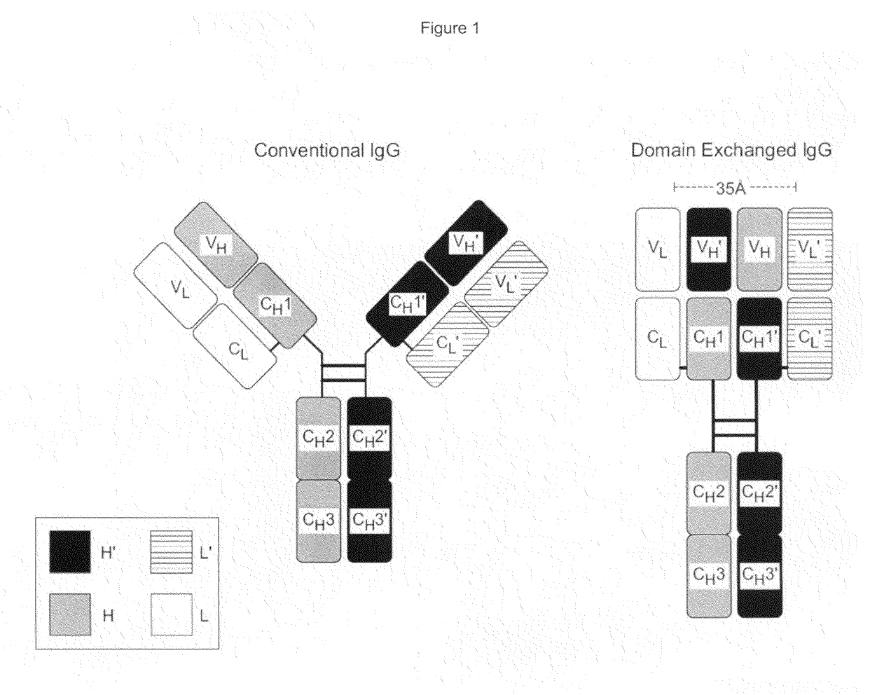

As used herein, a “domain-exchanged antibody” refers to any antibody (including any antibody fragment) that has a domain-exchanged three-dimensional structural configuration, characterized by the pairing of each heavy chain variable region with the opposite light chain variable region (and optionally the opposite light chain constant region), where the pairing is opposite as compared to heavy-light chain pairing in a conventional antibody, and by the formation of an interface (V_{H}-V_{H}′ interface) between adjacently positioned V_{H} domains (see, e.g. FIG. 1, comparing exemplary conventional and domain-exchanged full-length IgG antibodies), including any antibody fragment derived from such an antibody that retains the V_{H}-V_{H}′ interface and at least a portion of the antigen specificity of the antibody. This V_{H}-V_{H}′ interface can contain one or more non-conventional antibody combining sites. In one example, the opposite pairing and V_{H}-V_{H}′ interface are formed by interlocked heavy chains.

As used herein, a domain-exchanged Fab fragment is a domain-exchanged antibody fragment that contains two copies each of a light (V_{L}-C_{L}, V_{L}′-C_{L}′) chain and a heavy (V_{H}-C_{H}1, V_{H}′-C_{H}1′) chain, which are folded in the domain-exchanged configuration, where each heavy chain variable region pairs with the opposite light chain variable region compared to a conventional antibody, and an interface (V_{H}-V_{H}′) is formed between adjacently positioned V_{H} domains. Typically, the fragment contains two conventional antibody combining sites and at least one non-conventional antibody combining site (contributed to by residues at the V_{H}-V_{H}′ interface).

As used herein, a domain-exchanged single chain Fab fragment (scFab) is a domain-exchanged Fab fragment, further including peptide linkers between each V_{H} and V_{L}. In some examples of a domain-exchanged scFab fragment (e.g. domain-exchanged scFabΔC2 fragment), one or more cysteines are mutated compared to the native scFab fragment, to eliminate one or more disulfide bonds between constant regions.

As used herein, a domain-exchanged Fab hinge fragment is a domain-exchanged Fab fragment, further containing an antibody hinge region adjacent to each heavy chain constant region.

As used herein, a domain-exchanged scFv fragment is a domain-exchanged antibody fragment containing two chains, each of which contains one V_{H} and one V_{L} domain, joined by a peptide linker (V_{H}-linker-V_{L}). The two chains interact through the V_{H} domains, producing the V_{H}-V_{H}′ interface characteristic of the domain-exchanged configuration. Typically, the V_{H}-linker-V_{L} sequence of amino acids in each chain is identical.

A domain-exchanged scFv hinge fragment is a domain-exchanged scFv fragment further containing an antibody hinge region adjacent to each V_{H} domain.

As used herein, a domain-exchanged scFv tandem fragment refers to a domain-exchanged antibody fragment containing two V_{H} domains and two V_{L} domains, each in a single chain and separated by polypeptide linkers. The linear configuration of these domains is V_{L}-linker-V_{H}-linker-V_{H}-linker-V_{L}. In one example, for display on genetic packages, the fragment further includes a coat protein, e.g. a phage coat protein, at one or the other end of the molecule, adjacent or in close proximity to one of the V_{L} chains.

As used herein, 2G12 refers to the domain-exchanged human monoclonal IgG1 antibody produced from the hybridoma cell line CL2 (as described in U.S. Pat. No. 5,911,989; Buchacher et al., (1994) AIDS Research and Human Retroviruses, 10(4) 359-369; and Trkola et al., (1996) Journal of Virology, 70(2):1100-1108), and any synthetically, e.g. recombinantly, produced antibody having the identical sequence of amino acids, including any antibody fragment thereof having at least the antigen-binding portions of the heavy and light chain variable region domains to the full-length antibody, such as the 2G12 domain-exchanged Fab fragment (see, for example, Published U.S. Application, Publication No. US20050003347 and Calarese et al., (2003) Science, 300:2065-2071, including supplemental information). For example, 2G12 includes a full length antibody having a heavy chain set forth in SEQ ID NO:160 and a light chain set forth in SEQ ID NO:162. 2G12 antibodies also include antibody fragments thereof that at least include the variable heavy chain set forth in SEQ ID NO:154 and the variable light chain set forth in SEQ ID NO:155. For example, 2G12 Fab fragments have a heavy chain with a sequence of amino acids set forth in SEQ ID NO:161 and a light chain with a sequence of amino acids set forth in SEQ ID NO:162. It is understood that variation of the sequence of amino acids of any of SEQ ID NOS: 154, 155 or 160-162 can occur at the N- or C-terminus and still be a 2G12 antibody if binding of the antibody is retained. Thus, 1, 2, 3, or 4 amino acids can be varied, such as by substitution, addition, or deletion. For example, variation can occur as a result of cloning procedures or for other reasons. It also is understood that due to cloning artifacts or other variation introduced by cloning, that variation can also exist in other regions of the sequence. For purposes herein, reference to 2G12 is to an antibody that at least includes a variable heavy chain with the sequence of amino acids set forth as amino acids 4-120 of SEQ ID NO: 154 and a variable light chain with the sequence of amino acids set forth as amino acids 4-105 of SEQ ID NO:155. Thus, 2G12 includes an antibody having a variable heavy chain set forth in SEQ ID NO: 154 and a variable light chain set forth in SEQ ID NO:155, 176 or 667. 2G12 also includes a Fab form that has a heavy chain sequence set forth in SEQ ID NO: 10 and a light chain sequence set forth in SEQ ID NO: 11, 162 or 553. 2G12 also includes a full-length form that has a heavy chain sequence set forth in SEQ ID NO:160 or 210 and a light chain sequence set forth in SEQ ID NOS: 11, 162 or 553. 2G12 antibodies specifically bind HIV gp120 antigen.

As used herein, recitation that a domain-exchanged antibody is “not 2G12” means that the antibody does not have a sequence of amino acids that contains the same sequence of amino acids as the domain-exchanged human monoclonal IgG1 antibody produced from the hybridoma cell line CL2. Hence, a domain-exchanged antibody that is not 2G12 does not have a variable heavy chain containing the sequence of amino acids set forth as amino acids 4-120 of SEQ ID NO: 154 and a variable light chain containing the sequence of amino acids set forth as amino acids 4-105 of SEQ ID NO:155. A domain-exchanged antibody that is not 2G12 includes any domain-exchanged antibody that has a sequence that is different then 2G12. A domain-exchanged antibody that is not 2G12 includes a modified or variant 2G12 antibody.

As used herein, a “modified 2G12” or “variant 2G12” antibody refers to an antibody, or portion thereof, that contains one or more amino acid modifications in 2G12. An amino acid modification includes an amino acid deletion, replacement (or substitution), or addition (or insertion). A modified antibody can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, or more amino acid modifications. Typically, an amino acid modification is an amino acid replacement or addition. For purposes herein, modified 2G12 antibodies include antibodies that contain modifications in one or more complementarity determining regions (CDRs). It is understood that the 2G12 antibody also can contain modifications in other regions of the antibody, for example, any that are described herein or known to one of skill in the art.

As used herein, “gp120,” “HIV gp120” and “gp120 antigen” refer to the HIV envelope surface glycoprotein, epitopes of which are specifically recognized and bound by the 2G12 antibody. HIV gp120 (GENBANK gi:28876544) is one of two cleavage products resulting from cleavage of the gp160 precursor glycoprotein (GENBANK gi:9629363). gp120 can refer to the full-length gp120 or a fragment thereof containing epitopes bound by the 2G12 antibody.

As used herein, the term “derivative” refers to a polypeptide that contains an amino acid sequence of an anti-Candida antibody which has been modified, for example, by the introduction of amino acid residue substitutions, deletions or additions, by the covalent attachment of any type of molecule to the polypeptide (e.g., by glycosylation, acetylation, pegylation, phosphorylation, amidation, derivatization by known protecting/blocking groups, proteolytic cleavage, linkage to a cellular ligand or other protein). A derivative of an anti-Candida antibody can be modified by chemical modifications using techniques known to those of skill in the art, including, but not limited to, specific chemical cleavage, acetylation, formylation, metabolic synthesis of tunicamycin. Further, a derivative of an anti-Candida antibody can contain one or more non-classical amino acids. Typically, a polypeptide derivative possesses a similar or identical function as an anti-Candida antibody provided herein (e.g. inhibition of Candida infection).

As used herein, Candida spp. refers to any member of the Candida genus of yeast. The genus Candida includes species such as, but not limited to, C. albicans, C. ascalaphidarum, C. amphixiae, C. antarctica, C. atlantica, C. atmosphaerica, C. blattae, C. carpophila, C. cerambycidarum, C. chauliodes, C. corydali, C. dosseyi, C. dubliniensis, C. ergatensis, C. fructus, C. glabrata, C. fermentati, C. guilliermondii, C. haemulonii, C. insectamens, C. insectorum, C. intermedia, C. jeffresii, C. kefrr, C. krusei, C. lusitaniae, C. lyxosophila, C. maltosa, C. membranifaciens, C. milleri, C. oleophila, C. oregonensis, C. parapsilosis, C. quercitrusa, C. sake, C. shehatea, C. temnochilae, C. tenuis, C. tropicalis, C. tsuchiyae, C. sinolaborantium, C. sojae, C. viswanathii, and C. utilis.

As used herein, a “surface protein” of a pathogen is any protein that is located on external surface of the pathogen. The surface protein can be partially or entirely exposed to the external environment (i.e. outer surface). Exemplary of surface proteins are yeast cell wall proteins, such as, for example, a cell wall glycoprotein, such as a mannoprotein. Mannoproteins are generally located in the outermost layer of the cell wall and are highly antigenic.

As used herein, a mannoprotein is any protein that is modified by the oligosaccharide mannose. Mannoproteins are a major component of yeast cell wall.

As used herein, a “therapeutic antibody” refers to any antibody that is administered for treatment of an animal, including a human. Such antibodies can be prepared by any known methods for the production of polypeptides, and hence, include, but are not limited to, recombinantly produced antibodies, synthetically produced antibodies, and therapeutic antibodies extracted from cells or tissues and other sources. As isolated from any sources or as produced, therapeutic antibodies can be heterogeneous in length or differ in post-translational modification, such as glycosylation (i.e. carbohydrate content). Heterogeneity of therapeutic antibodies also can differ depending on the source of the therapeutic antibodies. Hence, reference to therapeutic antibodies refers to the heterogeneous population as produced or isolated. When a homogeneous preparation is intended, it will be so-stated. References to therapeutic antibodies herein are to their monomeric, dimeric or other multimeric forms, as appropriate.

As used herein, the phrase “derived from” when referring to antibody fragments derived from another antibody, such as a monoclonal antibody, refers to the engineering of antibody fragments (e.g., Fab, F(ab′), F(ab′)₂, single-chain Fvs (scFv), Fv, dsFv, diabody, Fd and Fd′ fragments) that retain the binding specificity of the original antibody. Such fragments can be derived by a variety of methods known in the art, including, but not limited to, enzymatic cleavage, chemical crosslinking, recombinant means or combinations thereof. Generally, the derived antibody fragment shares the identical or substantially identical heavy chain variable region (V_{H}) and light chain variable region (V_{L}) of the parent antibody, such that the antibody fragment and the parent antibody bind the same epitope.

As used herein, a “parent antibody” or “source antibody” refers the to an antibody from which an antibody fragment (e.g., Fab, F(ab′), F(ab′)₂, single-chain Fvs (scFv), Fv, dsFv, diabody, Fd and Fd′ fragments) is derived.

As used herein, a conjugate or chimeric polypeptide refers to a polypeptide that contains portions from at least two different polypeptides or from two non-contiguous portions of a single polypeptide. Thus, a chimeric polypeptide generally includes a sequence of amino acid residues from all or part of one polypeptide and a sequence of amino acids from all or part of another different polypeptide. The two portions can be linked directly or indirectly via a linker and can be linked via peptide bonds, other covalent bonds or other non-covalent interactions of sufficient strength to maintain the integrity of a substantial portion of the chimeric polypeptide under equilibrium conditions and physiologic conditions, such as in isotonic pH 7 buffered saline. For purposes herein, chimeric polypeptides include those containing all or part of an anti-Candida antibody linked to another polypeptide, such as, for example, a multimerization domain, a heterologous immunoglobulin constant domain or framework region, or a diagnostic or therapeutic polypeptide.

As used herein, a fusion protein is a polypeptide engineered to contain sequences of amino acids corresponding to two distinct polypeptides, which are joined together, such as by expressing the fusion protein from a vector containing two nucleic acids, encoding the two polypeptides, in close proximity, e.g. adjacent, to one another along the length of the vector. Generally, a fusion protein provided herein refers to a polypeptide that contains a polypeptide having the amino acid sequence of an antibody and a polypeptide or peptide having the amino acid sequence of a heterologous polypeptide or peptide, such as, for example, a diagnostic or therapeutic polypeptide. Accordingly, a fusion protein refers to a chimeric protein containing two or portions from two more proteins or peptides that are linked directly or indirectly via peptide bonds. The two molecules can be adjacent in the construct or separated by a linker, or spacer polypeptide. The spacer can encode a polypeptide that alters the properties of the polypeptide, such as solubility or intracellular trafficking.

As used herein, “linker” or “spacer” peptide refers to short sequences of amino acids that join two polypeptide sequences (or nucleic acid encoding such an amino acid sequence). “Peptide linker” refers to the short sequence of amino acids joining the two polypeptide sequences. Exemplary of polypeptide linkers are linkers joining a peptide transduction domain to an antibody or linkers joining two antibody chains in a synthetic antibody fragment such as an scFv fragment. Linkers are well-known and any known linkers can be used in the provided methods. Exemplary of polypeptide linkers are (Gly-Ser)ₙ amino acid sequences, with some Glu or Lys residues dispersed throughout to increase solubility. Other exemplary linkers are described herein; any of these and other known linkers can be used with the provided compositions and methods.

As used herein, humanized antibodies refer to antibodies that are modified to include “human” sequences of amino acids so that administration to a human does not provoke an immune response. A humanized antibody typically contains complementarily determining regions (CDRs) derived from a non-human species immunoglobulin and the remainder of the antibody molecule derived mainly from a human immunoglobulin. Methods for preparation of such antibodies are known. For example, DNA encoding a monoclonal antibody can be altered by recombinant DNA techniques to encode an antibody in which the amino acid composition of the non-variable regions is based on human antibodies. Methods for identifying such regions are known, including computer programs, which are designed for identifying the variable and non-variable regions of immunoglobulins.

As used herein, idiotype refers to a set of one or more antigenic determinants specific to the variable region of an immunoglobulin molecule.

As used herein, anti-idiotype antibody refers to an antibody directed against the antigen-specific part of the sequence of an antibody or T cell receptor. In principle an anti-idiotype antibody inhibits a specific immune response.

As used herein, an Ig domain is a domain, recognized as such by those in the art that is distinguished by a structure, called the Immunoglobulin (Ig) fold, which contains two beta-pleated sheets, each containing anti-parallel beta strands of amino acids connected by loops. The two beta sheets in the Ig fold are sandwiched together by hydrophobic interactions and a conserved intra-chain disulfide bond. Individual immunoglobulin domains within an antibody chain further can be distinguished based on function. For example, a light chain contains one variable region domain (V_{L}) and one constant region domain (C_{L}), while a heavy chain contains one variable region domain (V_{H}) and three or four constant region domains (C_{H}). Each V_{L}, C_{L}, V_{H}, and C_{H} domain is an example of an immunoglobulin domain.

As used herein, a variable domain or variable region is a specific Ig domain of an antibody heavy or light chain that contains a sequence of amino acids that varies among different antibodies. Each light chain and each heavy chain has one variable region domain, V_{L} and V_{H}, respectively. The variable domains provide antigen specificity, and thus are responsible for antigen recognition. Each variable region contains CDRs that are part of the antigen-binding site domain and framework regions (FRs).

As used herein, “antigen-binding domain,” “antigen-binding site,” “antigen combining site” and “antibody combining site” are used synonymously to refer to a domain within an antibody that recognizes and physically interacts with cognate antigen. A native conventional full-length antibody molecule has two conventional antigen-binding sites, each containing portions of a heavy chain variable region and portions of a light chain variable region. A conventional antigen-binding site contains the loops that connect the anti-parallel beta strands within the variable region domains. The antigen combining sites can contain other portions of the variable region domains. Each conventional antigen-binding site contains three hypervariable regions from the heavy chain and three hypervariable regions from the light chain. The hypervariable regions also are called complementarity-determining regions (CDRs). In one example, a domain-exchanged antibody further contains one or more non-conventional antibody combining sites formed by the interface between the two heavy chain variable regions. In this example, the domain-exchanged antibody contains two conventional and at least one non-conventional antibody combining site.

As used herein, an “antigen binding” portion or region of an antibody is a portion/region that contains at least the antibody combining site (either conventional or non-conventional) or a portion of the antibody combining site that retains the antigen specificity of the corresponding full-length antibody (e.g. a V_{H} portion of the antibody combining site).

As used herein, a non-conventional antibody combining site, antigen binding site, or antigen combining site refers to domain within an antibody that recognizes and physically interacts with cognate antigen but does not contain the conventional portions of one heavy chain variable region and one light chain variable region. Exemplary of non-conventional antibody combining sites is the non-conventional site comprised of regions of the two heavy chain variable regions in a domain-exchanged antibody.

As used herein, “hypervariable region,” “HV,” “complementarity-determining region” and “CDR” and “antibody CDR” are used interchangeably to refer to one of a plurality of portions within each variable region that together form an antigen-binding site of an antibody. Each variable region domain contains three CDRs, named CDR1, CDR2 and CDR3. The three CDRs are non-contiguous along the linear amino acid sequence, but are proximate in the folded polypeptide. The CDRs are located within the loops that join the parallel strands of the beta sheets of the variable domain. As described herein, one of skill in the art knows and can identify the CDRs based on Kabat or Chothia numbering (see e.g., Kabat, E. A. et al. (1991) Sequences of Proteins of Immunological Interest, Fifth Edition, U.S. Department of Health and Human Services, NIH Publication No. 91-3242, and Chothia; C. et al. (1987) J. Mol. Biol. 196:901-917).

As used herein, framework regions (FRs) are the domains within the antibody variable region domains that are located within the beta sheets; the FR regions are comparatively more conserved, in terms of their amino acid sequences, than the hypervariable regions.

As used herein, a “constant region” domain is a domain in an antibody heavy or light chain that contains a sequence of amino acids that is comparatively more conserved than that of the variable region domain. In conventional full-length antibody molecules, each light chain has a single light chain constant region (C_{L}) domain and each heavy chain contains one or more heavy chain constant region (C_{H}) domains, which include, C_{H}1, C_{H}2, C_{H}3 and C_{H}4. Full-length IgA, IgD and IgG isotypes contain C_{H}1, C_{H}2 C_{H}3 and a hinge region, while IgE and IgM contain C_{H}1, C_{H}2, C_{H}3 and C_{H}4. C_{H}1 and C_{L} domains extend the Fab arm of the antibody molecule, thus contributing to the interaction with antigen and rotation of the antibody arms. Antibody constant regions can serve effector functions, such as, but not limited to, clearance of antigens, pathogens and toxins to which the antibody specifically binds, e.g. through interactions with various cells, biomolecules and tissues.

As used herein, the term “epitope” refers to any antigenic determinant on an antigen to which the paratope of an antibody binds. Epitopic determinants typically comprise chemically active surface groupings of molecules such as amino acids or sugar side chains and typically have specific three dimensional structural characteristics, as well as specific charge characteristics. For example, 2G12 recognizes an α1→2 mannose epitope on HIV gp120. Similarly, 2G12 recognizes a similar α1-2-linked mannose epitope on Candida.

As used herein, “an epitope presented on Candida” refers to an antigenic determinant on the cell wall surface of a Candida. Antigenic determinants presented on Candida include, but are not limited to, mannoproteins, glucans and integrins. For example, Candida contains mannoproteins expressed on the cell surface that contain β-1,3-glucans, β-1,6-glucans, α1→6 mannans and α1→2 mannans. An exemplary epitope presented on Candida are al mannan oligosaccharides.

As used herein, a binding property is a characteristic of a molecule, e.g. a polypeptide, relating to whether or not, and how, it binds one or more binding partners. Binding properties include ability to bind the binding partner(s), the affinity with which it binds to the binding partner (e.g. high affinity), the avidity with which it binds to the binding partner, the strength of the bond with the binding partner and specificity for binding with the binding partner.

As used herein, affinity describes the strength of the interaction between two or more molecules, such as binding partners, typically the strength of the noncovalent interactions between two binding partners. The affinity of an antibody or antigen-binding fragment thereof for an antigen epitope is the measure of the strength of the total noncovalent interactions between a single antibody combining site and the epitope. Low-affinity antibody-antigen interaction is weak, and the molecules tend to dissociate rapidly, while high affinity antibody-antigen-binding is strong and the molecules remain bound for a longer amount of time. Methods for calculating affinity are well known, such as methods for determining association/dissociation constants. Affinity can be estimated empirically or affinities can be determined comparatively, e.g. by comparing the affinity of one antibody and another antibody for a particular antigen.

As used herein, a form of an antibody refers to particular structure of an antibody. Antibodies herein include full length and antigen binding forms, such as, for example, a Fab fragment, a full-length IgG, a domain-exchanged Fab fragment, a domain-exchanged IgG, or other antibody fragment or domain-exchanged fragment. Thus, a Fab is a particular form of an antibody.

Reference to a “corresponding form” of an antibody means that when comparing a property of two antibodies, the property is compared using the same form of the antibody. For example, if its stated that a first antibody that has greater affinity for Candida compared to the affinity of the corresponding form of a second antibody, that means that a particular form, such as a Fab of that antibody has greater affinity compared to the Fab form of the second antibody.

As used herein, antibody avidity refers to the strength of multiple interactions between a multivalent antibody and its cognate antigen, such as with antibodies containing multiple binding sites associated with an antigen with repeating epitopes or an epitope array. A high avidity antibody has a higher strength of such interactions compared with a low avidity antibody.

As used herein, “bind” refers to the participation of a molecule in any attractive interaction with another molecule, resulting in a stable association in which the two molecules are in close proximity to one another. Binding includes, but is not limited to, non-covalent bonds, covalent bonds (such as reversible and irreversible covalent bonds), and includes interactions between molecules such as, but not limited to, proteins, nucleic acids, carbohydrates, lipids, and small molecules, such as chemical compounds including drugs. Exemplary of bonds are antibody-antigen interactions and receptor-ligand interactions. When an antibody “binds” a particular antigen, bind refers to the specific recognition of the antigen by the antibody, through cognate antibody-antigen interaction, at antibody combining sites. Binding also can include association of multiple chains of a polypeptide, such as antibody chains which interact through disulfide bonds.

As used herein, “specifically bind” or “immunospecifically bind” with respect to an antibody or antigen-binding fragment thereof are used interchangeably herein and refer to the ability of the antibody or antigen-binding fragment to form one or more noncovalent bonds with a cognate antigen, by noncovalent interactions between the antibody combining site(s) of the antibody and the antigen. The antigen can be an isolated antigen or presented on the surface of a pathogen, such as a yeast cell (e.g. Candida). Typically, an antibody that immunospecifically binds (or that specifically binds) to a yeast cell is one that binds to a surface antigen on the yeast with an affinity constant (Ka) of about or 1×10⁷ M⁻¹ or 1×10⁸ M⁻¹ or greater (or a dissociation constant (K_{d}) of 1×10⁻⁷ M (100 nM) or 1×10⁻⁸ M (10 nM) or less). Affinity constants can be determined by standard kinetic methodology for antibody reactions, for example, immunoassays (e.g. ELISA), or surface plasmon resonance (SPR) of assembled glycolipid monolayers or glycosylated proteins (Luallen et al. (2009) Journal of Virology 83(10):4861-4870). Instrumentation and methods for real time detection and monitoring of binding rates are known and are commercially available (e.g., BiaCore 2000, Biacore AB, Upsala, Sweden and GE Healthcare Life Sciences; Malmqvist (2000) Biochem. Soc. Trans. 27:335). An antibody that immunospecifically binds to a yeast cell can bind to other peptides, polypeptides, or proteins, viruses, or yeast cells with equal or lower binding affinity. Typically, an antibody or antigen-binding fragment thereof provided herein that binds immunospecifically to Candida antigen does not cross-react with other antigens or cross reacts with substantially (at least 10-100 fold) lower affinity for such antigens. A particular antigen, such as a carbohydrate moiety, can be present on multiple glycoproteins, including glycoproteins from different species. For example, the carbohydrate moiety which is recognized by 2G12 is present on multiple glycoproteins, such as, but not limited to, HIV gp120, Candida cell wall mannoproteins, and Saccharomyces cerevisiae Pst1. Antibodies or antigen-binding fragments that immunospecifically bind to a particular Candida antigen (e.g. a carbohydrate moiety present on a Candida mannoprotein) can be identified, for example, by immunoassays, such as radioimmunoassays (RIA), enzyme-linked immunosorbent assays (ELISAs), surface plasmon resonance, or other techniques known to those of skill in the art. An antibody or antigen-binding fragment thereof that immunospecifically binds to an epitope on a Candida antigen typically is one the binds to the epitope (presented on the yeast) with a higher binding affinity than to any cross-reactive epitope as determined using experimental techniques, such as, but not limited to, immunoassays, surface plasmon resonance, or other techniques known to those of skill in the art. The affinity of the antibody for the antigen, such as a carbohydrate moiety, as presented on the surface of a yeast cell can be determined.

As used herein, the term “surface plasmon resonance” refers to an optical phenomenon that allows for the analysis of real-time interactions by detection of alterations in protein concentrations within a biosensor matrix, for example, using the BiaCore system (GE Healthcare Life Sciences).

As used herein, “affinity constant” refers to an association constant (Ka) used to measure the affinity of an antibody for an antigen. The higher the affinity constant the greater the affinity of the antibody for the antigen. Affinity constants are expressed in units of reciprocal molarity (i.e. M⁻¹) and can be calculated from the rate constant for the association-dissociation reaction as measured by standard kinetic methodology for antibody reactions (e.g., immunoassays, surface plasmon resonance, or other kinetic interaction assays known in the art).

As used herein, “off-rate” when referring to an antibody, refers to the dissociation rate constant (k_{off}), or rate at which the antibody dissociates from bound antigen. Off-rate can be compared to another antibody, for example, “low off rate” of a variant antibody polypeptide or modified antibody polypeptide can refer to an off-rate that is lower than the off-rate of the target or unmodified antibody.

As used herein, “on-rate,” when referring to an antibody, refers to the dissociation rate constant (kₒₙ), or rate at which the antibody associates (binds) to its antigen. On-rate can be compared to another antibody, for example, “high on-rate” of a variant antibody polypeptide or modified antibody polypeptide can refer to an on-rate that is greater than the on-rate of the target or unmodified antibody.

As used herein, the phrase “having the same binding specificity” when used to describe an antibody in reference to another antibody, means that the antibody specifically binds (immunospecifically binds or specifically binds to the yeast cell) to all or a part of the same antigenic epitope as the reference antibody. The epitope can be on the isolated protein (e.g. an isolated yeast cell wall glycoprotein) or expressed on the surface of the yeast. The ability of two antibodies to bind to the same epitope can be determined by known assays in the art such as, for example, surface plasmon resonance assays and antibody competition assays. Typically, antibodies that immunospecifically bind to the same epitope can compete for binding to the epitope, which can be measured, for example, by an in vitro binding competition assay (e.g. competition ELISA), using techniques known the art. Typically, a first antibody that immunospecifically binds to the same epitope as a second antibody can compete for binding to the epitope by about or 30%, 35%, 40%, 45%, 50%, 55%, 60%, 65%, 70%, 75%, 80%, 85%, 90%, 95%, 100%, where the percentage competition is measured ability of the second antibody to displace binding of the first antibody to the epitope. In exemplary competition assays, the antigen is incubated in the presence a predetermined limiting dilution of a labeled antibody (e.g., 50-70% saturation concentration), and serial dilutions of an unlabeled competing antibody. Competition is determined by measuring the binding of the labeled antibody to the antigen for any decreases in binding in the presence of the competing antibody. Variations of such assays, including various labeling techniques and detection methods including, for example, radiometric, fluorescent, enzymatic and colorimetric detection, are known in the art.

As used herein, “binding partner” refers to a molecule (such as a polypeptide, lipid, glycolipid, nucleic acid molecule, carbohydrate or other molecule), with which another molecule specifically interacts, for example, through covalent or noncovalent interactions, such as the interaction of an antibody with cognate antigen. The binding partner can be naturally or synthetically produced. In one example, desired variant polypeptides are selected using one or more binding partners, for example, using in vitro or in vivo methods. Exemplary of the in vitro methods include selection using a binding partner coupled to a solid support, such as a bead, plate, column, matrix or other solid support; or a binding partner coupled to another selectable molecule, such as a biotin molecule, followed by subsequent selection by coupling the other selectable molecule to a solid support. Typically, the in vitro methods include wash steps to remove unbound polypeptides, followed by elution of the selected variant polypeptide(s). The process can be repeated one or more times in an iterative process to select variant polypeptides from among the selected polypeptides.

As used herein, a “multivalent” antibody is an antibody containing two or more antigen-binding sites. Multivalent antibodies encompass bivalent, trivalent, tetravalent, pentavalent, hexavalent, heptavalent or higher valency antibodies.

As used herein, a “monospecific” is an antibody that contains two or more antigen-binding sites, where each antigen-binding site immunospecifically binds to the same epitope.

As used herein, a “multispecific” antibody is an antibody that contains two or more antigen-binding sites, where at least two of the antigen-binding sites immunospecifically bind to different epitopes.

As used herein, a “bispecific” antibody is a multispecific antibody that contains two or more antigen-binding sites and can immunospecifically bind to two different epitopes. A “trispecific” antibody is a multispecific antibody that contains three or more antigen-binding sites and can immunospecifically bind to three different epitopes. A “tetraspecific” antibody is a multispecific antibody that contains four or more antigen-binding sites and can immunospecifically bind to four different epitopes, and so on.

As used herein, a “heterobivalent” antibody is a bispecific antibody that contains two antigen-binding sites, where each antigen-binding site immunospecifically binds to a different epitope.

As used herein, a “homobivalent” antibody is a monospecific antibody that contains two antigen-binding sites, where each antigen-binding site immunospecifically binds to the same epitope. Homobivalent antibodies include, but are not limited to, conventional full length antibodies, engineered or synthetic full-length antibodies, any multimer of two identical antigen-binding fragments, or any multimer two antigen-binding fragments containing the same antigen-binding domain.

As used herein, “binds to the same epitope” with reference to two or more antibodies means that the antibodies compete for binding to an antigen and bind to the same, overlapping or encompassing continuous or discontinuous segments of amino acids. Those of skill in the art understand that the phrase “binds to the same epitope” does not necessarily mean that the antibodies bind to exactly the same amino acids. The precise amino acids to which the antibodies bind can differ. For example, a first antibody can bind to a segment of amino acids that is completely encompassed by the segment of amino acids bound by a second antibody. In another example, a first antibody binds one or more segments of amino acids that significantly overlap the one or more segments bound by the second antibody. For the purposes herein, such antibodies are considered to “bind to the same epitope.”

Antibody competition assays can be used to determine whether an antibody “binds to the same epitope” as another antibody. Such assays are well known on the art. Typically, competition of 70% or more, such as 70%, 71%, 72%, 73%, 74%, 75%, 80%, 85%, 90%, 95% or more, of an antibody known to interact with the epitope by a second antibody under conditions in which the second antibody is in excess and the first saturates all sites, is indicative that the antibodies “bind to the same epitope.” To assess the level of competition between two antibodies, for example, radioimmunoassays or assays using other labels for the antibodies, can be used. For example, an antigen can be incubated with a a saturating amount of a first antibody or antigen-binding fragment thereof conjugated to a labeled compound (e.g., ³H, ¹²⁵I, biotin, or rubidium) in the presence the same amount of a second unlabeled antibody. The amount of labeled antibody that is bound to the antigen in the presence of the unlabeled blocking antibody is then assessed and compared to binding in the absence of the unlabeled blocking antibody. Competition is determined by the percentage change in binding signals in the presence of the unlabeled blocking antibody compared to the absence of the blocking antibody. Thus, if there is a 70% inhibition of binding of the labeled antibody in the presence of the blocking antibody compared to binding in the absence of the blocking antibody, then there is competition between the two antibodies of 70%. Thus, reference to competition between a first and second antibody of 70% or more, such as 70%, 71%, 72%, 73%, 74%, 75%, 80%, 85%, 90%, 95% or more, means that the first antibody inhibits binding of the second antibody (or vice versa) to the antigen by 70%, 71%, 72%, 73%, 74%, 75%, 80%, 85%, 90%, 95% or more (compared to binding of the antigen by the second antibody in the absence of the first antibody). Thus, inhibition of binding of a first antibody to an antigen by a second antibody of 70%, 71%, 72%, 73%, 74%, 75%, 80%, 85%, 90%, 95% or more indicates that the two antibodies bind to the same epitope.

As used herein, a multimerization domain refers to a sequence of amino acids that promotes stable interaction of a polypeptide molecule with one or more additional polypeptide molecules, each containing a complementary multimerization domain, which can be the same or a different multimerization domain to form a stable multimer with the first domain. Generally, a polypeptide is joined directly or indirectly to the multimerization domain. Exemplary multimerization domains include the immunoglobulin sequences or portions thereof, leucine zippers, hydrophobic regions, hydrophilic regions, and compatible protein-protein interaction domains. The multimerization domain, for example, can be an immunoglobulin constant region or domain, such as, for example, the Fc domain or portions thereof from IgG, including IgG1, IgG2, IgG3 or IgG4 subtypes, IgA, IgE, IgD and IgM and modified forms thereof.

As used herein, dimerization domains are multimerization domains that facilitate interaction between two polypeptide sequences (such as, but not limited to, antibody chains). Dimerization domains include, but are not limited to, an amino acid sequence containing a cysteine residue that facilitates formation of a disulfide bond between two polypeptide sequences, such as all or part of a full-length antibody hinge region, or one or more dimerization sequences, which are sequences of amino acids known to promote interaction between polypeptides (e.g., leucine zippers, GCN4 zippers). In some examples of the provided methods and compositions, one or more dimerization domains is included in a domain exchanged antibody fragment, in order to promote interaction between chains, and thus stabilize the domain exchanged configuration.

As used herein, “Fc” or “Fc region” or “Fc domain” refers to a polypeptide containing the constant region of an antibody heavy chain, excluding the first constant region immunoglobulin domain. Thus, Fc refers to the last two constant region immunoglobulin domains of IgA, IgD, and IgE, or the last three constant region immunoglobulin domains of IgE and IgM. Optionally, an Fc domain can include all or part of the flexible hinge N-terminal to these domains. For IgA and IgM, Fc can include the J chain. For an exemplary Fc domain of IgG, Fc contains immunoglobulin domains Cγ2 and Cγ3, and optionally, all or part of the hinge between Cγ1 and Cγ2. The boundaries of the Fc region can vary, but typically, include at least part of the hinge region. In addition, Fc also includes any allelic or species variant or any variant or modified form, such as any variant or modified form that alters the binding to an FcR or alters an Fc-mediated effector function.

As used herein, a “tag” or an “epitope tag” refers to a sequence of amino acids, typically added to the N- or C-terminus of a polypeptide, such as an antibody provided herein. The inclusion of tags fused to a polypeptide can facilitate polypeptide purification and/or detection. Typically, a tag or tag polypeptide refers to polypeptide that has enough residues to provide an epitope recognized by an antibody or can serve for detection or purification, yet is short enough such that it does not interfere with activity of chimeric polypeptide to which it is linked. The tag polypeptide typically is sufficiently unique so an antibody that specifically binds thereto does not substantially cross-react with epitopes in the polypeptide to which it is linked. Suitable tag polypeptides generally have at least 5 or 6 amino acid residues and usually between about 8-50 amino acid residues, typically between 9-30 residues. The tags can be linked to one or more chimeric polypeptides in a multimer and permit detection of the multimer or its recovery from a sample or mixture. Such tags are well known and can be readily synthesized and designed. Exemplary tag polypeptides include those used for affinity purification and include, His tags, the influenza hemagglutinin (HA) tag polypeptide and its antibody 12CA5, (Field et al. (1988) Mol. Cell. Biol. 8:2159-2165); the c-myc tag and the 8F9, 3C7, 6E10, G4, B7 and 9E10 antibodies thereto (see, e.g., Evan et al. (1985) Molecular and Cellular Biology 5:3610-3616); and the Herpes Simplex virus glycoprotein D (gD) tag and its antibody (Paborsky et al. (1990) Protein Engineering 3:547-553 (1990). An antibody used to detect an epitope-tagged antibody is typically referred to herein as a secondary antibody.

As used herein, “polypeptide” refers to two or more amino acids covalently joined. The terms “polypeptide” and “protein” are used interchangeably herein.

As used herein, a “peptide” refers to a polypeptide that is from 2 to about or 40 amino acids in length.

As used herein, an “amino acid” is an organic compound containing an amino group and a carboxylic acid group. A polypeptide contains two or more amino acids.

For purposes herein, amino acids contained in the antibodies provided include the twenty naturally-occurring amino acids (Table 1), non-natural amino acids, and amino acid analogs (e.g., amino acids wherein the α-carbon has a side chain). As used herein, the amino acids, which occur in the various amino acid sequences of polypeptides appearing herein, are identified according to their well-known, three-letter or one-letter abbreviations (see Table 1). The nucleotides, which occur in the various nucleic acid molecules and fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3557-59 (1968) and adopted at 37 C.F.R. §§.1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glutamic Acid and/or Glutamine |
| W | Trp | Tryptophan |
| R | Arg | Arginine |
| D | Asp | Aspartic acid |
| N | Asn | Asparagine |
| B | Asx | Aspartic Acid and/or Asparagine |
| C | Cys | Cysteine |
| X | Xaa | Unknown or other |

All sequences of amino acid residues represented herein by a formula have a left to right orientation in the conventional direction of amino-terminus to carboxyl-terminus. In addition, the phrase “amino acid residue” is defined to include the amino acids listed in the Table of Correspondence (Table 1), modified, non-natural and unusual amino acids. Furthermore, a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al., Molecular Biology of the Gene, 4th Edition, 1987, The Benjamin/Cummings Pub. co., p. 224).

Such substitutions can be made in accordance with those set forth in Table 2 as follows:

TABLE 2

| Original | Conservative |
|---|---|
| residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative or non-conservative substitutions.

As used herein, “naturally occurring amino acids” refer to the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally occurring amino acids and include, but are not limited to, the D-isostereomers of amino acids. Exemplary non-natural amino acids are known to those of skill in the art, and include, but are not limited to, 2-Aminoadipic acid (Aad), 3-Aminoadipic acid (Baad), β-alanine/β-Amino-propionic acid (Bala), 2-Aminobutyric acid (Abu), 4-Aminobutyric acid/piperidinic acid (4Abu), 6-Aminocaproic acid (Acp), 2-Aminoheptanoic acid (Ahe), 2-Aminoisobutyric acid (Aib), 3-Aminoisobutyric acid (Baib), 2-Aminopimelic acid (Apm), 2,4-Diaminobutyric acid (Dbu), Desmosine (Des), 2,2′-Diaminopimelic acid (Dpm), 2,3-Diaminopropionic acid (Dpr), N-Ethyl glycine (EtGly), N-Ethylasparagine (EtAsn), Hydroxylysine (Hyl), allo-Hydroxylysine (Ahyl), 3-Hydroxyproline (3Hyp), 4-Hydroxyproline (4Hyp), Isodesmosine (Ide), allo-Isoleucine (Aile), N-Methylglycine, sarcosine (MeGly), N-Methylisoleucine (MeIle), 6-N-Methyllysine (MeLys), N-Methylvaline (MeVal), Norvaline (Nva), Norleucine (Nle) and Ornithine (Orn).

As used herein, a “native polypeptide” or a “native nucleic acid” molecule is a polypeptide or nucleic acid molecule, respectively, that can be found in nature. A native polypeptide or nucleic acid molecule can be the wild-type form of a polypeptide or nucleic acid molecule. A native polypeptide or nucleic acid molecule can be the predominant form of the polypeptide, or any allelic or other natural variant thereof. The variant polypeptides and nucleic acid molecules provided herein can have modifications compared to native polypeptides and nucleic acid molecules.

As used herein, the wild-type form of a polypeptide or nucleic acid molecule is a form encoded by a gene or by a coding sequence encoded by the gene. Typically, a wild-type form of a gene, or molecule encoded thereby, does not contain mutations or other modifications that alter function or structure. The term wild-type also encompasses forms with allelic variation as occurs among and between species. As used herein, a predominant form of a polypeptide or nucleic acid molecule refers to a form of the molecule that is the major form produced from a gene. A “predominant form” varies from source to source. For example, different cells or tissue types can produce different forms of polypeptides, for example, by alternative splicing and/or by alternative protein processing. In each cell or tissue type, a different polypeptide can be a “predominant form.”

As used herein, an “allelic variant” or “allelic variation” references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically the reference form of the gene encodes a wild type form and/or predominant form of a polypeptide from a population or single reference member of a species. Typically, allelic variants, which include variants between and among species typically have at least or about 80%, 85%, 90%, 95% or greater amino acid identity with a wild type and/or predominant form from the same species; the degree of identity depends upon the gene and whether comparison is interspecies or intraspecies. Generally, intraspecies allelic variants have at least or about 80%, 85%, 90% or 95% identity or greater with a wild type and/or predominant form, including 96%, 97%, 98%, 99% or greater identity with a wild type and/or predominant form of a polypeptide. Reference to an allelic variant herein generally refers to variations n proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include substitutions, deletions and insertions of nucleotides. An allele of a gene also can be a form of a gene containing a mutation.

As used herein, “species variants” refer to variants in polypeptides among different species, including different mammalian species, such as mouse and human, and species of microorganisms, such as yeast and bacteria.

As used herein, a polypeptide “domain” is a part of a polypeptide (a sequence of three or more, generally 5, 10 or more amino acids) that is a structurally and/or functionally distinguishable or definable. Exemplary of a polypeptide domain is a part of the polypeptide that can form an independently folded structure within a polypeptide made up of one or more structural motifs (e.g. combinations of alpha helices and/or beta strands connected by loop regions) and/or that is recognized by a particular functional activity, such as enzymatic activity, dimerization or antigen-binding. A polypeptide can have one or more, typically more than one, distinct domains. For example, the polypeptide can have one or more structural domains and one or more functional domains. A single polypeptide domain can be distinguished based on structure and function. A domain can encompass a contiguous linear sequence of amino acids. Alternatively, a domain can encompass a plurality of non-contiguous amino acid portions, which are non-contiguous along the linear sequence of amino acids of the polypeptide. Typically, a polypeptide contains a plurality of domains. For example, each heavy chain and each light chain of an antibody molecule contains a plurality of immunoglobulin (Ig) domains, each about 110 amino acids in length.

As used herein, a “property” of a polypeptide, such as an antibody, refers to any property exhibited by a polypeptide, including, but not limited to, binding specificity, structural configuration or conformation, protein stability, resistance to proteolysis, conformational stability, thermal tolerance, and tolerance to pH conditions. Changes in properties can alter an “activity” of the polypeptide. For example, a change in the binding specificity of the antibody polypeptide can alter the ability to bind an antigen, and/or various binding activities, such as affinity or avidity, or in vivo activities of the polypeptide.

As used herein, an “activity” or a “functional activity” of a polypeptide, such as an antibody, refers to any activity exhibited by the polypeptide. Such activities can be empirically determined. Exemplary activities include, but are not limited to, ability to interact with a biomolecule, for example, through antigen-binding, DNA binding, ligand binding, or dimerization, enzymatic activity, for example, kinase activity or proteolytic activity. For an antibody (including antibody fragments), activities include, but are not limited to, the ability to specifically bind a particular antigen, affinity of antigen-binding (e.g. high or low affinity), avidity of antigen-binding (e.g. high or low avidity), on-rate, off-rate, effector functions, such as the ability to promote antigen neutralization or clearance, and in vivo activities, such as the ability to prevent infection or invasion of a pathogen, or to promote clearance, or to penetrate a particular tissue or fluid or cell in the body. Activity can be assessed in vitro or in vivo using recognized assays, such as ELISA, flow cytometry, surface plasmon resonance or equivalent assays to measure on- or off-rate, immunohistochemistry and immunofluorescence histology and microscopy, cell-based assays, flow cytometry and binding assays (e.g. panning assays). For example, for an antibody polypeptide, activities can be assessed by measuring binding affinities, avidities, and/or binding coefficients (e.g. for on-/off-rates), and other activities in vitro or by measuring various effects in vivo, such as immune effects, e.g. antigen clearance, penetration or localization of the antibody into tissues, protection from disease, e.g. infection, serum or other fluid antibody titers, or other assays that are well known in the art. The results of such assays that indicate that a polypeptide exhibits an activity can be correlated to activity of the polypeptide in vivo, in which in vivo activity can be referred to as therapeutic activity, or biological activity. Activity of a modified polypeptide can be any level of percentage of activity of the unmodified polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more of activity compared to the unmodified polypeptide. Assays to determine functionality or activity of modified (e.g. variant) antibodies are well known in the art.

As used herein. “therapeutic activity” refers to the in vivo activity of a therapeutic polypeptide. Generally, the therapeutic activity is the activity that is used to treat a disease or condition. Therapeutic activity of a modified polypeptide can be any level of percentage of therapeutic activity of the unmodified polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more of therapeutic activity compared to the unmodified polypeptide.

As used herein, “exhibits at least one activity” or “retains at least one activity” refers to the activity exhibited by a modified polypeptide, such as a variant polypeptide produced according to the provided methods, such as a modified, e.g. variant antibody or other therapeutic polypeptide (e.g. a modified anti-Candida antibody), compared to the target or unmodified polypeptide, that does not contain the modification. A modified, or variant, polypeptide that retains an activity of a target polypeptide can exhibit improved activity or maintain the activity of the unmodified polypeptide. In some instances, a modified, or variant, polypeptide can retain an activity that is increased compared to an target or unmodified polypeptide. In some cases, a modified, or variant, polypeptide can retain an activity that is decreased compared to an unmodified or target polypeptide. Activity of a modified, or variant, polypeptide can be any level of percentage of activity of the unmodified or target polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more activity compared to the unmodified or target polypeptide. In other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times greater than unmodified or target polypeptide. Assays for retention of an activity depend on the activity to be retained. Such assays can be performed in vitro or in vivo. Activity can be measured, for example, using assays known in the art and described in the Examples below for activities such as but not limited to ELISA and panning assays. Activities of a modified, or variant, polypeptide compared to an unmodified or target polypeptide also can be assessed in terms of an in vivo therapeutic or biological activity or result following administration of the polypeptide.

As used herein, “screening” refers to identification or selection of an antibody or portion thereof from a plurality of antibodies, such as a collection or library of antibodies and/or portions thereof, based on determination of the activity or property of an antibody or portion thereof. Screening can be performed in any of a variety of ways and generally involves containing members of the collection with an antigen and assessing a property or activity, for example, by assays assessing direct binding (e.g. binding affinity) of the antibody to a target protein or by functional assays assessing modulation of an activity of a target protein.

As used herein, the term “assessing” is intended to include quantitative and qualitative determination in the sense of obtaining an absolute value for the activity of a protease, or a domain thereof, present in the sample, and also of obtaining an index, ratio, percentage, visual, or other value indicative of the level of the activity. Assessment can be direct or indirect and the chemical species actually detected need not of course be the proteolysis product itself but can for example be a derivative thereof or some further substance. For example, detection of a cleavage product of a complement protein, such as by SDS-PAGE and protein staining with Coomassie blue.

As used herein, the term “nucleic acid” refers to at least two linked nucleotides or nucleotide derivatives, including a deoxyribonucleic acid (DNA) and a ribonucleic acid (RNA), joined together, typically by phosphodiester linkages. Also included in the term “nucleic acid” are analogs of nucleic acids such as peptide nucleic acid (PNA), phosphorothioate DNA, and other such analogs and derivatives or combinations thereof. Nucleic acids also include DNA and RNA derivatives containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). The term also includes, as equivalents, derivatives, variants and analogs of either RNA or DNA made from nucleotide analogs, single (sense or antisense) and double-stranded nucleic acids. Deoxyribonucleotides include deoxyadenosine, deoxycytidine, deoxyguanosine and deoxythymidine. For RNA, the uracil base is uridine.

Nucleic acids can contain nucleotide analogs, including, for example, mass modified nucleotides, which allow for mass differentiation of nucleic acid molecules; nucleotides containing a detectable label such as a fluorescent, radioactive, luminescent or chemiluminescent label, which allow for detection of a nucleic acid molecule; or nucleotides containing a reactive group such as biotin or a thiol group, which facilitates immobilization of a nucleic acid molecule to a solid support. A nucleic acid also can contain one or more backbone bonds that are selectively cleavable, for example, chemically, enzymatically or photolytically cleavable. For example, a nucleic acid can include one or more deoxyribonucleotides, followed by one or more ribonucleotides, which can be followed by one or more deoxyribonucleotides, such a sequence being cleavable at the ribonucleotide sequence by base hydrolysis. A nucleic acid also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. (1997) Nucleic Acids Res. 25:2792-2799).

As used herein, the terms “polynucleotide” and “nucleic acid molecule” refer to an oligomer or polymer containing at least two linked nucleotides or nucleotide derivatives, including a deoxyribonucleic acid (DNA) and a ribonucleic acid (RNA), joined together, typically by phosphodiester linkages. Polynucleotides also include DNA and RNA derivatives containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). Polynucleotides (nucleic acid molecules), include single-stranded and/or double-stranded polynucleotides, such as deoxyribonucleic acid (DNA), and ribonucleic acid (RNA) as well as analogs or derivatives of either RNA or DNA. The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine. Polynucleotides can contain nucleotide analogs, including, for example, mass modified nucleotides, which allow for mass differentiation of polynucleotides; nucleotides containing a detectable label such as a fluorescent, radioactive, luminescent or chemiluminescent label, which allow for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically cleavable. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. (1997) Nucleic Acids Res. 25:2792-2799). Exemplary of the nucleic acid molecules (polynucleotides) provided herein are oligonucleotides, including synthetic oligonucleotides, oligonucleotide duplexes, primers, including fill-in primers, and oligonucleotide duplex cassettes.

As used herein, a “DNA construct” is a single or double stranded, linear or circular DNA molecule that contains segments of DNA combined and juxtaposed in a manner not found in nature. DNA constructs exist as a result of human manipulation, and include clones and other copies of manipulated molecules.

As used herein, a genetic element refers to a gene, or any region thereof, that encodes a polypeptide or protein or region thereof. In some examples, a genetic element encodes a fusion protein.

As used herein, regulatory region of a nucleic acid molecule means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, “operably linked” with reference to nucleic acid sequences, regions, elements or domains means that the nucleic acid regions are functionally related to each other. For example, nucleic acid encoding a leader peptide can be operably linked to nucleic acid encoding a polypeptide, whereby the nucleic acids can be transcribed and translated to express a functional fusion protein, wherein the leader peptide effects secretion of the fusion polypeptide. In some instances, the nucleic acid encoding a first polypeptide (e.g. a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant means by using recombinant DNA methods means the use of the well known methods of molecular biology for expressing proteins encoded by cloned DNA.

As used herein, “expression” refers to the process by which polypeptides are produced by transcription and translation of polynucleotides. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay and Bradford protein assay.

As used herein, a “host cell” is a cell that is used in to receive, maintain, reproduce and amplify a vector. A host cell also can be used to express the polypeptide encoded by the vector. The nucleic acid contained in the vector is replicated when the host cell divides, thereby amplifying the nucleic acids. In one example, the host cell is a genetic package, which can be induced to express the variant polypeptide on its surface. In another example, the host cell is infected with the genetic package. For example, the host cells can be phage-display compatible host cells, which can be transformed with phage or phagemid vectors and accommodate the packaging of phage expressing fusion proteins containing the variant polypeptides.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, a vector also includes “virus vectors” or “viral vectors.” Viral vectors are engineered viruses that are operatively linked to exogenous genes to transfer (as vehicles or shuttles) the exogenous genes into cells.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, the terms “oligonucleotide” and “oligo” are used synonymously. Oligonucleotides are polynucleotides that contain a limited number of nucleotides in length. Those in the art recognize that oligonucleotides generally are less than at or about two hundred fifty, typically less than at or about two hundred, typically less than at or about one hundred, nucleotides in length. Typically, the oligonucleotides are synthetic oligonucleotides. The synthetic oligonucleotides contain fewer than at or about 250 or 200 nucleotides in length, for example, fewer than about 20, 30, 40, 50, 60, 70, 80, 90, 100, 110, 120, 130, 140, 150, 160, 170, 180, 190 or 200 nucleotides in length. Typically, the oligonucleotides are single-stranded oligonucleotides. The ending “mer” can be used to denote the length of an oligonucleotide. For example, “100-mer” can be used to refer to an oligonucleotide containing 100 nucleotides in length. Exemplary of the synthetic oligonucleotides are positive and negative strand oligonucleotides, randomized oligonucleotides, reference sequence oligonucleotides, template oligonucleotides and fill-in primers.

As used herein, synthetic oligonucleotides are oligonucleotides produced by chemical synthesis. Chemical oligonucleotide synthesis methods are well known. Any of the known synthesis methods can be used to produce the oligonucleotides. For example, synthetic oligonucleotides typically are made by chemically joining single nucleotide monomers or nucleotide trimers containing protective groups. Typically, phosphoramidites, single nucleotides containing protective groups are added one at a time. Synthesis typically begins with the 3′ end of the oligonucleotide. The 3′ most phosphoramidite is attached to a solid support and synthesis proceeds by adding each phosphoramidite to the 5′ end of the last. After each addition, the protective group is removed from the 5′ phosphate group on the most recently added base, allowing addition of another phosphoramidite. Automated synthesizers generally can synthesize oligonucleotides up to about 150 to about 200 nucleotides in length. Typically, the oligonucleotides are designed and synthesized using standard cyanoethyl chemistry from phosphoramidite monomers. Synthetic oligonucleotides produced by this standard method can be purchased from Integrated DNA Technologies (IDT) (Coralville, Iowa) or TriLink Biotechnologies (San Diego, Calif.).

As used herein, “primer” refers to a nucleic acid molecule (more typically, to a pool of such molecules sharing sequence identity) that can act as a point of initiation of template-directed nucleic acid synthesis under appropriate conditions (for example, in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)—PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primary sequence” refers to the sequence of amino acid residues in a polypeptide or the sequence of nucleotides in a nucleic acid molecule.

As used herein, “similarity” between two proteins or nucleic acids refers to the relatedness between the sequence of amino acids of the proteins or the nucleotide sequences of the nucleic acids. Similarity can be based on the degree of identity of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

As used herein, when a polypeptide or nucleic acid molecule or region thereof contains or has “identity” or “homology” to another polypeptide or nucleic acid molecule or region, the two molecules and/or regions share greater than or equal to at or about 40% sequence identity, and typically greater than or equal to at or about 50% sequence identity, such as at least or about 60%, 65%, 70%, 75%, 80%, 85%, 90%, 95%, 96%, 97%, 98%, 99% or 100% sequence identity; the precise percentage of identity can be specified if necessary. A nucleic acid molecule, or region thereof, that is identical or homologous to a second nucleic acid molecule or region can specifically hybridize to a nucleic acid molecule or region that is 100% complementary to the second nucleic acid molecule or region. Identity alternatively can be compared between two theoretical nucleotide or amino acid sequences or between a nucleic acid or polypeptide molecule and a theoretical sequence.

Sequence “identity,” per se, has an art-recognized meaning and the percentage of sequence identity between two nucleic acid or polypeptide molecules or regions can be calculated using published techniques. Sequence identity can be measured along the full length of a polynucleotide or polypeptide or along a region of the molecule. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptides, the term “identity” is well known to skilled artisans (Carrillo, H. & Lipman, D., (1988) SIAM J Applied Math 48:1073).

Sequence identity compared along the full length of two polynucleotides or polypeptides refers to the percentage of identical nucleotide or amino acid residues along the full-length of the molecule. For example, if a polypeptide A has 100 amino acids and polypeptide B has 95 amino acids, which are identical to amino acids 1-95 of polypeptide A, then polypeptide B has 95% identity when sequence identity is compared along the full length of a polypeptide A compared to full length of polypeptide B. Alternatively, sequence identity between polypeptide A and polypeptide B can be compared along a region, such as a 20 amino acid analogous region, of each polypeptide. In this case, if polypeptide A and B have 20 identical amino acids along that region, the sequence identity for the regions is 100%. Alternatively, sequence identity can be compared along the length of a molecule, compared to a region of another molecule. As discussed below, and known to those of skill in the art, various programs and methods for assessing identity are known to those of skill in the art. High levels of identity, such as 90% or 95% identity, readily can be determined without software.

Whether any two nucleic acid molecules have nucleotide sequences that are at least or about 60%, 70%, 80%, 85%, 90%, 95%, 96%, 97%, 98% or 99% “identical” can be determined using known computer algorithms such as the “FASTA” program, using for example, the default parameters as in Pearson et al. (1988) Proc. Natl. Acad. Sci. USA 85:2444 (other programs include the GCG program package (Devereux, J. et al. (1984) Nucleic Acids Research 12(I):387), BLASTP, BLASTN, FASTA (Altschul, S. F. et al. (1990) J. Molec. Biol. 215:403; Guide to Huge Computers, Martin J. Bishop, ed., Academic Press, San Diego, 1994, and Carrillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

In general, for determination of the percentage sequence identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073). For sequence identity, the number of conserved amino acids is determined by standard alignment algorithms programs, and can be used with default gap penalties established by each supplier. Substantially homologous nucleic acid molecules specifically hybridize typically at moderate stringency or at high stringency all along the length of the nucleic acid of interest. Also contemplated are nucleic acid molecules that contain degenerate codons in place of codons in the hybridizing nucleic acid molecule.

Therefore, the term “identity,” when associated with a particular number, represents a comparison between the sequences of a first and a second polypeptide or polynucleotide or regions thereof and/or between theoretical nucleotide or amino acid sequences. As used herein, the term at least “90% identical to” refers to percent identities from 90 to 99.99 relative to the first nucleic acid or amino acid sequence of the polypeptide. Identity at a level of 90% or more is indicative of the fact that, assuming for exemplification purposes, a first and second polypeptide length of 100 amino acids are compared, no more than 10% (i.e., 10 out of 100) of the amino acids in the first polypeptide differs from that of the second polypeptide. Similar comparisons can be made between first and second polynucleotides. Such differences among the first and second sequences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleotide or amino acid residue substitutions, insertions, additions or deletions. At the level of homologies or identities above about 85-90%, the result is independent of the program and gap parameters set; such high levels of identity can be assessed readily, often by manual alignment without relying on software.

As used herein, alignment of a sequence refers to the use of homology to align two or more sequences of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

Related or variant polypeptides or nucleic acid molecules can be aligned by any method known to those of skill in the art. Such methods typically maximize matches, and include methods, such as using manual alignments and by using the numerous alignment programs available (e.g., BLASTP) and others known to those of skill in the art. By aligning the sequences of polypeptides or nucleic acids, one skilled in the art can identify analogous portions or positions, using conserved and identical amino acid residues as guides. Further, one skilled in the art also can employ conserved amino acid or nucleotide residues as guides to find corresponding amino acid or nucleotide residues between and among human and non-human sequences. Corresponding positions also can be based on structural alignments, for example by using computer simulated alignments of protein structure. In other instances, corresponding regions can be identified. One skilled in the art also can employ conserved amino acid residues as guides to find corresponding amino acid residues between and among human and non-human sequences.

As used herein, “analogous” and “corresponding” portions, positions or regions are portions, positions or regions that are aligned with one another upon aligning two or more related polypeptide or nucleic acid sequences (including sequences of molecules, regions of molecules and/or theoretical sequences) so that the highest order match is obtained, using an alignment method known to those of skill in the art to maximize matches. In other words, two analogous positions (or portions or regions) align upon best-fit alignment of two or more polypeptide or nucleic acid sequences. The analogous portions/positions/regions are identified based on position along the linear nucleic acid or amino acid sequence when the two or more sequences are aligned. The analogous portions need not share any sequence similarity with one another. For example, alignment (such that maximizing matches) of the sequences of two homologous nucleic acid molecules, each 100 nucleotides in length, can reveal that 70 of the 100 nucleotides are identical. Portions of these nucleic acid molecules containing some or all of the other non-identical 30 amino acids are analogous portions that do not share sequence identity. Alternatively, the analogous portions can contain some percentage of sequence identity to one another, such as at or about 50%, 55%, 60%, 65%, 70%, 75%, 80%, 85%, 90%, 95%, 96%, 97%, 98%, 99%, or fractions thereof. In one example, the analogous portions are 100% identical.

As used herein, a “modification” is in reference to modification of a sequence of amino acids of a polypeptide or a sequence of nucleotides in a nucleic acid molecule and includes deletions, insertions, and replacements of amino acids and nucleotides, respectively. Methods of modifying a polypeptide are routine to those of skill in the art, such as by using recombinant DNA methodologies.

As used herein, “deletion,” when referring to a nucleic acid or polypeptide sequence, refers to the deletion of one or more nucleotides or amino acids compared to a sequence, such as a target polynucleotide or polypeptide or a native or wild-type sequence.

As used herein, “insertion” or “addition” when referring to a nucleic acid or amino acid sequence, describes the inclusion of one or more additional nucleotides or amino acids, within a target, native, wild-type or other related sequence. Thus, a nucleic acid molecule that contains one or more insertions compared to a wild-type sequence, contains one or more additional nucleotides within the linear length of the sequence. As used herein, “additions,” to nucleic acid and amino acid sequences describe addition of nucleotides, or amino acids onto either termini compared to another sequence.

As used herein, “substitution” or “replacement” refers to the replacing of one or more nucleotides or amino acids in a native, target, wild-type or other nucleic acid or polypeptide sequence with an alternative nucleotide or amino acid, without changing the length (as described in numbers of residues) of the molecule. Thus, one or more substitutions in a molecule does not change the number of amino acid residues or nucleotides of the molecule. Substitution mutations compared to a particular polypeptide can be expressed in terms of the number of the amino acid residue along the length of the polypeptide sequence. For example, a modified polypeptide having a modification in the amino acid at the 19^{th} position of the amino acid sequence that is a substitution of Isoleucine (Ile; I) for cysteine (Cys; C) can be expressed as 119C, Ile19C, or simply C19, to indicate that the amino acid at the modified 19^{th} position is a cysteine. In this example, the molecule having the substitution has a modification at Ile 19 of the unmodified polypeptide.

As used herein, a disulfide bond (also called an S—S bond or a disulfide bridge) is a single covalent bond derived from the coupling of thiol groups. Disulfide bonds in proteins are formed between the thiol groups of cysteine residues, and stabilize interactions between polypeptide domains, such as antibody domains.

As used herein, “coupled” or “conjugated” means attached via a covalent or noncovalent interaction.

As used herein, the phrase “conjugated to an antibody” or “linked to an antibody” or grammatical variations thereof, when referring to the attachment of a moiety to an antibody or antigen-binding fragment thereof, such as a diagnostic or therapeutic moiety, means that the moiety is attached to the antibody or antigen-binding fragment thereof by any known means for linking peptides, such as, for example, by production of fusion protein by recombinant means or post-translationally by chemical means. Conjugation can employ any of a variety of linking agents to effect conjugation, including, but not limited to, peptide or compound linkers or chemical cross-linking agents.

As used herein, “phage display” refers to the expression of polypeptides on the surface of filamentous bacteriophage.

As used herein, a “phage-display compatible cell” or “phage-display compatible host cell” is a host cell, typically a bacterial host cell that can be infected by phage and thus can support the production of phage displaying fusion proteins containing polypeptides, e.g. variant polypeptides and can thus be used for phage display. Exemplary of phage display compatible cells include, but are not limited to, XL1-blue cells.

As used herein, “panning” refers to an affinity-based selection procedure for the isolation of phage displaying a molecule with a specificity for a binding partner, for example, a capture molecule (e.g. an antigen) or sequence of amino acids or nucleotides or epitope, region, portion or locus therein.

As used herein, “display protein” or “genetic package display protein” means any genetic package polypeptide for display of a polypeptide on the genetic package, such that when the display protein is fused to (e.g. included as part of a fusion protein with) a polypeptide of interest (e.g. a polypeptide for which reduced expression is desired), the polypeptide is displayed on the outer surface of the genetic package. The display protein typically is present on or within the outer surface or outer compartment of a genetic package (e.g. membrane, cell wall, coat or other outer surface or compartment) of a genetic package, e.g. a viral genetic package, such as a phage, such that upon fusion to a polypeptide of interest, the polypeptide is displayed on the genetic package.

As used herein, a coat protein is a display protein, at least a portion of which is present on the outer surface of the genetic package, such that when it is fused to the polypeptide of interest, the polypeptide is displayed on the outer surface of the genetic package. Typically, the coat proteins are viral coat proteins, such as phage coat proteins. A viral coat protein, such as a phage coat protein associates with the virus particle during assembly in a host cell. In one example, coat proteins are used herein for display of polypeptides on genetic packages; the coat proteins are expressed as portions of fusion proteins, which contain the coat protein sequence of amino acids and a sequence of amino acids of the displayed polypeptide. The coat protein can be a full-length coat protein or any portion thereof capable of effecting display of the polypeptide on the surface of the genetic package.

Exemplary of coat proteins are phage coat proteins, such as, but not limited to, (i) minor coat proteins of filamentous phage, such as gene III protein (gIIIp, cp3), and (ii) major coat proteins (which are present in the viral coat at 10 copies or more, for example, tens, hundreds or thousands of copies) of filamentous phage such as gene VIII protein (gVIIIp, cp8); fusions to other phage coat proteins such as gene VI protein, gene VII protein, or gene IX protein (see, e.g., WO 00/71694); and portions (e.g., domains or fragments) of these proteins, such as, but not limited to domains that are stably incorporated into the phage particle, e.g. such as the anchor domain of gIIIp, or gVIIIp. Additionally, mutants of gVIIIp can be used which are optimized for expression of larger peptides, such as mutants having improved surface display properties, such as mutant gVIIp (see, for example, Sidhu et al. (2000) J. Mol. Biol. 296:487-495).

As used herein, “disease or disorder” refers to a pathological condition in an organism resulting from cause or condition including, but not limited to, infections, acquired conditions, genetic conditions, and characterized by identifiable symptoms. Diseases and disorders of interest herein are those involving Candida infection or those that increase the risk of a Candida infection.

As used herein, “infection” and “Candida infection” refer to all stages of a Candida life cycle in a host, as well as the pathological state resulting from the invasion by Candida. The invasion by Candida includes, but is not limited to, adhesion to cells (e.g. epithelial or endothelial) or components of the extracellular matrix, germ tube formation, growth, and replication in the host.

As used herein, “treating” a subject with a disease or condition” means that the subject's symptoms are partially or totally alleviated, or remain static following treatment. Hence treatment encompasses prophylaxis, therapy and/or cure. Treatment also encompasses any pharmaceutical use of any antibody or antigen-binding fragment thereof provided or compositions provided herein.

As used herein, “prevention” or prophylaxis, and grammatically equivalent forms thereof, refers to methods in which the risk of developing disease or condition is reduced. Thus, prophylaxis refers to prevention of a potential disease and/or a prevention of worsening of symptoms or progression of a disease.

As used herein, a “pharmaceutically effective agent” includes any therapeutic agent or bioactive agents, including, but not limited to, for example, anesthetics, vasoconstrictors, dispersing agents, conventional therapeutic drugs, including small molecule drugs and therapeutic proteins.

As used herein, a “therapeutic effect” means an effect resulting from treatment of a subject that alters, typically improves or ameliorates the symptoms of a disease or condition or that cures a disease or condition.

As used herein, a “therapeutically effective amount” or a “therapeutically effective dose” refers to the quantity of an agent, compound, material, or composition containing a compound that is at least sufficient to produce a therapeutic effect following administration to a subject. Hence, it is the quantity necessary for preventing, curing, ameliorating, arresting or partially arresting a symptom of a disease or disorder.

As used herein, “therapeutic efficacy” refers to the ability of an agent, compound, material, or composition containing a compound to produce a therapeutic effect in a subject to whom the an agent, compound, material, or composition containing a compound has been administered.

As used herein, a “prophylactically effective amount” or a “prophylactically effective dose” refers to the quantity of an agent, compound, material, or composition containing a compound that when administered to a subject, will have the intended prophylactic effect, e.g., preventing or delaying the onset, or reoccurrence, of disease or symptoms, reducing the likelihood of the onset, or reoccurrence, of disease or symptoms, or reducing the incidence of fungal infection. The full prophylactic effect does not necessarily occur by administration of one dose, and can occur only after administration of a series of doses. Thus, a prophylactically effective amount can be administered in one or more administrations.

As used herein, the terms “immunotherapeutically” or “immunotherapy” in conjunction with antibodies provided denotes prophylactic as well as therapeutic administration. Thus, the therapeutic antibodies provided can be administered to a subject at risk of contracting a fungal infection (e.g. a Candida infection) in order to lessen the likelihood and/or severity of the disease, or administered to subjects already evidencing active fungal infection (e.g. a Candida infection).

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, the term “diagnostically effective” amount refers to the quantity of an agent, compound, material, or composition containing a detectable compound that is at least sufficient for detection of the compound following administration to a subject. Generally, a diagnostically effective amount of an anti-Candida, such as a detectably-labeled antibody or antigen-binding fragment thereof or an antibody or antigen-binding fragment thereof that can be detected by a secondary agent, administered to a subject for detection is quantity of the antibody or antigen-binding fragment thereof which is sufficient to enable detection of the site having the Candida antigen for which the antibody is specific. In using the antibodies provided herein for the in vivo detection of antigen, a detectably labeled antibody or antigen-binding fragment thereof is given in a dose which is diagnostically effective.

As used herein, a label or detectable moiety is a detectable marker (e.g., a fluorescent molecule, chemiluminescent molecule, a bioluminescent molecule, a contrast agent (e.g., a metal), a radionuclide, a chromophore, a detectable peptide, or an enzyme that catalyzes the formation of a detectable product) that can be attached or linked directly or indirectly to a molecule (e.g., an anti-Candida antibody provided herein) or associated therewith and can be detected in vivo and/or in vitro. The detection method can be any method known in the art, including known in vivo and/or in vitro methods of detection (e.g., imaging by visual inspection, magnetic resonance (MR) spectroscopy, ultrasound signal, X-ray, gamma ray spectroscopy (e.g., positron emission tomography (PET) scanning, single-photon emission computed tomography (SPECT)), fluorescence spectroscopy or absorption). Indirect detection refers to measurement of a physical phenomenon, such as energy or particle emission or absorption, of an atom, molecule or composition that binds directly or indirectly to the detectable moiety (e.g., detection of a labeled secondary antibody or antigen-binding fragment thereof that binds to a primary antibody (e.g., an anti-Candida antibody provided herein).

As used herein, the term “subject” refers to an animal, including a mammal, such as a human being.

As used herein, a patient refers to a human subject.

As used herein, animal includes any animal, such as, but are not limited to primates including humans, gorillas and monkeys; rodents, such as mice and rats; fowl, such as chickens; ruminants, such as goats, cows, deer, sheep; pigs and other animals. Non-human animals exclude humans as the contemplated animal. The enzymes provided herein are from any source, animal, plant, prokaryotic and fungal. Most enzymes are of animal origin, including mammalian origin.

As used herein, a “elderly,” refers to refers to a subject, who due to age has a decreased immune response and has a decreased response to vaccination. Typically, an elderly subject is one that is human that is sixty-five and greater years of age, more typically, 70 and greater years of age.

As used herein, a “human infant” refers to a human less than or about 24 months (e.g., less than or about 16 months, less than or about 12 months, less than or about 6 months, less than or about 3 months, less than or about 2 months, or less than or about 1 month of age). Typically, the human infant is born at more than 38 weeks of gestational age.

As used herein, a “human infant born prematurely” refers to a human born at less than or about 40 weeks gestational age, typically, less than or about 38 weeks gestational age.

As used herein, a “unit dose form” refers to physically discrete units suitable for human and animal subjects and packaged individually as is known in the art.

As used herein, a “single dosage formulation” refers to a formulation for direct administration.

As used herein, an “article of manufacture” is a product that is made and sold. As used throughout this application, the term is intended to encompass any of the compositions provided herein contained in articles of packaging.

As used herein, a “fluid” refers to any composition that can flow. Fluids thus encompass compositions that are in the form of semi-solids, pastes, solutions, aqueous mixtures, gels, lotions, creams and other such compositions.

As used herein, an isolated or purified polypeptide or protein (e.g. an isolated antibody or antigen-binding fragment thereof) or biologically-active portion thereof (e.g. an isolated antigen-binding fragment) is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification does not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound. As used herein, a “cellular extract” or “lysate” refers to a preparation or fraction which is made from a lysed or disrupted cell.

As used herein, isolated nucleic acid molecule is one which is separated from other nucleic acid molecules which are present in the natural source of the nucleic acid molecule. An “isolated” nucleic acid molecule, such as a cDNA molecule, can be substantially free of other cellular material, or culture medium when produced by recombinant techniques, or substantially free of chemical precursors or other chemicals when chemically synthesized. Exemplary isolated nucleic acid molecules provided herein include isolated nucleic acid molecules encoding an antibody or antigen-binding fragments provided.

As used herein, a “control” refers to a sample that is substantially identical to the test sample, except that it is not treated with a test parameter, or, if it is a plasma sample, it can be from a normal volunteer not affected with the condition of interest. A control also can be an internal control.

As used herein, a “composition” refers to any mixture. It can be a solution, suspension, liquid, powder, paste, aqueous, non-aqueous or any combination thereof.

As used herein, a “combination” refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, combination therapy refers to administration of two or more different therapeutics, such as two or more different anti-Candida antibodies or other anti-fungal antibodies or anti-fungal agents. The different therapeutic agents can be provided and administered separately, sequentially, intermittently, or can be provided in a single composition.

As used herein, a kit is a packaged combination that optionally includes other elements, such as additional reagents and instructions for use of the combination or elements thereof, for a purpose including, but not limited to, activation, administration, diagnosis, and assessment of a biological activity or property.

As used herein, the singular forms “a,” “an” and “the” include plural referents unless the context clearly dictates otherwise. Thus, for example, reference to a polypeptide, comprising “an immunoglobulin domain” includes polypeptides with one or a plurality of immunoglobulin domains.

As used herein, the term “or” is used to mean “and/or” unless explicitly indicated to refer to alternatives only or the alternatives are mutually exclusive.

As used herein, ranges and amounts can be expressed as “about” a particular value or range. About also includes the exact amount. Hence “about 5 amino acids” means “about 5 amino acids” and also “5 amino acids.”

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

### B. DOMAIN-EXCHANGED ANTIBODIES

Among the anti-Candida antibodies provided herein are domain-exchanged antibodies. Domain-exchanged antibodies are antibodies, including antibody fragments, having the domain-exchanged structure, which in general is characterized by a configuration having two interlocked V_{H} domains, with an interface forming between the interlocked V_{H} domains (V_{H}-V_{H}′ interface). Typically, the V_{H} domains interact with opposite V_{L} domains compared to the interaction in a conventional antibody (see, for example, U.S. Application Pub. No. US 2005/0003347). A full-length (e.g. intact IgG) domain-exchanged antibody exists as a monomer. FIG. 1 shows a schematic comparison of exemplary conventional and domain-exchanged IgG antibody structures. In this example, the full-length folded domain-exchanged antibody adopts an unusual structure, in which the two heavy chain variable regions swing away from their cognate light chains and pair instead with the “opposite” light chain variable regions. In the exemplary domain-exchanged full-length antibody illustrated in FIG. 1, the variable region of each heavy chain (V_{H} and V_{H}′, respectively) interacts with the variable region on the opposite light chain compared with the interactions between the constant regions of the molecule (C_{H}-C_{L}). A full-length (e.g. intact IgG) domain exchange antibody can exist as monomers or substantially as dimers (see e.g., West et al. (2009) J. Virol., 83:98-104). Domain-exchanged antibody fragments, for example Fab fragments, exist as dimers due to the interface formed by two interlocking V_{H} domains.

In conventionally structured IgG, IgD and IgA antibodies, the hinge regions between the C_{H}1 and C_{H}2 domains can provide flexibility, resulting in mobile antibody combining sites that can move relative to one another to interact with epitopes, for example, on cell surfaces. In domain-exchanged antibodies, by contrast, this flexible arrangement is not adopted. In one example, domain-exchanged antibodies can contain two conventional antibody combining sites and a non-conventional antibody combining site, which is formed by the interface between the two adjacently positioned heavy chain variable regions, all of which are in close proximity with one another and constrained in space, as illustrated in the exemplary IgG in FIG. 1.

Due to the structure of domain-exchanged antibodies, the separation between antigen-binding sites is smaller as compared to conventional antibodies. Conventional full-length antibodies, such as conventional full length IgG antibodies, generally contain two antigen-binding sites separated by distances that are greater than 120 Å, generally 150-170 Å. In contrast, domain-exchanged antibodies have at least two antigen-binding sites separated by a distance that is less than 120 Å. For example, the antigen-binding sites in 2G12 are separated by about 35 Å (see e.g., West et al. (2009) J. Virol., 83:98-104).

Also, because of their structure, domain-exchanged antibodies can contain multiple binding sites. The size and number of the antigen-binding sites means that domain-exchanged antibodies can specifically bind epitopes within densely packed and/or repetitive epitope arrays, such as sugar residues on yeast, bacterial or viral surfaces. The unusual domain-exchanged configuration can promote binding to such epitopes. In some examples, domain-exchanged antibodies can recognize and bind epitopes within high density arrays, which evolve, for example, in pathogens and tumor cells as means for immune evasion. Examples of such high density/repetitive epitope arrays include, but are not limited to, epitopes contained within yeast and bacterial cell wall carbohydrates and carbohydrates and glycolipids displayed on the surfaces of tumor cells or viruses. Such epitopes are not optimally recognized by conventional (non-domain-exchanged) antibodies. In one example, the high density and/or repetitiveness of epitopes can render simultaneous binding of both antibody-combining sites of a conventional antibody energetically disfavored.

Domain-exchanged antibodies can be identified based on their structure. As intact IgG molecules, domain-exchanged antibodies form a compact structure, monomeric or dimeric. Thus, unlike typical antibodies that form a Y- or T-shape, a full-length domain-exchanged antibody forms an extended linear conformation due to the parallel arrangement and intimate association formed by the V_{H}-V_{H}′ interface. Based on the unique features of full-length domain-exchanged antibodies, they can be identified by various methods known to one of skill in the art, including, but not limited to, size exclusion chromatography, in-line static light scattering and refractive index monitoring, electron microscopy, sedimentation equilibrium analytical ultracentrifugation, gel filtration, native gel electrophoresis, sedimentation coefficients and/or negative-stain electron microscopy (West et al. (2009) J. Virol., 83:98-104; Roux et al. (2004) Mol. Immunol., 41:1001-1011; Calarese et al. (2005) Science, 300:2065-2071; U.S. Application Publication No. US20050003347). For example, due to the compact structure, a full-length domain-exchanged antibody has a higher sedimentation coefficient then conventional antibodies. Also, due to the unique linear shape, domain-exchanged antibodies can be identified from typical Y- or T-shaped antibodies by methods such as electron microscopy.

In other antibody forms, such as antibody fragments of a full-length IgG, domain-exchanged antibodies exist as dimers due to the interface formed by two interlocking V_{H} domains. For example, in their Fab form, domain-exchanged binding molecules exist as Fab dimers. As described elsewhere herein, those of skill in the art are familiar with assays to assess the oligomeric state of proteins, such as antibodies, for example assays to assess the presence of a Fab dimer of a domain-exchanged binding molecule. Such assays include, for example, sedimentation equilibrium analytical ultracentrifugation, gel filtration, native gel electrophoresis, velocity sedimentation coefficients and/or negative-stain electron microscopy (Roux et al. (2004) Mol. Immunol., 41:1001-1011; Calarese et al. (2005) Science, 300:2065-2071; U.S. Publication No.: US20050003347; Gach et al. (2010) J. Biol. Chem., 285:1122-1127). For example, domain-exchanged antibodies, such as domain-exchanged Fabs, can be identified by methods such as gel filtration due to the higher molecular mass compared to typical antibodies.

Exemplary of a domain-exchanged antibody is 2G12. Domain-exchanged antibodies also include any antibody that has a domain-exchanged antibody configuration. These include, for example, any that adopt the domain-exchanged configuration due to mutation(s) in the heavy chains, such as within the joining region between the V_{H} and C_{H} regions (see, for example, U.S. Application Pub. No. US 2005/0003347). The mutant residues include isoleucine (Ile) at position 19, arginine (Arg) at position 57, phenylalanine (Phe) at position 77 and any amino acid residue at position 113 capable of forming a hydrophobic interaction with H84, for example, proline (Pro) or Serine, where numbering is based on Kabat numbering (see e.g. U.S. Application Pub. No. US 2005/0003347; Calarese et al. (2005) Science, 300:2065-2071; Gach et al. (2010) J. Biol. Chem., 285:1122-1127). Further residues for amino acid mutation include amino acid residues 39, 70, 72, 79, 81 and 84, based on Kabat numbering. In particular, the mutations are arginine (Arg) at position 39, serine (Ser) at position 70, aspartic acid (Asp) at position 72, tyrosine (Tyr) at position 79, glutamine (Gln) at position 81, or valine (Val) at position 84, based on Kabat numbering. In another example, mutations in the heavy chain that result in an antibody adopting a domain-exchanged structure include residues isoleucine (Ile) at position 19, arginine (Arg) at position 57 and glutamic acid (Glu) at position 75 in the V_{H}-V_{H}′ interface, residue arginine (Arg) at position 39 in the V_{H}-V_{L} interface and residues alanine (Ala) at position 14, valine (Val) at position 84 and proline (Pro) at position 113 in the elbow region, where numbering is based on Kabat numbering (see e.g. Huber et al., (2010) J. Virol August 11 (Epub)). One of skill in the art is able to identify a domain-exchanged binding molecule based on structural and other properties, for example, oligomerization state.

2G12 Domain-Exchanged Antibody

Exemplary of a domain-exchanged antibody is the 2G12 antibody. Hence, provided herein are modified 2G12 antibodies that bind to and inhibit one or more pathogenic activities of members of the genus Candida. As described in Section C, the modified 2G12 antibodies are modified in their complementarity determining region (CDR) compared to 2G12, thereby resulting in increased binding affinity for Candida compared to the corresponding form of 2G12.

2G12 is a broadly neutralizing anti-HIV antibody. Amino acid residues in the V_{H} domains of 2G12 (e.g. amino acids at positions 19 (Ile), 57 (Arg), 77 (Phe), 84 (Val) and 113 (Pro), based on Kabat numbering), which vary compared to analogous residues in conventional antibodies, promote and/or stabilize the domain-exchanged structure and stabilize the interface between the two V_{H} domains (see, for example, U.S. Application Pub. No. US 2005/0003347). With its domain-exchanged structure 2G12 binds with high affinity to oligomannose residues on the surface of HIV. 2G12 binds to an α1→2 mannose epitope on the outer face of HIV gp120 antigen (Calarese et al. (2003) Science 300:2065-2071). The mannoproteins expressed on the cell wall surface of C. albicans and Candida tropicalis contain a repeating Manα1-6Man backbone from which α1-2-linked mannose linkages branch (see, e.g., Cummings et al., in Essentials of Glycobiology, 2^{nd} edition, Cold Springs Harbor (NY): Cold Springs Harbor Laboratory Press, (2009), Chapter 21). 2G12 crossreacts with these α1-2-linked mannose epitopes (Dunlop et al. AIDS (2008) 22(16):2214-2217 and Dunlop et al., (2010) Glycobiology, 20(7):812-823).

2G12 antibodies include the domain-exchanged human monoclonal IgG1 antibody produced from the hybridoma cell line CL2 (as described in U.S. Pat. No. 5,911,989; Buchacher et al., (1994) AIDS Research and Human Retroviruses, 10(4) 359-369; and Trkola et al., (1996) Journal of Virology, 70(2) 1100-1108). 2G12 also includes any synthetically, e.g. recombinantly, produced antibody having the identical sequence of amino acids, and any antibody fragment thereof having identical heavy and light chain variable region domains to the full-length antibody. Typically, antibody fragments of 2G12 retain specific binding to the epitope(s) of the HIV gp120 antigen (e.g. as described in U.S. Pat. No. 5,911,989 and in U.S. Application Pub. No. US 2005/0003347). It is understood that 2G12 also can include other sequences that exhibit variation at the N- or C-terminus of the heavy or light chain sequence due to the addition, deletion or substitution of amino acids that results from cloning procedures and recombinant gene expression. Other variants of 2G12 exist or can be generated, so long as the antibody retains the domain-exchanged structure of 2G12 and retains binding characteristics of 2G12. Hence, it is understood that modified anti-Candida antibodies provided herein that are modified in their CDR, also can include other variations described herein or known to one of skill in the art. These include, for example, a 2G12 having a replacement of V5L and H230S in the heavy chain sequence (SEQ ID NO:167; see e.g. West et al. (2009) J. Virol., 83:98-104).

2G12 has a sequence of amino acids that includes at least the variable heavy and light chain of 2G12. For example, 2G12 antibodies have a sequence of amino acids for the V_{H} domain set forth in SEQ ID NO: 154; (VQLVESGGGLVKAGGSLILSCGVSNFRISAHTMNWVRRVPGGGLEWVASIST SSTYRDYADAVKGRFTVSRDDLEDFVYLQMHKMRVEDTAIYYCARKGSDRL SDNDPFDAWGPGTVVTVSP), and have a sequence of amino acids for the V_{L} domain set forth in SEQ ID NO:155; DVVMTQSPSTLSASVGDTITITCRASQSIETWLAWYQQKPGKAPKLLIYKAST LKTGVPSRFSGSGSGTEFTLTISGLQFDDFATYHCQHYAGYSATFGQGTRVEI K). As described in the examples herein, a 2G12 sequence based on the native light chain sequence VκI has a sequence of amino acids set forth in SEQ ID NO:162. As noted above, it is understood that 2G12 can include addition, deletion or substitution of amino acids that results from cloning procedures and recombinant gene expression. For example, the light chain of 2G12 pCAL IT* vector has an engineered SgrAI site for the cloning purpose encoding Ala Gly followed by 2 Val. Thus, the variable light chain sequence of 2G12 can include sequence of amino acids set forth in SEQ ID NO: 176, (AGVVMTQSPSTLSASVGDTITITCRASQSIETWLAWYQQKPGKAPKWYKA STLKTGVPSRFSGSGSGTEFTLTISGLQFDDFATYHCQHYAGYSATFGQGTRV EIK).

2G12 can exist as a full-length antibody. For example, 2G12 produced from the hybridoma cell line CL2 contains a kappa light C_{L} domain (set forth as amino acids 108-214 in SEQ ID NO:162) and an IgG1 C_{H}1 constant domain (SEQ ID NO: 284; set forth as amino acids 124-226 in SEQ ID NO:161). A full-length 2G12 that has a sequence of amino acids identical to that produced by the CL2 hybridoma cell line has a heavy chain amino acid sequence set forth in SEQ ID NO:160. With respect to SEQ ID NO: 160, the FR1 corresponds to amino acids 1-30; the CDR1 corresponds to amino acids 31-35; the FR2 corresponds to amino acids 36-49; the CDR2 corresponds to amino acids 50-66; the FR3 corresponds to amino acids 67-98; the CDR3 corresponds to amino acids 99-112; the FR4 corresponds to amino acids 113-123; the C_{H1} corresponds to amino acids 124-226; the hinge amino acids correspond to amino acids 227-242; and the C_{H}2-C_{H}3 amino acids correspond to amino acids 243-453. 2G12 has a light chain amino acid sequence set forth in SEQ ID NO:162. With respect to SEQ ID NO:162, the FR1 corresponds to amino acids 1-23; the CDR1 corresponds to amino acids 24-34; the FR2 corresponds to amino acids 35-49; the CDR2 corresponds to amino acids 50-56; the FR3 corresponds to amino acids 57-88; the CDR3 corresponds to amino acids 89-97; the FR4 corresponds to amino acids 98-107; and the C_{L} corresponds to amino acids 108-214. As noted, variation in the 2G12 sequence set forth in SEQ ID NO:160 can exist, for example, due to cloning procedures. Hence, in one example as described in Example 7 herein, a heavy chain sequence of 2G12 can have a sequence of amino acids set forth in SEQ ID NO:210.

2G12 also includes antibodies that include other constant domains and/or portions of constant domains. For example, a heavy chain amino acid sequence can include one or more of a C_{H}1, C_{H}2 or C_{H}3 from an IgG1. A 2G12 antibody also can be generated to contain a C_{H}1, C_{H}2, or C_{H}3 from an IgA, IgD, IgE, IgG or IgM class. If the antibody is of an IgE or IgM class it further can contain a C_{H}4. The heavy chain amino acid sequence of 2G12 also can further include a hinge region.

As a full-length antibody 2G12 exists in both monomeric and dimeric form. Mutations can be made in 2G12 that increases the 2G12 dimer/monomer ratio; dimers can be separately purified therefrom (see e.g. West et al. (2009) J. Virol., 83:98-104). Such dimers can exhibit increased potency and antigen-binding affinity. Exemplary of such mutations include hinge deletion mutants, including but not limited to, mutations corresponding to mutations in 2G12 heavy chain sequence set forth in SEQ ID NO:160 that include deletion of residue 230; deletion of residues 229 to 230; deletion of residues 228 to 230; deletion of residues 227 to 230; deletion of residues 227 to 232; and deletion of residues 227 to 232 and two proline to glycine substitutions at amino acid positions P233G and P234G. Such exemplary 2G12 mutants are set forth in SEQ ID NO:168-174. It is understood that any of the antibodies provided herein can further contain such mutations in the antibody to increase dimer formation of a full-length 2G12 antibody.

2G12 antibodies also include antibody fragments. For example, a 2G12 domain-exchanged Fab fragment (see, for example, U.S. Application Publication No. US20050003347 and Calarese et al., (2003) Science, 300, 2065-2071), contains a heavy chain (V_{H}-C_{H}1) having the sequence of amino acids set forth in SEQ ID NO: 161 (EVQLVESGGGLVKAGGSLILSCGVSNFRISAHTMNWVRRVPGGGLEWVASIS TSSTYRDYADAVKGRFTVSRDDLEDFVYLQMHKMRVEDTAIYYCARKGSDR LSDNDPFDAWGPGTVVTVSPASTKGPSVFPLAPSSKSTSGGTAALGCLVKDYF PEPVTVSWNSGALTSGVHTFPAVLQSSGLYSLSSVVTVPSSSLGTQTYICNVN HKPSNTKVDKKVEPKS); and a light chain (V_{L}-C_{L}) having the sequence of amino acids set forth in SEQ ID NO: 162 (DVVMTQSPSTLSASVGDTITITCRASQSIETWLAWYQQKPGKAPKWYKAST LKTGVPSRFSGSGSGTEFTLTISGLQFDDFATYHCQHYAGYSATFGQGTRVEI KRTVAAPSVFIFPPSDEQLKSGTASVVCLLNNFYPREAKVQWKVDNALQSGN SQESVTEQDSKDSTYSLSSTLTLSKADYEKHKVYACEVTHQGLSSPVTKSFNR GEC). Also, as noted above, 2G12 can include addition, deletion or substitution of amino acids that results from cloning procedures and recombinant gene expression. For example, a heavy chain Fab sequence can have a sequence of amino acids set forth in SEQ ID NO:10. In another example, a light chain Fab sequence can have a sequence of amino acids set forth in SEQ ID NO:11.

It is understood that other 2G12 domain-exchanged antibody fragments can be generated, for example, as described in Section F herein below. Any of the anti-Candida antibodies provided herein, for example 2G12 variants, can be generated as full-length antibodies or as antibodies that are less then full-length, for example, as a domain-exchanged antibody fragment, including, but not limited to, domain-exchanged Fab fragments, domain-exchanged scFv fragments, domain-exchanged Fab hinge fragments, domain-exchanged scFv tandem fragments, domain-exchanged scFv tandem fragments, and domain-exchanged single chain Fab fragments.

### C. ANTI-CANDIDA ANTIBODIES

Provided are anti-Candida antibodies, in particular anti-Candida domain-exchanged antibodies, that bind to one or more members of the genus Candida, but that is not the 2G12 domain-exchanged antibody. The anti-Candida antibodies provided herein exhibit a binding affinity for an epitope presented on the cell wall of a Candida pathogen or cell that is less than 100 nM. The anti-Candida antibodies provided herein recognize one or more cell wall antigens expressed on the surface of the yeast. In particular, the antibodies provided herein bind to carbohydrate moieties (mannose) present in the cell wall. The anti-Candida antibodies provided herein include those that can bind to the surface of Candida species, such as C. albicans, C. tropicalis, C. krusei and/or C. glabrata. For example, provided herein are anti-Candida antibodies that bind to carbohydrate moieties expressed on the surface of Candida, such as oligomannose moieties present on mannoproteins of the Candida cell wall.

In some examples, the anti-Candida antibodies provided herein exhibit an affinity for Candida that is less than 100 nM, and typically less than 10 nM, such as less than 1 nM, for example, 0.1 nM to 10 nM, 0.1 nM to 1 nM. For example, the anti-Candida domain-exchanged antibodies provided herein exhibit an affinity for a Candida, for example C. albicans, of about or 0.1 nM, about or 0.5 nM, about or 1 nM, about or 5 nM, about or 10 nM, about or 15 nM, about or 20 nM, about or 25 nM, about or 30 nM, about or 35 nM, about or 40 nM, about or 45 nM, about or 50 nM, about or 60 nM, about or 70 nM, about or 80 nM, about or 90 nM, about or 100 nM. It is understood that the binding affinity of an antibody can vary depending on the assay and conditions employed, although all assays for binding affinity provide a rough approximation. For example, as exemplified in the Examples, binding affinity can be assessed in an in vitro binding assay, such as a FACs or ELISA assay, which measure the binding of the antibody to the Candida yeast cell. By performing various assays under various conditions it is possible to estimate the binding affinity of an antibody. In addition, binding affinities can differ depending on the target source, such as the particular species of Candida, the source of Candida or the preparation of a Candida cell (e.g. fixed versus non-fixed cells). For example, the binding affinity of an anti-Candida domain-exchanged antibody for one species of Candida can differ from the binding affinity for a different species of Candida. The antibodies provided herein bind at least one species of Candida, and generally exhibit a binding affinity that less than 100 nM for at least one species of Candida in an in vitro binding affinity assay.

Further, binding affinities can differ depending on the structure of an antibody. Hence, a comparison of binding affinities between and among antibodies is to a corresponding form of the antibody.

In some examples, the anti-Candida antibodies provided herein bind to and inhibit one or more pathogenic activities of members of the genus Candida. Generally, the anti-Candida antibodies provided have the ability to inhibit or reduce one or more pathogenic activities of the yeast, such as, for example, inhibition of adhesion or germ tube formation, opsonization, neutralization of virulence-related enzyme or direct Candidacidal activity either alone or in combination with a therapeutic agent.

In some examples, an anti-Candida antibody provided herein inhibits the binding of Candida to a cell (e.g. an epithelial or endothelial cell) by at least or about 99%, at least or about 95%, at least or about 90%, at least or about 85%, at least or about 80%, at least or about 75%, at least or about 70%, at least or about 65%, at least or about 60%, at least or about 55%, at least or about 50%, at least or about 45%, at least or about 40%, at least or about 35%, at least or about 30%, at least or about 25%, at least or about 20%, at least or about 15%, or at least or about 10% relative to the binding of Candida to the cell in the absence of the anti-Candida antibody. In some examples, an anti-Candida antibody provided herein inhibits the Candida germ tube formation (i.e. germination) by at least or about 99%, at least or about 95%, at least or about 90%, at least or about 85%, at least or about 80%, at least or about 75%, at least or about 70%, at least or about 65%, at least or about 60%, at least or about 55%, at least or about 50%, at least or about 45%, at least or about 40%, at least or about 35%, at least or about 30%, at least or about 25%, at least or about 20%, at least or about 15%, or at least or about 10% relative to Candida germ tube formation in the absence of the anti-Candida antibody.

The antibodies provided can be employed for the prevention and/or spread of pathogenic disease, including, but not limited to the inhibition of transmission between subjects, inhibition of establishment of Candida infection in a host, and reduction of Candida colonization in a subject. The antibodies also can be employed for preventing, treating, and/or alleviating of one or more symptoms of a Candida infection or reduce the duration of a Candida infection. Accordingly, treatment of patients with antibodies provided can decrease the mortality and/or morbidity rate associated with Candida infection.

In some examples, the anti-Candida antibodies provided can be employed to direct therapeutic agents to sites of Candida infection for treatment. The anti-Candida antibodies provided also can be employed to increase the immune the response against a Candida infection.

Generally, the anti-Candida antibodies provided herein bind to Candida with increased affinity compared to the corresponding form of 2G12. The higher affinity of the anti-Candida antibodies provided herein permits the use of lower doses of the antibodies for the prevention and/or treatment of Candida infection. Lower doses of antibodies that immunospecifically bind to Candida reduces the likelihood of adverse effects of immunoglobulin therapy. In particular, provided herein are anti-Candida antibodies that bind to Candida or to an antigen or epitope thereof with higher binding affinity than the corresponding form of antibody 2G12 binds to the same strain or species of Candida or an antigen or epitope thereof. Accordingly, the antibodies provided herein exhibit increased affinity for C. albicans and other Candida species compared to 2G12.

Among the antibodies provided herein are domain-exchanged anti-Candida antibodies that are variants of 2G12. Hence, the anti-Candida antibodies provided herein are derived from modification of the anti-gp120 antibody, 2G12, which also binds to Candida species, such as Candida albicans. As described above, 2G12 is a domain-exchanged antibody (U.S. Application Pub. No. US2005/0003347) which can bind to oligomannose epitopes expressed on the surface of HIV gp120 and yeast, such as Candida and Saccharomyces. For example, amino acid modifications can occur in the a complementarity determining regions (CDRs). The modifications include amino acid replacement (substitution), deletion or insertion of one or more residues in a CDR of the V_{L} or V_{H} chain of 2G12. For example, one or more amino acid residues in CDRH1, CDRH2, CDRH3, CDRL1, CDRL2 and/or CDRL3 can be modified. Hence, anti-Candida antibodies provided herein include variant 2G12 antibodies that contain one or more modifications in the CDRH1, CDRH2, CDRH3, CDRL1, CDRL2 and/or CDRL3, and antibodies containing any one or more of the resulting modified CDRs. For example, also provided herein are other domain-exchanged antibodies that include any of the provided modified CDRs.

The anti-Candida antibodies provided herein can be isolated or identified using the methods described herein below in Section E and in U.S. Application Publication No. 2010/0093563. In one example, nucleic acid libraries encoding variant 2G12 antibodies can be generated and screened by phage display panning for specificity of binding to Candida, such as Candida albicans. Methods for generating such libraries are described herein in Section E. Assays for measuring the binding of the antibodies to yeast in vitro are described herein and are well-known in the art. Such assays can be used compare the binding affinity of a variant 2G12 antibody for Candida. For example, binding affinity can be assessed compared to the corresponding form of an unmodified 2G12.

The anti-Candida antibodies provided herein include full-length domain-exchanged antibodies or domain-exchanged antibody fragments, including, but not limited to, domain-exchanged Fab fragments, domain-exchanged scFv fragments, domain-exchanged Fab hinge fragments, domain-exchanged scFv tandem fragments, domain-exchanged scFv tandem fragments, and domain-exchanged single chain Fab fragments. Such fragments can be generated using standard recombinant methods and/or by enzymatic digestion as described in U.S. Provisional Application Nos. 61/192,960, 61/192,982 and U.S. Application Publication Nos. 2010/0093563 and 2010/0081575, and further herein in Section F. The V_{L} and V_{H} chains of the anti-Candida antibodies provided herein also can be used to generate full length IgG molecules as described elsewhere herein and in the Examples.

The anti-Candida antibodies provided herein can be further modified or prepared as conjugates or fusion proteins. For example, anti-Candida domain-exchanged antibodies provided herein can be further modified to increase half-life of the antibody. Hence, in some examples the anti-Candida antibodies provided herein have a half-life of 15 days or longer, 20 days or longer, 25 days or longer, 30 days or longer, 40 days or longer, 45 days or longer, 50 days or longer, 55 days or longer, 60 days or longer, 3 months or longer, 4 months or longer or 5 months or longer. Methods to increase the half-life of an antibody or antigen-binding fragment provided herein are known in the art. Such methods include for example, pegylation, glycosylation, and amino acid substitution as described elsewhere herein.

1. Variant 2G12 Anti-Candida Antibodies

Included among the anti-Candida antibodies provided herein are variant 2G12 antibodies that contain one or more modifications in the variable light chain (V_{L}) of 2G12. The variant or modified 2G12 antibodies provided herein can contain 1, 2, 3, 4, 5, 6, 7, 8, 9, 10, 11, 12, 13, 14, 15, 16, 17, 18, 19 or 20 amino acid modifications

(e.g. insertion, deletion or replacement of amino acids) in the V_{H} chain or V_{L} chain compared to 2G12. For example, amino acid modifications can occur in the a complementarity determining regions (CDRs). The modifications include amino acid replacement (substitution), deletion or insertion of one or more residues in a CDR of the V_{L} or V_{H} chain of 2G12. For example, one or more amino acid residues in CDRH1, CDRH2, CDRH3, CDRL1, CDRL2 and/or CDRL3 can be modified. Hence, anti-Candida antibodies provided herein include variant 2G12 antibodies that contain one or more modifications in the CDRH1, CDRH2, CDRH3, CDRL1, CDRL2 and/or CDRL3.

Exemplary of such modifications are variant 2G12 antibodies with one or more modifications in the 2G12 V_{L} CDR3. For example, the variant 2G12 anti-Candida antibodies provided herein can exhibit an increased binding affinity for Candida compared to the corresponding form of 2G12. For example, the one or more modifications can be at one or more amino acid positions in the 2G12 V_{L} CDR3, having the amino acid sequence QHYAGYSAT (SEQ ID NO: 2), which represents immunoglobulin amino acid positions L89-L97 in 2G12, according to Kabat numbering. The modifications can include one or more additions (insertions), deletions, or replacements (substitutions) of an amino acid at any position within the CDR3. For example, provided are domain-exchanged anti-Candida antibodies that contain one or more complementarity determining regions (CDRs) of 2G12 and a variable light chain (V_{L}) determining region 3 (CDR3) that is modified at one or more residues compared to the V_{L} CDR3 of 2G12. The modified antibodies include those that contain a CDRL3 that exhibit at least 30%, 40%, 50%, 60%, 65%, 70%, 75%, 80%, 85%, 90%, 95%, 96%, 97%, 98%, 99% sequence identity to a CDRL3 set forth in SEQ ID NO: 2.

In specific examples, the modification occurs at one, two, three, four, five, or six of amino acid positions L89, L90, L91, L92, L93, L94, L95, L96 and/or L97 (Gln, His, Tyr, Ala, Gly, Tyr, Ser, Ala, or Thr respectively, according to Kabat numbering). For example, modifications can be at one, two, three, four, five or six of amino acid positions L89, L90, L91, L92, L93, L94 and/or L95. For example, modifications include one, two, three, or all four of amino acid residues L89, L90, L91, L92. In another example, modifications include one, two, three or all four of amino acid residues L92, L93, L94 or L95. The modification(s) can include one or more additions, deletions, or replacements. Typically, modification includes an amino acid replacement or addition.

Exemplary amino acid replacements in the 2G12 V_{L} CDR3 provided herein include any set forth in Table 2B. Hence, anti-Candida antibodies provided herein include any variant 2G12 antibody that is modified in its V_{L} CDR3 by any of replacements at position L89 set forth in Table 2B, any of the replacements at position L90, any of the replacements at position L91, any of the replacements at position L92, any of the replacements at positions L93, any of the replacements at positions L94 and/or any of the replacements at positions L95. The resulting modified anti-Candida domain-exchanged antibodies exhibit a binding affinity for an epitope presented on Candida that is less than 100 nM. In some examples, the variant 2G12 antibody (or modified anti-Candida antibody) exhibits increased binding affinity for Candida compared to the corresponding form of unmodified 2G12.

TABLE 2B
CDRL3 amino acid replacements

| L89^{Gln} | L90^{His} | L91^{Tyr} | L92^{Ala} | L93^{Gly} | L94^{Tyr} | L95^{Ser} |
|---|---|---|---|---|---|---|
| Asn (N) | Pro (P) | Leu (L) | Lys (K) | Glu (E) | Trp (W) | Arg (R) |
| Ser (S) | Gly (G) | Ala (A) | Arg (R) | Ser (S) | Phe (F) | Trp (W) |
| Tyr (Y) | Gln (Q) | Ser (S) | Met (M) | Ala (A) | His (H) | Asn (N) |
| Gly (G) | Tyr (Y) | Gly (G) | Gln (Q) | Pro (P) | Ala (A) | His (H) |
| | Ser (S) | His (H) | Val (V) | Asp (D) | Ser (S) | Glu (E) |
| | | Trp (W) | Ser (S) | Asn (N) | Gly (G) | Gln (Q) |
| | | | Ile (I) | His (H) | Val (V) | Ala (A) |
| | | | Leu (L) | Glu (E) | Leu (L) | Lys (K) |
| | | | Glu (E) | Tyr (Y) | Thr (T) | Thr (T) |
| | | | His (H) | | Asn (N) | Pro (P) |
| | | | Thr (T) | | | Val (V) |
| | | | Tyr (Y) | | | Leu (L) |
| | | | Asp (D) | | | Gly (G) |
| | | | Asn (N) | | | Asp (D) |
| | | | Gly (G) | | | Tyr (Y) |
| | | | | | | Met (M) |
| | | | | | | Phe (F) |

In some examples, provided herein are modified 2G12 anti-Candida antibodies having 1, 2, 3 or all 4 of amino acid positions L92, L93, L94, or L95 in a 2G12, based on Kabat numbering, modified. In some examples, the A, G, Y, and S amino acid residues at positions L92, L93, L94, and L95, respectively, of the 2G12 light chain CDR3 are replaced. In some examples, the Ala residue at position L92 of the 2G12 V_{L} CDR3 is replaced with Lys, Arg, Met, Gln, Val, Ser, Ile, Leu, Glu or His. In some examples, the Gly residue at position L93 of the 2G12 V_{L} CDR3 is replaced with Glu, Pro, Ser, Ala or Asp. In some examples, the Tyr residue at position L94 of the 2G12 V_{L} CDR3 is replaced with Trp, Phe or His. In some examples, the Ser residue at position L95 of the 2G12 V_{L} CDR3 is replaced with Arg, Trp, Asn, Glu, Gly, Gln, His, Ala, Asp, Lys, Thr, Pro, Val, Leu or Met.

Exemplary modified V_{L} CDR3 provided herein include QHYKEWRAT (SEQ ID NO: 30), QHYKEWSAT (SEQ ID NO:31), QHYREWSAT (SEQ ID NO:32), QHYLAWSAT (SEQ ID NO:33), QHYKEWWAT (SEQ ID NO:34), QHYREWWAT (SEQ ID NO:35), QHYLSWSAT (SEQ ID NO:36), QHYKPFNAT (SEQ ID NO:37), QHYRPFNAT (SEQ ID NO:38), QHYMPFNAT (SEQ ID NO:39), QHYQPFNAT (SEQ ID NO:40), QHYLPFNAT (SEQ ID NO:41), QHYEPFNAT (SEQ ID NO:42), QHYKPFEAT (SEQ ID NO:43), QHYRPFEAT (SEQ ID NO:44), QHYKPFQAT (SEQ ID NO:45), QHYRPFQAT (SEQ ID NO:46), QHYQPFQAT (SEQ ID NO:47), QHYIPFQAT (SEQ ID NO:48), QHYKPFSAS (SEQ ID NO:49), QHYRPFSAT (SEQ ID NO:50), QHYQPFSAT (SEQ ID NO:51), QHYVPFSAT (SEQ ID NO:52), QHYHPFSAT (SEQ ID NO:53), QHYKPFHAT (SEQ ID NO:54), QHYRPFHAT (SEQ ID NO:55), QHYMPFHAT (SEQ ID NO:56), QHYEPFHAT (SEQ ID NO:57), QHYKPFRAT (SEQ ID NO:58), QHYVPFRAT (SEQ ID NO:59), QHYEPFRAT (SEQ ID NO:60), QHYKPFAAT (SEQ ID NO:61), QHYVPFAAT (SEQ ID NO:62), QHYIPFAAT (SEQ ID NO:63), QHYKPFDAT (SEQ ID NO:64), QHYMPFDAT (SEQ ID NO:65), QHYMPFKAT (SEQ ID NO:66), QHYMPFTAT (SEQ ID NO:67), QHYMPFPAT (SEQ ID NO:68), QHYQPFWAT (SEQ ID NO:69), QHYSPFWAT (SEQ ID NO:70), QHYMPYRAS (SEQ ID NO:71), QHYKPYRAT (SEQ ID NO:72), QHYMPYRAT (SEQ ID NO:73), QHYQPYRAT (SEQ ID NO:74), QHYLPYRAT (SEQ ID NO:75), QHYEPYRAT (SEQ ID NO:76), QHYKPYDAT (SEQ ID NO:77), QHYKPYSAT (SEQ ID NO:78), QHYQPYVAT (SEQ ID NO:79), QHYEPYKAT (SEQ ID NO:80), QHYLPYQAS (SEQ ID NO:81), QHYREWRAT (SEQ ID NO:218), QHYREFNAT (SEQ ID NO:219), QHYREFHAT (SEQ ID NO:220), QHYRPWSAT (SEQ ID NO:221), QHYMPFNAS (SEQ ID NO:222), QHYIPFEAT (SEQ ID NO:223), QHYLPFQAT (SEQ ID NO:224), QHYIPFSAT (SEQ ID NO:225), QHYQPFHAT (SEQ ID NO:226), QHYQPFRAT (SEQ ID NO:227), QHYVPFKAT (SEQ ID NO:228), QHYLPFKAT (SEQ ID NO:229), QHYKPFWAT (SEQ ID NO:230), QHYQPFVAT (SEQ ID NO:231), QHYRPWWAT (SEQ ID NO:232), QHYLPWWAT (SEQ ID NO:233), QHYMPYSAT (SEQ ID NO:234), QHYQPYSAT (SEQ ID NO:235), QHYEPYVAT (SEQ ID NO:236), QHYQPYKAS (SEQ ID NO:237), QHYMPYNAT (SEQ ID NO:238), QHYQPYNAT (SEQ ID NO:239), QHYLPYNAS (SEQ ID NO:240), QHYSPYWAT (SEQ ID NO:241), QHYLPYEAS (SEQ ID NO:242), QHYEPYLAT (SEQ ID NO:243), QHYLPFGAS (SEQ ID NO:244), QHYKDFSAT (SEQ ID NO:245), QHYMAYQAT (SEQ ID NO:246), QHYQAFNAT (SEQ ID NO:247), QHYKPFSAT (SEQ ID NO:280), QHYMPFRAT (SEQ ID NO:281), QHYKPFMAT (SEQ ID NO:678), QHYQPFDAT (SEQ ID NO:679), QHYREWHAT (SEQ ID NO:680), QHYLSYNAT (SEQ ID NO:681), QHYQPFKAT (SEQ ID NO:682) or QHYMAYDAT (SEQ ID NO:683). Also provided, are any modified CDR3 that exhibits at least 70%, 75%, 80%, 85%, 90%, 95% or more sequence identity to any of SEQ ID NOS: 30-81, 218-247, 280-281 or 678-683. Among the anti-Candida antibodies provided herein are anti-Candida domain-exchanged antibodies containing a V_{L} CDR3 selected from among QHYKEWRAT (SEQ ID NO: 30), QHYKEWSAT (SEQ ID NO:31), QHYREWSAT (SEQ ID NO:32), QHYLAWSAT (SEQ ID NO:33), QHYKEWWAT (SEQ ID NO:34), QHYREWWAT (SEQ ID NO:35), QHYLSWSAT (SEQ ID NO:36), QHYKPFNAT (SEQ ID NO:37), QHYRPFNAT (SEQ ID NO:38), QHYMPFNAT (SEQ ID NO:39), QHYQPFNAT (SEQ ID NO:40), QHYLPFNAT (SEQ ID NO:41), QHYEPFNAT (SEQ ID NO:42), QHYKPFEAT (SEQ ID NO:43), QHYRPFEAT (SEQ ID NO:44), QHYKPFQAT (SEQ ID NO:45), QHYRPFQAT (SEQ ID NO:46), QHYQPFQAT (SEQ ID NO:47), QHYIPFQAT (SEQ ID NO:48), QHYKPFSAS (SEQ ID NO:49), QHYRPFSAT (SEQ ID NO:50), QHYQPFSAT (SEQ ID NO:51), QHYVPFSAT (SEQ ID NO:52), QHYHPFSAT (SEQ ID NO:53), QHYKPFHAT (SEQ ID NO:54), QHYRPFHAT (SEQ ID NO:55), QHYMPFHAT (SEQ ID NO:56), QHYEPFHAT (SEQ ID NO:57), QHYKPFRAT (SEQ ID NO:58), QHYVPFRAT (SEQ ID NO:59), QHYEPFRAT (SEQ ID NO:60), QHYKPFAAT (SEQ ID NO:61), QHYVPFAAT (SEQ ID NO:62), QHYIPFAAT (SEQ ID NO:63), QHYKPFDAT (SEQ ID NO:64), QHYMPFDAT (SEQ ID NO:65), QHYMPFKAT (SEQ ID NO:66), QHYMPFTAT (SEQ ID NO:67), QHYMPFPAT (SEQ ID NO:68), QHYQPFWAT (SEQ ID NO:69), QHYSPFWAT (SEQ ID NO:70), QHYMPYRAS (SEQ ID NO:71), QHYKPYRAT (SEQ ID NO:72), QHYMPYRAT (SEQ ID NO:73), QHYQPYRAT (SEQ ID NO:74), QHYLPYRAT (SEQ ID NO:75), QHYEPYRAT (SEQ ID NO:76), QHYKPYDAT (SEQ ID NO:77), QHYKPYSAT (SEQ ID NO:78), QHYQPYVAT (SEQ ID NO:79), QHYEPYKAT (SEQ ID NO:80), or QHYLPYQAS (SEQ ID NO:81), QHYREWRAT (SEQ ID NO:218), QHYREFNAT (SEQ ID NO:219), QHYREFHAT (SEQ ID NO:220), QHYRPWSAT (SEQ ID NO:221), QHYMPFNAS (SEQ ID NO:222), QHYIPFEAT (SEQ ID NO:223), QHYLPFQAT (SEQ ID NO:224), QHYIPFSAT (SEQ ID NO:225), QHYQPFHAT (SEQ ID NO:226), QHYQPFRAT (SEQ ID NO:227), QHYVPFKAT (SEQ ID NO:228), QHYLPFKAT (SEQ ID NO:229), QHYKPFWAT (SEQ ID NO:230), QHYQPFVAT (SEQ ID NO:231), QHYRPWWAT (SEQ ID NO:232), QHYLPWWAT (SEQ ID NO:233), QHYMPYSAT (SEQ ID NO:234), QHYQPYSAT (SEQ ID NO:235), QHYEPYVAT (SEQ ID NO:236), QHYQPYKAS (SEQ ID NO:237), QHYMPYNAT (SEQ ID NO:238), QHYQPYNAT (SEQ ID NO:239), QHYLPYNAS (SEQ ID NO:240), QHYSPYWAT (SEQ ID NO:241), QHYLPYEAS (SEQ ID NO:242), QHYEPYLAT (SEQ ID NO:243), QHYLPFGAS (SEQ ID NO:244), QHYKDFSAT (SEQ ID NO:245), QHYMAYQAT (SEQ ID NO:246), QHYQAFNAT (SEQ ID NO:247) QHYKPFSAT (SEQ ID NO:280) or QHYMPFRAT (SEQ ID NO:281), QHYKPFMAT (SEQ ID NO:678), QHYQPFDAT (SEQ ID NO:679), QHYREWHAT (SEQ ID NO:680), QHYLSYNAT (SEQ ID NO:681), QHYQPFKAT (SEQ ID NO:682) or QHYMAYDAT (SEQ ID NO:683), or a CDR3 that exhibits at least 70%, 75%, 80%, 85%, 90%, 95% or more sequence identity to any of SEQ ID NOS: 30-81, 218-247, 280-281 or 678-683.

Modifications of amino acids also can include additions or insertions of amino acids into the light chain CDR3 set forth in SEQ ID NO: 2. For example, 1, 2, 3, or 4 amino acids can be inserted. Exemplary insertions provided herein include any that occur immediately before or immediately after any of amino acids L89, L90, L91, L92, L93, L94, L95, L96 and/or L97, based on Kabat numbering. The insertions can include insertion of any other amino acid. An exemplary insertion is the amino acid glycine (G). For example, as described herein below, modified 2G12 anti-Candida antibodies provided herein include a modified CDR3 containing insertion of the amino acid glycine after amino acid residue L95, based on Kabat numbering.

In some examples, provided herein are modified 2G12 anti-Candida antibodies having 1, 2, 3 or all 4 of amino acid positions L92, L93, L94, or L95 in a 2G12, based on Kabat numbering, modified, and one or more insertions of amino acids before or after any of L92, L93, L94, or L95. In some examples, the A, G, Y, and S amino acid residues at positions L92, L93, L94, and/or L95, respectively, of the 2G12 light chain CDR3 are replaced, and one or more amino acids is added after position L95. In some examples, the Ala residue at position L92 of the 2G12 V_{L} CDR3 is replaced with Arg or Thr. In some examples, the Gly residue at position L93 of the 2G12 V_{L} CDR3 is replaced with Pro, Ala or Asp. In some examples, the Tyr residue at position L94 of the 2G12 V_{L} CDR3 is replaced with His. In some examples, the Ser residue at position L95 of the 2G12 V_{L} CDR3 is replaced with Thr, Asp, Arg, His, Lys, Tyr, Asn, Phe or Ser. In some example, the amino acid Gly is added after position L95 of the 2G12 V_{L} CDR3.

Exemplary of such modified light chain CDR3 is QHYRPHTGAT (SEQ ID NO: 82), QHYTAHDGAT (SEQ ID NO:83), QHYTAHRGAT (SEQ ID NO:84), QHYRAHTGAT (SEQ ID NO:85), QHYTAHTGAT (SEQ ID NO:86), QHYTDHHGAT (SEQ ID NO:87), QHYTDHKGAT (SEQ ID NO:88), QHYTDHRGAT (SEQ ID NO:89), QHYTDHYGAT (SEQ ID NO:90), QHYTAHNGAT (SEQ ID NO:684), QHYTPHFGAT (SEQ ID NO:685) or QHYRAHSGAT (SEQ ID NO:686). Also provided, are any modified CDR3 that exhibits at least 70%, 75%, 80%, 85%, 90%, 95% or more sequence identity to any of SEQ ID NOS: 82-90 or 684-686. Among the anti-Candida antibodies provided herein are anti-Candida domain-exchanged antibodies containing a V_{L} CDR3 selected from among QHYRPHTGAT (SEQ ID NO: 82), QHYTAHDGAT (SEQ ID NO:83), QHYTAHRGAT (SEQ ID NO:84), QHYRAHTGAT (SEQ ID NO:85), QHYTAHTGAT (SEQ ID NO:86), QHYTDHHGAT (SEQ ID NO:87), QHYTDHKGAT (SEQ ID NO:88), QHYTDHRGAT (SEQ ID NO:89), QHYTDHYGAT (SEQ ID NO:90), QHYTAHNGAT (SEQ ID NO:684), QHYTPHFGAT (SEQ ID NO:685) or QHYRAHSGAT (SEQ ID NO:686), or a CDR3 that exhibits at least 70%, 75%, 80%, 85%, 90%, 95% or more sequence identity to any of SEQ ID NOS: 82-90 or 684-686.

In some examples, provided herein are modified 2G12 anti-Candida antibodies having 1, 2, 3 or all 4 of amino acid positions L89, L90, L91, or L92 in a 2G12, based on Kabat numbering, modified. Exemplary of a modified light chain CDR3 is NPLSGYSAT (SEQ ID NO:248). Also provided are any modified CDR3 that exhibits at least 70%, 75%, 80%, 85%, 90%, 95% or more sequence identity to SEQ ID NO: 248. Among the anti-Candida antibodies provided herein are anti-Candida domain-exchanged antibodies containing a V_{L} CDR3 that is NPLSGYSAT (SEQ ID NO:248), or a CDR3 that exhibits at least 70%, 75%, 80%, 85%, 90%, 95% or more sequence identity to SEQ ID NO: 248.

Hence, exemplary anti-Candida antibodies provided herein are modified 2G12 domain-exchanged antibodies that contain a V_{H} CDR1 having the amino acid sequence set forth in SEQ ID NO: 163, a V_{H} CDR2 having the amino acid sequence set forth in SEQ ID NO: 164, a V_{H} CDR3 having the amino acid sequence set forth in SEQ ID NO: 152, a V_{L} CDR1 having the amino acid sequence set forth in SEQ ID NO: 165, a V_{L} CDR2 having the amino acid sequence set forth in SEQ ID NO: 166, and a V_{L} CDR3 selected from among a V_{L} CDR3 having the amino acid sequence set forth in any of SEQ ID NOS: 30-90, 218-248, 280-281 or 678-686.

Exemplary anti-Candida variant light chains are set forth in Table 2C below, which indicates the sequence of the variant 2G12 CDRL3 and the SEQ ID NOS for each individual CDR and the SEQ ID NOS for corresponding variable light chains containing each variant CDRL3. The variant amino acids are indicated in boldface type. Provided herein are antibodies containing a light chain having a sequence of acids of any of the variant light chains set forth in Table 2C or a portion thereof, and a heavy chain of 2G12.

TABLE 2C
Anti-Candida variant light chains

| | | Light Chain | Light Chain |
|---|---|---|---|
| | CDRL3 | (AG) | (DVV) |
| SEQUENCE | SEQ ID NO | SEQ ID NO | SEQ ID NO |
| QHYKEWRAT | 30 | 91 | 457 |
| QHYKEWSAT | 31 | 92 | 458 |
| QHYREWSAT | 32 | 93 | 459 |
| QHYLAWSAT | 33 | 94 | 460 |
| QHYKEWWAT | 34 | 95 | 461 |
| QHYREWWAT | 35 | 96 | 462 |
| QHYLSWSAT | 36 | 97 | 463 |
| QHYKPFNAT | 37 | 98 | 464 |
| QHYRPFNAT | 38 | 99 | 465 |
| QHYMPFNAT | 39 | 100 | 466 |
| QHYQPFNAT | 40 | 101 | 467 |
| QHYLPFNAT | 41 | 102 | 468 |
| QHYEPFNAT | 42 | 103 | 469 |
| QHYKPFEAT | 43 | 104 | 470 |
| QHYRPFEAT | 44 | 105 | 471 |
| QHYKPFQAT | 45 | 106 | 472 |
| QHYRPFQAT | 46 | 107 | 473 |
| QHYQPFQAT | 47 | 108 | 474 |
| QHYIPFQAT | 48 | 109 | 475 |
| QHYKPFSAS | 49 | 110 | 476 |
| QHYRPFSAT | 50 | 111 | 477 |
| QHYQPFSAT | 51 | 112 | 478 |
| QHYVPFSAT | 52 | 113 | 479 |
| QHYHPFSAT | 53 | 114 | 480 |
| QHYKPFHAT | 54 | 115 | 481 |
| QHYRPFHAT | 55 | 116 | 482 |
| QHYMPFHAT | 56 | 117 | 483 |
| QHYEPFHAT | 57 | 118 | 484 |
| QHYKPFRAT | 58 | 119 | 485 |
| QHYVPFRAT | 59 | 120 | 486 |
| QHYEPFRAT | 60 | 121 | 487 |
| QHYKPFAAT | 61 | 122 | 488 |
| QHYVPFAAT | 62 | 123 | 489 |
| QHYIPFAAT | 63 | 124 | 490 |
| QHYKPFDAT | 64 | 125 | 491 |
| QHYMPFDAT | 65 | 126 | 492 |
| QHYMPFKAT | 66 | 127 | 493 |
| QHYMPFTAT | 67 | 128 | 494 |
| QHYMPFPAT | 68 | 129 | 495 |
| QHYQPFWAT | 69 | 130 | 496 |
| QHYQPFWAT | 70 | 131 | 497 |
| QHYMPYRAS | 71 | 132 | 498 |
| QHYKPYRAT | 72 | 133 | 499 |
| QHYMPYRAT | 73 | 134 | 500 |
| QHYQPYRAT | 74 | 135 | 501 |
| QHYLPYRAT | 75 | 136 | 502 |
| QHYEPYRAT | 76 | 137 | 503 |
| QHYKPYDAT | 77 | 138 | 504 |
| QHYKPYSAT | 78 | 139 | 505 |
| QHYQPYVAT | 79 | 140 | 506 |
| QHYEPYKAT | 80 | 141 | 507 |
| QHYLPYQAS | 81 | 142 | 508 |
| QHYRPHTGAT | 82 | 143 | 509 |
| QHYTAHDGAT | 83 | 144 | 510 |
| QHYTAHRGAT | 84 | 145 | 511 |
| QHYRAHTGAT | 85 | 146 | 512 |
| QHYTAHTGAT | 86 | 147 | 513 |
| QHYTDHHGAT | 87 | 148 | 514 |
| QHYTDHKGAT | 88 | 149 | 515 |
| QHYTDHRGAT | 89 | 150 | 516 |
| QHYTDHYGAT | 90 | 151 | 517 |
| QHYREWRAT | 218 | 249 | 518 |
| QHYREFNAT | 219 | 250 | 519 |
| QHYREFHAT | 220 | 251 | 520 |
| QHYRPWSAT | 221 | 252 | 521 |
| QHYMPFNAS | 222 | 253 | 522 |
| QHYIPFEAT | 223 | 254 | 523 |
| QHYLPFQAT | 224 | 255 | 524 |
| QHYIPFSAT | 225 | 256 | 525 |
| QHYQPFHAT | 226 | 257 | 526 |
| QHYQPFRAT | 227 | 258 | 527 |
| QHYVPFKAT | 228 | 259 | 528 |
| QHYLPFKAT | 229 | 260 | 529 |
| QHYKPFWAT | 230 | 261 | 530 |
| QHYQPFVAT | 231 | 262 | 531 |
| QHYRPWWAT | 232 | 263 | 532 |
| QHYLPWWAT | 233 | 264 | 533 |
| QHYMPYSAT | 234 | 265 | 534 |
| QHYQPYSAT | 235 | 266 | 535 |
| QHYEPYVAT | 236 | 267 | 536 |
| QHYMPYKAT | 237 | 268 | 537 |
| QHYMPYNAT | 238 | 269 | 538 |
| QHYQPYNAT | 239 | 270 | 539 |
| QHYLPYNAS | 240 | 271 | 540 |
| QHYSPYWAT | 241 | 272 | 541 |
| QHYLPYEAS | 242 | 273 | 542 |
| QHYEPYLAT | 243 | 274 | 543 |
| QHYLPFGAS | 244 | 275 | 544 |
| QHYKDFSAT | 245 | 276 | 545 |
| QHYMAYQAT | 246 | 277 | 546 |
| QHYQAFNAT | 247 | 278 | 547 |
| NPLSGYSAT | 248 | 279 | 548 |
| QHYKPFSAT | 280 | 282 | 549 |
| QHYMPFRAT | 281 | 283 | 550 |
| QHYKPFMAT | 678 | 687 | 696 |
| QHYQPFDAT | 679 | 688 | 697 |
| QHYREWHAT | 680 | 689 | 698 |
| QHYLSYNAT | 681 | 690 | 699 |
| QHYQPFKAT | 682 | 691 | 700 |
| QHYMAYDAT | 683 | 692 | 701 |
| QHYTAHNGAT | 684 | 693 | 702 |
| QHYTPHFGAT | 685 | 694 | 703 |
| QHYRAHSGAT | 686 | 695 | 704 |

For example, exemplary anti-Candida antibodies provided herein include variant 2G12 antibodies that at least contain a variant light chain having a sequence of amino acids set forth as amino acids 4-105 of any of SEQ ID NOS: 91-151, 249-279, 282-283 or 687-695. For example, exemplary anti-Candida antibodies provided herein include variant 2G12 antibodies that at least contain a variant light chain having a sequence of amino acids set forth as amino acid residues 1-108 of any of SEQ ID NOS: 91-142, 249-279, 282-283 or 687-692, as amino acid residues 1-109 of any of SEQ ID NOS: 143-151 or 693-695, as amino acid residues 1-107 of any of SEQ ID NOS: 457-508, 518-550 or 696-701, or as amino acid residues 1-108 of any of SEQ ID NOS: 509-517 or 702-704. Further, the antibody includes at least a 2G12 variable heavy chain set forth as amino acid residues 4-120 of SEQ ID NO: 154. For example, exemplary anti-Candida antibodies provided herein include variant 2G12 antibodies that contain a variable heavy chain having a sequence of amino acids set forth in SEQ ID NO: 154.

The variant 2G12 anti-Candida antibodies can be full-length antibodies or can be domain-exchanged antibody fragments thereof. Exemplary full-length variant 2G12 antibodies have a light chain that contains a sequence of amino acids set forth as at least amino acid residues 4-215 of SEQ ID NOS: 91-142, 249-279, 282-283 or 687-692, as amino acid residues 4-216 of SEQ ID NOS: 143-151 or 693-695, as amino acid residues 4-214 of SEQ ID NOS: 457-508, 518-550 or 696-701, or as amino acid residues 4-215 of SEQ ID NOS: 509-517 or 702-704; and a heavy chain that at least includes a sequence of amino acids set forth in SEQ ID NO:160, 209 or 210. For example, exemplary full-length variant 2G12 antibodies have a light chain that contains a sequence of amino acids set forth in any of SEQ ID NOS: 91-151, 249-279, 282-283, 457-550 or 687-704; and a heavy chain that contains a sequence of amino acids set forth in SEQ ID NO:160, 209 or 210.

Variant 2G12 anti-Candida domain-exchanged antibody fragments include domain-exchanged Fab fragments, domain-exchanged scFv fragments, domain-exchanged Fab hinge fragments, domain-exchanged scFv tandem fragments, domain-exchanged scFv tandem fragments, and domain-exchanged single chain Fab fragments. Exemplary variant 2G12 anti-Candida domain-exchanged Fab antibodies have a light chain that includes at least a sequence of amino acids set forth as at least amino acid residues 4-215 of SEQ ID NOS: 91-142, 249-279, 282-283 or 687-692, as amino acid residues 4-216 of SEQ ID NOS: 143-151 or 693-695, as amino acid residues 4-214 of SEQ ID NOS: 457-508, 518-550 or 696-701, or as amino acid residues 4-215 of SEQ ID NOS: 509-517 or 702-704; and a heavy chain that at least includes amino acid residues 4-225 of SEQ ID NO:10 or 161. For example, exemplary variant 2G12 anti-Candida domain-exchanged Fab fragments variant have a light chain that contains a sequence of amino acids set forth in any of SEQ ID NOS: 91-151, 249-279, 282-283, 457-550 or 687-704; and a heavy chain that contains a sequence of amino acids set forth in SEQ ID NO:161 or 10.

Exemplary anti-Candida antibodies provided herein are variant 2G12 domain-exchanged Fab antibodies that have a light chain set forth in SEQ ID NO:91, 102, 132, 143 or 145-151 and a heavy chain set forth in SEQ ID NO:10. Also provided herein are variant 2G12 full-length antibodies that have a light chain set forth in SEQ ID NO: 91, 102, 132, 143 or 145-151 and a heavy chain set forth in SEQ ID NO: 209 or 210.

Exemplary anti-Candida antibodies provided herein are variant 2G12 domain-exchanged Fab antibodies that have a light chain set forth in SEQ ID NOS:687-704 and a heavy chain set forth in SEQ ID NO:10. Also provided herein are variant 2G12-full-length antibodies that have a light chain set forth in SEQ ID NOS: 687-704 and a heavy chain set forth in SEQ ID NO: 160.

2. Other Anti-Candida Domain-Exchanged Antibodies

Provided herein are other domain-exchanged antibodies that include any of the provided CDRs above, including modified CDRs. The anti-Candida domain-exchanged antibody retains the ability to immunospecifically bind to Candida, for example, with a binding affinity that is less than 100 nM. Further, in some examples, the domain-exchanged antibodies exhibit a higher binding affinity than the corresponding form of antibody 2G12 binds to the same strain or species of Candida or an antigen or epitope thereof.

Domain-exchanged antibodies also can be created from conventional antibodies (see e.g. U.S. Patent Publication No. 2005/0003347). U.S. Patent Publication No. 2005/0003347 describes the structure and properties of an exemplary domain-exchanged antibodies. Using such teachings, one of skill in the art can generate other domain-exchanged antibodies from the sequences of conventional antibodies by incorporating these structural attributes into the conventional antibody. As discussed herein above, a domain-exchanged antibody generally contains at least one and up to all of amino acids isoleucine (Ile) at position 19, arginine (Arg) at position 57, phenylalanine (Phe) at position 77 and any amino acid residue at position 113 capable of forming a hydrophobic interaction with H84, for example, proline (Pro) or serine (Ser), where numbering is based on Kabat numbering. For example, a domain-exchanged antibody can be generated by introducing mutations into the conventional antibody at positions corresponding to amino acid positions 19, 57, 77 and 113 (based on Kabat numbering) of the heavy chain, to form and stabilization of the V_{H}-V_{H} interface. Further, position 38 of the light chain and position 39 of the heavy chain, which typically are conserved glutamine residues in conventional antibodies, can be modified to weaken the V_{H} and V_{L} interface. This can be desirable for the formation of domain-exchanged antibodies. Other amino acid positions that can be modified, such as by amino acid replacement, in a conventional antibody to generate a domain-exchanged antibody include, but are not limited to, amino acid positions 70, 72, 79, 81 and 84 of the heavy chain, according to Kabat numbering. In another example, a domain-exchanged antibody generally contains at least one and up to all of amino acids isoleucine (Ile) at position 19, arginine (Arg) at position 57, glutamic acid (Glu) at position 75, residue arginine (Arg) at position 39, alanine (Ala) at position 14, valine (Val) at position 84 and proline (Pro) at position 113, where numbering is based on Kabat numbering (see e.g. Huber et al., (2010) J. Virol August 11 (Epub)).

Accordingly, anti-Candida domain-exchanged antibodies can be generated by modifying domain-exchanged antibodies generated by the above methods of modifying the heavy chain. For example, a domain-exchanged antibody that is produced by modification of the heavy chain can be further modified by replacement of the V_{H} and V_{L} CDRs of the antibody with the corresponding V_{H} and V_{L} CDRs of any of the variant 2G12 anti-Candida antibodies provided. In another example, the V_{H} and V_{L} CDRs of a conventional antibody can first be replaced with the V_{H} and V_{L} CDRs of any of the variant 2G12 anti-Candida antibodies provided and then the antibody can be subsequently modified to adopt the domain-exchanged structure. For example, domain-exchanged antibodies provided herein include a V_{L} CDR3 having the amino acid sequence set forth in any of SEQ ID NOS: 30-90, 218-248, 280-281 or 678-686.

Exemplary anti-Candida antibodies provided herein are domain-exchanged antibodies that contain a V_{H} CDR1 having the amino acid sequence set forth in SEQ ID NO: 163, a V_{H} CDR2 having the amino acid sequence set forth in SEQ ID NO: 164, a V_{H} CDR3 having the amino acid sequence set forth in SEQ ID NO: 152, a V_{L} CDR1 having the amino acid sequence set forth in SEQ ID NO: 165, a V_{L} CDR2 having the amino acid sequence set forth in SEQ ID NO: 166, and a V_{L} CDR3 selected from among a V_{L} CDR3 having the amino acid sequence set forth in any of SEQ ID NOS: 30-90, 218-248, 280-281 or 678-686.

Exemplary anti-Candida antibodies provided herein are domain-exchanged antibodies that contain a V_{H} CDR1 having the amino acid sequence set forth in SEQ ID NO: 163 and a V_{L} CDR3 selected from among a V_{L} CDR3 having the amino acid sequence set forth in any of SEQ ID NOS: 30-90, 218-248, 280-281 or 678-686. Exemplary anti-Candida antibodies provided herein are domain-exchanged antibodies that contain a V_{H} CDR2 having the amino acid sequence set forth in SEQ ID NO: 164 and a V_{L} CDR3 selected from among a V_{L} CDR3 having the amino acid sequence set forth in any of SEQ ID NOS: 30-90, 218-248, 280-281 or 678-686. Exemplary anti-Candida antibodies provided herein are domain-exchanged antibodies that contain a V_{H} CDR3 having the amino acid sequence set forth in SEQ ID NO: 152 and a V_{L} CDR3 selected from among a V_{L} CDR3 having the amino acid sequence set forth in any of SEQ ID NOS: 30-90, 218-248, 280-281 or 678-686. Exemplary anti-Candida antibodies provided herein are domain-exchanged antibodies that contain a V_{L} CDR1 having the amino acid sequence set forth in SEQ ID NO: 165 and a V_{L} CDR3 selected from among a V_{L} CDR3 having the amino acid sequence set forth in any of SEQ ID NOS: 30-90, 218-248, 280-281 or 678-686. Exemplary anti-Candida antibodies provided herein are domain-exchanged antibodies that contain a V_{L} CDR2 having the amino acid sequence set forth in SEQ ID NO: 166 and a V_{L} CDR3 selected from among a V_{L} CDR3 having the amino acid sequence set forth in any of SEQ ID NOS: 30-90, 218-248, 280-281 or 678-686.

Exemplary anti-Candida antibodies provided herein are domain-exchanged antibodies that contain a V_{H} CDR1 having the amino acid sequence set forth in SEQ ID NO: 163, a V_{H} CDR2 having the amino acid sequence set forth in SEQ ID NO: 164, a V_{H} CDR3 having the amino acid sequence set forth in SEQ ID NO: 152, and a V_{L} CDR3 selected from among a V_{L} CDR3 having the amino acid sequence set forth in any of SEQ ID NOS: 30-90, 218-248, 280-281 or 678-686.

Exemplary anti-Candida antibodies provided herein are domain-exchanged antibodies that contain a V_{L} CDR1 having the amino acid sequence set forth in SEQ ID NO: 165, a V_{L} CDR2 having the amino acid sequence set forth in SEQ ID NO: 166, and a V_{L} CDR3 selected from among a V_{L} CDR3 having the amino acid sequence set forth in any of SEQ ID NOS: 30-90, 218-248, 280-281 or 678-686.

### D. ADDITIONAL MODIFICATIONS OF ANTI-CANDIDA ANTIBODIES

The anti-Candida antibodies provided herein can be further modified. Modifications of an anti-Candida antibody can improve one or more properties of the antibody, including, but not limited to, decreasing the immunogenicity of the antibody, improving the half-life of the antibody, such as reducing the susceptibility to proteolysis and/or reducing susceptibility to oxidation, and altering or improving of the binding properties of the antibody. Exemplary modifications include, but are not limited to, modifications of the primary amino acid sequence of the anti-Candida antibody and alteration of the post-translational modification of the anti-Candida antibody. Exemplary post-translational modifications include, for example, glycosylation, acetylation, pegylation, phosphorylation, amidation, derivatization with protecting/blocking group, proteolytic cleavage, and/or linkage to a cellular ligand or other protein. Other exemplary modifications include attachment of one or more heterologous peptides to the anti-Candida antibody to alter or improve one or more properties of the antibody and/or to facilitate purification.

Generally, the modifications do not result in increased immunogenicity of the antibody or antigen-binding fragment thereof or significantly negatively affect the binding of the antibody to Candida. Methods of assessing the binding of the modified antibodies to Candida are provided herein and known in the art and described elsewhere herein. For example, modified antibodies can be assayed for binding to Candida by methods such as, but not limited to, ELISA or FACS binding assays.

Modification of the anti-Candida antibodies produced herein can include one or more amino acid substitutions, deletions or additions, either from natural mutation or human manipulation from the parent antibody from which it was derived. Methods for modification of polypeptides, such as antibodies, are known in the art and can be employed for the modification of any antibody or antigen-binding fragment provided herein. Standard techniques known to those skill in the art can be used to introduce mutations in the nucleotide molecule encoding an antibody or an antigen-binding fragment provided herein in order to produce an polypeptide with one or more amino acid substitutions. Exemplary techniques for introducing mutations include, but are not limited to, site-directed mutagenesis and PCR-mediated mutagenesis.

1. Modifications to Reduce Immunogenicity

In some examples, the antibodies provided herein can be further modified to reduce the immunogenicity in a subject, such as a human subject. For example, one or more amino acids in the antibody can be modified to alter potential epitopes for human T-cells in order to eliminate or reduce the immunogenicity of the antibody when exposed to the immune system of the subject. Exemplary modifications include substitutions, deletions and insertion of one or more amino acids, which eliminate or reduce the immunogenicity of the antibody. Generally, such modifications do not alter the binding specificity of the antibody for its respective antigen. Reducing the immunogenicity of the antibody can improve one or more properties of the antibody, such as, for example, improving the therapeutic efficacy of the antibody and/or increasing the half-life of the antibody in vivo.

2. Fc Modifications

In some examples, the pharmacokinetic properties of the anti-Candida antibodies provided can be enhanced through Fc modifications by techniques known to those skilled in the art. The anti-Candida antibodies provided herein can contain an IgG1 constant domain (e.g. set forth in SEQ ID NO:160 or SEQ ID NO:210) or other immunoglobulin class or modified constant region thereof. For example, the constant region containing the Fc region can be modified to alter one or more properties of the Fc polypeptide. For example, the Fc region can be modified to alter (i.e. more or less) effector functions compared to the effector function of an Fc region of a wild-type immunoglobulin heavy chain. The Fc regions of an antibody interacts with a number of Fc receptors, and ligands, imparting an array of important functional capabilities referred to as effector functions. Fc effector functions include, for example, Fc receptor binding, complement fixation, and T cell depleting activity (see e.g., U.S. Pat. No. 6,136,310). Methods of assaying T cell depleting activity, Fc effector function, and antibody stability are known in the art. For example, the Fc region of an IgG molecule interacts with the FcγRs. These receptors are expressed in a variety of immune cells, including for example, monocytes, macrophages, neutrophils, dendritic cells, eosinophils, mast cells, platelets, B cells, large granular lymphocytes, Langerhans' cells, natural killer (NK) cells, and γδ T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. Recognition of and lysis of bound antibody on target cells by cytotoxic cells that express FcγRs is referred to as antibody dependent cell-mediated cytotoxicity (ADCC). Other Fc receptors for various antibody isotypes include FcεRs (IgE), FcαRs (IgA), and FcμRs (IgM).

Thus, a modified constant region containing modification in the Fc region can have altered affinity, including but not limited to, increased or low or no affinity for the Fc receptor. For example, the different IgG subclasses have different affinities for the FcγRs, with IgG1 and IgG3 typically binding substantially better to the receptors than IgG2 and IgG4. In addition, different FcγRs mediate different effector functions. FcγR1, FcγRIIa/c, and FcγRIIIa are positive regulators of immune complex triggered activation, characterized by having an intracellular domain that has an immunoreceptor tyrosine-based activation motif (ITAM). FcγRIIb, however, has an immunoreceptor tyrosine-based inhibition motif (ITIM) and is therefore inhibitory. Thus, altering the affinity of an Fc region for a receptor can modulate the effector functions induced by the Fc domain.

In one example, an Fc region is used that is modified for optimized binding to certain FcγRs to better mediate effector functions, such as for example, antibody-dependent cellular cytotoxicity, ADCC. Such modified Fc regions can contain modifications in an IgG at one or more of amino acid residues (according to the Kabat numbering scheme, Kabat et al. (1991) Sequences of Proteins of Immunological Interest, U.S. Department of Health and Human Services), including, but not limited to, amino acid positions 249, 252, 259, 262, 268, 271, 273, 277, 280, 281, 285, 287, 296, 300, 317, 323, 343, 345, 346, 349, 351, 352, 353, and 424. For example, modifications in an Fe region can be made corresponding to any one or more of G119S, G119A, S122D, S122E, S122N, S122Q, S122T, K129H, K129Y, D132Y, R138Y, E141Y, T143H, V1471, S150E, H151D, E155Y, E155I, E155H, K157E, G164D, E166L, E166H, S181A, S181D, S187T, S207G, S307I, K209T, K209E, K209D, A210D, A213Y, A213L, A2131,1215D, 1215E, 1215N, 1215Q, E216Y, E216A, K217T, K217F, K217A, and P279L of the exemplary IgG1 sequence set forth in SEQ ID NO:284, or combinations thereof. A modified Fc containing these mutations can have enhanced binding to an FcR such as, for example, the activating receptor FcγIIIa and/or can have reduced binding to the inhibitory receptor FcγRIIb (see e.g., OS 2006/0024298). Fc regions modified to have increased binding to FcRs can be more effective in facilitating the destruction of the fungal cells in patients.

In some examples, the antibodies or antigen-binding fragments provided herein can be further modified to improve the interaction of the antibody with the FcRn receptor in order to increase the in vivo half-life and pharmacokinetics of the antibody (see, e.g. U.S. Pat. No. 7,217,797, U.S. Pat. Pub. Nos. 2006/0198840 and 2008/0287657). FcRn is the neonatal FcR, the binding of which recycles endocytosed antibody from the endosomes back to the bloodstream. This process, coupled with preclusion of kidney filtration due to the large size of the full length molecule, results in favorable antibody serum half-lives ranging from one to three weeks. Binding of Fc to FcRn also plays a role in antibody transport.

Exemplary modifications of the Fc region include but are not limited to, mutation of the Fc described in U.S. Pat. No. 7,217,797; U.S. Pat. Pub. Nos. 2006/0198840, 2006/0024298 and 2008/0287657, and International Patent Pub. No. WO 2005/063816, such as mutations at one or more of amino acid residues (Kabat numbering, Kabat et al. (1991)) 251-256, 285-90, 308-314, in the C_{H}2 domain and/or amino acids residues 385-389 and 428-436 in the C_{H}3 domain of the IgG1 heavy chain constant region, where the modification alters Fc receptor binding affinity and/or serum half-life relative to unmodified antibody. In some examples, the IgG constant domain is modified in the Fc region at one or more of amino acid positions 250, 251, 252, 254, 255, 256, 263, 308, 309, 311, 312 and 314 in the C_{H}2 domain and/or amino acid positions 385, 386, 387, 389, 428, 433, 434, 436, and 459 in the C_{H}3 domain of the IgG heavy chain constant region. Such modifications correspond to amino acids Gly120, Pro121, Ser122, Phe124, Leu125, Phe126, Thr133, Pro174, Arg175, Glu177, Gln178, and Asn180 in the C_{H}2 domain and amino acids Gln245, Val246, Ser247, Thr249, Ser283, Gly285, Ser286, Phe288, and Met311 in the C_{H}3 domain in an exemplary IgG1 sequence set forth in SEQ ID NO:284. In some examples, the modification is at one or more surface-exposed residues, and the modification is a substitution with a residue of similar charge, polarity or hydrophobicity to the residue being substituted.

In particular examples, a IgG heavy chain constant region is modified in the Fc at one or more of amino acid positions 251, 252, 254, 255, and 256 (Kabat numbering), where position 251 is substituted with Leu or Arg, position 252 is substituted with Tyr, Phe, Ser, Trp or Thr, position 254 is substituted with Thr or Ser, position 255 is substituted with Leu, Gly, Ile or Arg, and/or position 256 is substituted with Ser, Arg, Gln, Glu, Asp, Ala, Asp or Thr. In some examples, a IgG heavy chain constant region is modified in the Fc at one or more of amino acid positions 308, 309, 311, 312, and 314, where position 308 is substituted with Thr or Ile, position 309 is substituted with Pro, position 311 is substituted with serine or Glu, position 312 is substituted with Asp, and/or position 314 is substituted with Leu. In some examples, a IgG heavy chain constant region is modified in the Fc at one or more of amino acid positions 428, 433, 434, and 436, where position 428 is substituted with Met, Thr, Leu, Phe, or Ser, position 433 is substituted with Lys, Arg, Ser, Ile, Pro, Gln, or His, position 434 is substituted with Phe, Tyr, or His, and/or position 436 is substituted with His, Asn, Asp, Thr, Lys, Met, or Thr. In some examples, a IgG heavy chain constant region is modified in the Fc at one or more of amino acid positions 263 and 459, where position 263 is substituted with Gln or Glu and/or position 459 is substituted with Leu or Phe.

In some examples, a Fc heavy chain constant region can be modified to enhance binding to the complement protein C1q. In addition to interacting with FcRs, Fc also interact with the complement protein C1q to mediate complement dependent cytotoxicity (CDC). C1q forms a complex with the serine proteases C1r and C1s to form the C1 complex. C1q is capable of binding six antibodies, although binding to two IgGs is sufficient to activate the complement cascade. Similar to Fc interaction with FcRs, different IgG subclasses have different affinity for C1q, with IgG1 and IgG3 typically binding substantially better than IgG2 and IgG4. Thus, a modified Fc having increased binding to C1q can mediate enhanced CDC, and can enhance destruction of fungal cells. Exemplary modifications in an Fc region that increase binding to C1q include, but are not limited to, amino acid modifications at positions 345 and 353 (Kabat numbering). Exemplary modifications include those corresponding to K209W, K209Y, and E216S in an exemplary IgG1 sequence set forth in SEQ ID NO:284.

In another example, a variety of Fc mutants with substitutions to reduce or ablate binding with FcγRs also are known. Such muteins are useful in instances where there is a need for reduced or eliminated effector function mediated by Fc. This is often the case where antagonism, but not killing of the cells bearing a target antigen is desired. Exemplary of such an Fc is an Fc mutein described in U.S. Pat. No. 5,457,035, which is modified at amino acid positions 248, 249 and 251 (Kabat numbering). In an exemplary IgG1 sequence set forth in SEQ ID NO:284, amino acid 117 is modified from Leu to Ala, amino acid 118 is modified from Leu to Glu, and amino acid 120 is modified from Gly to Ala. Similar mutations can be made in any Fc sequence such as, for example, the exemplary Fc sequence. This mutein exhibits reduced affinity for Fc receptors.

The antibodies provided herein can be engineered to contain modified Fc regions. For example, methods for fusing or conjugating polypeptides to the constant regions of antibodies (i.e. making Fc fusion proteins) are known in the art and described in, for example, U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al. (1991) Proc. Natl. Acad. Sci. USA 88:10535-10539; Traunecker et al. (1988) Nature 331:84-86; Zheng et al. (1995) J. Immunol. 154:5590-5600; and Vil et al. (1992) Proc. Natl. Acad. Sci. USA 89:11337-11341 (1992) and described elsewhere herein. In some examples, a modified Fc region having one or more modifications that increases the FcRn binding affinity and/or improves half-life can be fused to an anti-Candida antibody provided herein.

3. Pegylation

The anti-Candida antibodies provided herein can be conjugated to polymer molecules such as high molecular weight polyethylene glycol (PEG) to increase half-life and/or improve their pharmacokinetic profiles. Hence, provided herein are PEGylated anti-Candida domain-exchanged antibodies, for example, variant 2G12 antibodies. Conjugation can be carried out by techniques known to those skilled in the art. Conjugation of therapeutic antibodies with PEG has been shown to enhance pharmacodynamics while not interfering with function (see, e.g., Deckert et al., (2000) Int. J. Cancer 87: 382-390; Knight et al., (2004) Platelets 15: 409-418; Leong et al., (2001) Cytokine 16: 106-119; and Yang et al., (2003) Protein Eng. 16: 761-770). PEG can be attached to the antibodies or antigen-binding fragments with or without a multifunctional linker either through site-specific conjugation of the PEG to the N- or C-terminus of the antibodies or antigen-binding fragments or via epsilon-amino groups present on lysine residues. Linear or branched polymer derivatization that results in minimal loss of biological activity can be used. The degree of conjugation can be monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography. PEG-derivatized antibodies can be tested for binding activity to Candida antigens as well as for in vivo efficacy using methods known to those skilled in the art, for example, by immunoassays described herein.

4. Modification with Other Heterologous Peptides

In some examples, the antibodies can be recombinantly fused to a heterologous polypeptide at the N-terminus or C-terminus or chemically conjugated, including covalent and non-covalent conjugation, to a heterologous polypeptide or other composition. The anti-Candida antibodies provided herein can be modified, for example by the covalent attachment of any type of molecule, such as a diagnostic or therapeutic molecule, to the antibody such that covalent attachment does not prevent the antibody from binding to its corresponding epitope. For example, an anti-Candida antibody provided herein can be further modified by covalent attachment of a molecule such that the covalent attachment does not prevent the antibody from binding to Candida.

The anti-Candida antibodies provided herein can be modified by the attachment of a heterologous peptide to facilitate purification. Generally such peptides are expressed as a fusion protein containing the antibody fused to the peptide at the C- or N-terminus of the antibody. Exemplary peptides commonly used for purification include, but are not limited to, hexa-histidine peptides, hemagglutinin (HA) peptides, and flag tag peptides (see e.g., Wilson et al. (1984) Cell 37:767; Witzgall et al. (1994) Anal Biochem 223(2):291-298). The fusion does not necessarily need to be direct, but can occur through a linker peptide. In some examples, the linker peptide contains a protease cleavage site which allows for removal of the purification peptide following purification by cleavage with a protease that specifically recognizes the protease cleavage site.

The anti-Candida antibodies and fragments thereof provided herein also can be modified by the attachment of a heterologous polypeptide that targets the antibody or antigen-binding fragment to a particular cell type, either in vitro or in vivo. In some examples an anti-Candida antibody provided herein can be targeted to a particular cell type by fusing or conjugating the antibody to an antibody specific for a particular cell surface receptors or other polypeptide that interacts with a specific cell receptor.

The anti-Candida antibodies provided herein can be modified by the attachment of diagnostic and/or therapeutic moiety to the antibody. For example, the heterologous polypeptide or composition can be a diagnostic polypeptide or other diagnostic moiety or a therapeutic polypeptide or other therapeutic moiety. Exemplary diagnostic and therapeutic moieties include, but are not limited to, drugs, radionucleotides, toxins, fluorescent molecules (see, e.g. International PCT Publication Nos. WO 92/08495; WO 91/14438; WO 89/12624; U.S. Pat. No. 5,314,995; and EP 396,387).

Diagnostic polypeptides or diagnostic moieties can be used, for example, as labels for in vivo or in vitro detection. The detectable moieties can be employed, for example, in diagnostic methods for detecting exposure to Candida or localization of Candida or binding assays for determining the binding affinity of the anti-Candida antibody for Candida. The detectable moieties also can be employed in methods of preparation of the anti-Candida antibodies, such as, for example, purification of the antibody. The detectable moiety can be any material having a detectable physical or chemical property. Such detectable labels have been well-developed in the field of immunoassays and, in general, most any label useful in such methods can be applied in the methods provided. Thus, a label is any composition detectable by spectroscopic, photochemical, biochemical, immunochemical, electrical, optical or chemical means. Useful labels include, but are not limited to, fluorescent dyes (e.g., fluorescein isothiocyanate, Texas red, rhodamine, and the like), radiolabels (e.g., ³H, ¹²⁵I, ³⁵S, ¹⁴C, or ³²P), in particular, gamma and positron emitting radioisotopes (e.g., ¹⁵⁷Gd, ⁵⁵Mn, ¹⁶²Dy, ⁵²Cr, and ⁵⁶Fe), metallic ions (e.g., ¹¹¹In, ⁹⁷Ru, ⁶⁷Ga, ⁶⁸Ga, ⁷²As, ⁸⁹Zr, and ²⁰¹Tl), enzymes (e.g., horse radish peroxidase, alkaline phosphatase and others commonly used in an ELISA), electron transfer agents (e.g., including metal binding proteins and compounds); luminescent and chemiluminescent labels (e.g., luciferin and 2,3-dihydrophthalazinediones, e.g., luminol), magnetic beads (e.g., DYNABEADS™), and colorimetric labels such as colloidal gold or colored glass or plastic beads (e.g., polystyrene, polypropylene, latex, etc.). For a review of various labeling or signal producing systems that can be used, see e.g. U.S. Pat. No. 4,391,904.

Therapeutic polypeptides or therapeutic moieties can be used, for example, for therapy of a fungal infection, such as Candida infection, or for treatment of one or more symptoms of a fungal infection. Exemplary therapeutic moieties include, but are not limited to, a cytotoxin (e.g., a cytostatic or cytocidal agent), a therapeutic agent or a radioactive metal ion (e.g., alpha-emitters). Exemplary cytotoxin or cytotoxic agents include, but are not limited to, any agent that is detrimental to cells, such as, but not limited to, paclitaxel, cytochalasin B, gramicidin D, ethidium bromide, emetine, mitomycin, etoposide, teniposide, vincristine, vinblastine, colchicine, doxorubicin, daunorubicin, dihydroxyanthracine dione, mitoxantrone, mithramycin, actinomycin D, 1-dehydrotestosterone, glucocorticoids, procaine, tetracaine, lidocaine, propranolol, and puromycin and analogs or homologs thereof. Exemplary therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa, chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), and antifungal agents, such as, but not limited to, fluconazole, itraconazole, voriconazole, ketoconazole, miconazole, terconazole, clotrimazole, econazole, fenticonazole, sulconazole, tioconazole, isoconazole, omoconazole, oxiconazole, flutrimazole, butoconazole, amphotericin, nystatin, flucytosine, caspofungin, terbinafine, and gentian violet.

In some examples, the anti-Candida antibodies and antibody fragments provided herein can be further modified by conjugation to a therapeutic moiety that is a therapeutic polypeptide. Exemplary therapeutic polypeptides include, but are not limited to, a toxin, such as abrin, ricin A, Pseudomonas exotoxin, or diphtheria toxin; or an immunostimulatory agent, such as a cytokine, such as, but not limited to, an interferon (e.g., IFN-α, β, γ, ω), a lymphokine, a hematopoietic growth factor, such as, for example, GM-CSF (granulocyte macrophage colony stimulating factor), Interleukin-2 (IL-2), Interleukin-3 (IL-3), Interleukin-4 (IL-4), Interleukin-7 (IL-7), Interleukin-10 (IL-10), Interleukin-12 (IL-12), Interleukin-14 (IL-14), and Tumor Necrosis Factor (TNF).

The anti-Candida antibodies can also be attached to solid supports, which are useful for immunoassays or purification of the target antigen. Exemplary solid supports include, but are not limited to, glass, cellulose, polyacrylamide, nylon, polystyrene, polyvinyl chloride or polypropylene.

### E. METHODS OF IDENTIFYING ANTI-CANDIDA DOMAIN-EXCHANGED ANTIBODIES

The anti-Candida domain-exchanged antibodies provided herein can be identified by any method known to one of skill in the art used to identify antigen-specific antibodies. In one example, such methods include, for example, immunization and hybridoma screening methods. In another example, such methods include using combinatorial libraries of variant domain-exchanged antibodies or fragments thereof to screen for antibodies or fragments thereof for the desired activity or activities, for example, binding to Candida.

Exemplary of methods for identifying antibodies against particular pathogens, for example Candida, are screening assays of variants of existing domain-exchanged antibodies from combinatorial variant domain-exchanged antibody libraries. The libraries can be used to screen for antibodies that specifically bind to desired pathogens or cells, in particular viral, fungal or bacterial or other cellular pathogen that contains high density/repetitive epitopes such as carbohydrates and glycolipids. For example, the libraries can be used to screen for antibodies against Candida. Hence, methods of identifying anti-Candida antibodies, including any provided herein, include screening combinatorial variant libraries of 2G12 or a fragment thereof, for example, variant 2G12 Fab libraries. For example, the libraries or collections can be used to identify variant domain-exchanged antibodies, for example variant 2G12 antibodies, that exhibit increased affinity for Candida compared to the corresponding form of 2G12.

1. Domain-Exchanged Antibody Libraries

In generating the libraries, any domain-exchanged antibody can serve as the template for generating variant members of the libraries. As discussed elsewhere herein, one of skill in the art is able to identify and/or generate a domain-exchanged antibody based on structural and other properties. Exemplary of a domain-exchanged antibody is 2G12 or an antigen fragment thereof. Hence, 2G12 can be used as a scaffold in generating the antibodies or libraries. Therefore, provided herein are libraries or collections that contain a plurality of variant domain-exchanged antibodies or antibody fragments, in particular a plurality of variant 2G12 antibodies or antibody fragments thereof. Alternatively, any other domain-exchanged antibody can be used as the scaffold, template or starting antibody. As discussed elsewhere herein, a domain-exchanged antibody includes any antibody containing at least one or all of the amino acid residues isoleucine (Ile) at position 19, arginine (Arg) at position 57, phenylalanine (Phe) at position 77 and any amino acid residue at position 113 capable of forming a hydrophobic interaction with H84, for example, proline (Pro) or serine (Ser), where numbering is based on Kabat numbering. Further residues for amino acid mutation include amino acid residues 39, 70, 72, 79, 81 and 84 based on Kabat numbering. In particular, the mutations are arginine (Arg) at position 39, serine (Ser) at position 70, aspartic acid (Asp) at position 72, tyrosine (Tyr) at position 79, glutamine (Gln) at position 81 and valine (Val) at position 84, based on Kabat numbering. In another particular example, a domain-exchanged antibody includes any antibody containing at least one or all of amino acids isoleucine (Ile) at position 19, arginine (Arg) at position 57, glutamic acid (Glu) at position 75, residue arginine (Arg) at position 39, alanine (Ala) at position 14, valine (Val) at position 84 and proline (Pro) at position 113, where numbering is based on Kabat numbering (see e.g. Huber et al., (2010) J. Virol August 11 (Epub)). As discussed elsewhere herein, one of skill in the art is able to identify a domain-exchanged antibody based on structural and other properties, for example, oligomerization state.

In generating the libraries, variant antibodies can be generated by randomizing residues in the variable heavy chain and/or variable light chain of any of the above scaffold antibodies. Libraries containing a plurality of antibodies randomized or diversified at specified amino acid positions (e.g. mutated at all or a subset of the other 19 amino acids) can be generated. It is understood that the variant antibodies are designed such that they retain the domain-exchanged structure. For example, residues that are required for domain-exchange are not randomized in generating the libraries. Thus, for example, amino acid residues isoleucine (Ile) at position 19, arginine (Arg) at position 57, phenylalanine (Phe) at position 77 and proline (Pro) or serine (Ser) at position 113 are not changed in the libraries herein. In particular residue Arg at position 57 is not changed.

Exemplary template or scaffold antibodies for use in designing the antibodies and libraries herein do not bind to the target antigen that is to be selected against in the screening methods. For example, this ensures that when the libraries are created, the members of the library include minimal carryover of the backbone template vector. Where such carryover does exist, the template backbone vector is non-binding and will not be selected in screening or selection methods herein. For example, non-binding backbone vectors can include alanine mutations at amino acid residue positions associated with antigen-binding. Typically, one or more alanine mutations are included in the variable heavy chain and/or variable light chain that itself is being varied at other amino acid residues. For example, if residues in the variable heavy chain are being varied, than a non-binding scaffold used in the design of libraries herein includes alanine mutations at a heavy chain residue associated with antigen binding. In another example, if residues in the variable light chain are being varied, than a non-binding scaffold used in the design of libraries herein includes alanine mutations at a light chain residue associated with antigen binding. Typically, the alanine mutations are made directly in the CDR that is being randomized, including in the randomized residues. Exemplary of amino acid residues in 2G12 that can be replaced with an alanine for use as a non-binding backbone scaffold include, for example, light chain residues Tyr^{L91}, Tyr^{L94} and/or Ser^{L95} or heavy chain residues Leu^{L100}, Ser^{L100a} and/or Asn^{L100c}, each based on Kabat numbering. For example, exemplary of a non-binding backbone domain-exchanged antibody binding molecule is a 2G12 antibody or fragment thereof containing alanine mutations in the CDR H3 of the variable heavy chain (designated 3-ALA) at amino acid residues 104, 105 and 107 corresponding to amino acid residues in the V_{H} domain set forth in SEQ ID NO:156. Exemplary of a non-binding backbone domain-exchanged antibody binding molecule is a 2G12 antibody or fragment thereof containing alanine mutations in the CDR L3 of the variable light chain (designated 3-ALA LC) at amino acid residues 91, 94 and 95 corresponding to amino acid residues in the V_{H} domain set forth in SEQ ID NO:177. Additionally, amino acid residues 91, 94 and 95 of SEQ ID NO:177 correspond to amino acid residues 92, 95 and 96 of SEQ ID NO:157. Non-binding vectors also include vectors that contain the sequence of a domain-exchanged antibody that is identified as being non-binding. For example, clone F7 was identified in a screening assay not to bind to gp120. Table 2D lists exemplary 2G12 non-binding scaffold antibody vectors that can be used in the design of variant 2G12 libraries. It is within the level of one of skill in the art to design similar non-binding vectors for other variant domain-exchanged libraries. The non-binding backbone vectors do not bind gp120 or Candida antigen.

TABLE 2D
Non-Binding Vectors

| | SEQ | |
|---|---|---|
| | ID | |
| Backbone vector | NO | Amino acid change |
| 2G123Ala LC pCAL IT* | 23 | Y91A; Y94A; S95A |
| 2G12 SapI 3Ala LC | 552 | Y91A; Y94A; S95A |
| pCAL IT* | | |
| 2G12 3Ala LC pCAL | 196 | Y91A; Y94A; S95A |
| G13 | | |
| 2G12 DVV 3Ala LC | 338 | Y91A; Y94A; S95A |
| pCAL G13 | | |
| 2G12 3Ala pCAL IT* | 4 | L100A, S100aA, N100cA |
| 2G12 3Ala-1 pCAL | 195 | L100A, S100aA, N100cA |
| G13 | | |
| 2G12 3Ala pCAL IT* | 333 | L100A, S100aA, N100cA |
| SacI | | |
| Clone F7 | 419 | A31A; T33S; N35Y; K95G; |
| | | G96S; S97Y; R99G; L100Y; |
| | | S100aY; D100bA; D100dY |

The libraries or collections can be used to screen for or identify variant domain-exchanged antibodies, such as variant 2G12 antibodies, that exhibit increased binding affinity for a particular pathogen compared to the corresponding form of the domain-exchanged antibody not including the modification (e.g. wildtype). It is understood that the binding affinity of an antibody can vary depending on the assay and conditions employed, although all assays for binding affinity provide a rough approximation. By performing various assays under various conditions it is possible to estimate the binding affinity of an antibody. In addition, binding affinities can differ depending on the structure of an antibody. For example, binding affinities can differ between a domain-exchanged Fab fragment and a full-length domain-exchanged antibody. Thus, comparisons of binding affinities or specificities is generally between and among corresponding forms of an antibody. Finally, binding affinities and specificities can differ depending on the particular form of a antigen. Depending on the particular form of an antigen (e.g. whole cell recombinant antigen) binding affinities can vary by orders of magnitude. Accordingly, comparison of binding affinities or specificities is to the same form of antigen.

Typical of screening methods are high throughput screening of antibody libraries. Any known methods for generating libraries containing variant polynucleotides and/or polypeptides can be used. For example, any method described herein and/or known to one of skill in the art, for example, methods described in U.S. Provisional Application No. 61/192,916 and U.S. Publication No. 2010/0081575, can be used to generate domain-exchanged antibody libraries. The libraries can be used in screening assays to select variant domain-exchanged antibodies from the library for any antigen, including, for example, any Candida antigen described herein. To facilitate screening, antibody libraries typically are screened using a display technique, such that there is a physical link between the individual molecules of the library (phenotype) and the genetic information encoding them (genotype). These methods include, but are not limited to, cell display, including bacterial display, yeast display and mammalian display, phage display (Smith, G. P. (1985) Science 228:1315-1317), mRNA display, ribosome display and DNA display.

a. Variant Libraries

Variant domain-exchanged antibodies are generated herein such that each antibody or antibody member contains two variable heavy chains and two variable light chains, or a sufficient portion thereof to bind to antigen. Generally, domain-exchanged antibody libraries are generated from nucleic acid molecule(s) encoding two V_{H} chains and two V_{L} chains, whereby the V_{H} domains interact producing a V_{H}-V_{H}′ interface characteristic of the domain-exchanged configuration. The nucleic acid molecules can be generated separately, such that upon expression a domain-exchanged antibody is formed. For example, variant nucleic molecules can be generated encoding a V_{H} chain of a domain-exchanged antibody and/or variant nucleic acid molecules can be generated encoding a V_{L} chain of a domain-exchanged antibody. Upon co-expression of the nucleic acid molecules in a cell, a variant-domain-exchanged antibody is generated. Alternatively, a single nucleic acid molecule can be generated that encodes both the variant V_{H} and V_{L} chains of a domain-exchanged antibody. This is exemplified herein, for example, using a pCAL vector or variant or mutant thereof. In such a vector, a single nucleic acid molecule encodes both the heavy and light chain domains of a domain-exchanged antibody, for example, 2G12.

In any of the libraries herein, the nucleic acid molecules also can further contain nucleotides for the hinge region and/or constant regions (e.g. C_{L} or C_{H}1, C_{H}2 and/or C_{H}3) of the domain-exchanged antibody. Further, the nucleic acid molecules optionally can include nucleotides encoding peptide linkers. Methods to generate and express antibodies are described herein in Section F, and can be adapted for use in generating any domain-exchanged antibody library. Hence, the domain-exchanged antibody libraries can include members that are full-length antibodies, or that are antibody fragments thereof. Generally, domain-exchanged antibody libraries are Fab libraries. Further, it is understood that upon screening and selection of an antibody from the library, the selected member can be generated in any form, such as a full-length antibody or as an antibody fragment.

The libraries provided herein include a plurality of variant domain-exchanged antibodies. The libraries can be generated through diversification or randomization methods as described herein below. In one example, a domain-exchanged antibody library includes variant light chain libraries, whereby each member contains variant residues only in the light chain. In another example, a domain-exchanged antibody includes variant heavy chain libraries, whereby each member contains variant residues only in the heavy chain of the domain-exchanged antibody. In a further example, domain-exchanged antibody libraries include libraries where members include variant residues in both the heavy and light chains of the library. In all examples, the libraries of domain-exchanged antibodies are diverse, and contain least at or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, or more, different members. Typically, antibody libraries provided herein contain at least or about 10⁷, 10⁸, 10⁹, 10¹⁰, 10¹¹, 10¹², 10¹³, 10¹⁴, 10¹⁵, 10¹⁶, 10¹⁷, 10¹⁸, or more different members. In some examples, and as described further below, the libraries are designed so that the libraries do not contain members that have the sequence of a wild-type domain-exchanged antibody or that have a frequency of wild-type members that is less than 1%, for example, 0.1%, 0.01%, 0.001% or less. The libraries herein can be provided as display libraries, such as phage display libraries.

i. Selecting Residues

Libraries can be generated by diversification of amino acid residues in a scaffold domain-exchanged antibody, such as 2G12 or a non-binding 2G12. Hence, the libraries provided herein include members that have one or more amino acid variations compared to a scaffold domain-exchanged antibody. Generally, only the variable heavy chain and/or variable light chain of the antibody is diversified (e.g. varied) compared to the scaffold template. For example, any one or more residues in the CDR L1, L2, L3, H1, H2 and/or H3 can be varied to generate a plurality of antibodies that are mutated compared to the starting scaffold antibody. Diversification also can be effected in amino acid residues in the framework regions or hinge regions. For example, diversification of any one or more up to all residues in 2G12 can be effected, for example, amino acid residues in the CDR H1 (amino acid residues 31-35 of SEQ ID NO:154); CDR H2 (amino acid residues 50-66 of SEQ ID NO:154); CDR H3 (amino acid residues 99-112 of SEQ ID NO:154); CDRL1 (amino acid residues 24-34 of SEQ ID NO:155); CDR L2 (amino acid residues 50-56 of SEQ ID NO:155) and/or CDR L3 (amino acid residues 89-97 of SEQ ID NO:155). For example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more residues are selected for variation in the libraries herein. One of skill in the art knows and can identify the CDRs and FR based on Kabat or Chothia numbering (see e.g., Kabat, E. A. et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). As noted above, it is understood that residues involved in domain exchange as described elsewhere herein are not varied. Thus, for example, residue 57 in CDRH2 of 2G12 is not varied.

Exemplary of residues selected for diversification are those that are directly involved in antigen-binding. In one example, residues involved in antigen-binding can be identified empirically, for example, by mutagenesis experiments directly assessing binding to an antigen. In another example, residues involved in antigen-binding can be elucidated by analysis of crystal structures of the domain-exchanged binding molecule with the antigen or a related antigen or other antigen. For example, crystal structures of 2G12 complexed with various antigens can be used to elucidate and identify potential antigen-binding residues. It is contemplated that such residues may be involved in binding to diverse antigens, including Candida.

For example, based on crystal structure analysis of 2G12 binding to various antigens, exemplary antigen binding residues include, but are not limited to, L93 to L94 in CDR L3; H31, H32 and H33 in CDRH1; H52a in CDRH2; and H95, H96, H97, H98, H99, H100 in CDR H3, where residues are based on Kabat numbering (Calarese et al. (2005) Science, 300:2065). Other residues for diversification include L89, L90, L91, L92, L95, L96 and L97 in CDR L3; H35 in CDRH1; and H96, H100, H100a, H100b, H100c, H100d, H100e of CDRH3, based on Kabat numbering. Thus, exemplary residues for diversification and randomization herein include, but are not limited to, L89, L90, L91, L92, L93, L94, L95, L96, L97, H31, H32, H33, H35, H52a, H95, H96, H97, H98, H99, H100, H100a, H100b, H100c, H100d and/or H100e, based on Kabat numbering. In some examples, diversification of a CDR can include the addition of 1, 2, 3 or 4 amino acids within the CDR, which are similarly subjected to mutagenesis. For example, residues L95a and/or L95b can be added and subjected to mutagenesis as described herein in the Examples. Any one or more of the above residues can be randomized in designing the libraries herein. For example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more residues are randomized. Hence, 2G12 libraries provided herein are varied at any of the above residues. In some examples, 2G12 libraries include heavy chain libraries where only residues in the heavy chain are varied. In other examples, 2G12 libraries include light chain libraries where only residues in the light chain are varied. Other libraries provided herein include combination libraries where residues in the heavy and light chain are varied.

For examples, 2G12 variant libraries provided herein include 2G12 heavy chain variant libraries. In particular, an exemplary heavy chain library includes members having amino acid residues in the CDRH1 and/or CDRH3 varied. For example, an exemplary heavy chain library includes members having amino acid residues His^{H32}, Thr^{H33}, Gly^{H96}, Leu^{H100a}, Asn^{H100c} and Asp^{H100d}, based on Kabat numbering, diversified among members of the library. In another example, an exemplary heavy chain library includes members having amino acid residues Ala^{H31}, His^{H32}, Thr^{H33}, Asn^{H35}, Lys^{H95}, Gly^{H96}, Ser^{H97}, Asp^{H98}, Arg^{H99}, Leu^{H100}, Ser^{H100a}, Asp^{H1001}, Asn^{H100c}, Asp^{H100d}, Pro^{H100}e, based on Kabat numbering, diversified among members of the library. In a further example, an exemplary heavy chain library includes members having amino acid residues Lys^{H95}, Gly^{H96}, Asp^{H100b} and Asp^{H100d}, based on Kabat numbering, diversified among members of the library. In another example of a library, an exemplary heavy chain library includes members having amino acid residues His^{H32} and Thr^{H33}, based on Kabat numbering, diversified among members of the library. In other examples, an exemplary heavy chain library includes members having amino acid residues Gly^{H96}, Leu^{H100}, Ser^{H100a}, Asn^{H100c}, and Asp^{H100d}, based on Kabat numbering, diversified among members of the library. In an additional example, an exemplary heavy chain library includes members having amino acid residues Lys^{H95}, Gly^{H96}, Ser^{H97}, Asp^{H98}, Arg^{H99}, Leu^{H100}, Ser^{H100a}, Asp^{H100b}, Asn^{H100c} and Asp^{H100d}, based on Kabat numbering, diversified among members of the library. In a further example, an exemplary heavy chain library includes members having amino acid residues Ala^{H31}, His^{H32}, Thr^{H33}, Asn^{H35}, Lys^{H95}, Gly^{H96}, Ser^{H97}, Asp^{H98}, Arg^{H99}, Leu^{H100}, Ser^{H100a}, Asp^{H100b}, Asn^{H100c} and Asp^{H100d}, based on Kabat numbering, diversified among members of the library. In one example, an exemplary heavy chain library includes members having amino acid residues Ala^{H31}, His^{H32}, Thr^{H33}, Asn^{H35}, diversified among members of the library.

In other examples, 2G12 variant libraries provided herein include 2G12 light chain variant libraries. For example, an exemplary heavy chain library includes members having amino acid residues in the CDRL3 varied. In particular, an exemplary library includes a light chain library having amino acid residues Ala^{L92}, Gly^{L93}, Tyr^{L94} and Ser^{L95}, based on Kabat numbering, diversified among members of the library. In a further example, an exemplary library includes a light chain library having amino acid residues Gln^{L89}, His^{L90}, Tyr^{L91} and Ala^{L92}, based on Kabat numbering, diversified among members of the library. In another example, an exemplary library includes a light chain library having amino acid residues Tyr^{L91}, Ala^{L92}, Tyr^{L94}, Ser^{L95} and Ala^{L96}, based on Kabat numbering, diversified among members of the library. In a further example, an exemplary library includes a light chain library having amino acid residues Tyr^{L91}, Ala^{L92}, Ser^{L95} and Ala^{L96}, based on Kabat numbering, diversified among members of the library. In other examples, an exemplary library includes a light chain library having amino acid residues Gln^{L89}, His^{L90}, Tyr^{L91}, Ala^{L92}, Gly^{L93}, Tyr^{L94}, Ser^{L95}, and Ala^{L96}, based on Kabat numbering, diversified among members of the library. In a further example, an exemplary library includes a light chain library having amino acid residues His^{L90}, Tyr^{L91}, Ala^{L92}, Gly^{L93}, Tyr^{L94}, Ser^{L95}, and Ala^{L96}, based on Kabat numbering, diversified among members of the library.

In examples of light chain libraries provided herein, an exemplary library includes a light chain library having amino acid residues added into the CDRL1, for example, at residues 95a and/or 95b. For example, an exemplary library includes a light chain library having amino acid residues Ala^{L92}, Gly^{L93}, Tyr^{L94} and Ser^{L95}, and additionally amino acid residues L95a and/or L95b, based on Kabat numbering, diversified among members of the library. In another example, an exemplary library includes a light chain library having amino acid residues His^{L90}, Tyr^{L91}, Ala^{L92}, Gly^{L93}, Tyr^{L94}, Ser^{L95}, and additionally amino acid residues L95a and/or L95b, based on Kabat numbering, diversified among members of the library.

In additional examples, 2G12 libraries provided herein include 2G12 hybrid variant libraries that include combinations of variant heavy chain and variant light chain libraries. In particular, an exemplary combination library includes members having amino acid residues Gln^{L89}, His^{L90}, Tyr^{L91}, Ala^{L92}, Gly^{L93}, Tyr^{L94}, Ser^{L95} and Ala^{L96} in the light chain varied among members of the library and amino acid residues Ala^{H31}, His^{H32}, Thr^{H33}, Asn^{H35}, Lys^{H95}, Gly^{H96}, Ser^{H97}, Asp^{H98}, Arg^{H99}, Leu^{H100}, Ser^{H100a}, Asp^{H100b}, Asn^{H100c} Asp^{H100d} and Pro^{H100e} in the heavy chain varied among members of the library. In another example, an exemplary combination library includes members having amino acid residues Ala^{L92}, Gly^{L93}, Tyr^{L94} and Ser^{L95} in the light chain varied among members of the library and amino acid residues Ala^{H31}, His^{H32}, Thr^{H33}, Asn^{H35}, Lys^{H95}, Gly^{H96}, Ser^{H97}, Asp^{H98}, Arg^{H99}, Leu^{H100}, Ser^{H100a}, Asp^{H100b}, Asn^{H100c} and Asp^{H100d} in the heavy chain varied among members of the library. In a further example, an exemplary combination library includes members having amino acid residues Gln^{L89}, His^{L90}, Tyr^{L91}, Ala^{L92}, Gly^{L93}, Tyr^{L94}, Ser^{L95} and Ala^{L96} in the light chain varied among members of the library and amino acid residues Ala^{H31}, His^{H32}, Thr^{H33}, Asn^{H35}, Lys^{H95}, Gly^{H96}, Ser^{H97}, Asp^{H98}, Arg^{H99}, Leu^{H100}, Ser^{H100a}, Asp^{H100b}, Asn^{H100c} and Asp^{H100d} in the heavy chain varied among members of the library. In an additional example, an exemplary combination library includes members having amino acid residues Ala^{L92}, Gly^{L93}, Tyr^{L94} and Ser^{L95} in the light chain varied among members of the library and amino acid residues Ala^{H31}, His^{H32}, Thr^{H33}, Asn^{H35}, Lys^{H95}, Gly^{H96}, Ser^{H97}, Asp^{H98}, Arg^{H99}, Leu^{H100}, Ser^{H100a}, Asp^{H100b}, Asn^{H100c} Asp^{H100d} and Pro^{H100e} in the heavy chain varied among members of the library. In other examples, an exemplary combination library includes members having amino acid residues His¹⁹⁰, Tyr^{L91}, Ala^{L92}, Gly^{L93}, Tyr^{L94}, Ser^{L95} in the light chain varied among members of the library and amino acid residues Lys^{H95}, Gly^{H96}, Ser^{H97}, Asp^{H98}, Arg^{H99}, Leu^{H100}, Ser^{H100a}, Asp^{H100b}, Asn^{H100c} and Asp^{H100d} in the heavy chain varied among members of the library.

ii. Randomization and Diversification Methods

Methods for generating nucleic acid libraries and resulting antibody libraries and for creating diversity in the libraries are well known in the art and can be employed to generate nucleic acid libraries and resulting antibody libraries of domain-exchanged antibody variants that can be screened for binding to a desired pathogen or cell. For example, the libraries can be used to screen for binding to Candida. Approaches for generating diversity include targeted and non-targeted approaches well known in the art.

Known approaches for generating diverse nucleic acid and polypeptide libraries include, but are not limited to:

non-targeted approaches (whereby diversity is introduced at random) such as recombination approaches (e.g. chain shuffling, (Marks et al., (1991) J. Mol. Biol. 222:581-597; Barbas et al., (1991) Proc. Natl. Acad. Sci. USA 88:7978-7982; Lu et al., (2003) Journal of Biological Chemistry 278(44):43496-43507; Clackson et al., (1991) Nature 352:624-628; Barbas et al., (1992) Proc. Natl. Acad. Sci. USA 89:10164; U.S. Pat. Nos. 6,291,161, 6,291,160, 6,291,159, 6,680,192, 6,291,158, and 6,969,586); and “sexual PCR” (Stemmer, (1994) Nature 340:389-391; Stemmer, (1994) Proc. Natl. Acad. Sci. USA 10747-10751; and U.S. Pat. No. 6,576,467; Boder et al., (2000) PNAS 97(20):10701-10705); and error-prone PCR (Zhou et al., (1991) Nucleic Acids Research 19(21):6052; Gram et al. (1992) Proc. Natl. Acad. Sci. USA 89:3576-3580; Rice et al., (1992) Proc. Natl. Acad. Sci. USA 89:5467-5471; Fromant et al., (1995) Analytical Biochemistry 224(1):347-353; Mondon et al., (2007) Biotechnol. J. 2:76-82; U.S. Application Publication No. 2004/0110294; Low et al., (1996) J. Mol. Biol. 260(3):359-368; Orencia et al., (2001) Nature Structural Biology 8(3):238-242; and Coia et al., (2001) J Immunol Methods 251(1-2):187-193);

targeted approaches (for mutating particular positions or portions), such as cassette mutagenesis (Wells et al., (1985) Gene 34:315-323; Oliphant et al., (1986) Gene 44:177-183; Borrego et al., (1995) Nucleic Acids Research 23, 1834-1835; Baca et al., (1997) The Journal of Biological Chemistry 272(16):10678-10684; Breyer and Sauer (1989) Journal of Biological Chemistry 264(22):13355-13360; Oliphant and Strul (1989) Proc. Natl. Acad. Sci. USA 86:9094-9098; U.S. Pat. No. 7,175,996; Borrego et al., (1995) Nucleic Acids Research 23:1834-1835; and Wells et al., (1985) Gene 34:315-323); mutual primer extension (Oliphant et al., (1986) Gene 44:177-183; Bryer and Sauer (1989) Journal of Biological Chemistry 264(22):13355-13360; Oliphant and Strul (1989) Proc. Natl. Acad. Sci. USA 86:9094-9098); template-assisted ligation and extension (Baca et al., (1997) The Journal of Biological Chemistry 272(16):10678-10684); codon cassette mutagenesis (Kegler-Ebo et al., (1994) Nucleic Acids Research, 22(9):1593-1599; Kegler-Ebo et al., (1996) Methods Mol. Biol., 57:297-310); oligonucleotide-directed mutagenesis (Brady and Lo, (2004) Methods Mol. Biol. 248:319-26; Rosok et al., (1998) The Journal of Immunology, 160:2353-2359); and amplification using degenerate oligonucleotide primers (U.S. Pat. Nos. 5,545,142, 6,248,516, and 7,189,841; Barbas et al., (1992) Proc. Natl. Acad. Sci. USA 89:4557-4461; Pini et al., (1998) The Journal of Biological Chemistry 273(34):21769-21776; Ho et al., (2005) The Journal of Biological Chemistry 280(1):607-617), including overlap and two-step PCR (Higuchi et al., (1988) Nucleic Acids Research 16(15):7351-7367; Jang et al., (1998) Molecular Immunology 35:1207-1217; Brady and Lo, (2004) Methods Mol Biol 248:319-26; Burks et al., (1997) Proc. Natl. Acad. Sci. USA 94:412-417; Dubreuil et al., (2005) The Journal of Biological Chemistry 280(26):24880-24887); and

combined approaches, such as combinatorial multiple cassette mutagenesis (CMCM) and related techniques (Crameri and Stemmer, (1995) Biotechniques, 18(2):194-6; US2007/0077572; De Kruif et al., (1995) J. Mol. Biol. 248:97-105; Knappik et al., (2000) J. Mol. Biol. 296(1):57-86; and U.S. Pat. No. 6,096,551).

Exemplary of methods for generating diverse nucleic acid libraries, and resulting antibody libraries, include the design, synthesis and assembly of oligonucleotides, such as any method are described in related U.S. Provisional Application Nos. 61/192,916, 61/192,982 and U.S. Publication Nos. 2010/0081575 and 2010/0093563 and International PCT Application Publication Nos. WO2010/033237 and WO2010/033229.

(1) Design, Synthesis and Assembly of Randomized Oligonucleotides

Variant antibodies can be generated from designed and generated synthetic oligonucleotides, whereby diversity is generated at selected amino acid residues using various doping strategies, such as biased or non-biased doping strategies. For example, one or more oligonucleotides can be synthesized based on a reference template sequence of a domain-exchanged antibody, for example, 2G12 or a non-binding 2G12. Generally, the oligonucleotides are generated such that they can be assembled in subsequent steps to form assembled duplex cassettes. For example, an oligonucleotide corresponding to a selected CDR desired to be varied (e.g. CDRH1, CDRH3 or CDRL3) is randomized and duplexes generated. Oligonucleotide duplexes are also generated for the portions of the reference or template (e.g. wildtype) sequence that is not being randomized. All the pieces can be ligated together to generate the entire variant light chain or variant heavy chain sequence. Generated sequences can be ligated into a vector for expression of the domain-exchanged antibody as described below.

Biased and non-biased doping strategies can be used during synthesis of randomized portions in pools of randomized oligonucleotides. One of skill in the art is familiar with strategies to introduce such doping strategies. Exemplary of a non-biased doping strategy is “NNN,” one whereby each of the four nucleotide monomers (A, G, T and C) is added at an equal proportion during synthesis of each nucleotide position in a randomized portion. Exemplary of biased doping strategies include NNK, NNB and NNS, and NNW; NNM, NNH; NND; NNV doping strategies and an NNT, NNA, NNG and NNC doping strategy. For example, in an NNK doping strategy, randomized portions of positive strands are synthesized using an NNK pattern and negative strand portions are synthesized using an MNN pattern, where N is any nucleotide (for example, A, C, G or T), K is T or G and M is A or C. Thus, using this doping strategy, each nucleotide in the randomized portion of the positive strand is a T or G.

Other doping strategies include randomization strategies that function on an amino acid level rather than a nucleic acid level and involve the simultaneous randomization of an entire three nucleotide codon to another fixed three nucleotide codon. In such a strategy, the three nucleotides composing the residue targeted for randomization are substituted for a specified codon using trimer phosphoramidites for any up to all of the amino acids. In one example, YGS or YGS+ randomization, the three nucleotides composing the residue targeted for randomization are substituted for a specified codon, for example tyrosine (Y=TAC), glycine (G=GGT) or serine (S=TCT) (or any other amino acids). The trimer phosphoramidites can be added to an oligonucleotide during its standard chemical synthesis. In this manner, randomization of a given residue is restricted to a desired set of amino acid substitutions. Additionally, this doping strategy prevents the insertion of stop codons thereby eliminating sequence truncations.

The randomized oligonucleotides can be assembled using any method known to one of skill in the art, in particular any method described in U.S. Provisional Application No. 61/192,960 and U.S. Publication No. 2010/0081575 and International PCT Application Publication No. WO2010/033237. For example, following oligonucleotide synthesis, synthetic oligonucleotides and/or duplexes generated from the oligonucleotides are used to generate duplexes, including intermediate duplexes and assembled duplexes, including assembled duplex cassettes. Synthetic oligonucleotides and/or duplexes from two or more, typically three or more, pools are assembled to form assembled duplexes. In one example, the assembled duplexes are large assembled duplexes. The large assembled duplexes can be generated by hybridization, polymerase reactions, amplification reactions, ligation, and/or combinations thereof.

In one example, oligonucleotides are assembled using random cassette mutagenesis and assembly (RCMA), whereby the oligonucleotide duplexes are generated by hybridizing positive and negative strand synthetic oligonucleotides. In another example, assembled duplexes are formed using an oligonucleotide fill-in and assembly (OFIA), whereby synthetic template oligonucleotides and synthetic oligonucleotide primers are hybridized, followed by polymerase extension. In another approach, assembled duplexes can be generated by duplex oligonucleotide ligation/single primer amplification (DOLSPA), whereby a plurality of synthetic oligonucleotides are combined to assemble intermediate duplexes by hybridization and ligation, which are used in an amplification reaction to form assembled duplexes. In a further example, assembled duplexes can be generated by modified fragment assembly and ligation/single primer amplification (mFAL-SPA), whereby pools of variant duplexes are generated containing restriction site overhangs that are compatible with restriction site overhangs contained in pools of reference sequence duplex that also are generated. The pools of duplexes are combined in a fragment assembly and ligation (FAL) step to form pools of intermediate duplexes that are assembled from the compatible overhangs, which are amplified using a single primer amplification (SPA) reaction. The assembled duplexes can be ligated into vectors for expression of antibody members. Example 2 herein exemplifies generation of a CDR L3 light chain library of 2G12 variants by diversification of amino acid residues using the mFAL-SPA method.

In another example, duplexes can be generated by overlap extension reaction/single primer amplification (OER-SPA) whereby two or more PCR products, amplified using primers containing randomized nucleotides, can be joined together by polymerase mediated extension of each product. In OER reactions, small segments of overlap between the PCR products allow each product to be extended, generating a duplex that incorporates each of the PCR products. These assembled PCR products can then be amplified using a single primer amplification (SPA) reaction. The potential for bias in OER reactions, due to the use of randomized oligonucleotides in the amplification of PCR products, can be avoided by other methods, such as randomization by extension reaction (RER). In RER, gene specific PCR primers are used to amplify regions flanking the region to be randomized but not incorporating the region. In a subsequent step, the PCR product or products can be combined with a randomized oligonucleotide that contains sequence overlapping the flanking PCR products. In the presence of polymerase, the PCR strands are extended to incorporate the randomized oligonucleotide. Multiple RER products containing randomized oligonucleotides can also be assembled together via the extension of each product via small overlapping regions. In another example, duplexes can be generated by overlap PCR (Overlap PCR), a variation on OER-SPA whereby the overlap extension reaction and single primer amplification are combined into one step. In another example, randomized oligonucleotide duplexes are generated using a fill-in reaction whereby oligonucleotides containing mutations in a CDR and flanking restriction sites are amplified by fill-in reaction with a single primer. These randomized CDR duplexes are digested with restriction enzymes and ligated directly into a similarly digested backbone vector.

A desired variant nucleic acid molecule can be introduced into any chosen vector or display vector, e.g. a pCAL vector described herein, using standard recombinant DNA techniques. The variant nucleic acids can be generated to contain nucleic acid restriction sites that are compatible with the vector for ease of swapping and replacing nucleic acid sequences encoding variant heavy and/or light chain sequences or portions thereof (e.g. CDR). It is within the level of the skill in the art to select a restriction site sequence to include, which depends primarily on the chosen vector.

b. Quick Libraries and Hybrid Libraries

The use of restrictions sites included in the variant oligonucleotides or nucleic acid molecules also permits the generation of “quick libraries,” whereby variant oligonucleotides can be quickly swapped out between and among vectors, thereby increasing the diversity of the library. For example, a nucleic acid molecule or oligonucleotide encoding a variant CDRH3 can be designed to include 5′ (e.g. BssHII) and 3′ (e.g. ApaI) restriction sites. In another example, variant nucleic acid molecules encoding a variant CDRH1 can be designed to include 5′ (e.g. BstBI) and 3′ (e.g. MluI) restriction sites. In a further example, variant nucleic acid molecules encoding a variant CDRL3 can be designed to include 5′ (e.g. SnaBI) and 3′ (e.g. MluI) restriction sites. The resulting nucleic acid molecules can be digested and ligated into vectors having compatible restriction sites. This permits generating “quick libraries,” whereby combinations of various permutations of variant nucleic acids are ligated into the same vector for expression and generation of variant antibodies varied only in the light chain, only in the heavy chain, or in both the heavy and light chains. For example, libraries can be generated containing only residues varied in a CDR of the light chain (e.g. CDRL3), or only residues in a CDR of the heavy chain (e.g. CDRH1 or CDRH3). Then, based on the presence of compatible restriction sites, quick libraries can be quickly generated by ligating into the same vector the nucleic acid portion for the CDRL3 and the CDRH1 and/or CDRH3.

Libraries of oligonucleotides encoding variant heavy chains or variant light chains can be quickly swapped out between and among libraries though the use of restriction sites included in the variant oligonucleotides or nucleic acid molecules, thereby generating hybrid libraries. For example, a nucleic acid molecule or oligonucleotide encoding a variant light chain can be designed to include 5′ (e.g., MfeI) and 3′ (e.g., Pad) restriction sites. In another example, variant nucleic acid molecules encoding a variant heavy chain can be designed to include 5′ (e.g., NotI) and 3′ (e.g., SalI) restriction sites. This permits generation of hybrid libraries whereby variant light chains can be ligated into a vector containing a library of variant heavy chains, or whereby variant heavy chains can be ligated into a vector containing a library of variant light chains. For example, hybrid libraries can be generated which contain the variant light chains from one nucleic acid library cloned into a vector containing the variant heavy chains from another nucleic acid library.

Hybrid libraries also can be generated from initial Hits identified in the selection and screening described below. For example, nucleic acid molecules encoding one or more variant light chains that were selected by screening can be pooled to generate a library of variant light chains that bind a specific target, i.e., C. albicans. The nucleic acid molecules encoding these variant light chains can be swapped directly into a library of nucleic acid molecules encoding variant heavy chains through the use of compatible restriction sites. In another example, nucleic acid molecules encoding one or more variant heavy chains that bind a specific target can be pooled to generate a library of variant heavy chains that can be digested and ligated directly into a library of nucleic acid molecules encoding variant light chains. In one example, a hybrid library is generated from pool of nucleic acid molecules encoding 93 different variant 2G12 light chains that bind to C. albicans (set forth in SEQ ID NOS: 91-143, 145-151, 249-279 and 282-283) and a vector containing a library of variant heavy chains.

2. Display Libraries And Screening

Following generation of diverse libraries of variant polynucleotides, the variant polynucleotide members are ligated into vectors for expression in host cells to generate libraries of cells containing variant polynucleotides for expression of antibodies therefrom. Typically, variant polynucleotides are expressed in vectors that permit display on host cells. Antibody libraries herein include display libraries, which permit efficient and rapid screening and selection of antibody members against target antigens. Exemplary of display libraries are phage display libraries. In display libraries, each host cell or system displays a different member of the library permitting screening of the entire antibody repertoire in the library or a large sample therefrom.

Also, for purposes of display libraries herein, the vectors and host cells chosen permit display of the antibody in its domain-exchanged configuration. Typically, prior to selection of polypeptides from a collection, e.g. a phage display library, one or more methods is used to determine successful expression and/or display of the variant polypeptides. Such methods are well-known and include phage enzyme-linked immunosorbent assays (ELISAs) for detection of binding to a binding partner, and/or detection of an epitope tag on the expressed polypeptides, such as a His6 tag, which can be detected by binding to metal-chelating matrices or anti-His antibodies bound to solid supports.

a. Vectors

Generally, in display libraries, repertoires containing variable heavy chain and variable light genes can be separately cloned into a vector for expression on the surface of a host cell or system. In some examples, the variable heavy chain and light chain are cloned into different vectors. For example, variable heavy and variable light chain repertoires are cloned separately. The two vector libraries are then combined by co-transformation into a host cell so that each cell contains a different combination. In such examples, the variable heavy and light chains can recombine randomly to increase the diversity repertoire of the libraries. In other examples, the variable heavy chain and light chain are cloned into the same vector. The vectors typically are designed such that encoded polypeptides are fused to a protein that is expressed on the surface of a cell, such as a membrane protein or cell-surface associated protein, coat protein, or other similar protein or agent that facilitates display on a host cell.

For purposes of display libraries herein, the vectors and host cells chosen permit display of variant domain-exchanged antibody members in the library. Generally with conventional display methods, such as phage display methods, the displayed antibody fragment typically contains a single antibody combining site. By contrast, domain-exchanged antibodies contain an interface between the two interlocked V_{H} domains (V_{H}-V_{H}′ interface). Vectors are needed for displaying domain-exchanged fragments with two interlocked heavy chain variable regions (V_{H}), each paired with a light chain variable region (V_{L}).

Generally, the vectors include vectors containing stop codons, such as amber stop codons (UAG or TAG), ochre stop codons (UAA or TAA) and opal stop codons (UGA or TGA). Generally, such vectors contain a nucleic acid encoding a domain-exchanged antibody light chain operably linked at its 5′ end to the 3′ end of a leader sequence into which a stop codon has been introduced, and nucleic acid encoding an domain-exchanged antibody heavy chain operably linked at its 5′ end to the 3′ end of a leader sequence into which a stop codon has been introduced. In some examples, as discussed herein below, such vectors can be used to express soluble domain-exchanged antibodies, whereby the antibody or fragments are produced as non-fusion proteins by not including in the vector nucleic acid encoding a display protein, such as a phage-coat protein. For example, vectors that do not contain a stop codon in the leader sequence but that do contain a stop codon between the nucleic acid encoding the antibody and the coat protein, can be introduced into a non-suppressor host cell strain. Upon expression, there is no readthrough of the stop codon, so that only soluble antibody chains are expressed. Alternatively, to express the domain-exchanged antibody in the absence of fusion to a coat protein, the antibody chains can be introduced into any suitable vector that does not contain a coat protein.

For display purposes, the vector generally contains nucleic acid encoding a tag and a display protein to effect display of the antibody, for example a phage coat protein, downstream of the nucleic acid encoding the heavy chain. The nucleic acid encoding the tag is followed by a stop codon. The vector also can include nucleic acid encoding a light chain. Thus, when introduced into an appropriate partial suppressor cell, the heavy chain is expressed as a soluble protein (with a tag) and as a fusion protein with the tethering protein (e.g. phage coat protein), and the light chain is expressed only as a soluble protein. Exemplary of such a vectors include phagemid vectors, such as pCAL vectors described in U.S. Provisional Application Nos. 61/192,960, 61/192,982 and U.S. Publication Nos. 2010/0081575 and 2010/0093563.

Thus, typically, the vector contains a stop codon, generally an amber stop codon, between the nucleic acid encoding the antibody or fragment thereof and the nucleic acid encoding the display coat protein (e.g. cp3). Expression in a partial suppressor strain (e.g. a partial amber suppressor strain), however, results in “read-through,” translation that continues without being halted by the stop codon. Typically, depending on the suppressor strain, this “read-through” occurs only a certain percentage of the time. This partial read-through of the amber-stop results in a mixed collection of polypeptides. The mixed collection contains some polypeptide fusion proteins (i.e. antibody-coat protein fusion proteins) and some soluble polypeptides (i.e. soluble antibody), which are not part of coat protein fusions. In this case, a single genetic element encodes both the antibody and the coat protein, thus resulting in a single mRNA transcript that encodes both these polypeptides. Translation of the resulting transcript in a partial suppressor strain, therefore, produces a full length antibody-coat protein fusion protein when there is read through of the stop codon, and also a soluble antibody (i.e. truncated compared to the full length fusion protein because it does not contain the coat protein), is produced if there is no read through and translation terminates at the stop codon in the leader sequence. Thus, two copies of the antibody, e.g. two copies of an antibody fragment chain (e.g., two copies of the V_{H}-C_{H}1 chain or the V_{H}-linker-V_{L} chain), are expressed, one of which is part of a fusion protein and the other of which is a soluble protein. In the case of domain-exchanged antibodies, the soluble and fusion-protein chains interact on the surface of the genetic package, through conventional and/or artificial interactions (e.g. hydrophobic interactions, disulfide bonds and/or dimerization domains), to display domain-exchanged antibodies with two conventional antigen combining sites. Such suppressor host strains are well known and described (see, for example, Bullock et al., (1987) Biotechniques 5:376-379).

The vector optionally can contain additional stop codon(s), such as for expression of the domain-exchanged antibodies with reduced toxicity compared to the absence of the additional stop codons. For example, the polynucleotide encoding the protein of interest can be operably linked at the 5′ end to the 3′ end of a leader sequence in the vector, and a stop codon introduced into the leader sequence. This single genetic element encoding both the leader peptide and the domain-exchanged antibody or fragment thereof is operably linked to a promoter, thus resulting in a single mRNA transcript. Translation of the resulting transcript in a partial suppressor strain, therefore, produces a full length leader peptide-antibody fusion protein when there is read through of the stop codon, and a truncated leader peptide, without the antibody, when antibody there is no read through and translation terminates at the stop codon in the leader sequence. Thus, the antibody is translated and expressed only part of the time. In further examples, the vector contains two or more nucleic acid regions, each encoding a an antibody fragment, such as a heavy or light chain, for which reduced expression is desired, wherein each nucleic acid region is linked to a separate leader sequence and a stop codon is introduced into each leader sequence. For example, the vectors provided herein can contain nucleic acid encoding for a domain-exchanged antibody light chain that is operably linked to a leader sequence (e.g. the PelB leader sequence) and nucleic acid encoding for a domain-exchanged antibody heavy chain that is operably linked to another leader sequence (e.g. the OmpA leader sequence), wherein each leader sequence contains an amber stop codon. Thus, when introduced into a partial amber suppressor cell, expression of both the leader peptide-heavy chain fusion protein and leader peptide-light chain fusion protein is reduced compared to expression when the leader sequences do not contain the amber stop codons. The leader sequences are then cleaved from the light and heavy chains by bacterial peptidases following translocation across the cytoplasmic membrane.

The vectors can be used to display full-length domain-exchanged-antibodies, or domain-exchanged antibodies that are less then full-length, for example Fabs. Exemplary of the provided domain-exchanged fragments are fragments in which two chains (e.g. two V_{H}-C_{H}1 heavy chains or two V_{H}-linker-V_{L} single chains), encoded by the same genetic element (e.g. nucleotide sequence), are expressed on one phage as part of the domain-exchanged antibody fragment. Typically, in this example, one of the chains is expressed as a soluble, non-fusion protein (e.g. V_{H}-C_{H}1 or V_{H}-V_{L}) and the other is expressed as a phage coat protein fusion protein (e.g. V_{H}-C_{H}1-cp3 or V_{L}-V_{H}-cp3); in this example, however, the antibody chain portion of the two polypeptides is identical as they are encoded by the same genetic element. Exemplary of such domain-exchanged fragments are domain-exchanged Fab fragments and domain-exchanged scFv fragments. Also exemplary of the provided fragments are those (e.g. scFv tandem), containing multiple domains (e.g. V_{H}, V_{L}, C_{H}1, C_{L}) that are connected with peptide linkers to form the two heavy chain and two light chain domains of the domain-exchanged configuration. Exemplary of such fragments are domain-exchanged single chain Fab fragments and domain-exchanged scFv tandem fragments.

For example, for expression of Fab antibodies, vectors are generated containing a nucleic acid encoding a variable light chain domain operably linked at its 5′ end to the 3′ end of a leader sequence into which a stop codon has been introduced, and a nucleic acid encoding a variable heavy chain and C_{H}1 constant region operably linked at its 5′ end to the 3′ end of a leader sequence into which a stop codon has been introduced. Alternatively, other domain-exchanged antibody fragments can be expressed. For example, such additional fragments include a Fab hinge fragment, which contains an additional hinge sequence operably linked at the 5′ end of the Fab heavy chain sequence. Exemplary of such a fragment is a 2G12 Fab hinge fragment expressed from a vector containing the nucleotide sequence set forth in SEQ ID NO:179, or a variant thereof. In another example, an additional antibody fragment includes a scFv fragment, which contains one variable heavy chain (V_{H}) encoding sequence and one variable light (V_{L}) encoding sequence, followed by an amber stop codon, promoting formation of a domain-exchanged scFv fragment with two conventional antibody combining sites. Exemplary of such a fragment is a 2G12 scFv fragment expressed from a vector containing the nucleotide sequence set forth in SEQ ID NO:180, or a variant thereof. In an additional example, an antibody fragment includes an scFv tandem fragment, which includes the sequence for an additional V_{H} and an additional V_{L} region, separated by a linker sequence, for expression of two heavy chain variable domains and two light chain variable region domains from the single vector. Exemplary of a nucleic acid encoding a peptide linker are any that have the nucleotide sequence set forth in any of SEQ ID NOS: 181-187. Exemplary of a scFv tandem fragment is a 2G12 scFv tandem fragment expressed from a vector containing the nucleotide sequence set forth in SEQ ID NO:188, or a variant thereof. In a further example, an antibody fragment includes scFv hinge and scFv hinge(ΔE) fragments, each of which contains the sequence of the scFv encoding vector, with an additional hinge-region encoding sequence, to promote interaction between the two single chains in the fragment. Exemplary of an scFv hinge and scFv hinge(ΔE) fragments is a 2G12 domain-exchanged scFv hinge and scFv hinge(ΔE) fragments expressed from the vector containing the nucleotide sequence set forth in SEQ ID NO: 189, and SEQ ID NO: 190, respectively, or variants thereof. Further exemplary antibody fragments are 2G12 antibody fragments containing a mutation in the heavy chain variable region at residue 19 resulting in an Ile-Cys mutation to promote interaction of the two heavy chain variable regions. Such antibody fragments include, for example, 2G12 domain-exchanged Fab Cys19 fragment (expressed from the vector containing the nucleotide sequence set forth in SEQ ID NO: 191, or variants thereof containing Cys19, which contains a mutation in the heavy chain of the Fab fragment, resulting in an Ile-Cys mutation to promote interaction of the two heavy chain variable regions of the Fab fragment); the 2G12 domain-exchanged scFab ΔC²Cys19 (expressed from the vector containing the nucleotide sequence set forth in SEQ ID NO: 192, or variants thereof containing Cys 19, which contains the same mutation in the heavy chain of the Fab fragment, resulting in an Ile-Cys mutation, and contains a sequence encoding a linker between the heavy and light chains); and the 2G12 domain-exchanged scFv Cys19 fragment (expressed from the vector containing the nucleotide sequence set forth in SEQ ID NO: 193 or variants thereof containing Cys19, which contains the sequence of the scFv fragment with the mutation in the heavy chain variable region, resulting in an Ile-Cys mutation to promote interaction of the two heavy chain variable regions of the scFv fragment).

The vectors generally also contain an origin of replication; one or more selectable markers; other regulatory elements to enhance protein expression and regulation, for example, transcriptional enhance sequences, translational enhancer sequences, promoters, activators, translational start and stop signals, transcription terminators, cistronic regulators, polycistronic regulators; tag sequences to facilitate identification, separation, purification and/or isolation of the expressed polypeptide; or a ribosome binding site. One of skill in the art is familiar with components that can be used in vectors to control and/or enhance expression of encoded polypeptides therefrom. Such vectors can be generated by standard cloning and recombinant techniques well known in the art.

Also, for purposes of display, vectors can include phagemid vectors, which do not contain a sufficient set of phage genes for production of stable phage particles after transformation of host cells. Thus, in some examples, a library of polynucleotide variants can be incorporated into a phagemid vector. In a phagemid system, the nucleic acid encoding the display protein is provided on a phagemid vector, typically of length less than 6000 nucleotides. The phagemid vector includes a phage origin of replication so that the plasmid is incorporated into bacteriophage particles when bacterial cells bearing the plasmid are infected with helper phage, e.g. M13K01 or M13VCS or are transfected into analogous cells containing helper phage DNA, for example, DH5α VCSM13 dsDNA cells. Phagemids, however, lack a sufficient set of genes in order to produce stable phage particles after infection. These phage genes can be provided by a helper phage or analogous cells. For example, the necessary phage genes typically are provided by co-infection of the host cell with helper phage, for example M13K01 or M13VCS. Typically, the helper phage or analogous cells provides an intact copy of the gene III coat protein and other phage genes required for phage replication and assembly. Because the helper phage has a defective origin of replication, the helper phage genome is not efficiently incorporated into phage particles relative to the plasmid that has a wild type origin. Thus, the phagemid vector includes a bacterial origin of replication and a phage origin of replication so that the plasmid is incorporated into bacteriophage particles when bacterial cells bearing the plasmid are infected with helper phage, e.g. M13K01 or M13VCS. See, e.g., U.S. Pat. No. 5,821,047. The phagemid genome typically contains a selectable marker gene, e.g. Amp^{R} or Kan^{R} (for ampicillin or kanamycin resistance, respectively) for the selection of cells that are infected by a member of the library. Exemplary phagemid vectors include, but are not limited to, pBluescript, pBK-CMV®(Stratagene) and pCAL vectors, which contain a sequence of nucleotides encoding the C-terminal domain of filamentous phage M13 Gene III coat protein.

i. pCAL Phagemid Vectors

pCAL vectors contain nucleic acid encoding part (e.g. the C terminus) of the filamentous phage M13 gene III coat protein. The vectors also include a leader sequence encoding a leader peptide, a multiple cloning site, an amber stop codon (TAG) and a tag upstream of the coat protein, such that nucleic acid, such as nucleic acid encoding an antibody or fragment thereof, can be inserted at the cloning site 5′ to the amber stop codon, which is 5′ to the tag and the coat protein. Thus, upon expression in a partial amber suppressor cell, both antibody-coat protein fusion protein (with a tag) and soluble antibody proteins are produced facilitating display of domain-exchanged antibodies, including domain-exchanged antibody fragments. The encoded domain-exchanged antibody can be full-length, or less then full-length, for example, a Fab, a Fab hinge fragment, an scFv fragment, scFv tandem fragment, scFv hinge and scFv hinge(ΔE), or mutations thereof (for example, Cys19 in 2G12). The vectors can contain one or more leader sequences, for the secretion of one or more polypeptides.

In some examples, the pCAL vectors further contain a stop codon, such as an amber stop codon, in the leader sequence encoding a leader peptide. For example, a pCAL vector can contain a nucleic acid encoding a domain-exchanged antibody light chain operably linked at its 5′ end to the 3′ end of a leader sequence into which a stop codon has been introduced, and nucleic acid encoding an domain-exchanged antibody heavy chain operably linked at its 5′ end to the 3′ end of a leader sequence into which a stop codon has been introduced. When introduced into an appropriate partial suppressor cell, the heavy chain is expressed as a soluble protein (with a tag) and as a fusion protein with the phage coat protein, and the light chain is expressed as a soluble protein. Inclusion of the stop codon in the leader sequences linked to the nucleic acid encoding the heavy and light chains facilitates reduced expression of the these proteins in corresponding partial suppressor cells (i.e. amber partial suppressor cells if amber stop codons is introduced), thus reducing the toxicity of these proteins to the host cell. Examples of leader sequences include the PelB leader sequence (the nucleic acid encoding the leader peptides from the pectate lyase protein from Erwinia carotovora and the OmpA leader sequence (the nucleic acid sequence encoding the leader peptide from the E. coli outer membrane protein). Generally, the leader sequences of the heavy and light chain are different. In other example, the vectors include just one leader sequence. For example, a vector can include one leader sequence 5′ to nucleic acid encoding a V_{H}-linker-V_{L} single chain. From such a vector, domain-exchanged scFv fragments can be displayed.

Vectors also can include further sequences that control expression. For example, the single genetic element containing these leader and antibody chain sequences can be operably linked to the lactose promoter and operator, such that their expression is regulated by lactose or an appropriate lactose substitute, such as IPTG. In addition, vectors can include a truncated or full length lac I gene, which encodes the lactose repressor molecule. In the absence of lactose or another suitable inducer, such as IPTG, the repressor binds to the operator and interferes with binding of the RNA polymerase to the promoter, inhibiting transcription of the operably linked heavy and light chain genes. In the presence of lactose or a suitable equivalent, such as IPTG, the lactose metabolite allolactose binds to the repressor, causing a conformational change that renders the repressor unable to bind to the operator, thereby allowing binding of the RNA polymerase and transcription of a single transcript encoding the domain-exchanged antibody light and heavy chains. Ribosome binding sites also can be included upstream of leader sequences.

Different pCAL vectors provided herein can result in different amounts of readthrough through the amber-stop codon. For example, the pCAL G13 vector (SEQ ID NO:5) contains a guanine residue at the position just 3′ of the amber stop codon, while the pCAL A1 vector contains an adenine at this position. Choice of vector can determine how the relative amount of read-through that occurs through the stop codon, e.g. when using a partial suppressor strain, and thus can regulate the relative amount of fusion versus non-fusion target/variant polypeptide translated from the vector, as well as the amount of overall expression in instances where a stop codon is present in the leader sequence encoding the leader peptide. Additionally, as described below, different partial suppressor cells can have varying suppression efficiency, such that more or less read-through is observed depending on the strain of partial suppressor cell into which the vector is introduced.

A desired variant nucleic acid molecule can be introduced into a pCAL vector using standard recombinant DNA techniques. Exemplary pCAL vectors include those having the sequence of nucleic acids set forth in any of SEQ ID NOS: 5 (pCAL G13), 194 (pCAL A1), 6 (2G12 pCAL G13), 195 (3-ALA 2G12 pCAL G13), 196 (3-ALA LC 2G12 pCAL G13), 197 (2G12 pCAL A 1), 1 (2G12 pCAL IT*) and 12 (2G12 pCAL ITPO). In some instances, the variant nucleic acid molecules, such as variant heavy and/or light chains, are introduced into the multiple cloning site of pCAL G13 (SEQ ID NO:5) or pCAL A1 (SEQ ID NO:194). In other instances, the variant nucleic acid molecules, such as variant heavy and/or light chains, are introduced by replacement of the existing heavy and/or light chain nucleic acid molecules in any of 2G12 pCAL G13 (SEQ ID NO:6), 3-ALA 2G12 pCAL G13 (SEQ ID NO:195), 3-ALA LC 2G12 pCAL G13 (SEQ ID NO:196), 2G12 pCAL A1 (SEQ ID NO:197), 2G12 pCAL IT* (SEQ ID NO:1) and 2G12 pCAL ITPO (SEQ ID NO:12). For example, the variant nucleic acids can be generated to contain nucleic acid restriction sites that are compatible with the vector for ease of swapping and replacing nucleic acid sequences encoding heavy and/or light chain sequences. Example 2 exemplifies replacement of variant light chains into the 2G12 3 Ala LC pCAL IT* vector (set forth in SEQ ID NO:23) by digestion of the vector with MfeI and PacI and ligation of compatible variant light chain duplex assembled oligonucleotide fragments. Following ligation, vector libraries are generated each containing a randomized 2G12 encoding nucleic acid members, whereby variation is in the CDR L3 region of the light chain. Similarly, other pCAL vectors, such as 2G12 pCAL IT*, 2G12 pCAL, or 2G12 pCAL IT* 3-Ala can similarly be digested with Mfe I and Pac I for insertion of assembled duplex oligonucleotides encoding variant light chains. In another example, 2G12 pCAL IT* vectors or the 2G12 pCAL vector, or any 3-ALA variant thereof, can be digested with Sal I and Not I for ligation of assembled duplex oligonucleotides encoding variant heavy chains.

To express the protein(s) from the provided vectors that contain stop codon nucleic acids, the vectors are transformed into an appropriate partial suppressor host cell strain. Different partial suppressor cells can have varying suppression efficiency, such that more or less read-through is observed depending on the strain of partial suppressor cell into which the vector is introduced. The host cell chosen generally depends on the stop codon incorporated into the vector. For example, if one or more amber stop codons are introduced into the vector, then the vector is transformed into a partial amber suppressor strain that harbors an amber suppressor tRNA molecule. If one or more ochre stop codons are introduced into vector, the vector is transformed into a partial ochre suppressor strain that harbors an ochre suppressor tRNA molecule. Further, a host cell typically is chosen in which the suppressor tRNA molecule will incorporate the desired amino acid residue when read through of the stop codon occurs (such as the wild-type amino acid or another desired amino acid). For example, if the vector contains an amber stop codon that was introduced in place of a glutamine codon (or where a glutamine is desired), then the vector can be introduced into a partial amber suppressor strain that expresses an amber suppressor tRNA that incorporates a glutamine residue at the TAG codon. It is within the level of one of skill in the art to choose the appropriate host cell. Exemplary amber suppressor cells (including partial amber suppressor cells) include, but are not limited to, XL1-Blue, DB3.1, DH5α, DH5αF′, DH5αF′IQ, DH5α-MCR, DH21, EB5α, HB101, RR1, JM101, JM103, JM106, JM107, JM108, JM109, JM110, LE392, Y1088, C600, C600hfl, MM294, NM522, Stb13 and K802 cells.

The vector can be introduced into the partial amber suppressor cell using any method known in the art, including, but not limited to, electroporation and chemical transformation. Following transformation into an appropriate partial suppressor strain, in some instances, expression of the polypeptides can be induced in the host cells. For example, if transcription is under control of a regulatable promoter, then the appropriate conditions can be generated to induce transcription. Further, in some examples, the host cells are phage-display compatible host cells, and are used to display the protein(s) of interest on the surface of a bacteriophage, for example, in a phage display library. By generating phage display libraries, the proteins displayed on the phage can be screened, analyzed and selected for based on various properties, such as binding activities. such as described in more detail below.

b. Display Methods

Methods for displaying polypeptides on the surface of genetic packages, e.g. in libraries, are well known and include, for example, phage display (see, e.g., Barbas, C. F., 3rd et al., 2001. Phage Display: A Laboratory Manual. Cold Spring Harbor Laboratory Press, Cold Spring Harbor, N.Y.; Clackson et al., (1991) Nature, 352:624-628) and methods for display on other genetic packages. Domain-exchanged antibodies can be displayed on the surface of any genetic package. Exemplary genetic packages include, but are not limited to, bacterial cells, bacterial spores, viruses, including bacterial DNA viruses, for example, bacteriophages, typically filamentous bacteriophages, for example, Ff, M13, fd, and f1 (see, e.g., Barbas, C. F., 3rd et al., 2001. Phage Display: A Laboratory Manual. Cold Spring Harbor Laboratory Press, Cold Spring Harbor, N.Y.; Clackson et al., (1991) Nature, 352:624-628; Glaser et al. (1992) J. Immunol., 149:3903 3913; Hoogenboom et al. (1991) Nucleic Acids Res., 19:4133-41370; Clackson and Lowman, Phage Display: A Practical Approach; (2004) Oxford University Press (Chapter 1, Russel et al., An introduction to Phage Biology and Phage Display, p. 1-26; Chapter 2, Sidhu and Weiss Constructing Phage display libraries by oligonucleotide-directed mutagenesis, p 27-41)), baculoviruses (see, e.g., Boublik et al., (1995) Bio/Technology, 13:1079-1084). Typically, polypeptides are displayed on genetic packages in collections of genetic packages, such as phage display libraries, which can be used to select particular polypeptides from the collections using the provided methods. Display of the polypeptides on genetic packages allows selection of polypeptides having desired properties, for example, the ability to bind with a particular binding partner.

i. Phage Display

Typically, the genetic packages are phage, and the polypeptides are expressed with phage display. Methods for generating phage display libraries are well known (see Barbas, C. F., 3rd et al., 2001. Phage Display: A Laboratory Manual. Cold Spring Harbor Laboratory Press, Cold Spring Harbor, N.Y.; Clackson and Lowman, Phage Display: A Practical Approach; (2004) Oxford University Press (Clackson and Lowman, Phage Display: A Practical Approach; (2004) Oxford University Press (Chapter 1, Russel et al., An introduction to Phage Biology and Phage Display, p. 1-26; Chapter 2, Sidhu and Weiss Constructing Phage display libraries by oligonucleotide-directed mutagenesis, p 27-41)).

For phage display, libraries of domain-exchanged antibodies, including domain-exchanged antibody fragments can be expressed on the surfaces of bacteriophages, such as, but not limited to, M13, fd, f1, T7, and λ phages (see, e.g., Santini (1998) J. Mol. Biol. 282:125-135; Rosenberg et al. (1996) Innovations 6:1-6; Houshmand et al. (1999) Anal Biochem 268:363-370, Zanghi et al. (2005) Nuc. Acid Res. 33(18)e160:1-8). Phage display is described, for example, in Ladner et al., U.S. Pat. No. 5,223,409; Rodi et al. (2002) Curr. Opin. Chem. Biol. 6:92-96; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem. 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982.

Phage display systems typically utilize filamentous phage, such as M13, fd, and f1. In some examples using filamentous phage, the display protein is fused to a phage coat protein anchor domain. The fusion protein can be co-expressed with another polypeptide having the same anchor domain, e.g., a wild-type or endogenous copy of the coat protein. Phage coat proteins that can be used for protein display include (i) minor coat proteins of filamentous phage, such as the bacteriophage M13 gene III protein (also called gIIIp, cp3, g3p; GENBANK g.i. 59799327, having the amino acid sequence set forth in SEQ ID NO: 198). Portions of these phage proteins also can be used. In one example, the anchor domain of gIIIp is used (see, e.g., U.S. Pat. No. 5,658,727). In another example, gVIIIp is used (see, e.g., U.S. Pat. No. 5,223,409), which can be a mature, full-length gVIIIp fused to the display protein. Generally, to produce the fusion protein, polypeptides are fused to bacteriophage coat proteins with covalent, non-covalent, or non-peptide bonds. (See, e.g., U.S. Pat. No. 5,223,409, Crameri et al. (1993) Gene 137:69 and WO 01/05950). For example, nucleic acids encoding the variant polypeptides can be fused to nucleic acids encoding the coat proteins (e.g. by introduction into a vector encoding the coat protein) to produce a polypeptide-coat protein fusion protein, where the polypeptide is displayed on the surface of the bacteriophage. Additionally, the fusion protein can include a flexible peptide linker or spacer, a tag or detectable polypeptide, a protease site, or additional amino acid modifications to improve the expression and/or utility of the fusion protein. For example, addition of a protease site can allow for efficient recovery of desired bacteriophages following a selection procedure. Exemplary tags and detectable proteins are known in the art and include for example, but not limited to, a histidine tag, a hemagglutinin tag, a myc tag or a fluorescent protein.

For display of polypeptides on phage, host cells capable of phage infection and packaging are transformed with phage vectors, typically phagemid vectors, containing polynucleotides encoding the polypeptides. Any phagemid vector can be used, including for example, any discussed above. In one example, the host cells are partial suppressor cells, such as any of the cells described above, provided the cells are compatible with phage display. Any method of transformation known to one of skill in the art can be used, for example, electroporation or other known transformation methods.

The transformed cells can be grown for amplification of the vector nucleic acids, for example, for subsequence sequence analysis or pooling for re-transformation. Following amplification, phage packaging and protein expression is induced, typically by co-infection with a helper phage. Exemplary of helper phage is VCS M13 helper phage. Methods for transformation, growth and phage packaging and propagation are well-known (see Clackson and Lowman, Phage Display: A Practical Approach; (2004) Oxford University Press (Chapter 2, Constructing Phage display libraries by oligonucleotide-directed mutagenesis, Sidhu and Weiss, p. 27-41). Any phage display method can be used. In general, host cells transformed with the vector nucleic acids are incubated in medium. Helper phage is added and the cells are incubated. Typically, polypeptide expression is induced, for example, by IPTG. Generally, the polypeptides are exported to the periplasm (e.g. as part of a fusion protein) for assembly into phage during phage packaging. Following phage packaging, the polypeptides are expressed on the surface of phage, typically as part of fusion proteins, each containing a polypeptide of interest and a portion of a phage coat protein. The phage can be isolated, for example, by precipitation, and then assayed and/or used for selection of desired variant polypeptides.

ii. Other Display Methods

Other known display methods can be used. Display systems include, for example, prokaryotic or eukaryotic cells. Exemplary of systems for cell surface expression include, but are not limited to, bacteria, yeast, insect cells, avian cells, plant cells, and mammalian cells (Chen and Georgiou (2002) Biotechnol Bioeng 79: 496-503). In one example, the bacterial cells for expression are Escherichia coli.

(1) Cell Surface Display

Polypeptides can be displayed as part of a fusion protein with a protein that is expressed on the surface of the cell, such as a membrane protein or cell surface-associated protein. For example, a polypeptide can be expressed in E. coli as a fusion protein with an E. coli outer membrane protein (e.g. OmpA), a genetically engineered hybrid molecule of the major E. coli lipoprotein (Lpp) and the outer membrane protein OmpA or a cell surface-associated protein (e.g. pili and flagellar subunits). Generally, when bacterial outer membrane proteins are used for display of heterologous peptides or proteins, expression is achieved through genetic insertion into permissive sites of the carrier proteins. Expression of a heterologous peptide or protein is dependent on the structural properties of the inserted protein domain, since the peptide or protein is more constrained when inserted into a permissive site as compared to fusion at the N- or C-terminus of a protein. Modifications to the fusion protein can be done to improve the expression of the fusion protein, such as the insertion of flexible peptide linker or spacer sequences or modification of the bacterial protein (e.g. by mutation, insertion, or deletion, in the amino acid sequence). Enzymes, such as β-lactamase and the Cex exoglucanase of Cellulomonas fimi, have been successfully expressed as Lpp-OmpA fusion proteins on the surface of E. coli (Francisco J. A. and Georgiou G. (1994) Ann N Y Acad. Sci. 745:372-382 and Georgiou G. et al. (1996) Protein Eng. 9:239-247). Other peptides of 15-514 amino acids have been displayed in the second, third, and fourth outer loops on the surface of OmpA (Samuelson et al. (2002) J. Biotechnol. 96: 129-154). Thus, outer membrane proteins can carry and display heterologous gene products on the outer surface of bacteria.

In another example, polypeptides are fused to autotransporter domains of proteins such as the N. gonorrhoeae IgA1 protease, Serratia marcescens serine protease, the Shigella flexneri VirG protein, and the E. coli adhesin AIDA-I (Klauser et al. (1990) EMBO J. 1991-1999; Shikata S, et al. (1993) J Biochem. 114:723-731; Suzuki T et al. (1995) J Biol. Chem. 270:30874-30880; and Maurer J et al. (1997) J Bacteriol. 179:794-804). Other autotransporter proteins include those present in gram-negative species (e.g. E. coli, Salmonella serovar Typhimurium, and S. flexneri). Enzymes, such as β-lactamase, have been successful expressed on the surface of E. coli using this system (Lattemann C T et al. (2000) J. Bacteriol. 182(13): 3726-3733).

Bacteria can be recombinantly engineered to express a fusion protein, such a membrane fusion protein. Polynucleotides encoding the polypeptides for display can be fused to nucleic acids encoding a cell surface protein, such as, but not limited to, a bacterial OmpA protein. The nucleic acids encoding the polypeptides can be inserted into a permissible site in the membrane protein, such as an extracellular loop of the membrane protein. Additionally, a nucleic acid encoding the fusion protein can be fused to a nucleic acid encoding a tag or detectable protein. Such tags and detectable proteins are known in the art and include for example, but not limited to, a histidine tag, a hemagglutinin tag, a myc tag or a fluorescent protein. The nucleic acids encoding the fusion proteins can be operably linked to a promoter for expression in the bacteria, For example nucleic acid can be inserted in a vectors or plasmid, which can carry a promoter for expression of the fusion protein and optionally, additional genes for selection, such as for antibiotic resistance. The bacteria can be transformed with such plasmids, such as by electroporation or chemical transformation. Such techniques are known to one of ordinary skill in the art.

Proteins in the outer membrane or periplasmic space usually are synthesized in the cytoplasm as premature proteins, which are cleaved at a signal sequence to produce the mature protein that is exported outside the cytoplasm. Exemplary signal sequences used for secretory production of recombinant proteins for E. coli are known. The N-terminal amino acid sequence, without the Met extension, can be obtained after cleavage by the signal peptidase when a gene of interest is correctly fused to a signal sequence. Thus, a mature protein can be produced without changing the amino acid sequence of the protein of interest (Choi and Lee, (2004) Appl. Microbiol. Biotechnol. 64: 625-635).

Other known cell surface display methods can be used, including, but not limited to, ice nucleation protein (Inp)-based bacterial surface display system (Lebeault J. M., (1998) Nat. Biotechnol. 16:576-580), yeast display (e.g. fusions with the yeast Aga2p cell wall protein; see U.S. Pat. No. 6,423,538), insect cell display (e.g. baculovirus display; see Ernst et al. (1998) Nucleic Acids Research, 26(7):1718-1723), mammalian cell display, and other eukaryotic display systems (see e.g. U.S. Pat. No. 5,789,208 and WO 03/029456). The vectors provided herein can be used in any of these systems to display a domain-exchanged antibody, provided that the host cells contain an appropriate functional suppressor tRNA and that the vectors contain the appropriate elements for replication, amplification, transcription and translation in the host cell.

(2) Other Display Systems

Other display formats also can be used. Exemplary other display formats include nucleic acid-protein fusions, ribozyme display (see e.g. Hanes and Pluckthun (1997) Proc. Natl. Acad. Sci. U.S.A. 13:4937-4942), bead display (Lam, K. S. et al., (1991) Nature 354, 82-84; Houghten, R. A. et al., (1991) Nature, 354:84-86; Furka, A. et al., (1991) Int. J. Peptide Protein Res. 37:487-493; Lam, K. S., et al., (1997) Chem. Rev., 97:411-448; U.S. Published Pat. No. 2004-0235054) and protein arrays (see e.g. Cahill (2001) J. Immunol. Meth. 250:81-91, WO 01/40803, WO 99/51773, and US2002-0192673-A1).

In specific other cases, it can be advantageous to instead attach the polypeptides, or phage libraries or cells expressing variant polypeptides, to a solid support. For example, in some examples, cells expressing polypeptides can be naturally adsorbed to a bead, such that a population of beads contains a single cell per bead (Freeman et al. (2004) Biotechnol. Bioeng. 86:196-200). Following immobilization to a glass support, microcolonies can be grown and screened with a chromogenic or fluorogenic substrate. In another example, variant polypeptides or phage libraries or cells expressing variant polypeptides can be arrayed into titer plates and immobilized.

c. Screening

The variant domain-exchanged antibody libraries provided herein, for example variant 2G12 antibody libraries, can be used to screen for antibodies that specifically bind to epitopes on'pathogens or cells, for example, carbohydrate or glycolipid epitopes. Unlike conventional antibodies, the antigen-binding site of domain-exchanged antibodies are suited for binding to such epitopes. Hence, members of the domain-exchanged variant antibody libraries are Candidates for recognizing carbohydrate and glycolipid epitopes on the surface of pathogens (e.g. bacterial, viral or fungal) and cells. The libraries can be used in screening assays against such antigens. The identified antibodies can be used for therapeutic, diagnostic and research purposes against the selected antigen.

Any pathogenic cell or other cell can be used in the screening assays herein to identify or select antibodies. Exemplary pathogens include any that are involved in or associated with a disease or condition. Such pathogens are known to one of skill in the art. It is within the level of skill in the art to select any pathogenic cell to use in the screening assays herein for use in identifying or selecting antibodies specific thereto. Pathogens can be obtained by any method known to one of skill in the art, for example, isolated from serum and/or blood of an infected individual or obtained as a clinical isolate or other stable pathogen stock from ATCC or other similar depository. The pathogens can be activated or inactivated. In addition, as described below, whole pathogenic cells can be used in the screening assays. Exemplary of such methods provided herein are methods of screening to select for domain-exchanged antibodies against Candida.

Methods for selection of domain-exchanged antibodies from antibody libraries for identification of any bind Candida can be adapted based on screening methods well-known in the art. Generally, an antigen or epitope of Candida or whole Candida cells are presented to the collection of library of variant domain-exchanged antibodies and the collection enriched for members that bind, for example, with high affinity, to

Candida. For purposes herein, any strain of Candida can be used, including but not limited to, Candida albicans (serotype A or B), Candida krusei, Candida tropicalis or Candida glabrata. Such strains can be obtained from American Type Culture Collection (ATCC). For example, exemplary strains include ATCC No. 10231; ATCC No. 12061; ATCC No. 44373; and ATCC No. 36803. Alternatively, strains can be obtained as clinical isolates. Typically, selection is done on fixed whole cells. Alternatively, selection can be done on cell wall preparations of Candida. Example 4 describes an exemplary screening assay of an exemplary variant light chain 2G12 domain-exchanged antibody library against formalin-fixed C. albicans cells as the target antigen. The target antigen can be incubated with the library, such as a phage display library, for a sufficient time to permit binding. Following incubation, the cell-library mixture is washed and non-binding library members are washed away using one or more wash buffers, generally supplemented with polysorbate 20 (Tween 20). The stringency of the wash can be adjusted according to methods well known to the skilled artisan. Conditions of the binding and washing steps can be varied to adjust stringency, according to various parameters, for example, affinity of the target or desired polypeptide for the binding partner.

After, washing to remove unbound library members, for example, unbound phage, bound library members can be eluted. For example, after washing to remove non-bound phage, the phage-expressing variant domain-exchanged antibodies that have bound to the Candida target antigen are eluted using one of several well known elution methods, typically by reduction of the pH of the solution, recovery of phage, and neutralization, or addition of a competing polypeptide which can compete for binding to the binding partner. Exemplary of the elution step is reduction of the pH to approximately 2 (e.g. 2.2) by incubation of the bound phage with 10-100 mM hydrochloric acid (HCl), pH 2.2, or with 0.2 M glycine, (e.g. for 10 minutes at room temperature (e.g. 25° C.)), followed by removal of the eluate and addition of 1-2 M Tris-base (pH 8.0-9.0) to neutralize the pH. In some examples, multiple elution steps are carried out and the eluates pooled for subsequent steps.

Domain-exchanged antibodies selected in the screening can be amplified for analysis and/or use in subsequent screening steps. The amplification step amplifies the genome of the genetic package, e.g. phage. This amplification can be useful for expressing the polypeptide encoded by the selected phage, for example, for use in analysis steps or subsequent panning steps in iterative selection processes, and for identification of the variant polypeptide and polynucleotide encoding the polypeptide, such as by subsequent nucleic acid sequencing. For example, following elution, the phage nucleic acids are amplified in an appropriate host cell. In one example, the selected phage is incubated with an appropriate host cell (e.g. XL1-Blue cells) to allow phage adsorption (for example, by incubation of eluted phage with cells having an O.D. between 0.3 and 0.6 for 20 minutes at room temperature). After this incubation to allow phage adsorption, a small volume of nutrient broth is added and the culture agitated to facilitate phage DNA replication in the multiplying host cell. After this incubation, the culture typically is supplemented with an antibiotic and/or inducer and the cells grown until a desired optical density is reached. The phage genome can contain a gene encoding resistance to an antibiotic to allow for selective growth of the cells that maintain the phage vector DNA. The amplification of the display source, such as in a bacterial host cell, can be optimized in a variety of ways. For example, the host cells can be added in vast excess to the genetic packages recovered by elution, thereby ensuring quantitative transduction of the genetic package genome. The efficiency of transduction optionally can be measured when phage are selected.

Selected domain-exchanged antibodies can be purified and analyzed. For example, following infection of E. coli host cells with selected phage as set forth above, the individual clones can be picked and grown up for plasmid purification using any method known to one of skill in the art. The purified plasmid can used for nucleic acid sequencing to identify the sequence of the variant polynucleotide and, by extrapolation, the sequence of the variant polypeptide, or can be used to transfect into any cell for expression, such as by not limited to, a mammalian expression system. Purified proteins also can be tested in subsequent in vitro or in vivo assays for binding activity.

The method of screening collections of displayed polypeptides can be performed in an iterative process by multiple rounds of panning. Such iteration permits identification of antibodies with improved binding affinity. Generally, selected polypeptides are used in multiple additional rounds of screening, by pooling the selected polypeptides (e.g. eluted phage), propagation of nucleic acids encoding the polypeptides in host cells, expression (e.g. phage display) of the selected polypeptides, and a subsequent round of panning. Multiple rounds, e.g. 2, 3, 4, 5, 6, 7, 8, or more rounds, of screening can be performed. In this example of iterative screening, the variant polypeptide collection used in the successive round of screening includes the polypeptides selected in the previous round. Alternatively, the multiple rounds of screening can be performed using the initial collection of polypeptides. In an alternative example of iterative screening, a new polypeptide collection can be generated, that has been further varied. In one such example, one or more selected variant polypeptides is/are used as target polypeptides for variation.

Following selection and identification, selected antibodies can be further characterized for binding to antigen as it exists in its natural state, typically whole cells. Binding of antibody to pathogens, cells or other source can be determined by established methodologies, for example, ELISA, flow cytometry and immunohistochemistry as described herein below. Binding affinity also can be determined. Any method known to one of skill in the art can be used to measure the binding affinity of an antibody. For example, the binding properties of an antibody can be assessed by performing a saturation binding assay, for example, a saturation ELISA, whereby binding to an antigen is assessed with increasing amounts of antibody. In such experiments, it is possible to assess whether the binding is dose-dependent and/or saturable. In addition, the binding affinity can be extrapolated from the 50% binding signal. Typically, apparent binding affinity is measured in terms of its association constant (Ka) or dissociation constant (Kd) and determined using Scatchard analysis (Munson et al., (1980) Anal. Biochem., 107:220). For example, binding affinity to a target protein can be assessed in a competition binding assay in where increasing concentrations of unlabeled protein is added, such as by radioimmunoassay (RIA) or ELISA. It is understood that the binding affinity of an antibody can vary depending on the assay and conditions employed, although all assays for binding affinity provide a rough approximation. By performing various assays under various conditions it is possible to estimate the binding affinity of an antibody.

Further, selected antibodies also can be assessed for their neutralization capabilities or other inhibitory activity against the particular antigen selected against. Neutralization assays and inhibitory assays for different antigens (e.g. virus, bacterial or fungal antigens) are known in the art. For example, exemplary virus neutralization assays to assess activity against a virus membrane-associated antigen include, but not limited to, virus attachment to cell membranes and penetration in cells, virus uncoating, virus nucleic acid synthesis, viral protein synthesis and maturation, assembly and release of infectious particles, targeting the membrane of infected cells resulting in a signal that leads to no replication or a lower replication rate of the virus. In another example, exemplary fungal neutralization assays to assess activity against a fungal antigen include measurement of the inhibition or amelioration of infection. Methods for measuring inhibition or amelioration of infection include, but are not limited to, metabolic activity assays, adhesion and invasion assays, biofilm formation assays, growth assays, hyphae formation assays, opsonization assays and gene transcription assays. Such assays can be employed to assess, for example, inhibition of yeast viability, adherence to and invasion of host cells, biofilm formation, growth and virulence, the promotion of phagocytosis by macrophage and the expression of cytokines such as IL-8. In an additional example, exemplary bacterial neutralization assays to assess activity against a bacterial antigen include, for example, bacterial attachment to cell membranes and penetration (e.g. phagocytosis) into cells and gene transcription assays. Exemplary assays to test selected antibodies against Candida are described herein below in Section G.

Functional Screening

Screening also can be performed to verify that the nucleic acid molecule(s) encodes a functional antibody. A desired variant nucleic acid molecule can be introduced into a functional screening vector using standard recombinant DNA techniques. Functional screening vectors generally contain the gene of interest, in this instance the antibody heavy chain or light chain, fused to gene encoding a protein that allows for detection of a function, such as, for example, a reporter protein, a binding protein or a protein that confers antibody resistance. In one example, the functional screening vector contains the gene encoding the Fab antibody fused to a gene encoding beta-lactamase (bla). In this example, expression of the bla fusion protein serves to protect the cells against the effects of carbenicillin, thereby causing a retention in viable cells when grown in the presence of carbenicillin. Thus, if the antibody heavy or light chain is expressed in frame, the fusion protein is produced and the cells survive. Out of frame expression or the presence of stop codons will result in the death of the cells.

### F. METHODS OF PRODUCTION OF ANTIBODIES

Domain-exchanged antibodies can be expressed in host cells and produced therefrom. The antibodies that provided herein and/or that are identified against selected antigens (e.g. Candida) in the selection and screening methods above can be made by recombinant DNA methods that are within the purview of those skilled in the art. DNA encoding the antibodies can be readily isolated and sequenced using conventional procedures (e.g., by using oligonucleotide probes that are capable of binding specifically to genes encoding the heavy and light chains of the domain-exchanged antibodies). For example, the cell source used in the particular selection or screening method employed to identify the antibody (e.g. the phage) can serve as a preferred source of such DNA. In another example, once the sequence of the DNA encoding the antibodies is determined, nucleic acid sequences can be constructed using gene synthesis techniques.

The DNA also can be modified. For example, gene synthesis or routine molecular biology techniques can be used to effect insertion, deletion, addition or replacement of nucleotides. For example, additional nucleotide sequences can be joined to a nucleic acid sequence, for example, by covalently joining to the immunoglobulin coding sequence all or part of the coding sequence for a non-immunoglobulin polypeptide. In one example linker sequences can be added, such as sequences containing restriction endonuclease sites for the purpose of cloning the synthetic gene into a vector, for example, a protein expression vector or a vector designed for the amplification of the antibody constant region coding DNA sequences. Furthermore, additional nucleotide sequences specifying functional DNA elements can be operatively linked to a recombined germline encoding nucleic acid molecule. Examples of such sequences include, but are not limited to, promoter sequences designed to facilitate intracellular protein expression, and leader peptide sequences designed to facilitate protein secretion. Additional nucleotide sequences such as sequences specifying protein binding regions also can be linked to nucleic acid sequences. Such regions include, but are not limited to, sequences to facilitate uptake of recombined antibodies or fragments thereof into specific target cells, or otherwise enhance the pharmacokinetics of the synthetic gene.

The domain-exchanged antibodies can be expressed as full-length domain-exchanged antibodies, or as antibodies that are less then full length, for example, as domain-exchanged antibody fragments, including, but not limited to Fabs, Fab hinge fragment, scFv fragment, scFv tandem fragment and scFv hinge and scFv hinge(ΔE) fragments. Thus, for example, it is understood that any of the antibodies provided herein can be produced in any form so long as the resulting antibodies are domain-exchanged antibodies, which have a particular structure containing an interface formed by two interlocking V_{H} domains (V_{H}-V_{H}′ interface) and exhibit binding to Candida. For example, domain-exchanged antibodies provided herein generally contain at least two V_{H} chains and two V_{L} chains, whereby the V_{H} domains interact producing a V_{H}-V_{H}′ interface characteristic of the domain-exchanged configuration. The antibodies can further be produced to contain a hinge region, constant region or linkers.

2. Methods of Expression of Domain-Exchanged Antibodies

The nucleic acids encoding antibody polypeptides are typically cloned into a vector before transformation into prokaryotic or eukaryotic cells. Choice of vector can depend on the desired application. For example, after insertion of the nucleic acid, the vectors typically are used to transform host cells, for example, to amplify the recombined antibody genes for replication and/or expression thereof. In such examples, a vector suitable for high level expression is used.

In one example, nucleic acid encoding the heavy chain of a domain-exchanged antibody is ligated into a first expression vector and nucleic acid encoding the light chain of a domain-exchanged antibody is ligated into a second expression vector. The expression vectors can be the same or different, although generally they are sufficiently compatible to allow comparable expression of proteins (heavy and light chain) therefrom. For example, to generate a domain-exchanged Fab, sequences encoding the V_{H}-C_{H}1 can be cloned into a first expression vector and sequences encoding the V_{L}-C_{L} domains can be cloned into a second expression vector. An exemplary expression vector includes pTT5 (NRC Biotechnology Research) for expression in HEK293-6E cells. Other expression vectors and host cells are described below. The first and second expression vectors are co-transfected into host cells, typically at a 1:1 ratio. Upon expression of two copies of an antibody fragment chain (e.g., two copies of the V_{H}-C_{H}1 chain and V_{L}-C_{L}), two heavy chain variable regions (V_{H}) interlock and further pair with a light chain variable region (V_{L}) to generate domain-exchanged Fab dimers.

If desired, the vectors also can contain further sequences encoding additional constant region(s) or hinge regions to generate other antibody forms. For example, a full-length domain-exchanged antibody can be generated including in a first expression vector nucleotides encoding the heavy gene, sequences for the hinge and IgG constant regions; exemplary of such regions in 2G12 are amino acids 226-236 of SEQ ID NO:160 (hinge) and amino acids 237-454 of SEQ ID NO:160 (IgG1 Fc). Upon co-expression with the second expression vector encoding the V_{L}-C_{L} domains a full-length domain-exchanged antibody is expressed. Using these exemplified methods, it is within the level of one of skill in the art to generate other antibody forms, including other antibody fragment forms of domain-exchanged antibodies.

In another example, nucleic acid molecules encoding both the heavy and light chain of a domain-exchanged antibodies are expressed from the same vector. This is exemplified above with respect to display vectors (e.g. pCAL vectors). It is understood that any of the display vectors, for example, any pCAL vector, described above in Section E.b.1 can be used to produce soluble protein. For example, such vectors can be modified to not include the display protein (e.g. coat protein). Alternatively, vectors that do not contain a stop codon in the leader sequence but that do contain a stop codon between the nucleic acid encoding the antibody and the coat protein, can be introduced into a non-suppressor host cell strain. Upon expression, there is no readthrough of the stop codon, so that only soluble antibody chains are expressed without fusion to a coat protein.

Using either of the above methods, one of skill in the art can generate a full-length domain-exchanged antibody, or a domain-exchanged antibody fragment such as any described herein below.

a. Domain-Exchanged Fab Fragment

The domain-exchanged Fab fragment can be generated using a vector containing a nucleic acid encoding the V_{H}-C_{H}1 chain, followed by a nucleic acid encoding a stop codon (e.g. the amber stop codon (TAG)), and optionally followed by a nucleic acid encoding a coat protein. In one example, the vector also includes the nucleic acid encoding a light chain (V_{L}-C_{L}). Alternatively, the light chain can be expressed from another vector, which is used to transform the same host cell. The vectors for display of the domain-exchanged Fab antibody are designed such that, when expressed, for example in a non-suppressor cell, two copies of the soluble heavy chain assemble, along with two soluble light chains produced by the same vector or a different vector, to form the domain-exchanged “Fab” antibody having two conventional antibody combining sites. The Fab exists as a dimer.

b. Domain-Exchanged scFv Fragment

A domain-exchanged scFv fragment contains two chains, each of which contains one V_{H} and one V_{L} domain, joined by a peptide linker (V_{H}-linker-V_{L}). In the folded domain-exchanged scFv fragment, the two chains interact through the V_{H} domains, providing the interlocked domain-exchanged configuration. The domain-exchanged scFv fragment can be generated with a vector containing a nucleic acid encoding the V_{H}-linker-V_{L} single chain, followed by a sequence encoding a stop codon (e.g. the amber stop codon (TAG)), and optionally followed by a sequence encoding a coat protein. Such a vector is designed so that, when expressed, for example in a non-suppressor cell, a soluble single chain (V_{H}-linker-V_{L}) is produced. Upon interaction with other produced proteins a scFv fragment is produced having two chains and two conventional antibody combining sites.

c. Domain-Exchanged Fab Hinge Fragment

A domain-exchanged Fab hinge fragment is generated by inserting a nucleic acid encoding a hinge region into the domain-exchanged Fab fragment vector, between the nucleic acid encoding the C_{H}1 domain and, if included, the nucleic acid encoding the coat protein. Thus, the domain-exchanged Fab hinge fragment is identical to the domain-exchanged Fab fragment, except that each heavy chain further includes a hinge region in each heavy chain following the C_{H}1 region, which promotes interaction between the two heavy chains.

d. Domain-Exchanged scFv Tandem Fragment

In the nucleic acid molecule encoding a domain-exchanged scFv fragment, three nucleic acids encoding peptide linkers are inserted between the nucleic acids encoding a first V_{L} and first V_{H} chain, between the nucleic acids encoding the first V_{H} and a second V_{H} chain, and between nucleic acids encoding the second V_{H} and a second V_{L} chain. Thus, the scFv tandem vector carries two copies each of identical nucleic acid molecules encoding the light chain and heavy chain variable region domains, all four of which are joined by nucleic acids encoding peptide linkers. Thus, in the fragment, two heavy and two light chain variable region domains are joined by peptide linkers.

e. Domain-Exchanged Single Chain Fab Fragments

To generate a single chain Fab fragment, the domain-exchanged Fab fragment is modified by inserting sequences encoding peptide linkers between the V_{L}-C_{L} sequence and the V_{H}-C_{H}1, thereby generating upon expression, for example in a non-suppressor host cell, one soluble V_{L}-C_{L}-linker-V_{H}-C_{H}1 chain to form a single chain Fab (scFab) fragment, such as the scFab ΔC², having the domain-exchanged configuration. In the scFab ΔC² fragment, two cysteines are mutated to ablate formation of the disulfide bonds between the constant regions, as the presence of the linkers makes these disulfide bonds unnecessary for stabilizing the folded antibody fragment. A modified scFab ΔC² fragment, the scFab ΔC²Cys19 fragment, is described below.

f. Domain-Exchanged Fab Cys19

The domain-exchanged Fab Cys 19 fragment is identical to the domain-exchanged Fab fragment, but carries this Ile-Cys mutation; the domain-exchanged scFab ΔC²Cys19 is identical to the domain-exchanged scFab ΔC² fragment but further carries this mutation; and the scFv Cys19 is identical to the domain-exchanged scFv fragment, but carries this additional mutation. Nucleic acid sequences of exemplary vectors encoding domain-exchanged 2G12 Fab Cys19, scFab ΔC²Cys19, and scFv Cys19 fragments are set forth in SEQ ID NOS: 191, 192, and 193, respectively.

g. Domain-Exchanged scFv Hinge

A domain-exchanged scFv hinge fragment is generated by inserting into the vector encoding the domain-exchanged scFv fragment a nucleic acid encoding a hinge region between the nucleic acids encoding the V_{H} and, if included, the coat protein. Thus, the domain-exchanged scFv hinge fragment is identical to the domain-exchanged Fab fragment, with the exception that a hinge region is included in each chain, promoting formation of a disulfide bridge, which can stabilize the configuration of the domain-exchanged fragment.

3. Vectors

Many expression vectors are available and known to those of skill in the art for the expression of antibodies or portions thereof. The choice of an expression vector is influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vectors in the cells. Vectors also generally can contain additional nucleotide sequences operably linked to the ligated nucleic acid molecule (e.g. His tag, Flag tag). For purposes herein, vectors generally include sequences encoding the constant region. Thus, antibodies or portions thereof also can be expressed as protein fusions. For example, a fusion can be generated to add additional functionality to a polypeptide. Examples of fusion proteins include, but are not limited to, fusions of a signal sequence, an epitope tag such as for localization, e.g. a his₆ tag or a myc tag, or a tag for purification, for example, a GST fusion, and a sequence for directing protein secretion and/or membrane association.

For example, expression of the proteins can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application. Promoters which can be used include but are not limited to eukaryotic expression vectors containing the SV40 early promoter (Bernoist and Chambon, (1981) Nature 290:304-310), the promoter contained in the 3′ long terminal repeat of Rous sarcoma virus (Yamamoto et al. (1980) Cell 22:787-797), the herpes thymidine kinase promoter (Wagner et al., (1981) Proc. Natl. Acad. Sci. USA 78:1441-1445), the regulatory sequences of the metallothionein gene (Brinster et al., (1982) Nature 296:39-42); prokaryotic expression vectors such as the β-lactamase promoter (Jay et al., (1981) Proc. Natl. Acad. Sci. USA 78:5543) or the tac promoter (DeBoer et al., (1983) Proc. Natl. Acad. Sci. USA 80:21-25); see also “Useful Proteins from Recombinant Bacteria”: in Scientific American 242:79-94 (1980)); plant expression vectors containing the nopaline synthetase promoter (Herrara-Estrella et al., (1984) Nature 303:209-213) or the cauliflower mosaic virus 35S RNA promoter (Gardner et al., (1981) Nucleic Acids Res. 9:2871), and the promoter of the photosynthetic enzyme ribulose bisphosphate carboxylase (Herrera-Estrella et al., (1984) Nature 310:115-120); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., (1984) Cell 38:639-646; Ornitz et al., (1986) Cold Spring Harbor Symp. Quant. Biol. 50:399-409; MacDonald, (1987) Hepatology 7:425-515); insulin gene control region which is active in pancreatic beta cells (Hanahan et al., (1985) Nature 315:115-122), immunoglobulin gene control region which is active in lymphoid cells (Grosschedl et al., (1984) Cell 38:647-658; Adams et al., (1985) Nature 318:533-538; Alexander et al., (1987) Mol. Cell. Biol. 7:1436-1444), mouse mammary tumor virus control region which is active in testicular, breast, lymphoid and mast cells (Leder et al., (1986) Cell 45:485-495), albumin gene control region which is active in liver (Pinckert et al., (1987) Genes and Devel. 1:268-276), alpha-fetoprotein gene control region which is active in liver (Krumlauf et al., (1985) Mol. Cell. Biol. 5:1639-1648; Hammer et al., (1987) Science 235:53-58), alpha-1 antitrypsin gene control region which is active in liver (Kelsey et al., (1987) Genes and Devel. 1:161-171), beta globin gene control region which is active in myeloid cells (Magram et al., (1985) Nature 315:338-340; Kollias et al., (1986) Cell 46:89-94), myelin basic protein gene control region which is active in oligodendrocyte cells of the brain (Readhead et al., (1987) Cell 48:703-712), myosin light chain-2 gene control region which is active in skeletal muscle (Shani, (1985) Nature 314:283-286), and gonadotrophic releasing hormone gene control region which is active in gonadotrophs of the hypothalamus (Mason et al., (1986) Science 234:1372-1378).

In addition to the promoter, the expression vector typically contains a transcription unit or expression cassette that contains all the additional elements required for the expression of the antibody, or portion thereof, in host cells. A typical expression cassette contains a promoter operably linked to the nucleic acid sequence encoding the antibody chain and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or may be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Vectors can be provided that contain a sequence of nucleotides that encodes a constant region of an antibody operably linked to the nucleic acid sequence encoding the variable region of the antibody. The vector can include the sequence for one or all of a C_{H}1, C_{H}2, hinge, C_{H}3 or C_{H}4 and/or C_{L}. Generally, such as for expression of Fabs, the vector contains the sequence for a C_{H}1 or C_{L} (kappa or lambda light chains). The sequences of constant regions or hinge regions are known to one of skill in the art (see e.g. U.S. Published Application No. 20080248028) and described herein.

Exemplary expression vectors include any mammalian expression vector such as, for example, pCMV. For bacterial expression, such vectors include pBR322, pUC, pSKF, pET23D, and fusion vectors such as MBP, GST and LacZ. Other eukaryotic vectors, for example any containing regulatory elements from eukaryotic viruses can be used as eukaryotic expression vectors. These include, for example, SV40 vectors, papilloma virus vectors, and vectors derived from Epstein-Bar virus. Other exemplary eukaryotic vectors include pMSG, pAV009/A+, pMT010/A+, pMAMneo-5, baculovirus pDSCE, and any other vector allowing expression of proteins under the direction of the CMV promoter, SV40 early promoter, SV40 late promoter, metallothionein promoter, murine mammary tumor virus promoter, Rous sarcoma virus promoter, polyhedron promoter, or other promoters shown effective for expression in eukaryotes.

Exemplary vectors for expression of domain-exchanged antibodies include the pCAL vectors described in Section E.b.1 above.

Any methods known to those of skill in the art for the insertion of DNA fragments into a vector can be used to construct expression vectors containing a nucleic acid encoding an antibody chain. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

4. Cells and Expression Systems

Cells containing the vectors also are provided. Generally, any cell type that can be engineered to express heterologous DNA and has a secretory pathway is suitable. Expression hosts include prokaryotic and eukaryotic organisms such as bacterial cells (e.g. E. coli), yeast cells, fungal cells, Archea, plant cells, insect cells and animal cells including human cells. Expression hosts can differ in their protein production levels as well as the types of post-translational modifications that are present on the expressed proteins. Further, the choice of expression host is often related to the choice of vector and transcription and translation elements used. For example, the choice of expression host is often, but not always, dependent on the choice of precursor sequence utilized. For example, many heterologous signal sequences can only be expressed in a host cell of the same species (i.e., an insect cell signal sequence is optimally expressed in an insect cell). In contrast, other signal sequences can be used in heterologous hosts such as, for example, the human serum albumin (hHSA) signal sequence which works well in yeast, insect, or mammalian host cells and the tissue plasminogen activator pre/pro sequence which has been demonstrated to be functional in insect and mammalian cells (Tan et al., (2002) Protein Eng. 15:337). The choice of expression host can be made based on these and other factors, such as regulatory and safety considerations, production costs and the need and methods for purification. Thus, the vector system must be compatible with the host cell used.

Expression in eukaryotic hosts can include expression in yeast such as Saccharomyces cerevisiae and Pichia pastoris, insect cells such as Drosophila cells and lepidopteran cells, plants and plant cells such as tobacco, corn, rice, algae, and Lemna. Eukaryotic cells for expression also include mammalian cells lines such as Chinese hamster ovary (CHO) cells or baby hamster kidney (BHK) cells. Eukaryotic expression hosts also include production in transgenic animals, for example, including production in serum, milk and eggs.

Expression vectors can be introduced into host cells via, for example, transformation, transfection, infection, electroporation and sonoporation, so that many copies of the gene sequence are generated. Generally, standard transfection methods are used to produce bacterial, mammalian, yeast, or insect cell lines that express large quantity of antibody chains, which is then purified using standard techniques (see e.g., Colley et al. (1989) J. Biol. Chem., 264:17619-17622; Guide to Protein Purification, in Methods in Enzymology, vol. 182 (Deutscher, ed.), 1990). Transformation of eukaryotic and prokaryotic cells are performed according to standard techniques (see, e.g., Morrison (1977) J. Bact. 132:349-351; Clark-Curtiss and Curtiss (1983) Methods in Enzymology, 101, 347-362). For example, any of the well-known procedures for introducing foreign nucleotide sequences into host cells can be used. These include the use of calcium phosphate transfection, polybrene, protoplast fusion, electroporation, biolistics, liposomes, microinjection, plasma vectors, viral vectors and any other the other well known methods for introducing cloned genomic DNA, cDNA, synthetic DNA or other foreign genetic material into a host cell. Generally, for purposes herein, host cells are transfected with a first vector encoding at least a V_{H} chain and a second vector encoding at least a V_{L} chain. Thus, it is only necessary that the particular genetic engineering procedure used be capable of successfully introducing at least both genes into the host cell capable of expressing antibody polypeptide.

Transformation of host cells with recombinant DNA molecules that incorporate the variable region gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA. Generally, after the expression vector is introduced into the cells, the transfected cells are cultured under conditions favoring expression of the germline chain, which is recovered from the culture using standard purification techniques identified below.

Antibodies and portions thereof can be produced using a high throughput approach by any methods known in the art for protein production including in vitro and in vivo methods such as, for example, the introduction of nucleic acid molecules encoding antibodies or portions thereof into a host cell or host animal and expression from nucleic acid molecules encoding recombined antibodies in vitro. Prokaryotes, especially E. coli, provide a system for producing large amounts of antibodies or portions thereof, and are particularly desired in applications of high-throughput expression and purification of proteins. Transformation of E. coli is a simple and rapid technique well known to those of skill in the art. E. coli host strains for high throughput expression include, but are not limited to, BL21 (EMD Biosciences) and LMG194 (ATCC). Exemplary of such an E. coli host strain is BL21. Vectors for high throughput expression include, but are not limited to, pBR322 and pUC vectors. Automation of expression and purification can facilitate high-throughput expression

a. Prokaryotic Expression

Prokaryotes, especially E. coli, provide a system for producing large amounts of antibodies or portions thereof. Transformation of E. coli is a simple and rapid technique well known to those of skill in the art. Expression vectors for E. coli can contain inducible promoters that are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λP_{L} promoter.

Antibodies or portions thereof can be expressed in the cytoplasmic environment of E. coli. The cytoplasm is a reducing environment and for some molecules, this can result in the formation of insoluble inclusion bodies. Reducing agents such as dithiothreitol and β-mercaptoethanol and denaturants (e.g., such as guanidine-HCl and urea) can be used to resolubilize the proteins. An exemplary alternative approach is the expression of recombined antibodies or fragments thereof in the periplasmic space of bacteria which provides an oxidizing environment and chaperonin-like and disulfide isomerases leading to the production of soluble protein. Typically, a leader sequence is fused to the protein to be expressed which directs the protein to the periplasm. The leader is then removed by signal peptidases inside the periplasm. There are three major pathways to translocate expressed proteins into the periplasm, namely the Sec pathway, the SRP pathway and the TAT pathway. Examples of periplasmic-targeting leader sequences include the pelB leader from the pectate lyase gene, the StII leader sequence, and the DsbA leader sequence. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility. Typically, temperatures between 25° C. and 37° C. are used. Mutations also can be used to increase solubility of expressed proteins. Typically, bacteria produce aglycosylated proteins. Thus, if proteins require glycosylation for function, glycosylation can be added in vitro after purification from host cells.

b. Yeast

Yeasts such as Saccharomyces cerevisiae, Schizosaccharomyces pombe, Yarrowia lipolytica, Kluyveromyces lactis, and Pichia pastoris are useful expression hosts for antibodies or portions thereof. Yeast can be transformed with episomal replicating vectors or by stable chromosomal integration by homologous recombination. Typically, inducible promoters are used to regulate gene expression. Examples of such promoters include AOX1, GAL1, GAL7, and GAL5 and metallothionein promoters such as CUP1. Expression vectors often include a selectable marker such as LEU2, TRP1, HIS3, and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisiae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insects

Insect cells, particularly using baculovirus expression, are useful for expressing antibodies or portions thereof. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter and p10 promoter of baculovirus. Commonly used baculovirus systems include the baculoviruses such as Autographa californica nuclear polyhedrosis virus (AcNPV), and the Bombyx mori nuclear polyhedrosis virus (BmNPV) and an insect cell line such as Sf9 derived from Spodoptera frugtiperda and TN derived from Trichoplusia ni. For high-level expression, the nucleotide sequence of the molecule to be expressed is fused immediately downstream of the polyhedrin initiation codon of the virus. To generate baculovirus recombinants capable of expressing human antibodies, a dual-expression transfer, such as pAcUW51 (PharMingen) is utilized. Mammalian secretion signals are accurately processed in insect cells and can be used to secrete the expressed protein into the culture medium

An alternative expression system in insect cells is the use of stably transformed cells. Cell lines such as Sf9 derived cells from Spodoptera frugiperda and TN derived cells from Trichoplusia ni can be used for expression. The baculovirus immediate early gene promoter IE1 can be used to induce consistent levels of expression. Typical expression vectors include the pIE1-3 and pI31-4 transfer vectors (Novagen). Expression vectors are typically maintained by the use of selectable markers such as neomycin and hygromycin.

d. Mammalian Cells

Mammalian expression systems can be used to express antibodies or portions thereof. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high-level expression, for example the SV40 promoter-enhancer, the human cytomegalovirus (CMV) promoter and the long terminal repeat of Rous sarcoma virus. These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Antibodies are typically produced using a NEO^{R}/G418 system, a dihydrofolate reductase (DHFR) system or a glutamine synthetase (GS) system. The GS system uses joint expression vectors, such as pEE12/pEE6, to express both heavy chain and light chain. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. One such example is the serum free EBNA-1 cell line (Pham et al., (2003) Biotechnol. Bioeng. 84:332-42.)

e. Plants

Transgenic plant cells and plants can be used to express proteins such as any antibody or portion thereof described herein. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus CaMV 35S promoter, the nopaline synthase promoter, the ribose bisphosphate carboxylase promoter and the maize ubiquitin-1 (ubi-1) promoter promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce proteases or modified proteases (see for example, Mayfield et al. (2003) PNAS 100:438-442). Because plants have different glycosylation patterns than mammalian cells, this can influence the choice of protein produced in these hosts.

5. Purification

Antibodies and portions thereof are purified by any procedure known to one of skill in the art. The domain-exchanged antibodies can be purified to substantial purity using standard protein purification techniques known in the art including but not limited to, SDS-PAGE, size fraction and size exclusion chromatography, ethanol precipitation, reverse phase HPLC, ammonium sulfate precipitation, chelate chromatography, ionic exchange chromatography or column chromatography. For example, antibodies can be purified by column chromatography. Exemplary of a method to purify antibodies is by using column chromatography, wherein a solid support column material is linked to Protein G, a cell surface-associated protein from Streptococcus, that binds immunoglobulins with high affinity. Protein A also can be used to purified antibodies. The antibodies can be purified to 60%, 70%, 80% purity and typically at least 90%, 91%, 92%, 93%, 94%, 95%, 96%, 97%, 98% or 99% purity. Purity can be assessed by standard methods such as by SDS-PAGE and Coomassie staining.

In one aspect, Protein A immobilized on a solid phase is used for immunoaffinity purification of full-length domain-exchanged antibodies. Protein A is a 41 kD cell wall protein from Staphylococcus aureus, which binds with a high affinity to the Fc region of antibodies (Lindmarke et al. (1983) J. Immunol. Method., 62:1-13). The solid phase to which Protein A is immobilized is generally a column containing a glass or silica surface, generally a controlled pore glass column or a silica acid column. In some applications, the column has been coated with a reagent, such as glycerol, in an attempt to prevent nonspecific adherence of contaminants. The antibody can be recovered from the solid phase by elution.

Since domain-exchanged antibodies exist as dimers, and sometimes as larger ordered oligomers, purification schemes can be effected to select particular structures. For example, purification schemes can involve two size exclusion chromatography steps. In an exemplary example, the first step can include running a first concentrated protein A eluate over a Superdex 200 16/60 column, and then the monomer and dimer fractions can be separately placed over a Superdex 200 10/30 column (see e.g. West et al. (2009) J. Virol., 83:98-104).

Methods for purification of domain-exchanged antibodies from host cells depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary further the proteins can be extracted and further purified using standard methods in the art.

When antibodies are expressed by transformed bacteria in large amounts, typically after promoter induction, although expression can be constitutive, the polypeptides can form insoluble aggregates. There are several protocols that are suitable for purification of polypeptide inclusion bodies known to one of skill in the art. Numerous variations will be apparent to those of skill in the art.

For example, in one method, the cell suspension is generally centrifuged and the pellet containing the inclusion bodies resuspended in buffer which does not dissolve but washes the inclusion bodies, e.g., 20 mM Tris-HCL (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies can be resuspended in an appropriate buffer (e.g., 20 mM sodium phosphate, pH 6.8, 150 mM NaCl). Other appropriate buffers are apparent to those of skill in the art.

Alternatively, antibodies can be purified from bacteria periplasm. Where the polypeptide is exported into the periplasm of the bacteria, the periplasmic fraction of the bacteria can be isolated by cold osmotic shock in addition to other methods known to those of skill in the art. For example, in one method, to isolate recombinant polypeptides from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant polypeptides present in the supernatant can be separated from the host proteins by standard separation techniques well known to those of skill in the art. These methods include, but are not limited to, the following steps: solubility fractionation, size differential filtration, and column chromatography.

### F. ASSESSING ANTI-CANDIDA ANTIBODY PROPERTIES AND ACTIVITIES

The anti-Candida antibodies provided herein can be characterized in a variety of ways well-known to one of skill in the art. For example, the anti-Candida antibodies provided herein can be assayed for the ability to immunospecifically bind to Candida, such as for example binding to a surface antigen of Candida. Such assays can be performed, for example, in solution (e.g., Houghten (1992) Bio/Techniques 13:412-421), on beads (Lam (1991) Nature 354:82-84), on chips (Fodor (1993) Nature 364:555-556), on bacteria (U.S. Pat. No. 5,223,409), on spores (U.S. Pat. Nos. 5,571,698; 5,403,484; and 5,223,409), on plasmids (Cull et al. (1992) Proc. Natl. Acad. Sci. USA 89:1865-1869) or on phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310). Antibodies that have been identified to immunospecifically bind to a Candida antigen also can be assayed for their specificity and affinity for a Candida antigen. In addition, in vitro assays and in vivo animal models using the anti-Candida antibodies provided herein can be employed for measuring the level of Candida inhibition effected by contact or administration of the anti-Candida antibodies.

1. Binding Assays

The anti-Candida antibodies provided herein can be assessed for their ability to bind a selected target (e.g., whole Candida yeast or an isolated Candida-expressed protein) and the specificity for such targets by any method known to one of skill in the art. Exemplary assays are described herein below. Binding assays can be performed in solution, suspension or on a solid support. For example, whole yeast cells can be incubated first with the anti-Candida antibodies followed by a second incubation with a secondary antibody that recognizes the primary antibody and is conjugated with a label such as FITC. After labeling, the yeast cells can be counted with a flow cytometer to analyze the antibody binding (see, U.S. Pat. Appl. No. 2004/0142385). In another example, target antigens can be immobilized to a solid support (e.g. a carbon or plastic surface, a tissue culture dish or chip) and contacted with antibody. Unbound antibody or target protein can be washed away and bound complexes can then be detected. Binding assays can be performed under conditions to reduce nonspecific binding, such as by using a high ionic strength buffer (e.g. 0.3-0.4 M NaCl) with nonionic detergent (e.g. 0.1% Triton X-100 or Tween 20) and/or blocking proteins (e.g. bovine serum albumin or gelatin). Negative controls also can be included in such assays as a measure of background binding. Binding affinities can be determined using Scatchard analysis (Munson et al., (1980) Anal. Biochem., 107:220), surface plasmon resonance, isothermal calorimetry, or other methods known to one of skill in the art.

Exemplary immunoassays which can be used to analyze immunospecific binding and cross-reactivity include, but are not limited to, competitive and non-competitive assay systems using techniques such as, but not limited to, western blots, radioimmunoassays, ELISA (enzyme linked immunosorbent assay), Meso Scale Discovery (MSD, Gaithersburg, Md.), “sandwich” immunoassays, immunoprecipitation assays, ELISPOT, precipitin reactions, gel diffusion precipitin reactions, immunodiffusion assays, agglutination assays, complement-fixation assays, immunoradiometric assays, fluorescent immunoassays, protein A immunoassays, immunohistochemistry, or immuno-electron microscopy. Such assays are routine and well known in the art (see, e.g., Ausubel et al., Eds, 1994, Current Protocols in Molecular Biology, Vol. 1, John Wiley & Sons, Inc., New York, which is incorporated by reference herein in its entirety). Other assay formats include liposome immunoassays (LIA), which use liposomes designed to bind specific molecules (e.g., antibodies) and release encapsulated reagents or markers. The released chemicals are then detected according to standard techniques (see Monroe et al., (1986) Amer. Clin. Prod. Rev. 5:34-41). Exemplary immunoassays not intended by way of limitation are described briefly below.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody or antigen-binding fragment thereof of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody of interest to immunoprecipitate a particular antigen can be assessed by, e.g., western blot analysis. One of skill in the art is knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., Eds, 1994, Current Protocols in Molecular Biology, Vol. 1, John Wiley & Sons, Inc., New York at 10.16.1.

Western blot analysis generally includes preparing yeast extract samples or yeast cell wall extracts, electrophoresis of the samples in a polyacrylamide gel (e.g., 8%-20% SDS-PAGE depending on the molecular weight of the antigen) or via 2-D gel electrophoresis (see, e.g., WO 04/043276), transferring the sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), probing the membrane with primary antibody (i.e. the antibody of interest) diluted in blocking buffer, washing the membrane in washing buffer, probing the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art is knowledgeable as to the parameters that can be modified to increase the signal detected and to reduce the background noise. For further discussion regarding western blot protocols see, e.g., Ausubel et al., Eds, 1994, Current Protocols in Molecular Biology, Vol. 1, John Wiley & Sons, Inc., New York at 10.8.1.

ELISAs include preparing antigen, coating the well of a 96-well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs, the antibody of interest does not have to be conjugated to a detectable compound; instead, a second antibody (which recognizes the antibody of interest) conjugated to a detectable compound can be added to the well. Further, instead of coating the well with the antigen, the antibody can be coated to the well. In this case, a second antibody conjugated to a detectable compound can be added following the addition of the antigen of interest to the coated well. One of skill in the art is knowledgeable as to the parameters that can be modified to increase the signal detected as well as other variations of ELISAs known in the art. For further discussion regarding ELISAs see, e.g., Ausubel et al., Eds, 1994, Current Protocols in Molecular Biology, Vol. 1, John Wiley & Sons, Inc., New York at 11.2.1.

Immunohistochemistry comprises preparing a tissue sample (e.g. from a Candida infected animal), fixing the tissue to preserve protein molecules in their native conformation, bathing the sample in a permeabilization reagent (e.g. Tween, Nonidet P40) to penetrate the tissue, blocking the sample with blocking solution (e.g., PBS with 3% BSA or non-fat milk), washing the sample in washing buffer (e.g., PBS-Tween 20), probing the sample with primary antibody (i.e. the antibody of interest) diluted in blocking buffer, washing the sample in washing buffer, probing the sample with a secondary antibody (which recognizes the primary antibody, e.g., an anti-human antibody) conjugated to a fluorescent dye (e.g. fluorescein isothiocyanate, Alexa fluor, rhodamine) diluted in blocking buffer, washing the sample in wash buffer, and detecting the presence of the antigen via fluorescent microscopy. One of skill in the art is knowledgeable as to the parameters that can be modified to increase the signal detected and to reduce the background noise.

The binding affinity of an antibody to an antigen and the off-rate of an antibody-antigen interaction can be determined, for example, by competitive binding assays. One example of a competitive binding assay is a radioimmunoassay involving the incubation of labeled antigen (e.g., ³H or ¹²⁵I) with the antibody of interest in the presence of increasing amounts of unlabeled antigen, and the detection of the antibody bound to the labeled antigen. The affinity of an anti-Candida antibody provided herein for a Candida antigen and the binding off-rates can be determined from the data by Scatchard plot analysis. Competition with a second antibody can also be determined using radioimmunoassays. In this case, a Candida antigen is incubated with an anti-Candida antibody provided herein conjugated to a labeled compound (e.g., ³H or ¹²⁵I) in the presence of increasing amounts of an unlabeled second antibody. In some examples, surface plasmon resonance (e.g., BiaCore 2000, Biacore AB, Upsala, Sweden and GE Healthcare Life Sciences; Malmqvist et al. (1993) Curr Opin Immunol. 5(2):282-6; Garcia-Ojeda et al. (2004) Infect Immun. 72(6):3451-60) kinetic analysis can be used to determine the binding on and off rates of antibodies to a Candida antigen. Surface plasmon resonance kinetic analysis comprises analyzing the binding and dissociation of a Candida antigen from chips with immobilized antibodies on their surface.

2. In Vitro Assays for Analyzing Candida Inhibitory Effects of Antibodies

The anti-Candida antibodies provided herein can be analyzed by any suitable method known in the art for measurement of the inhibition or amelioration of Candida infection. Methods for measuring inhibition or amelioration of infection include, but are not limited to, metabolic activity assays, adhesion and invasion assays, biofilm formation assays, growth assays, hyphae formation assays, opsonization assays and gene transcription assays. Such assays can be employed to assess, for example, inhibition of yeast viability, adherence to and invasion of host cells, biofilm formation, growth and virulence, the promotion of phagocytosis by macrophage and the expression of cytokines such as IL-8 (see, e.g. Hasan et al. (2009) Microbes Infect. 11(8-9):753-61; Arseculeratne et al. (2007) Indian J Med Microbiol. 25(3):267-71; Calderone et al. (2009) Methods Mol. Biol. 499:85-93; Sohn et al. (2009) Methods Mol. Biol. 470:95-104; Hernandez et al. (2009) Methods Mol Biol. 470:105-23; Jayatilake et al. (2008) Mycopathologia 165(6):373-80; Torosantucci et al. (2009) PLoS One. 4(4):e5392; Dorocka-Bobkowska et al. (2009) Med Sci Monit. 15(9):BR262-9; Hollmer et al. (2006) Infect Dis Obstet Gynecol. 2006:98218; Maki et al. (2007) Microbiol Immunol. 51(11):1053-9; Mathews et al. (1998) J Med Microbiol. 47(11):1007-14; González-Novo et al. (2008) Mol Biol Cell. 19(4):1509-18; Calderone (2009) Methods Mol Biol. 499:85-93; WO 99/52922; WO 06/097689). One of skill in the art can identify any assay capable of measuring inhibition or amelioration of Candida infection.

Metabolic assays can be used to measure the ability of the anti-Candida antibodies to reduce the number of viable cells present in a culture or sample. Metabolic assays include, for example, XTT reduction assays and MTT reduction assays. Reduction of XTT or MTT only occurs when reductase enzymes are active, therefore these assays measure the viability of Candida by measuring the extent of their metabolic activity. In exemplary XTT or MTT reduction assays, MTT (3-(4,5-Dimethylthiazol-2-yl)-2,5-diphenyltetrazolium bromide, a tetrazole) or XTT (2,3-bis (2-methoxy-4-nitro-5-sulfophenyl)-5-[(phenylamino) carbonyl]-2H-tetrazolium hydroxide) are reduced to form a colored formazan in living cells. The formazan derivative of MTT is insoluble while the formazan derivative of XTT is soluble; thus, XTT reduction can be measured in Candida supernatants. One of skill in the art can determine which reduction assay is best based on the Candida sample to be measured. In both assays, the colored formazan can be easily quantified colorimetrically allowing the colored derivative to be predictive of the extent of metabolic activity and thus yeast viability. A decrease in the formation of the derivative indicates the ability of the antibody to reduce the number of live cells present in the culture or sample.

Adhesion assays can be used to measure the ability of the anti-Candida antibodies to inhibit the adhesion of Candida to host epithelial or endothelial cells. These assays can utilize a variety of cell line monolayers that include, but are not limited to, Hep-2, HeLa, and HS3 cells. One of skill in the art can identify appropriate cell lines for use in this assay. In exemplary adhesion assays, Candida cultures are pre-incubated with or without anti-Candida antibodies. Epithelial cell monolayers are then incubated with the Candida for a predetermined period of time after which, non-adherent fungal cells are washed away with PBS. Adherent fungal cells can then be removed with PBS+Triton and counted using standard CFU (colony forming units) assays that are known in the art. Alternatively, the Candida can be pre-labeled with fluorescein isothiocyanate or another compound (e.g. rhodamine, phycoerythrin, phycocyanin, allophycocyanin, o-phthaladehyde and fluorescamine) or a radioactive label (e.g., ³²P, ³⁵S, and ¹²⁵I) and adherent fungal cells counted via the detectable compound (e.g. flow cytometry, scintillation). Using still another alternative, monolayers with bound fungal cells can be air dried, gram stained and mounted on a glass slide. The number of attached fungal cells can be counted visually using a light microscope to count the yeast on fifty fields to extrapolate the total number of bound yeast cells. A reduction in the number of adherent fungal cells in samples pretreated with anti-Candida antibodies indicates that the antibodies are capable of preventing adhesion of Candida to host cells.

Other adhesion assays can be used to measure the ability of the anti-Candida antibodies to inhibit the adhesion of Candida to host epithelial or endothelial cells. These assays can utilize a variety of primary human cell samples that include, but are not limited to, human buccal epithelial cells (BEC) and human umbilical vein endothelial cells (HUVEC). Candida cells can be radiolabeled with ³⁵S-methionine and mixed in solution with BECs. After an incubation period, the BECs can be filtered through polycarbonate filters and the radioactivity quantitated in a scintillation counter. A decrease in radioactivity on the filter indicates a decrease in Candida binding to the BECs. Adhesion assays can also be performed by measuring the adherence of Candida to polystyrene plastic plates. Candida cells can be incubated with anti-Candida antibodies then poured into 6-well polystyrene plates. After washing to remove the non-adherent cells, Sabourad dextrose agar is poured into the wells and allowed to solidify. Colonies that appear after 24 hr of incubation at 37° C. are counted. A decrease in the number of colonies indicates the ability of the anti-Candida antibodies to inhibit adherence to plastic.

Tissue invasion assays can be used to measure the ability of the anti-Candida antibodies to inhibit the tissue invasion abilities of Candida. Exemplary tissue invasion assays can use a variety of tissues and cell lines that include, but are not limited to, tissues derived from human carcinomas, rabbit tongue mucosal explants, and reconstituted human oral epithelium. Alternatively, epithelial cells grown on a collagen gel to build models of intestinal (Caco-2 cells), vaginal (A431 cells) and oral (TR146 cells) mucosa tissues can be used as invasion matrices. Candida cultures can be pre-incubated with the anti-Candida antibodies and then introduced to the tissue models. These assays utilize light or scanning electron microscopy with immunohistological processing to observe the invasiveness of Candida into tissue models. A reduction in tissue invasiveness of Candida pre-treated with anti-Candida antibodies indicates that the anti-Candida antibodies have the ability to reduce the invasiveness of the fungal cells.

Biofilms are structures of microbial communities that are attached to a surface and encased in a matrix of extracellular polymeric substances. The formation of biofilms has been associated with the virulence of Candida. Thus, biofilm formation assays can be used to measure the ability of the anti-Candida antibodies to inhibit the formation of the pathogenically important biofilms. In exemplary biofilm formation assays, the Candida can be pre-treated with anti-Candida antibodies followed by the incubation of the Candida fungal cells with a suitable adherent material to allow biofilm formation. One of skill in the art can determine the optimal material and conditions for biofilm formation based on known examples in the art. The intensity of the staining of the biofilm with Crystal Violet provides a visible measurement of the ability of the anti-Candida antibodies to block biofilm formation. Alternatively, the XTT reduction assay (see above) can also measure the viability of the resultant biofilm as an indicator of the ability of the anti-Candida antibodies to block biofilm formation.

Growth assays can be used to measure the ability of the anti-Candida antibodies to inhibit growth of Candida in vitro. In exemplary growth assays, Candida cells can be collected from an overnight culture and added to a single well of a 96-well dish. Hyphal forms of Candida can be obtained by modifying growth conditions in ways known in the art. Anti-Candida antibodies are then incubated with Candida during their growth over a predetermined period of time. Tritiated uridine is then added to the growth culture and the amount of uridine incorporated into the Candida RNA can be counted via scintillation. A reduction in the amount of uridine incorporated into the Candida RNA indicates the ability of the anti-Candida antibodies to inhibit the growth of Candida. Alternatively, Candida can be incubated with anti-Candida antibodies and allowed to grow over a predetermined period of time after which their growth is measured by a classical plate dilution CFU (colony forming units) assay which counts the number of colonies that are capable of growing on Sabouraud agar plates per unit volume of inoculum. A variety of CFU assays are known in the art and can be used to measure the number of yeast cells in a sample after a given growth period.

Hyphae formation is a property associated with Candida virulence; thus, hyphae formation assays can be used to assess the virulence of Candida. To measure the ability of anti-Candida antibodies to inhibit the virulence of Candida via the inhibition of hyphae formation, hyphae formation assays can be used. In exemplary hyphae formation assays, Candida are allowed to grow overnight in liquid non-filament-inducing YEPD (yeast extract peptone-dextrose) media at 30° C. Candida are then incubated for a predetermined period of time with anti-Candida antibodies and then plated on a solid filament-inducing media. Filament-inducing medias can include, but are not limited to, Spider media, Lee's pH 6.8 media and YEPD+Serum (grown at 37° C.). The inhibition or reduction of hyphae as observed under microscope with magnification indicates the ability of the antibodies to inhibit hyphae formation and thus reduce virulence. Candida can alternatively be grown in liquid filament-inducing media and temperatures, fixed in 4.5% formaldehyde then visualized by Nomarski/DIC optics. The anti-Candida antibodies can also be assayed for their ability to inhibit or downregulate the expression of certain Candida polypeptides known to be involved in virulence and filament formation. Techniques known to those of skill in the art, including, but not limited to, Western blot analysis, Northern blot analysis and RT-PCR can be used to measure the expression of particular polypeptides.

Opsonization assays can be used to measure the ability of the anti-Candida antibodies to opsonize fungal cells thus leading to their phagocytosis by macrophage. In exemplary opsonization assays, the Candida are FITC-labeled and incubated for a predetermined period of time with the anti-Candida antibodies. After incubation, Candida are exposed to a macrophage cell line such as, but not limited to, J774 cells. Macrophage are allowed to contact the fungal cells to induce phagocytosis. After a predetermined period of time, the macrophage can be lysed and live Candida cells counted via methods known to those of skill in the art. A reduction in the number of live Candida cells indicates that the antibody is capable of opsonizing Candida cell leading to their phagocytosis by the macrophage. Alternatively, macrophage can be imaged via confocal microscopy and the number of Candida cells internalized by the macrophage can be counted.

Gene transcription assays can be used to measure the ability of the anti-Candida antibodies to inhibit the expression of genes involved in host tissue responses to Candida infection. In exemplary gene transcription assays, Candida cells are incubated with the anti-Candida antibodies for a predetermined period of time after which they are used to infect epithelial tissues. The amount of IL-8 secreted by the tissues in response to Candida infection is detected using sandwich ELISA assays with an biotin-labeled anti-IL-8 monoclonal antibody and avidin-alkaline phosphatase. The ability of the anti-Candida antibodies to reduce the expression of IL-8, as quantitated via optical measurements at 405 nm, indicates their ability to inhibit the tissue damage correlated with increased expression of IL-8 in response to Candida infection.

3. In Vivo Animal Models for Assessing Antibody Efficacy

In vivo studies using animal models can be performed to assess the efficacy of the anti-Candida antibodies provided herein. A variety of assays, such as those employing in vivo animal models, are available to those of skill in the art for evaluating the ability of the anti-Candida antibodies to inhibit or treat Candida yeast infection. The therapeutic effect of the anti-Candida antibodies can be assessed using animal models of the pathogenic infection, including, but not limited to, animal models of oral, vaginal, gastrointestinal, systemic, and corneal yeast infection and septic arthritis. Such animal models are known in the art, and include, but are not limited to, animal models for Candida infection in nematode, silkworm, drosophila, rat, inbred mouse, and rabbit (see, e.g., Pukkila-Worley et al. (2009) Eukaryot Cell. 8(11):1750-8; Pukkila-Worley et al. (2009) Curr Med. Chem. 16(13):1588-95; Hanaoka et al. (2008) Eukaryot Cell. 7(10):1640-8; Chamilos et al. (2009) J Infect Dis. 200(1):152-7; Naglik et al. (2008) FEMS Microbiol Lett. 283(2):129-39; Saleem et al. (2007) West Indian Med J 56(6):526-9; Amanai et al. (2008) Mycopathologia. 166(3):133-41; Clancy et al. (2009) Methods Mol Biol. 499:65-76; Lazzell et al. (2009) J Antimicrob Chemother. 64(3):567-70; Asmundsdottir et al. (2009) Clin Microbiol Infect. 15(6):576-85; Hasan et al. (2009) Microbes Infect. 11(8-9):753-61; Banerjee et al. (2008) Mol Biol Cell. 19(4):1354-65; US 2009/0081196; US 2007/0141088;). For in vivo testing of an antibody or composition's toxicity, any animal model system known in the art can be used, including, but not limited to, rats, mice, cows, monkeys, and rabbits.

Efficacy in treating or preventing Candida infection can be demonstrated by detecting the ability of anti-Candida antibodies provided herein to reduce the incidence of Candida infection, or to prevent, ameliorate or alleviate one or more symptoms associated with Candida infection. The treatment is considered therapeutic if there is, for example, amelioration of one or more symptoms or a decrease in mortality and/or morbidity following administration of antibodies provided herein.

Invertebrate models of Candida infection (e.g. Caenorhabiditis elegans, Silkworms, Drosophila) can be used in some instances to test anti-Candida antibodies for the ability to reduce mortality or increase survival time after the injection of a dose of Candida yeast that have been pre-incubated with anti-Candida antibodies. The extent of tissue destruction at a predetermined time after injection of antibody-treated Candida can be measured, for example, by histological and microscopic observation. Additionally, a reduction in mortality rate or an increase in survival time of animals injected with antibody-treated Candida cells can also be measured qualitatively. Decreases in mortality or increases in survival time indicate an ability of the anti-Candida antibodies to reduce the virulence of the fungal cells.

Rodent models of Candida infection (e.g. rats, mice, rabbits) can be used to test anti-Candida antibodies for the ability to reduce mortality after a lethal injection of Candida (see e.g., Clancy et al. (2009) Methods Mol Bio 499:65-76). For example, rodents can be pre-treated by passive immunization via a single intraperitoneal injection of the anti-Candida antibodies followed by intravenous injection of a lethal dose of yeast cells. The mortality of the animals can be plotted using Kaplan-Meier curves to examine the significance of any increase in survival of animals pretreated with anti-Candida antibodies.

The anti-Candida antibodies provided herein can be tested in vivo for the ability to prevent or ameliorate Candida infection of the eyes, joints, kidneys or oral, vaginal or gastrointestinal mucosa in rodents. Rodents can be pre-treated by passive immunization via a single intraperitoneal injection of the anti-Candida antibodies followed by inoculation of specific tissues (e.g. vaginal, oral or gastrointestinal mucosa) with yeast cells or injection of yeast cells into tissues (e.g. corneal stroma) or intravenously. Measurement of the severity of the infection (e.g. corneal ulceration, osteoarthritis as measured by micro-CT, immunohistochemistry of organ samples, CFU assessment of vaginal fluid or of homogenized kidney, liver, or spleen), techniques for which are known to those of skill in the art, can be used to assess the ability of the antibodies to prevent infection or ameliorate the symptoms of Candida infection.

Animal model studies using the anti-Candida antibodies provided herein can be extrapolated to humans and are useful for demonstrating the prophylactic and/or therapeutic utility of the anti-Candida antibodies.

### G. DIAGNOSTIC USES

The anti-Candida antibodies provided herein can be used in diagnostic assays for the detection, purification, and/or inhibition of Candida. Exemplary diagnostic assays include in vitro and in vivo detection of Candida. For example, assays using the anti-Candida antibodies provided herein for qualitatively and quantitatively measuring levels of Candida in an isolated biological sample (e.g., mucosal, urine, or blood sample) or in vivo are provided.

As described herein, the anti-Candida antibodies can be conjugated to a detectable moiety for in vitro or in vivo detection. Such antibodies can be employed, for example, to evaluate the localization and/or persistence of the anti-Candida antibody at an in vivo site, such as, for example, a mucosal site or internal site of infection. The anti-Candida antibodies which are coupled to a detectable moiety can be detected in vivo by any suitable method known in the art. The anti-Candida antibodies which are coupled to a detectable moiety also can be detected in isolated biological samples, such as tissue or fluid samples obtained from the subject following administration of the antibody.

1. In Vitro Detection of Pathogenic Infection

In general, Candida can be detected in a subject or patient based on the presence of one or more Candida antigens (e.g. cell wall glycoproteins) and/or polynucleotides encoding such proteins in a biological sample (e.g., blood, sera, sputum urine and/or other appropriate cells or tissues) obtained from a subject or patient. Such proteins can be used as markers to indicate the presence or absence of Candida in a subject or patient. The anti-Candida antibodies provided herein can be employed for detection of the level of antigen and/or epitope that binds to the agent in the biological sample.

A variety of assay formats are known to those of ordinary skill in the art for using an anti-Candida to detect polypeptide markers in a sample (see, e.g., Harlow and Lane, Antibodies: A Laboratory Manual, Cold Spring Harbor Laboratory, 1988). In general, the presence or absence of Candida in a subject or patient can be determined by contacting a biological sample obtained from a subject or patient with an anti-Candida provided and detecting in the sample a level of antigen that binds to the anti-Candida antibody or antigen-binding fragment thereof.

In some examples, the assay involves the use of an anti-Candida antibody provided immobilized on a solid support to bind to and remove the target polypeptide from the remainder of the sample. The bound polypeptide can then be detected using a detection reagent that contains a reporter group and specifically binds to the antibody/polypeptide complex. Such detection reagents can comprise, for example, a binding agent that specifically binds to the polypeptide or an antibody or other agent that specifically binds to the binding agent.

In some examples, a competitive assay can be utilized, in which a polypeptide

(e.g. cell wall glycoprotein) is labeled with a reporter group and allowed to bind to the immobilized anti-Candida antibody or antigen-binding fragment thereof after incubation of the anti-Candida antibody thereof with the sample. The extent to which components of the sample inhibit the binding of the labeled polypeptide to the anti-Candida antibody is indicative of the reactivity of the sample with the immobilized anti-Candida antibody. Suitable polypeptides for use within such assays include full length Candida mannoprotein proteins that have been glycosylated.

The solid support can be any material known to those of ordinary skill in the art to which the protein can be attached. For example, the solid support can be a test well in a microtiter plate or a nitrocellulose or other suitable membrane. The support also can be a bead or disc, such as glass, fiberglass, latex or a plastic material such as polystyrene or polyvinylchloride. The support also can be a magnetic particle or a fiber optic sensor, such as those disclosed, for example, in U.S. Pat. No. 5,359,681. The anti-Candida antibody can be immobilized on the solid support using a variety of techniques known to those of skill in the art. The anti-Candida antibody can be immobilized by adsorption to a well in a microtiter plate or to a membrane. In such cases, adsorption can be achieved by contacting the anti-Candida antibody, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of anti-Candida antibody ranging from about 10 ng to about 10 μg, and typically about 100 ng to about 1 μg, is sufficient to immobilize an adequate amount of anti-Candida antibody.

Covalent attachment of anti-Candida antibody to a solid support can generally be achieved by first reacting the support with a bifunctional reagent that will react with the support and a functional group, such as a hydroxyl or amino group, on the anti-Candida antibody. For example, the anti-Candida antibody can be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In some examples, the assay is performed in a flow-through or strip test format, wherein the anti-Candida antibody is immobilized on a membrane, such as nitrocellulose. In the flow-through test, polypeptides within the sample bind to the immobilized anti-Candida antibody as the sample passes through the membrane. A second, labeled binding agent then binds to the anti-Candida antibody-polypeptide complex as a solution containing the second binding agent flows through the membrane.

Additional assay protocols exist in the art that are suitable for use with the Candida glycoproteins or anti-Candida antibodies provided. The above descriptions are intended to be exemplary only. For example, it will be apparent to those of ordinary skill in the art that the above protocols can be readily modified to use Candida polypeptides to detect antibodies that bind to such polypeptides in a biological sample. The detection of such protein-specific antibodies can allow for the identification of Candida infection.

To improve sensitivity, multiple Candida protein markers can be assayed within a given sample. It will be apparent that anti-Candida antibodies specific for different Candida polypeptides and/or carbohydrate moieties (i.e. oligomannose moieties and/or glucans) can be combined within a single assay. Further, multiple primers or probes can be used concurrently. The selection of Candida protein markers can be based on routine experiments to determine combinations that results in optimal sensitivity. In addition, or alternatively, assays for Candida proteins provided herein can be combined with assays for other known Candida antigens.

2. In Vivo Detection of Pathogenic Infection

The anti-Candida antibodies provided herein can be employed as an in vivo diagnostic agent. For example, the anti-Candida antibodies can provide an image of infected tissues (e.g., Candida infection in the lungs) using detection methods such as, for example, magnetic resonance imaging, X-ray imaging, computerized emission tomography and other imaging technologies. For the imaging of Candida infected tissues, for example, the antibody portion of the anti-Candida antibody generally will bind to Candida cells, and the imaging agent will be an agent detectable upon imaging, such as a paramagnetic, radioactive or fluorescent agent that is coupled to the anti-Candida antibody. Generally, for use as a diagnostic agent, the anti-Candida antibody is coupled directly or indirectly to the imaging agent.

Many appropriate imaging agents are known in the art, as are methods for their attachment to the anti-Candida antibodies (see, e.g., U.S. Pat. Nos. 5,021,236 and 4,472,509). Exemplary attachment methods involve the use of a metal chelate complex employing, for example, an organic chelating agent such a DTPA attached to the antibody or antigen-binding (U.S. Pat. No. 4,472,509). The antibodies also can be reacted with an enzyme in the presence of a coupling agent such as glutaraldehyde or periodate. Conjugates with fluorescein markers are prepared in the presence of such coupling agents or by reaction with an isothiocyanate.

For in vivo diagnostic imaging, the type of detection instrument available is considered when selecting a given radioisotope. The radioisotope selected has a type of decay which is detectable for a given type of instrument. Another factor in selecting a radioisotope for in vivo diagnosis is that the half-life of the radioisotope be long enough so that it is still detectable at the time of maximum uptake by the target, but short enough so that deleterious radiation with respect to the host is minimized. Typically, a radioisotope used for in vivo imaging will lack a particle emission, but produce a large number of photons in the 140-250 keV range, which can be readily detected by conventional gamma cameras.

For in vivo diagnosis, radioisotopes can be bound to the antibodies provided either directly or indirectly by using an intermediate functional group. Exemplary intermediate functional groups which can be used to bind radioisotopes, which exist as metallic ions, to antibodies include bifunctional chelating agents, such as diethylene-triaminepentaacetic acid (DTPA) and ethylenediaminetetraacetic acid

(EDTA) and similar molecules. Examples of metallic ions which can be bound to the anti-Candida antibodies provided include, but are not limited to, ⁷²Arsenic, ²¹¹Astatine, ¹⁴Carbon, ⁵¹Chromium, ³⁶Chlorine, ⁵⁷Cobalt, ⁵⁸Cobalt, ⁶⁷Copper, ¹⁵²Europium, ⁶⁷Gallium, ⁶⁸Gallium, ³Hydrogen, ¹²³Iodine, ¹²⁵Iodine, ¹³¹Iodine, ¹¹¹Indium, ⁵⁹Iron, ³²Phosphorus, ¹⁸⁶Rhenium, ¹⁸⁸Rhenium, ⁹⁷Ruthenium, ⁷⁵Selenium, ³⁵Sulphur, ^{99m}Technetium, ²⁰¹Thallium, ⁹⁰Yttrium and ⁸⁹Zirconium.

The anti-Candida antibodies provided herein can be labeled with a paramagnetic isotope for purposes of in vivo diagnosis, as in magnetic resonance imaging (MRI) or electron spin resonance (ESR). In general, any conventional method for visualizing diagnostic imaging can be utilized. Generally, gamma and positron emitting radioisotopes are used for camera imaging and paramagnetic isotopes for MRI. Elements which are particularly useful in such techniques include, but are not limited to, ¹⁵⁷Gd, ⁵⁵Mn, ¹⁶²Dy, ⁵²Cr, and ⁵⁶Fe.

Exemplary paramagnetic ions include, but are not limited to, chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III). Ions useful, for example, in X-ray imaging, include but are not limited to lanthanum (III), gold (III), lead (II), and bismuth (III).

The concentration of detectably labeled anti-Candida antibody which is administered is sufficient such that the binding to Candida is detectable compared to the background. Further, it is desirable that the detectably labeled anti-Candida antibody be rapidly cleared from the circulatory system in order to give the best target-to-background signal ratio.

The dosage of detectably labeled anti-Candida antibody for in vivo diagnosis will vary depending on such factors as age, sex, and extent of disease of the individual. The dosage of a human monoclonal antibody can vary, for example, from about 0.01 mg/m² to about 500 mg/m², 0.1 mg/m² to about 200 mg/m², or about 0.1 mg/m² to about 10 mg/m². Such dosages can vary, for example, depending on whether multiple injections are given, tissue, and other factors known to those of skill in the art.

3. Monitoring Infection

The anti-Candida antibodies provided herein can be used in vitro and in vivo to monitor the course of pathogenic disease therapy. Thus, for example, the increase or decrease in the number of cells infected with Candida or changes in the concentration of the Candida virus particles present in the body or in various body fluids can be measured. Using such methods, the anti-Candida antibodies can be employed to determine whether a particular therapeutic regimen aimed at ameliorating the pathogenic disease is effective.

### H. PROPHYLACTIC AND THERAPEUTIC USES

The anti-Candida antibodies provided herein and pharmaceutical compositions containing anti-Candida antibodies provided herein can be administered to a subject for prophylaxis and therapy. For example, the anti-Candida antibodies provided can be administered for treatment of a disease or condition, such as a Candida infection. In some examples, the antibodies provided can be administered to a subject for prophylactic uses, such as the prevention and/or spread of Candida infection, including, but not limited to the inhibition of establishment of Candida infection in a host or inhibition of Candida transmission between subjects. The antibodies or antigen-binding fragments thereof also can be administered to a subject for preventing, treating, and/or alleviating of one or more symptoms of a Candida infection or reduce the duration of a Candida infection.

In some examples, administration of an anti-Candida antibody provided herein inhibits the incidence of Candida infection by at least or about 99%, at least or about 95%, at least or about 90%, at least or about 85%, at least or about 80%, at least or about 75%, at least or about 70%, at least or about 65%, at least or about 60%, at least or about 55%, at least or about 50%, at least or about 45%, at least or about 40%, at least or about 35%, at least or about 30%, at least or about 25%, at least or about 20%, at least or about 15%, or at least or about 10% relative to the incidence of Candida infection in the absence of the anti-Candida antibody. In some examples, administration of an anti-Candida antibody provided herein decreases the severity of one or more symptoms of Candida infection by at least or about 99%, at least or about 95%, at least or about 90%, at least or about 85%, at least or about 80%, at least or about 75%, at least or about 70%, at least or about 65%, at least or about 60%, at least or about 55%, at least or about 50%, at least or about 45%, at least or about 40%, at least or about 35%, at least or about 30%, at least or about 25%, at least or about 20%, at least or about 15%, or at least or about 10% relative to the severity of the one or more symptoms of Candida infection in the absence of the anti-Candida antibody.

1. Subjects for Therapy

A subject or candidate for therapy with an anti-Candida antibody provided herein includes, but is not limited to, a subject, such as a human patient, that has been exposed to a Candida, a subject, such as a human patient, who exhibits one or more symptoms of a Candida infection and a subject, such as a human patient, who is at risk of a Candida infection. Exemplary Candida infections include those caused by Candida, such as, but not limited to, as oropharyngeal candidiasis (thrush), vulvovaginal candidiasis (vaginal candidiasis), candidemia, oral candidiasis, mucocutaneous candidiasis, or forms of disseminated candidiasis in organs such as, but not limited to brain, eye, heart, liver, spleen, kidney, or bone marrow. In some examples, the Candida infection is caused by C. albicans. In some examples, the Candida infection is caused by other Candida species such as, but not limited to, C. tropicalis, C. parapsilosis, C. krusei, C. glabrata, C. lusitaniae, C. dubliniensis or C. guilliermondii.

In some examples, the subject for therapy with an anti-Candida antibody provided herein is a mammal. In some examples, the subject for therapy with an anti-Candida antibody provided herein is a primate. In particular examples, the subject therapy with an anti-Candida antibody provided herein is a human.

The anti-Candida antibodies provided can be administered to a subject, such as a human patient, for the treatment of any Candida-mediated disease. For example, the anti-Candida antibodies provided can be administered to a subject to alleviate one or more symptoms or conditions associated with a Candida infection, including, but not limited to, oral ulceration, periodontitis, esophagitis, vaginitis and paronychia. Such diseases and condition are well known and readily diagnosed by physicians or ordinary skill.

The anti-Candida antibodies provided can be administered to a subject, such a human patient, having a Candida infection for the maintenance or suppression therapy of recurring Candida-mediated disease.

The anti-Candida antibodies provided can be administered to a subject, such as a human patient, at risk of a Candida infection, including, but not limited to, a prematurely born (pre-term) infant (e.g., a human infant born less than 38 weeks of gestational age, such as, for example, 29 weeks, 30 weeks, 31 weeks, 32 weeks, 33 weeks, 34 weeks, 35 weeks, 36 weeks, or 37 weeks gestational age); an infant (e.g., a human infant born more than 37 weeks gestational age), pregnant women, a subject having diabetes mellitus, congenital immunodeficiency, or acquired immunodeficiency (e.g., an AIDS patient), leukemia, non-Hodgkin lymphoma, an immunosuppressed patient, such as, for example, a recipient of a transplant (e.g. a bone marrow transplant or a kidney transplant), patients treated in intensive care units (ICUs), cancer patients receiving chemotherapy, elderly subjects, including individuals in nursing homes or rehabilitation centers, patients receiving invasive devices (catheters, artificial joints and valves), or patients receiving broad spectrum and/or large amounts of antibiotics.

Tests for various pathogens and pathogenic infection are known in the art and can be employed for the assessing whether a subject is a candidate for therapy with an anti-Candida antibody provided herein. For example, tests for Candida infection, are known and include for example, yeast culture assays, antigen detection test, microscopic examination, polymerase chain reaction (PCR) tests, and various antibody serological tests (e.g. measurement of D-arabinitol levels in the serum or urine). Tests for fungal infection can be performed on samples obtained from tissue or fluid samples, such as saliva, blood, urine, vaginal mucous, lung sputum, lavage or lymph sample.

2. Dosages

The anti-Candida antibody is administered in an amount sufficient to exert a therapeutically useful effect in the absence of undesirable side effects on the patient treated. The therapeutically effective concentration of an anti-Candida antibody can be determined empirically by testing the polypeptides in known in vitro and in vivo systems such as by using the assays provided herein or known in the art.

An effective amount of antibody to be administered therapeutically will depend, for example, upon the therapeutic objectives, the route of administration, and the condition of the patient. In addition, the attending physician takes into consideration various factors known to modify the action of drugs, including severity and type of disease, patient's health, body weight, sex, diet, time and route of administration, other medications and other relevant clinical factors. Accordingly, it will be necessary for the therapist to titer the dosage of the antibody or antigen-binding fragment thereof and modify the route of administration as required to obtain the optimal therapeutic effect. Typically, the clinician will administer the antibody until a dosage is reached that achieves the desired effect. The progress of this therapy is easily monitored by conventional assays. Exemplary assays for monitoring treatment of a fungal infection are known in the art and include for example, antigen detection or yeast culture assays.

Generally, the dosage ranges for the administration of the anti-Candida antibodies provided herein are those large enough to produce the desired effect in which the symptom(s) of the pathogen-mediated disease (e.g. fungal disease) are ameliorated or the likelihood of fungal infection is decreased. In some examples, the anti-Candida antibodies provided herein are administered in an amount effective for inducing an immune response in the subject. The dosage is not so large as to cause adverse side effects, such as hyperviscosity syndromes, pulmonary edema or congestive heart failure. Generally, the dosage will vary with the age, condition, sex and the extent of the disease in the patient and can be determined by one of skill in the art. The dosage can be adjusted by the individual physician in the event of the appearance of any adverse side effect. Exemplary dosages for the prevention or treatment of a Candida infection and/or amelioration of one or more symptoms of a Candida infection include, but are not limited to, about or 0.01 mg/kg to about or 300 mg/kg, such as for example, about or 0.01 mg/kg, about or 0.1 mg/kg, about or 0.5 mg/kg, about or 1 mg/kg, about or 5 mg/kg, about or 10 mg/kg, about or 15 mg/kg, about or 20 mg/kg, about or 25 mg/kg, about or 30 mg/kg, about or 35 mg/kg, about or 40 mg/kg, about or 45 mg/kg, about or 50 mg/kg, about or 100 mg/kg, about or 150 mg/kg, about or 200 mg/kg, about or 250 mg/kg, about or 300 mg/kg.

For treatment of a fungal infection, the dosage of the anti-Candida antibodies can vary depending on the type and severity of the disease. The anti-Candida antibodies can be administered single dose, in multiple separate administrations, or by continuous infusion. For repeated administrations over several days or longer, depending on the condition, the treatment can be repeated until a desired suppression of disease symptoms occurs or the desired improvement in the patient's condition is achieved. Repeated administrations can include increased or decreased amounts of the anti-Candida antibody depending on the progress of the treatment. Other dosage regimens also are contemplated.

In some examples, the anti-Candida antibodies are administered one time, two times, three times, four times, five times, six times, seven times, eight times, nine times, ten times or more per day or over several days. In particular examples, the anti-Candida antibodies provided herein are administered one time, two times, three times, four times, five times, six time, seven times, eight times, nine times, ten times or more for the prevention or treatment of a Candida infection and/or amelioration of one or more symptoms of a Candida infection.

In some examples, the anti-Candida antibodies are administered in a sequence of two or more administrations, where the administrations are separated by a selected time period. In some examples, the selected time period is at least or about 1 day, 2 days, 3 days, 4 days, 5 days, 6 days, 1 week, 2 weeks, 3 weeks, 1 month, 2 months, or 3 months.

Therapeutic efficacy of a particular dosage or dosage regimen also can be assessed, for example, by measurement of fungal antigens in the subject prior to and following administration of one or more doses of the anti-Candida antibody thereof. Dosage amounts and/or frequency of administration can be modified depending on the desired rate of clearance of the Candida in the subject.

As will be understood by one of skill in the art, the optimal treatment regimen will vary and it is within the scope of the treatment methods to evaluate the status of the disease under treatment and the general health of the patient prior to, and following one or more cycles of therapy in order to determine the optimal therapeutic dosage and frequency of administration. It is to be further understood that for any particular subject, specific dosage regimens can be adjusted over time according to the individual need and the professional judgment of the person administering or supervising the administration of the pharmaceutical formulations, and that the dosages set forth herein are exemplary only and are not intended to limit the scope thereof. The amount of an anti-Candida antibody to be administered for the treatment of a disease or condition, for example a fungal infection (e.g. a Candida infection), can be determined by standard clinical techniques (e.g. fungal antigen detection assays). In addition, in vitro assays and animal models can be employed to help identify optimal dosage ranges. Such assays can provide dosages ranges that can be extrapolated to administration to subjects, such as humans. Methods of identifying optimal dosage ranges based on animal models are well known by those of skill in the art.

3. Routes of Administration

The anti-Candida antibodies provided herein can be administered to a subject by any method known in the art for the administration of polypeptides, including for example systemic or local administration. The anti-Candida antibodies can be administered by routes, such as parenteral (e.g., intradermal, intramuscular, intraperitoneal, intravenous, subcutaneous, or intracavity), topical, epidural, or mucosal (e.g. intranasal, oral, vaginally, vulvovaginal, esophageal, oroesophageal, bronchial, or pulmonary). The anti-Candida antibodies can be administered externally to a subject, at the site of the disease for exertion of local or transdermal action. Compositions containing anti-Candida antibodies or antigen-binding fragments can be administered by any convenient route, for example by infusion or bolus injection, by absorption through epithelial or mucocutaneous linings (e.g., oral mucosa, vaginal, rectal and intestinal mucosa). Compositions containing anti-Candida antibodies or antigen-binding fragments can be administered together with other biologically active agents. The mode of administration can include topical or other administration of a composition on, in or around areas of the body that may come on contact with fluid, cells, or tissues that are infected, contaminated or have associated therewith a fungus, such as Candida. The anti-Candida antibodies provided can be administered by topical or aerosol routes for delivery directly to target organs, such as the lungs. In some examples, the anti-Candida antibodies provided can be administered as a controlled release formulation as such as by a pump (see, e.g., Langer (1990) Science 249:1527-1533; Sefton (1987) CRC Crit. Ref. Biomed. Eng. 14:20; Buchwald et al. (1980) Surgery 88:507; Saudek et al. (1989) N. Engl. J. Med. 321:574) or via the use of various polymers known in the art and described elsewhere herein.

In particular examples, the anti-Candida antibodies are administered by pulmonary delivery (see, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903). Exemplary methods of pulmonary delivery are known in the art and include, but are not limited to, aerosol methods, such as inhalers (e.g., pressurized metered dose inhalers (MDI), dry powder inhalers (DPI), nebulizers (e.g., jet or ultrasonic nebulizers) and other single breath liquid systems), intratracheal instillation and insufflation. In some examples, pulmonary delivery can be enhanced by co-administration of or administration of a co-formulation containing the anti-Candida antibodies provided and a permeation enhancer, such as, for example, surfactants, fatty acids, saccharides, chelating agents and enzyme inhibitors, such as protease inhibitors.

Appropriate methods for delivery, can be selected by one of skill in the art based on the properties of the dosage amount of the anti-Candida antibody or the pharmaceutical composition containing the antibody or antigen-binding fragment thereof. Such properties include, but are not limited to, solubility, hygroscopicity, crystallization properties, melting point, density, viscosity, flow, stability and degradation profile.

In some examples, the anti-Candida antibodies provided herein increase the efficacy mucosal immunization against Candida. Thus, in particular examples the anti-Candida antibodies are administered to a mucosal surface. For example, the anti-Candida antibodies can be delivered via routes such as oral (e.g., buccal, sublingual), ocular (e.g., corneal, conjunctival, intravitreally, intra-aqueous injection), intranasal, genital (e.g., vaginal), rectal, pulmonary, stomachic, or intestinal. The anti-Candida antibodies provided herein can be administered systemically, such as parenterally, for example, by injection or by gradual infusion over time or enterally (i.e. digestive tract). The anti-Candida antibodies provided herein also can be administered topically, such as for example, by topical installation or application (e.g., intratracheal instillation and insufflation) of liquid solutions, gels, pastes, creams, ointments, powders or by inhalation (e.g., nasal sprays, inhalers (e.g., pressurized metered dose inhalers (MDI), dry powder inhalers (DPI), nebulizers (e.g., jet or ultrasonic nebulizers) and other single breath liquid systems)). Administration can be effected prior to exposure to Candida or subsequent to exposure to Candida.

4. Combination Therapies

The anti-Candida antibodies provided herein can be administered alone or in combination with one or more therapeutic agents or therapies for the prophylaxis and/or treatment of a disease or condition. For example, the anti-Candida antibodies provided can be administered in combination with one or more antifungal agents for the prophylaxis and/or treatment of a fungal infection, such as a respiratory fungal infection. In some examples, the respiratory fungal infection is a Candida infection. The antifungal agents can include agents to decrease and/or eliminate the pathogenic infection or agents to alleviate one or more symptoms of a pathogenic infection. In some examples, a plurality of antibodies or antigen-binding fragments thereof (e.g. one or more antifungal antibodies) also can be administered in combination, where at least one of the antibodies is an anti-Candida antibody provided herein. In some examples, a plurality of antibodies can be administered in combination for the prophylaxis and/or treatment of a Candida infection or multiple fungal infections, where at least one of the antibodies is an anti-Candida antibody provided herein. In some examples, the anti-Candida antibodies provided can be administered in combination with one or more antifungal antibodies, which bind to and inhibit Candida. In some examples, the anti-Candida antibodies provided can be administered in combination with one or more antibodies, which can inhibit or alleviate one or more symptoms of a fungal infection, such as a Candida infection. In some examples, two or more of the anti-Candida antibodies provided herein are administered in combination.

The one or more additional agents can be administered simultaneously, sequentially or intermittently with the anti-Candida antibody thereof. The agents can be co-administered with the anti-Candida antibody thereof, for example, as part of the same pharmaceutical composition or same method of delivery. In some examples, the agents can be co-administered with the anti-Candida antibody at the same time as the anti-Candida antibody thereof, but by a different means of delivery. The agents also can be administered at a different time than administration of the anti-Candida antibody thereof, but close enough in time to the administration of the anti-Candida antibody to have a combined prophylactic or therapeutic effect. In some examples, the one or more additional agents are administered subsequent to or prior to the administration of the anti-Candida antibody separated by a selected time period. In some examples, the time period is 1 day, 2 days, 3 days, 4 days, 5 days, 6 days, 1 week, 2 weeks, 3 weeks, 1 month, 2 months, or 3 months. In some examples, the one or more additional agents are administered multiple times and/or the anti-Candida antibody provided herein is administered multiple times.

In some examples, administration of the combination inhibits the incidence of Candida infection by at least or about 99%, at least or about 95%, at least or about 90%, at least or about 85%, at least or about 80%, at least or about 75%, at least or about 70%, at least or about 65%, at least or about 60%, at least or about 55%, at least or about 50%, at least or about 45%, at least or about 40%, at least or about 35%, at least or about 30%, at least or about 25%, at least or about 20%, at least or about 15%, or at least or about 10% relative to the incidence of Candida infection in the absence of the combination. In some examples, administration of the combination decreases the severity of one or more symptoms of Candida infection by at least or about 99%, at least or about 95%, at least or about 90%, at least or about 85%, at least or about 80%, at least or about 75%, at least or about 70%, at least or about 65%, at least or about 60%, at least or about 55%, at least or about 50%, at least or about 45%, at least or about 40%, at least or about 35%, at least or about 30%, at least or about 25%, at least or about 20%, at least or about 15%, or at least or about 10% relative to the severity of the one or more symptoms of Candida infection in the absence of the combination.

In some examples, the combination inhibits the binding of Candida to its host cell receptor by at least or about 99%, at least or about 95%, at least or about 90%, at least or about 85%, at least or about 80%, at least or about 75%, at least or about 70%, at least or about 65%, at least or about 60%, at least or about 55%, at least or about 50%, at least or about 45%, at least or about 40%, at least or about 35%, at least or about 30%, at least or about 25%, at least or about 20%, at least or about 15%, or at least or about 10% relative to the binding of Candida to its host cell receptor in the absence of the combination. In some examples, the combination inhibits Candida replication by at least or about 99%, at least or about 95%, at least or about 90%, at least or about 85%, at least or about 80%, at least or about 75%, at least or about 70%, at least or about 65%, at least or about 60%, at least or about 55%, at least or about 50%, at least or about 45%, at least or about 40%, at least or about 35%, at least or about 30%, at least or about 25%, at least or about 20%, at least or about 15%, or at least or about 10% relative to Candida replication in the absence of the combination.

Any therapy which is known to be useful, or which is or has been used for the prevention, management, treatment, or amelioration of a Candida infection or one or more symptoms thereof can be used in combination with anti-Candida antibody provided herein (see, e.g., Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (Eds.), 17th Ed., Merck Sharp & Dohme Research Laboratories, Rahway, N.J., 1999; Cecil Textbook of Medicine, 20th Ed., Bennett and Plum (Eds.), W.B. Saunders, Philadelphia, 1996, for information regarding therapies (e.g., prophylactic or therapeutic agents) which have been or are used for preventing, treating, managing, or ameliorating a Candida infection or one or more symptoms thereof). Examples of such agents include, but are not limited to, immunomodulatory agents, anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steroids, non-steroidal anti-inflammatory drugs (e.g. aspirin, ibuprofen, diclofenac, and COX-2 inhibitors), pain relievers, leukotriene antagonists (e.g., montelukast, methyl xanthines, zafirlukast, and zileuton), bronchodilators, such as β₂-agonists (e.g., bambuterol, bitolterol, clenbuterol, fenoterol, formoterol, indacaterol, isoetharine, metaproterenol, pirbuterol, procaterol, reproterol, rimiterol, salbutamol (Albuterol, Ventolin), levosalbutamol, salmeterol, tulobuterol and terbutaline) and anticholinergic agents (e.g., ipratropium bromide and oxitropium bromide), sulphasalazine, penicillamine, dapsone, antihistamines, anti-malarial agents (e.g., hydroxychloroquine), antifungal agents and antifungal agents.

Exemplary antifungal agents for the treatment of fungal infections that can be administered in combination with the anti-Candida antibodies provided include, but are not limited to, fluconazole, itraconazole, voriconazole, ketoconazole, miconazole, terconazole, clotrimazole, econazole, fenticonazole, sulconazole, tioconazole, isoconazole, omoconazole, oxiconazole, flutrimazole, butoconazole, amphotericin, nystatin, flucytosine, caspofungin, terbinafine, and gentian violet.

The anti-Candida antibodies provided herein also can be administered in combination with one or more therapies for the treatment of a fungal infection, such as an HIV infection. In exemplary therapies, administration anti-Candida antibodies with one or more fungal agents can treat the fungal infection and decrease the risk of opportunistic fungal infections by Candida. Exemplary antifungal agents that can be selected for combination therapy with an anti-Candida antibody provided herein include, but are not limited to, antifungal compounds, antifungal proteins, antifungal peptides, antifungal protein conjugates and antifungal peptide conjugates, including, but not limited to, nucleoside analogs, nucleotide analogs, immunomodulators (e.g. interferons) and immunostimulants. Combination therapy using antibodies and/or anti-Candida antibodies and antigen-binding fragments provided herewith are contemplated as is combination with the antibodies and/or anti-Candida antibodies and antigen-binding fragments provided herein with other anti-Candida antibodies and anti-Candida antibodies and antigen-binding fragments.

The anti-Candida antibodies provided herein also can be administered in combination with one or more agents capable of stimulating cellular immunity, such as cellular mucosal immunity. Any agent capable of stimulatory cellular immunity can be used. Exemplary immunostimulatory agents include, cytokines, such as, but not limited to, interferons (e.g., IFN-α, β, γ, ω), lymphokines and hematopoietic growth factors, such as, for example, GM-CSF (granulocyte macrophage colony stimulating factor), Interleukin-2 (IL-2), Interleukin-3 (IL-3), Interleukin-4 (IL-4), Interleukin-7 (IL-7), Interleukin-10 (IL-10), Interleukin-12 (IL-12), Interleukin-14 (IL-14), and Tumor Necrosis Factor (TNF).

For combination therapies with anti-pathogenic agents, dosages for the administration of such compounds are known in the art or can be determined by one skilled in the art according to known clinical factors (e.g., subject's species, size, body surface area, age, sex, immunocompetence, and general health, duration and route of administration, the kind and stage of the disease, and whether other treatments, such as other anti-pathogenic agents, are being administered concurrently).

The anti-Candida antibodies provided herein can be administered in combination with one or more additional antibodies or antigen-binding fragments thereof. In some examples, the one or more additional antibodies are antifungal antibodies. In some examples, the one or more additional antibodies bind to a fungal antigen, such as a Candida antigen. In some examples, the one or more additional antibodies bind to a Candida antigen that is a surface protein, such as a cell wall glycoprotein (e.g. a mannoprotein) or other cell wall component (e.g. glucans).

Antibodies for use in combination with an anti-Candida antibody provided herein include, but are not limited to, monoclonal antibodies, multispecific antibodies, synthetic antibodies, human antibodies, humanized antibodies, chimeric antibodies, intrabodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies provided herein), and epitope-binding fragments of any of the above. The antibodies for use in combination with an anti-Candida antibody provided herein can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. Antibodies for use in combination with an anti-Candida antibody or antigen-binding fragment thereof provided herein can be from any animal origin, including birds and mammals (e.g., human, murine, donkey, sheep, rabbit, goat, guinea pig, camel, horse, or chicken). The antibodies for use in combination with an anti-Candida antibody provided herein can be monospecific, bispecific, trispecific or of greater multispecificity. In some examples, the antibody for use in combination with an anti-Candida antibody provided herein includes, but is not limited to, anti-glucan antibodies, anti-mannoprotein antibodies, anti-integrin-like protein antibodies, and antibodies that bind to Candida secretory aspartic proteases In some examples, the antibody for use in combination with an anti-Candida antibody provided herein includes, but is not limited to, anti-Candida antibodies described in, for example, U.S. Pat. Nos. 4,670,382, 6,488,929, 6,774,219, and 7,138,502; U.S. Pat. Pub. Nos. 2004/0142385, 2006/0034849, 2005/0287146, 2007/019378, 2006/0251680, 2007/0116706, 2008/0193450, 2009/0214566, and 2009/0081196; PCT Pub. Nos. WO 86/02365, WO 99/52922, WO 04/03622, WO 03/089607, WO 2005/060713, WO 2006/097689, WO 2006/050343, and Maragues et al. (2003) Infection and Immunity 71(9):5273-5279.

5. Gene Therapy

In some examples, nucleic acids comprising sequences encoding the anti-Candida antibodies and/or derivatives thereof, are administered to treat, prevent or ameliorate one or more symptoms associated with Candida infection, by way of gene therapy. Gene therapy refers to therapy performed by the administration to a subject of an expressed or expressible nucleic acid. In this example, the nucleic acids produce their encoded antibody or antigen-binding fragment thereof that mediates a prophylactic or therapeutic effect.

Any of the methods for gene therapy available in the art can be employed for administration of nucleic acid encoding the anti-Candida antibodies and/or derivatives thereof. Exemplary methods are described below.

For general reviews of the methods of gene therapy, see, for example, Goldspiel et al. (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; Morgan and Anderson (1993) Ann. Rev. Biochem. 62:191-217; and TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In some examples, a composition provided herein contains nucleic acids encoding an anti-Candida antibody and/or derivative thereof, where the nucleic acids are part of an expression vector that expresses the anti-Candida antibody and/or derivative thereof in a suitable host. In particular, such nucleic acids have promoters, such as heterologous promoters, operably linked to the antibody coding region, said promoter being inducible or constitutive, and, optionally, tissue-specific. In another particular examples, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies (1989) Proc. Natl. Acad. Sci. USA 86:8932-8935; Zijlstra et al. (1989) Nature 342:435-438). In some examples, the expressed antibody molecule is a single chain antibody. In some examples, the nucleic acid sequences include sequences encoding the heavy and light chains, or fragments thereof, of the antibody. In a particular example, the nucleic acid sequences include sequences encoding an anti-Candida domain-exchanged Fab fragment. In a particular example, the nucleic acid sequences include sequences encoding a full-length anti-Candida domain-exchanged antibody. In some examples, the encoded anti-Candida antibody is a chimeric antibody.

Delivery of the nucleic acids into a subject can be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In some examples, the nucleic acid sequences are directly administered in vivo, where it is expressed to produce the encoded product. This can be accomplished by any of numerous methods known in the art, for example, by constructing them as part of an appropriate nucleic acid expression vector and administering it so that they become intracellular, for example, by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu (1987) J. Biol. Chem. 262:4429-4432) which can be used, for example, to target cell types specifically expressing the receptors. In some examples, nucleic acid-ligand complexes can be formed in which the ligand comprises a fusogenic viral peptide to disrupt endosomes, allowing the nucleic acid to avoid lysosomal degradation. In some examples, the nucleic acid can be targeted in vivo for cell specific uptake and expression, by targeting a specific receptor (see, e.g., PCT Publications WO 92/06180; WO 92/22635; WO92/203 16; WO93/14188, WO 93/20221). Alternatively, the nucleic acid can be introduced intracellularly and incorporated within host cell DNA for expression, by homologous recombination (Koller and Smithies (1989) Proc. Natl. Acad. Sci. USA 86:8932-8935; and Zijlstra et al. (1989) Nature 342:435-438).

In a some examples, viral vectors that contains nucleic acid sequences encoding an anti-Candida antibody and/or derivatives thereof are used. For example, a retroviral vector can be used (see, e.g., Miller et al. (1993) Meth. Enzymol. 217:581-599). Retroviral vectors contain the components necessary for the correct packaging of the viral genome and integration into the host cell DNA. The nucleic acid sequences encoding the antibody or antigen-binding fragment thereof to be used in gene therapy are cloned into one or more vectors, which facilitates delivery of the gene into a subject. More detail about retroviral vectors can be found, for example, in Boesen et al. (1994) Biotherapy 6:291-302. Other references illustrating the use of retroviral vectors in gene therapy include, for example, Clowes et al. (1994) J. Clin. Invest. 93:644-651; Klein et al. (1994) Blood 83:1467-1473; Salmons and Gunzberg (1993) Human Gene Therapy 4:129-141; and Grossman and Wilson (1993) Curr. Opin. in Genetics and Devel. 3:110-114.

Adenoviruses also are viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems include the liver, central nervous system, endothelial cells, and muscle. Adenoviruses have the advantage of being capable of infecting non-dividing cells. Kozarsky and Wilson (1993) Current Opinion in Genetics and Development 3:499-503 present a review of adenovirus-based gene therapy. Bout et al. (1994) Human Gene Therapy 5:3-10 demonstrated the use of adenovirus vectors to transfer genes to the respiratory epithelia of rhesus monkeys. Other instances of the use of adenoviruses in gene therapy can be found, for examples, in Rosenfeld et al. (1991) Science 252:431-434; Rosenfeld et al. (1992) Cell 68:143-155; Mastrangeli et al. (1993) J. Clin. Invest. 91:225-234; PCT Publication WO94/12649; and Wang et al. (1995) Gene Therapy 2:775-783. In a particular example, adenovirus vectors are used to deliver nucleic acid encoding the an anti-Candida antibodies and/or derivatives thereof provided herein.

Adeno-associated virus (AAV) also can be used in gene therapy (Walsh et al. (1993) Proc. Soc. Exp. Biol. Med. 204:289-300; and U.S. Pat. No. 5,436,146). In a particular example, adeno-associated virus (AAV) vectors are used to deliver nucleic acid encoding the anti-Candida antibodies and/or derivatives thereof provided herein.

Another approach to gene therapy involves transferring a gene to cells in tissue culture by such methods as electroporation, lipofection, calcium phosphate mediated transfection, or viral infection. Generally, the method of transfer includes the transfer of a selectable marker to the cells. The cells are then placed under selection to isolate those cells that have taken up and are expressing the transferred gene. The cells expressing the gene are then delivered to a subject.

In some examples, the nucleic acid encoding an anti-Candida antibody and/or derivative thereof provided herein is introduced into a cell prior to administration in vivo of the resulting recombinant cell. Such introduction can be carried out by any method known in the art, including, but not limited to, transfection, electroporation, microinjection, infection with a viral or bacteriophage vector containing the nucleic acid sequences, cell fusion, chromosome-mediated gene transfer, microcell-mediated gene transfer, and spheroplast fusion. Numerous techniques are known in the art for the introduction of foreign genes into cells (see, e.g., Loeffler and Behr (1993) Meth. Enzymol. 217:599-618; Cohen et al. (1993) Meth. Enzymol. 217:618-644; Cline, M. J., Pharma. Ther. 29:69-92 (1985)) and can be used for the administration of nucleic acid encoding an anti-Candida antibody and/or derivatives thereof provided herein, provided that the necessary developmental and physiological functions of the recipient cells are not disrupted. The technique provides for the stable transfer of the nucleic acid to the cell, so that the nucleic acid is expressible by the cell and typically heritable and expressible by its cell progeny.

The resulting recombinant cells can be delivered to a subject by various methods known in the art. Recombinant blood cells (e.g., hematopoietic stem or progenitor cells) can be administered intravenously. The amount of cells for administration depends on various factors, including, for example, the desired prophylactic and/or therapeutic effect and patient state, and can be determined by one skilled in the art.

Cells into which a nucleic acid can be introduced for purposes of gene therapy encompass any desired, available cell type, and include, but are not limited to, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, for example, as obtained from bone marrow, umbilical cord blood, peripheral blood, and fetal liver. In particular examples, the cell used for gene therapy is autologous to the subject.

In some examples in which recombinant cells are used in gene therapy, nucleic acid sequences encoding an anti-Candida and/or derivatives thereof provided herein are introduced into the cells such that they are expressible by the cells or their progeny, and the recombinant cells are then administered in vivo for therapeutic effect. In a particular example, stem or progenitor cells are used. Any stem and/or progenitor cells which can be isolated and maintained in vitro can be used (see e.g., PCT Publication WO 94/08598; Stemple and Anderson (1992) Cell 7 1:973-985; Rheinwald (1980) Meth. Cell Bio. 21A:229; and Pittelkow and Scott (1986) Mayo Clinic Proc. 61:771).

In a particular example, the nucleic acid to be introduced for purposes of gene therapy contains an inducible promoter operably linked to the coding region, such that expression of the nucleic acid is controllable by controlling the presence or absence of the appropriate inducer of transcription.

### I. PHARMACEUTICAL COMPOSITIONS, COMBINATIONS AND ARTICLES OF MANUFACTURE/KITS

1. Pharmaceutical Compositions

Provided herein are pharmaceutical compositions containing an anti-Candida antibody or antigen-binding fragment thereof provided herein. The pharmaceutical composition can be used for therapeutic, prophylactic, and/or diagnostic applications. The anti-Candida antibodies provided herein can be formulated with a pharmaceutical acceptable carrier or diluent. Generally, such pharmaceutical compositions utilize components which will not significantly impair the biological properties of the antibody, such as the binding of to its specific epitope (e.g. binding to an epitope on a Candida cell, e.g. a mannoprotein). Each component is pharmaceutically and physiologically acceptable in the sense of being compatible with the other ingredients and not injurious to the patient. The formulations can conveniently be presented in unit dosage form and can be prepared by methods well known in the art of pharmacy, including but not limited to, tablets, pills, powders, liquid solutions or suspensions (e.g., including injectable, ingestible and topical formulations (e.g., eye drops, gels, pastes, creams, or ointments), aerosols (e.g., nasal sprays), liposomes, suppositories, pessaries, injectable and infusible solution and sustained release forms. See, e.g., Gilman, et al., (eds. 1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa.; Avis, et al. (eds. 1993) Pharmaceutical Dosage Forms: Parenteral Medications Dekker, NY; Lieberman, et al. (eds. 1990) Pharmaceutical Dosage Forms: Tablets Dekker, NY; and Lieberman, et al., (eds. 1990) Pharmaceutical Dosage Forms: Disperse Systems Dekker, NY. When administered systematically, the therapeutic composition is sterile, pyrogen-free, generally free of particulate matter, and in a parenterally acceptable solution having due regard for pH, isotonicity, and stability. These conditions are known to those skilled in the art. Methods for preparing parenterally administrable compositions are well known or will be apparent to those skilled in the art and are described in more detail in, e.g., “Remington: The Science and Practice of Pharmacy (Formerly Remington's Pharmaceutical Sciences)”, 19th ed., Mack Publishing Company, Easton, Pa. (1995).

Pharmaceutical compositions provided herein can be in various forms, e.g., in solid, semi-solid, liquid, powder, aqueous, or lyophilized form. Examples of suitable pharmaceutical carriers are known in the art and include but are not limited to water, buffering agents, saline solutions, phosphate buffered saline solutions, various types of wetting agents, sterile solutions, alcohols, gum arabic, vegetable oils, benzyl alcohols, gelatin, glycerin, carbohydrates such as lactose, sucrose, amylose or starch, magnesium stearate, talc, silicic acid, viscous paraffin, perfume oil, fatty acid monoglycerides and diglycerides, pentaerythritol fatty acid esters, hydroxy methylcellulose, powders, among others. Pharmaceutical compositions provided herein can contain other additives including, for example, antioxidants, preservatives, antimicrobial agents, analgesic agents, binders, disintegrants, coloring, diluents, excipients, extenders, glidants, solubilizers, stabilizers, tonicity agents, vehicles, viscosity agents, flavoring agents, emulsions, such as oil/water emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as crystalline cellulose, microcrystalline cellulose, citric acid, dextrin, dextrose, liquid glucose, lactic acid, lactose, magnesium chloride, potassium metaphosphate, starch, among others (see, generally, Alfonso R. Gennaro (2000) Remington: The Science and Practice of Pharmacy, 20th Edition. Baltimore, Md.: Lippincott Williams & Wilkins). Such carriers and/or additives can be formulated by conventional methods and can be administered to the subject at a suitable dose. Stabilizing agents such as lipids, nuclease inhibitors, polymers, and chelating agents can preserve the compositions from degradation within the body.

Pharmaceutical compositions suitable for use include compositions wherein one or more anti-Candida antibodies are contained in an amount effective to achieve their intended purpose. Determination of a therapeutically effective amount is well within the capability of those skilled in the art. Therapeutically effective dosages can be determined by using in vitro and in vivo methods as described herein. Accordingly, an anti-Candida antibody provided herein, when in a pharmaceutical preparation, can be present in unit dose forms for administration.

An anti-Candida antibody provided herein can be lyophilized for storage and reconstituted in a suitable carrier prior to use. This technique has been shown to be effective with conventional immunoglobulins and protein preparations and art-known lyophilization and reconstitution techniques can be employed.

An anti-Candida antibody provided herein can be provided as a controlled release or sustained release composition. Polymeric materials are known in the art for the formulation of pills and capsules which can achieve controlled or sustained release of the antibodies provided herein (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas (1983) J. Macromol. Sci. Rev. Macromol. Chem. 23:61; see also Levy et al. (1985) Science 228:190; During et al. (1989) Ann. Neurol. 25:351; Howard et al. (1989) J. Neurosurg. 7 1:105; U.S. Pat. Nos. 5,679,377, 5,916,597, 5,912,015, 5,989,463, 5,128,326; and PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. Generally, the polymer used in a sustained release formulation is inert, free of leachable impurities, stable on storage, sterile, and biodegradable. Any technique known in the art for the production of sustained release formulation can be used to produce a sustained release formulation containing one more anti-Candida antibodies provided herein.

In some examples, the pharmaceutical composition contains an anti-Candida antibody provided herein and one or more additional antibodies. In some examples, the one or more additional antibodies includes, but is not limited to, anti-Candida antibodies described in, for example, U.S. Pat. Nos. 4,670,382, 6,488,929, 6,774,219, and 7,138,502; U.S. Pat. Pub. Nos. 2004/0142385, 2006/0034849, 2005/0287146, 2007/019378, 2006/0251680, 2007/0116706, 2008/0193450, 2009/0214566, and 2009/0081196; PCT Pub. Nos. WO 86/02365, WO 99/52922, WO 04/03622, WO 03/089607, WO 2005/060713, WO 2006/097689, WO 2006/050343, and Maragues et al. (2003) Infection and Immunity 71(9):5273-5279.

2. Articles of Manufacture/Kits

Pharmaceutical compositions of anti-Candida antibodies or nucleic acids encoding anti-Candida antibodies, or a derivative or a biologically active portion thereof can be packaged as articles of manufacture containing packaging material, a pharmaceutical composition which is effective for prophylaxis (i.e. vaccination, passive immunization) and/or treating Candida infection or Candida mediated disease or disorder, and a label that indicates that the antibody or nucleic acid molecule is to be used for vaccination and/or treating the infection, disease or disorder. The pharmaceutical compositions can be packaged in unit dosage forms contain an amount of the pharmaceutical composition for a single dose or multiple doses. The packaged compositions can contain a lyophilized powder of the pharmaceutical compositions containing the anti-Candida antibodies provided, which can be reconstituted (e.g. with water or saline) prior to administration.

The articles of manufacture provided herein contain packaging materials. Packaging materials for use in packaging pharmaceutical products are well known to those of skill in the art. Examples of pharmaceutical packaging materials include, but are not limited to, blister packs, bottles, tubes, inhalers, inhalers (e.g., pressurized metered dose inhalers (MDI), dry powder inhalers (DPI), nebulizers (e.g., jet or ultrasonic nebulizers) and other single breath liquid systems), pumps, bags, vials, containers, syringes, bottles, and any packaging material suitable for a selected formulation and intended mode of administration and treatment. The pharmaceutical composition also can be incorporated in, applied to or coated on a barrier or other protective device that is used for contraception from infection or applied to or coated on invasive devices, such as catheters, artificial joints and valves.

The anti-Candida antibodies, nucleic acid molecules encoding the antibodies thereof, pharmaceutical compositions or combinations provided herein also can be provided as kits. Kits can optionally include one or more components such as instructions for use, devices and additional reagents (e.g., sterilized water or saline solutions for dilution of the compositions and/or reconstitution of lyophilized protein), and components, such as tubes, containers and syringes for practice of the methods. Exemplary kits can include the anti-Candida antibodies provided herein, and can optionally include instructions for use, a device for administering the anti-Candida antibodies to a subject, a device for detecting the anti-Candida antibodies in a subject, a device for detecting the anti-Candida antibodies in samples obtained from a subject, and a device for administering an additional therapeutic agent to a subject.

The kit can, optionally, include instructions. Instructions typically include a tangible expression describing the anti-Candida antibodies and, optionally, other components included in the kit, and methods for administration, including methods for determining the proper state of the subject, the proper dosage amount, dosing regimens, and the proper administration method for administering the anti-Candida antibodies. Instructions also can include guidance for monitoring the subject over the duration of the treatment time

Kits also can include a pharmaceutical composition described herein and an item for diagnosis. For example, such kits can include an item for measuring the concentration, amount or activity of the selected anti-Candida antibody in a subject.

In some examples, the anti-Candida antibody is provided in a diagnostic kit for the detection of Candida in an isolated biological sample (e.g., a fluid sample, such as blood, sputum, vaginal mucous, lavage, lung intubation sample, saliva, urine or lymph obtained from a subject). In some examples, the diagnostic kit contains a panel of one or more anti-Candida antibodies and/or one or more control antibodies (i.e. non-Candida binding antibodies), where one or more antibodies in the panel is an anti-Candida antibody provided herein.

Kits provided herein also can include a device for administering the anti-Candida antibodies to a subject. Any of a variety of devices known in the art for administering medications to a subject can be included in the kits provided herein. Exemplary devices include, but are not limited to, an inhaler (e.g., pressurized metered dose inhaler (MDI), dry powder inhaler (DPI), nebulizer (e.g., jet or ultrasonic nebulizers) and other single breath liquid system), a hypodermic needle, an intravenous needle, a catheter, and a liquid dispenser such as an eyedropper. Typically the device for administering the anti-Candida of the kit will be compatible with the desired method of administration of the anti-Candida antibodies.

3. Combinations

Provided are combinations of the anti-Candida antibodies provided herein and a second agent, such as a second anti-Candida antibody or other therapeutic or diagnostic agent. A combination can include any anti-Candida antibody or reagent for effecting therapy thereof in accord with the methods provided herein. For example, a combination can include any anti-Candida antibody and an antifungal agent. Combinations also can include an anti-Candida antibody provided herein with one or more additional therapeutic antibodies. Combinations of the anti-Candida antibodies thereof provided also can contain pharmaceutical compositions containing the anti-Candida antibodies or host cells containing nucleic acids encoding the anti-Candida antibodies as described herein. The combinations provided herein can be formulated as a single composition or in separate compositions.

### J. EXAMPLES

The following examples are included for illustrative purposes only and are not intended to limit the scope of the invention.

### Example 1

### Design and Production of Vectors for Phage Display of Variant 2G12 Domain-Exchanged Antibody Libraries

Antibodies described herein that have an improved affinity for Candida albicans compared to 2G12 were identified by screening phage display libraries of variant 2G12 domain-exchanged antibodies. The template vector employed for the production of variant 2G12 domain-exchanged antibodies was generated from the 2G12 pCAL IT* vector (SEQ ID NO: 1) described in U.S. Provisional Application No. 61/192,982 and U.S. Patent Pub. No. 2010-0093563.

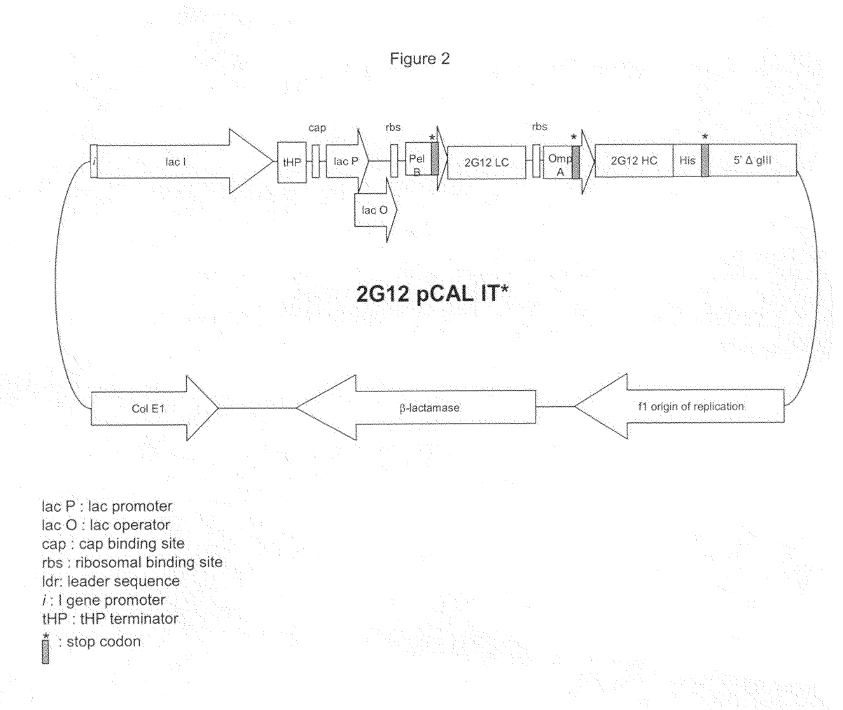

The 2G12 pCAL IT* vector was generated using a series of vector modifications (pCAL G13→2G12 pCAL 13→2G12 pCAL ITPO→2G12 pCAL IT*) designed to optimize the expression and decrease the toxicity of domain-exchanged antibodies in order to improve the phage display of the antibody fragments. The sequential vector modifications are briefly described below and in further detail in U.S. Provisional Application No. 61/192,982 and U.S. Patent Pub. No. 2010-0093563. As described in Example 2 below, the phage display libraries of variant 2G12 domain-exchanged antibodies employed were based on randomization of light chain complementarity determining region 3 (CDR3) of 2G12. In order to assist with the construction of the library, the 2G12 pCAL IT* vector was further modified to introduce three alanine substitutions in the light chain CDR3 of 2G12. The modification changed the light chain CDR3 from QHYAGYSAT (SEQ ID NO: 2) to QHAAGAAAT (SEQ ID NO: 3). The resulting vector, 2G12 3Ala LC pCAL IT* (SEQ ID NO:23, see also FIG. 2), was employed as the template vector for randomization of the 2G12 light chain CDR3. Construction of this vector is described in detail below.

1. pCAL G13

The phagemid vector pCAL G13 (SEQ ID NO: 5) vector is a vector that can be used for display of peptides, such as antibody polypeptides, particularly for display of domain-exchanged antibody fragments. The vector contains a truncated (C-terminal) M13 phage gene III sequence and an amber stop codon (TAG), upstream of the gene III sequence.

Using standard recombinant methods, pCAL G13 was constructed by insertion of (1) nucleic acid containing a lacZ promoter with cloning sites for insertion of the heavy and light chain antibody genes and (2) nucleic acid encoding the gene III minor coat protein from M13 mp18 phage (PCR fragment amplified from M13 mp18 DNA (New England Biolabs) with primers for introduction of an amber stop codon (TAG) upstream of the gene III sequence) into the SapI and PvuI sites of the pBlueScript II KS(+) vector (Stratagene). The identity of the resulting vector, pCAL G13, was confirmed by restriction enzyme analysis and sequencing.

2. 2G12 pCAL G13

The pCAL G13 phagemid vector (SEQ ID NO: 5) then was used to generate the 2G12 pCAL G13 vector (SEQ ID NO: 6) for display of the 2G12 domain-exchanged Fab fragment. The 2G12 pCAL G13 vector contains nucleic acid encoding a 2G12 light chain fragment (V_{L} and C_{L}) (SEQ ID NO: 7), and a 2G12 heavy chain fragment (V_{H} and C_{H}1) (SEQ ID NO: 8). The 2G12 heavy chain-encoding polynucleotide in the vector is directly upstream of an amber stop codon (TAG). This design of the vector resulted in a vector for expression of both 2G12 heavy chain-gene III fusion polypeptide and soluble 2G12 heavy chain (V_{H}/C_{H}1) polypeptides from the same genetic element, which can be used for display of the domain-exchanged antibodies on phage.

The 2G12 pCAL G13 vector was made by inserting a nucleic acid encoding a light chain domain of the 2G12 antibody (SEQ ID NO: 7) and heavy chain domain of the same antibody (SEQ ID NO: 8) into the pCAL G13 vector (SEQ ID NO: 5), described above, along with a sequence of nucleotides encoding an HA tag (SEQ ID NO: 9: YPYDVPDYA). The 2G12 heavy and light chains encoded by these nucleic acids contained the sequence of amino acids set forth in SEQ ID NOS: 10 and 11, respectively. The resulting 2G12 pCAL G13 vector contained the nucleic acid sequence set forth in SEQ ID NO: 6.

3. 2G12 pCAL ITPO Vector

The 2G12 pCAL G13 vector (SEQ ID NO: 6) was then modified by replacement of the 5′-truncated lac I gene with the lac I gene promoter (i) and the entire lac I gene, tHP terminator, and lac promoter/operon gene to create the 2G12 pCAL ITPO vector (SEQ ID NO: 12) in order to restore IPTG inducible gene expression.

The lac I gene promoter and lac I gene, the tHP terminator gene, and the Lac promoter and operon gene were amplified in three separate PCR reactions and then combined into a single PCR fragment using overlapping PCR. The resulting amplified product was inserted into the 2G12 pCAL G13 vector (digested with SapI/SgrAI to release the 5′-truncated lac I gene). The identity of the resulting 2G12 pCAL ITPO vector (SEQ ID NO:12) was confirmed by DNA sequencing.

4. 2G12 pCAL IT* Vector

To reduce the toxicity of the domain-exchanged Fab fragments expressed from the vector, and thereby increase stability of the phagemids displaying the Fab fragments, the 2G12 pCAL IT* vector (SEQ ID NO:1) was generated, in which an additional amber stop codon (TAG) was introduced into each of the leader sequences upstream of the polynucleotides encoding the heavy and light chain fragments. This phagemid vector was made by modifying the 2G12 pCAL ITPO vector (SEQ ID NO:12).

The 2G12 pCAL IT* vector can be used for repressed expression of the 2G12 Fab fragments in non-supE44 amber suppressor strains (such as, for example, NEB 10-beta cells and TOP10F′ cells), and modest expression in supE44 cells (e.g. XL1-Blue cells), for reduced expression and thus reduced toxicity of domain-exchanged Fab fragments in amber-suppressor strains such as XL1-Blue.

To generate the 2G12 pCAL IT* vector, the 2G12 pCAL ITPO vector (SEQ ID NO:12) was modified by introducing amber stop codons (TAG) at the 3′ end of the Pel B and Omp A bacterial leader sequences. The TAG amber stop codons were introduced to replace the wild-type CAG codon for glutamine.

Overlapping PCR mutagenesis was employed for the introduction the amber stop codons. Briefly, the Pel B and Omp A bacterial leader sequences were individually amplified using PCR primers, which contained the TAG mutation. Overlapping PCR was then employed to join the fragments. The resulting PCR product was then inserted into the 2G12 pCAL ITPO vector (digested with KasI and NotI), replacing the existing Pel B and Omp A bacterial leader sequences. The identity of the resulting 2G12 pCAL IT* vector was confirmed by DNA sequencing.

5. 2G12 3Ala LC pCAL IT*

As described above, the 2G12 pCAL IT* vector was further modified by the introduction of three alanine amino acid substitutions in the light chain CDR3 of 2G12. The modification of the 2G12 pCAL IT* vector was carried out using overlapping PCR mutagenesis and cloning at the SgrAI and PacI sites of the 2G12 pCAL IT* vector to produce the 2G12 3Ala LC pCAL IT* vector (SEQ ID NO:23).

TABLE 3
2G12 3Ala LC pCAL IT* primers

| | | | SEQ |
|---|---|---|---|
| | | | ID |
| Name | nt | Sequences | NO |
| 2G12LCF1 | 42 | GCCGCTGTGCCATCGCTCAGTAACcaattgaa | 199 |
| | | ttaaggagga | |
| 2G12LCR1 | 35 | ggcggcgctcttcTAGCGAAGTCGTCGAACTG | 200 |
| | | CAG | |
| 2G12ALCF2 | 54 | GCTACCTACCACTGCCAGCACGCCGCGGGTGC | 201 |
| | | GGCCGCGACCTTCGGTCAGGGT | |
| 2G12ALCR2 | 54 | GGTACCCTGACCGAAGGTCGCGGCCGCACCCG | 202 |
| | | CGGCGTGCTGGCAGTGGTAGGT | |
| 2G12LCF3 | 35 | ggcggcgctcttcTACCCGTGTTGAAATCAAA | 203 |
| | | CGT | |
| 2G12LCR3 | 48 | GCCGCTGTGCCATCGCTCAGTAACTTAATTAA | 19 |
| | | TTAGCATTCACCACGG | |

The 2G12ALCF2 and 2G12ALCR2 primers contain a 5′ phosphate.

The 2G12ALCF2 and 2G12ALCR2 primers contain three codons (underlined and bold in Table 3 above) that mutate two tyrosines and one serine to alanine. In order to form the CDRL3 3ALA duplex, 50 μL 2G12ALCF2 (100 μM) and 50 μL 2G12ALCR2 (100 μM) were mixed with 1 μL of 5M NaCl. The mixture was denatured at 95° C. for 5 min and slowly cooled to ambient temperature (25° C.) on a heat block covered with a Styrofoam® box to allow duplex formation.

PCR amplification was carried out to generate two 2G12 light chain fragment duplexes. Duplexes in pool 1 (LC1) were 387 nucleotides in length, and duplexes in pool 2 (LC3) were 388 nucleotides in length. For this process, two pools of forward oligonucleotide primers (2G12LCF1 and 2G12LCF3) and two pools of reverse oligonucleotide primers (2G12LCR1 and 2G12LCR3) were synthesized. The sequences of the primers in each pool are set forth in Table 3, above.

Two of the primers, 2G12LCR1 and 2G12LCF3, contained a 5′ sequence of nucleotides corresponding to a SapI restriction endonuclease cleavage site (GCTCTTC) (SEQ ID NO: 29). This enzyme cuts duplex polynucleotides to leave a 3-nucleotide overhang of any sequence at its 5′ end, beginning at one nucleotide in the 3′ direction from this recognition sequence. The restriction endonuclease recognition site is indicated in italics in Table 3, above, while the three-nucleotide overhang in each primer pool is indicated in bold. The oligonucleotides were designed such that the potential three nucleotide overhang of each primer pool was complementary to one of the three nucleotide overhangs generated in the light chain fragment duplexes. The oligonucleotides were designed in this manner to facilitate ligation in a subsequent step.

Primers in the 2G12LCF1 pool contained a sequence of nucleotides corresponding to a MfeI restriction endonuclease recognition site. Primers in the 2G12LCR3 pool contained a sequence of nucleotides corresponding to a Pad restriction endonuclease site (the MfeI and PacI restriction sites are indicated in bold in Table 3). These restriction endonuclease recognition sites facilitated ligation of the assembled duplexes into vectors in subsequent steps.

Further, the forward primer pool 2G12LCF1 and the reverse primer pool 2G12LCR3 contained a non gene-specific sequence region that is identical to the CALX24 primer (SEQ ID NO:21) at the 5′ ends of the primers. Thus, the reference sequence duplexes LC1 and LC3, generated by PCR with these primers/oligonucleotides, contained a duplex of these regions at each end of the reference sequence duplex. These regions served as templates for the primer CALX24, which was used in the subsequent single primer amplification (SPA) step, described below.

To form duplexes using these primers, the 2G12 pCAL IT* vector was used as a template in three separate PCR amplifications. For these reactions, primer pair pools, 2G12LCF1/2G12LCR1 and 2G12LCF3/2G12LCR3, were used to amplify duplex pool LC1 and duplex pool LC3 (Table 4). For each reaction, 4 μL of each primer, 4 μL of the 2G12 pCAL IT* vector template incubated in the presence of 4 μL Advantage HF2 Polymerase Mix (Clontech), 20 μL of 10c HF2 reaction buffer, 20 μL of 10×dNTP mixture, 144 μL PCR grade water in a 200 μL reaction volume. The PCR was carried out using the following reaction conditions: 1 minute denaturation at 95° C., followed by 30 cycles of 5 seconds of denaturation at 95° C., 10 seconds of annealing at 50° C., and 30 seconds of extension at 68° C., then finishing with a 3 minute incubation at 68° C. The amplified fragments were gel-purified using a Gel Extraction Kit (Qiagen) according to the manufacturer's instruction. The purified products were run on 1 agarose gel and each fragment was gel-purified with Gel Extraction Kit (Qiagen) according to the manufacturer's instruction.

TABLE 4
Primer pairs for duplex pools

| Fragment | LC1 | LC3 |
|---|---|---|
| 5′ primer | 2G12LCF1 | 2G12LCF3 |
| 3′ primer | 2G12LCR1 | 2G12LCR3 |
| Size (bp) | 384 | 388 |

After amplification by PCR, 2 μg of LC1 (384 bp) and LC3 (388 bp) were digested with SapI (New England Biolabs). The digested fragments were purified with PCR purification column (Qiagen) according to the manufacturer's instruction.

The digested light chain duplexes and the 3ALA duplex were hybridized and ligated to form intermediate duplexes. This process was carried out as follows. The 3ALA duplex was mixed in equimolar amounts with both reference duplexes, LC1 and LC3, in the presence of 5×T4 DNA ligase buffer and ligated with T4 DNA Ligase in a 20 μL volume, at room temperature (˜25° C.) overnight. The reaction was purified with PCR purification column and run on 1% agarose gel and each fragment was gel purified (Qiagen) according to the manufacturer's instruction.

Following the formation of the intermediate duplexes, a single primer amplification (SPA) reaction was used to generate amplified randomized assembled duplexes. Amplification was carried out using 2 μL of the intermediate duplex and 1.2 μL CALX24 primer (100 μmol), in the presence of 2 μL Advantage HF2 Polymerase Mix, 10 μL 10×HF2 buffer, 10 μL 10×dNTP, 74.8 μL of PCR grade water in a 100 μL reaction volume. The PCR was carried out using the following reaction conditions: denaturation at 95° C. for 1 min, followed by 30 cycles of denaturation at 95° C. for 5 seconds, annealing and extension at 68° C. for 1 min, then finished with an incubation at 68° C. for 3 min. The resulting amplified assembled duplex was column purified with a PCR purification column (Qiagen) and run on 1% agarose gel and purified with Gel Extraction Kit (Qiagen) according to the manufacturer's instruction.

The 3ALA LC duplex cassette was digested with SgrAI and PacI restriction enzymes and purified over a PCR purification column (Qiagen), according to the manufacturer's instruction. The vector DNA, 2G12 pCAL IT*, also was digested with SgrAI and Pad, run on a 0.7% agarose gel, and purified using Gel Extraction Kit (Qiagen). The SgrAI/PacI digested vector and 3ALA LC duplex cassette were ligated in the presence of T4 DNA ligase (Invitrogen) and 5× ligation reaction buffer (Invitrogen) in a 20 μL reaction volume at ambient temperature (22-25° C.) overnight.

The ligated DNA was electroporated into NEB 10-beta cells (New England Biolabs) at 2000 V/0.1 cm and titrated onto LB agar plates containing 100 μg/mL of carbenicillin and 20 mM glucose. Single colonies were selected and amplified. Miniprep DNA were analyzed by DNA sequencing and the clone SP2 was selected for Maxiprep DNA preparation from a single bacterial colony on a LB agar plate containing 100 μg/mL of carbenicillin and 20 mM glucose.

### Example 2

### Generation of Variant 2G12 Nucleic Acid Libraries for Display of Collections of Variant 2G12 Domain-Exchanged Fab Fragments

To generate phage display libraries for selection of phage displayed domain-exchanged antibodies that have an increased affinity for C. albicans, nucleic acid libraries were generated by randomizing nucleotides encoding four of the nine amino acids in the CDR3 region of the 2G12 light chain. Specifically, the libraries were designed to separately randomize two different regions containing four sequential amino acid residues, namely A, G, Y, and S of the light chain CDR3QHYAGYSAT (SEQ ID NO: 2) and Q, H, Y, and A of the light chain of CDR3QHYAGYSAT (SEQ ID NO: 2). The nucleic acid libraries can be used to make phage display libraries containing variant polypeptides with diversity in portions of the CDR3 of the light chain variable region of a 2G12 domain-exchanged Fab target polypeptide.

Two methods of randomization were employed. The first method used overlap PCR mutagenesis with Single Primer Amplification, which involved PCR amplification of overlapping segments of the 2G12 light chain using randomized nucleic acid primers, which contain randomized positions within the 2G12 light chain CDR3 encoding region. The second method employed modified Fragment Assembly and Ligation/Single Primer Amplification (mFAL-SPA) (as described in U.S. Patent Pub. No. 2010-0081575), which involved generating a collection of duplex cassettes containing randomized nucleic acids, which have randomized positions within the 2G12 light chain CDR3 encoding region. Both methods are described in detail below.

As described in subsections of this example below, the nucleic acid encoding the 2G12 light chain in the 2G12 3Ala LC pCAL IT* vector described in Example 1 was replaced with either the randomized PCR fragments produced by overlap PCR mutagenesis or the collection of randomized cassettes produced by the mFAL-SPA method to generate the nucleic acid libraries.

### A. Randomization of 2G12 Light Chain CDR3 by Overlap PCR Mutagenesis/Single Primer Amplification

1. AGYS Libraries

Overlap PCR generally involves PCR amplification of two or more overlapping segments of the gene of interest that can be subsequently recombined using an overlap fill-in reaction to reconstitute the full length gene. The process can be used to randomize a region of the gene by using oligonucleotide primers in the PCR amplification step which contain randomized nucleotides in addition to the nucleotides complementary to the template. Overlap PCR mutagenesis and Single Primer Amplification was used to diversify four amino acid positions in the 2G12 Fab by randomization of the 2G12 light chain CDR3 as follows.

a. Generation Overlapping Segments by PCR

Three AGYS nucleic acid libraries were generated by overlap PCR. For each library, a set of two overlapping segments of the 2G12 light chain were generated by PCR amplification. The oligonucleotide primers employed for the PCR amplifications are shown in Table 5.

A first segment, containing the nucleic acid encoding framework 1, CDR1, framework 2, CDR2, framework 3, and the first three amino acids of the CDR3 of the wild-type 2G12 light chain, was amplified as described below with a first oligonucleotide primer complementary to a region directly upstream of the 2G12 light chain in the 2G12 3Ala LC pCAL IT* vector (2G12LCF (SEQ ID NO: 13)) and a second oligonucleotide primer complementary to the region encoding several amino acids upstream of the CDR3 and the first three amino acids of the CDR3 (L3R (SEQ ID NO: 14)). This first segment does not contain any mutations relative to wild-type 2G12 and was used for all three libraries. The sequences of the primers used to amplify the first segment are set forth in Table 5. A MfeI restriction site (CAATTG) (SEQ ID NO: 20; shown in bold in Table 5) was designed in the 2G12LCF oligonucleotide to facilitate ligation of the library into vectors in subsequent steps. The underlined portion of the 2G12LCF oligonucleotide shown in Table 5 indicates a non gene-specific sequence that is identical to the CALX24 primer (SEQ ID NO: 21), which was used for the single primer amplification step described below.

A second segment, containing the nucleic acid encoding the entire CDR3 region of the 2G12 light chain and light chain constant region (C_{L}) was amplified as described below using a first oligonucleotide primer selected from those set forth in Table 5 containing randomized nucleotides in the light CDR3 region and a second oligonucleotide primer complementary to a region encoding the C-terminus of the 2G12 light chain (2G12LCR (SEQ ID NO: 19)). A PacI restriction site (TTAATTAA) (SEQ ID NO: 22; shown in bold in Table 5) was designed in the 2G12LCR oligonucleotide to facilitate to facilitate ligation of the library into vectors in subsequent steps. The underlined portion of the 2G12LCR oligonucleotide shown in Table 5 indicates a non gene-specific sequence that is identical to the CALX24 primer (SEQ ID NO: 21), which was used for the single primer amplification step described below.

Three pools of randomized oligonucleotides (AGYS, AGYS+1, and AGYS+2) were designed and generated for use in PCR amplification. The sequences of these randomized oligonucleotides are set forth in Table 5, below. Each oligonucleotide in each of these randomized pools was synthesized based on a reference sequence (which contained part of the native 2G12 light chain CDR3 nucleotide sequence), but contained randomized portions, represented in underlined type in Table 5. The CDR3 region is represented in bold type. The reference wild-type 2G12 sequence used to design the AGYS, AGYS+1, and AGYS+2 pools of randomized oligonucleotides is listed in Table 5. The region encoding the light chain CDR3 is indicated in bold.

The randomized portions of the oligonucleotides were synthesized using the NNK or NNT doping strategy. An NNK doping strategy minimizes the frequency of stop codons and ensures that each amino acid position encoded by a codon in the randomized portion could be occupied by any of the 20 amino acids. With this doping strategy, nucleotides were incorporated using an NKK pattern and a MNN pattern, during synthesis of the positive and negative strand randomized portions respectively, where N represents any nucleotide, K represents T or G, and M represents A or C. An NNT strategy eliminates stop codons and the frequency of each amino acid is less biased but omits Q, E, K, M, and W. The nucleotides in the randomized pools were labeled with 5′ phosphate groups.

TABLE 5
PCR mutagenesis/Single Primer Amplification
Primers

| | | | SEQ |
|---|---|---|---|
| | | | ID |
| Name | nt | Sequences | NO |
| 2G12LCF | 41 | GCCGCTGTGCCATCGCTCAGTAACaattgaattaa | 13 |
| | | ggagga | |
| L3R | 20 | ATAGTGCTGGCAGTGGTAGG | 14 |
| 2G12 | 55 | CTACCTACCACTGCCAGCACTACGCTGGTTACTCT | 15 |
| reference | | GCTACCTTCGGTCAGGGTAC | |
| sequence | | | |
| AGYS | 55 | CTACCTACCACTGCCAGCACTATNNKNNKNNKNNK | 16 |
| | | GCTACCTTCGGTCAGGGTAC | |
| AGYS+1 | 58 | CTACCTACCACTGCCAGCACTATNNKNNKNNKNNK | 17 |
| | | NNKGCTACCTTCGGTCAGGGTAC | |
| AGYS+2 | 61 | CTACCTACCACTGCCAGCACTATNNKNNKNNKNNK | 18 |
| | | NNKNNKGCTACCTTCGGTCAGGGTAC | |
| 2G12LCR | 48 | GCCGCTGTGCCATCGCTCAGTAACTTAATTAATTA | 19 |
| | | GCATTCACCACGG | |

The 2G12LCF, L3R and 2G12LCR primers were purified by HPLC. The AGYS, AGYS+1 and AGYS+2 primers contain a 5′ phosphate.

PCR amplification of the overlapping segments was performed using the primer pairs shown in Table 6. Each fragment was amplified using 10 ng of 2G12 3Ala LC pCAL IT* (SEQ ID NO: 23) (10 μL of 100 ng/μL stock) as a template with 10 μL of 20 μM 5′ and 3′ primers listed in Table 6 below in the presence of 10 μL of Advantage® HF2 Polymerase Mix (Clontech), 50 μL of 10×HF2 reaction buffer (Clontech), 50 μL of 10×dNTP mixture, and 360 μL of PCR grade water in a 500 reaction volume.

Each of the PCR amplifications (PCR 1a, 1b, 1b+1, 1b+2) included a denaturation step at 95° C. for 1 min, followed by 20 cycles of denaturation at 95° C. for 5 seconds, annealing at 50° C. for 10 seconds, and extension at 68° C. for 30 seconds, and finished with incubation at 68° C. for 1 min.

TABLE 6
PCR primers and resulting fragment sizes

| Fragment | PCR1a | PCR1b | PCR1b + 1 | PCR1b + 2 |
|---|---|---|---|---|
| 5′ primer | 2G12LCF | AGYS | AGYS + 1 | AGYS + 2 |
| 3′ primer | L3R | 2G12LCR | 2G12LCR | 2G12LCR |
| Size (bp) | 390 | 427 | 430 | 433 |

The amplified products from the PCR reactions were purified on a single PCR purification column (Qiagen). The purified products were run on 1% agarose gel and each fragment was gel-purified with Gel Extraction Kit (Qiagen) according to the manufacturer's instructions.

b. Overlap Fill-in Reaction

The overlapping segments generated from the PCR amplifications were rejoined to produce the nucleic acid library encoding full-length light chains, which contain the randomized CDR3 regions. The full-length nucleic acids were reconstructed by denaturation of the PCR amplified segments, annealing of the overlapping the nucleic acid, followed by an overlap fill-in reaction. Each library was constructed using 50 μL of PCR1Mix as shown in Table 7 for each library, 2 μL of Advantage® HF2 Polymerase Mix (Clontech), 10 μL of 10×HF2 reaction Buffer, 10 μL of 10×dNTP mixture, and 28 μL of PCR grade water in a 100 μL reaction volume. The calculated volumes for each of the PCR samples used in the fill-in reactions is shown in Table 7.

Each of the overlap reactions (AGYS, AGYS+1, and AGYS+2) included a denaturation step at 95° C. for 1 min, followed by 40 cycles of denaturation at 95° C. for 5 seconds, annealing at 60° C. for 10 seconds, and extension at 68° C. for 1 min, and finished with incubation at 68° C. for 3 min. The amplified products were run on 1% agarose gel and each fragment was purified with Gel Extraction Kit (Qiagen) according to the manufacturer's protocol

TABLE 7
Calculated volumes for PCR samples

| | Length | Amount needed for | Volume for |
|---|---|---|---|
| | of PCR | reaction: | fill-in |
| | fragment | 6.08 pmol (μg) | reaction |
| | (bp) | (3.64 × 10¹² molecules) | (μL) |
| PCR1a | 390 | 1.560 | 26.85 |
| PCR1b | 427 | 1.708 | 16.03 |
| PCR1b + 1 | 430 | 1.720 | 10.23 |
| PCR1b + 2 | 433 | 1.732 | 12.42 |

TABLE 8
PCR1 Mix for Overlap Reactions

| Library | AGYS | AGYS + 1 | AGYS + 2 |
|---|---|---|---|
| PCR1a (μL) | 26.85 | 26.85 | 26.85 |
| PCR1b (μL) | 16.03 | 0 | 0 |
| PCR1b + 1 (μL) | 0 | 10.23 | 0 |
| PCR1b + 2 (μL) | 0 | 0 | 12.42 |
| PCR grade water | 7.12 | 12.92 | 10.73 |
| (μL) | | | |
| Total (μL) | 50 | 50 | 50 |
| Size of combined | 794 | 797 | 800 |
| fragment (bp) | | | |

c. Single Primer Amplification (SPA)

SPA was performed by mixing 244 μL of PCR grade water, 50 μL of 10×HF2 buffer, 50 μL of 10×dNTP, 6 μL of CALX24 primer (100 μm) (SEQ ID NO: 21), 140 μL of each overlap fill-in reaction (AGYS, AGYS+1 or AGYS+2), and 10 μL of Advantage® HF2 Polymerase Mix in a 500 μL reaction volume.

Each of the SPA reactions included a denaturation step at 95° C. for 1 min, followed by 20 cycles of denaturation at 95° C. for 5 seconds, annealing and extension at 68° C. for 1 min, and finished with incubation at 68° C. for 3 min. The amplified products were column purified and run on 1% agarose gel and purified with Gel Extraction Kit (Qiagen).

d. Formation of the Variant 2G12 Nucleic Acid Libraries

Five μg of each library (AGYS, AGYS+1 or AGYS+2) was digested with MfeI and Pad restriction enzymes and purified over a PCR purification column (Qiagen). The vector DNA, 2G12 3Ala LC pCAL IT* (60 μg), also was digested with MfeI and Pad, run on a 0.7% agarose gel, and the 5139 bp vector fragment was purified using Gel Extraction Kit (Qiagen).

The MfeI/PacI digested vector and library fragments were ligated in the presence of 10 μL T4 DNA ligase (10 units) (Invitrogen) and 5× ligation reaction buffer (Invitrogen) in a 200 μL reaction volume at ambient temperature (22-25° C.) overnight. The ng and pmol amounts of the vector and library fragments used in the ligation reactions are shown in Table 9.

TABLE 9
Amounts of vector and library fragments used in ligation reactions

| Library | Amount | AGYS | AGYS + 1 | AGYS + 2 |
|---|---|---|---|---|
| Vector | ng | 1066.77 | 1066.77 | 8139.06 |
| | pmol | 0.316 | 0.315 | 2.405 |
| Fragment | ng | 385.58 | 387.142 | 2965.63 |
| | pmol | 0.789 | 0.790 | 6.026 |

e. Transformation

The ligation reactions were purified over PCR purification column (Qiagen) and electroporated into NEB 10-beta cells (New England Biolabs) at 2000 V in cuvettes with 0.1 cm gap. The cells were resuspended in SOC medium and incubated at 37° C. for 1 hr. Thirty mL of SuperBroth medium containing 20 μg/mL of carbenicillin and 20 mM of glucose were added to the culture and titrated on to LB agar plates containing 100 μg/mL of carbenicillin and 20 mM of glucose. The cells were incubated at 37° C. for 1 hr and added to 200 mL of SuperBroth medium with 50 μg/mL of carbenicillin and 20 mM of glucose. The culture was incubated overnight at 37° C. Maxiprep DNA was prepared from the overnight culture using HiSpeed Maxiprep Kit (Qiagen) according to the manufacturer's protocol.

The size of each library was 3.64×10⁸ for AGYS, 2.84×10⁸ for AGYS+1, and 1.59×10⁹ for AGYS+2.

2. QHYA Library

a. Generation Overlapping Segments by PCR

A QHYA nucleic acid library was generated by overlap PCR in which two overlapping segments of the 2G12 light chain were generated by PCR amplification. The oligonucleotide primers employed for the PCR amplifications are shown in Table 10.

A first segment, containing the nucleic acid encoding framework 1, CDR1, framework 2, CDR2, and framework 3 of the wild-type 2G12 light chain, was amplified as described below with a first oligonucleotide primer complementary to a region directly upstream of the 2G12 light chain in the 2G12 3Ala LC pCAL IT* vector (2G12LCF1 (SEQ ID NO: 213)) and a second oligonucleotide primer complementary to the region encoding several amino acids upstream of the CDR3 (QHYA-R (SEQ ID NO: 214)). This first segment does not contain any mutations relative to wild-type 2G12. The sequences of the primers used to amplify the first segment are set forth in Table 10. An MfeI restriction site (CAATTG) (SEQ ID NO: 20; shown in bold in Table 10) was designed in the 2G12LCF1 oligonucleotide to facilitate ligation of the library into vectors in subsequent steps. The underlined portion of the 2G12LCF1 oligonucleotide shown in Table 10 indicates a non gene-specific sequence that is identical to the CALX24 primer (SEQ ID NO: 21), which was used for the single primer amplification step described below.

A second segment, containing the nucleic acid encoding the entire CDR3 region of the 2G12 light chain and light chain constant region (C_{L}) was amplified as described below using a first oligonucleotide primer selected from those set forth in Table 10 containing randomized nucleotides in the light CDR3 region and a second oligonucleotide primer complementary to a region encoding the C-terminus of the 2G12 light chain (2G12LCR3 (SEQ ID NO:217)). A Pad restriction site (TTAATTAA) (SEQ ID NO: 22; shown in bold in Table 10) was designed in the 2G12LCR3 oligonucleotide to facilitate ligation of the library into vectors in subsequent steps. The underlined portion of the 2G12LCR3 oligonucleotide shown in Table 10 indicates a non gene-specific sequence that is identical to the CALX24 primer (SEQ ID NO: 21), which was used for the single primer amplification step described below.

A randomized oligonucleotide (LCDR3-QHYA5′ (SEQ ID NO:216)) was designed and generated for use in PCR amplification. The sequence of the randomized oligonucleotide is set forth in Table 10, below. The oligonucleotide was synthesized based on a reference sequence (SEQ ID NO:215, which contained part of the native 2G12 light chain CDR3 nucleotide sequence), but contained a randomized portion, represented in underlined type in Table 10. The CDR3 region is represented in bold type. The reference wild-type 2G12 sequence used to design the randomized oligonucleotides is listed in Table 10. The region encoding the light chain CDR3 is indicated in bold. The randomized portion of the oligonucleotide was synthesized using the NNK doping strategy, as described in Section A.1.a above.

TABLE 10
PCR mutagenesis/Single Primer Amplification
Primers

| | | | SEQ |
|---|---|---|---|
| | | | ID |
| Name | nt | Sequences | NO |
| 2G12LCF1 | 42 | GCCGCTGTGCCATCGCTCAGTAACcaattgaa | 213 |
| | | ttaaggagga | |
| QHYA-R | 24 | GCAGTGGTAGGTAGCGAAGTCGTC | 214 |
| 2G12 | 70 | GACGACTTCGCGACCTACCACTGCCAGCACTA | 215 |
| reference | | CGCTGGTTACTCTGCTACCTTCGGTCAGGGTA | |
| sequence | | CCCGTG | |
| LCDR3- | 70 | GACGACTTCGCGACCTACCACTGCNNKNNKNN | 216 |
| QHYA5′ | | KNNKGGTTACTCTGCTACCTTCGGTCAGGGTA | |
| | | CCCGTG | |
| 2G12LCR3 | 48 | GCCGCTGTGCCATCGCTCAGTAACTTAATTAA | 217 |
| | | TTAGCATTCACCACGG | |

The 2G12LCF1 and 2G12LCR3 primers were purified by HPLC.

PCR amplification of the overlapping segments was performed using the primer pairs shown in Table 11. Each fragment was amplified using 10 ng of 2G12 3Ala LC pCAL IT* (SEQ ID NO: 23) as a template with 1 μL of 20 μM 5′ and 3′ primers listed in Table 11 below in the presence of 1 μL of Advantage® HF2 Polymerase Mix (Clontech), 5 μL of 10×HF2 reaction buffer (Clontech), 5 μL of 10×dNTP mixture, and 36 μL of PCR grade water in a 50 μL reaction volume.

Each of the PCR amplifications included a denaturation step at 95° C. for 1 min, followed by 30 cycles of denaturation at 95° C. for 5 seconds, annealing at 50° C. for 10 seconds, and extension at 68° C. for 1 min, and finished with incubation at 68° C. for 3 min.

TABLE 11
PCR primers and resulting fragment sizes

| Fragment | PCR1a | PCR1b |
|---|---|---|
| 5′ primer | 2G12LCF1 | LCDR3-QHYA5′ |
| 3′ primer | QHYA-R | 2G12LCR3 |
| Size (bp) | 382 | 437 |

The amplified products were run on 1% agarose gel and each fragment was gel-purified with Gel Extraction Kit (Qiagen) according to the manufacturer's instructions.

b. Overlap Fill-in Reaction and Single Primer Amplification (SPA)

The overlapping segments generated from the PCR amplifications were rejoined to produce the nucleic acid library encoding full-length light chain, which contain the randomized CDR3 region. Full-length nucleic acid was reconstructed by denaturation of the PCR amplified segments, annealing of the overlapping the nucleic acid, and an overlap fill-in reaction followed by SPA. Full length fragment was constructed using 20 μL of PCR1a, 20 μL of PCR1b, 20 μl of Advantage® HF2 Polymerase Mix (Clontech), 100 μL of 10×HF2 reaction Buffer, 100 μL of 10×dNTP mixture, 20 μL of CALX24 primer (100 μM) and 720 μL of PCR grade water in a 1000 μL reaction volume.

Overlap fill-in and SPA reaction included a denaturation step at 95° C. for 1 min, followed by 30 cycles of denaturation at 95° C. for 5 seconds, annealing and extension at 68° C. for 1 min, and finished with incubation at 68° C. for 3 min. The amplified products were column purified and run on 1% agarose gel and purified with Gel Extraction Kit (Qiagen), according to the manufacturer's instructions.

TABLE 12
PCR1 Mix for Overlap Reactions

| Library | QHYA |
|---|---|
| PCR1a (μL) | 20 |
| PCR1b (μL) | 20 |
| PCR grade water (μL) | 720 |
| Total (μL) | 1000 |
| Size of combined fragment (bp) | 795 |

c. Formation of the Variant 2G12 Nucleic Acid Libraries

Eight μg of amplified fragment was digested with MfeI and Pad restriction enzymes and purified over a PCR purification column (Qiagen). The vector DNA, 2G12 3Ala LC pCAL IT* (60 μg), also was digested with MfeI and PacI, run on a 0.7% agarose gel, and the 5139 bp vector fragment was purified using Gel Extraction Kit (Qiagen).

The MfeI/PacI digested vector and library fragments were ligated in the presence of 20 μL T4 DNA ligase (20 units) (Invitrogen) and 5× ligation reaction buffer (Invitrogen) in a 400 μL reaction volume at ambient temperature (22-25° C.) overnight. The ng and pmol amounts of the vector and library fragment used in the ligation reaction are shown in Table 13.

TABLE 13
Amounts of vector and library fragments used in ligation reactions

| Library | Amount | QHYA |
|---|---|---|
| Vector (5139 bp) | ng | 2000 |
| | pmol | 0.592 |
| Fragment (743 bp) | ng | 722.9 |
| | pmol | 1.479 |

d. Transformation

The ligation reactions were purified over PCR purification column (Qiagen) and electroporated into NEB 10-beta cells (New England Biolabs) at 2000 V in cuvettes with 0.1 cm gap. The cells were resuspended in SOC medium and incubated at 37° C. for 1 hr. Thirty mL of SuperBroth medium containing 20 μg/mL of carbenicillin and 20 mM of glucose were added to the culture and titrated on to LB agar plates containing 100 μg/mL of carbenicillin and 20 mM of glucose. The cells were incubated at 37° C. for 1 hr and added to 200 mL of SuperBroth medium with 50 μg/mL of carbenicillin and 20 mM of glucose. The culture was incubated overnight at 37° C. Maxiprep DNA was prepared from the overnight culture using HiSpeed Maxiprep Kit (Qiagen) according to the manufacturer's protocol.

The size of the QHYA library was 2.0×10⁸.

B. Randomization of 2G12 Light Chain CDR3 by Modified Fragment Assembly and Ligation/Single Primer Amplification (mFAL-SPA)

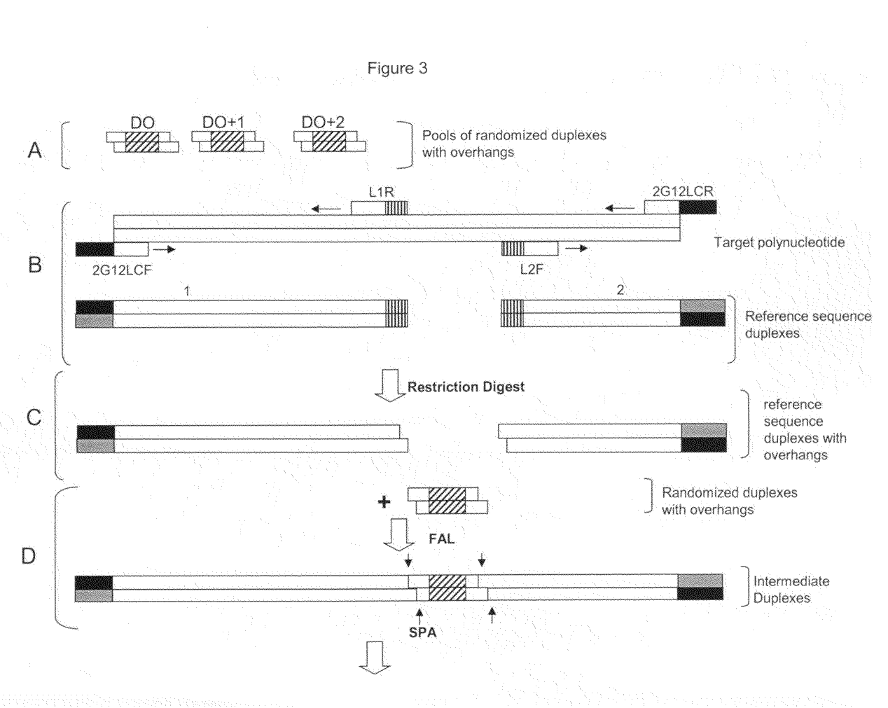

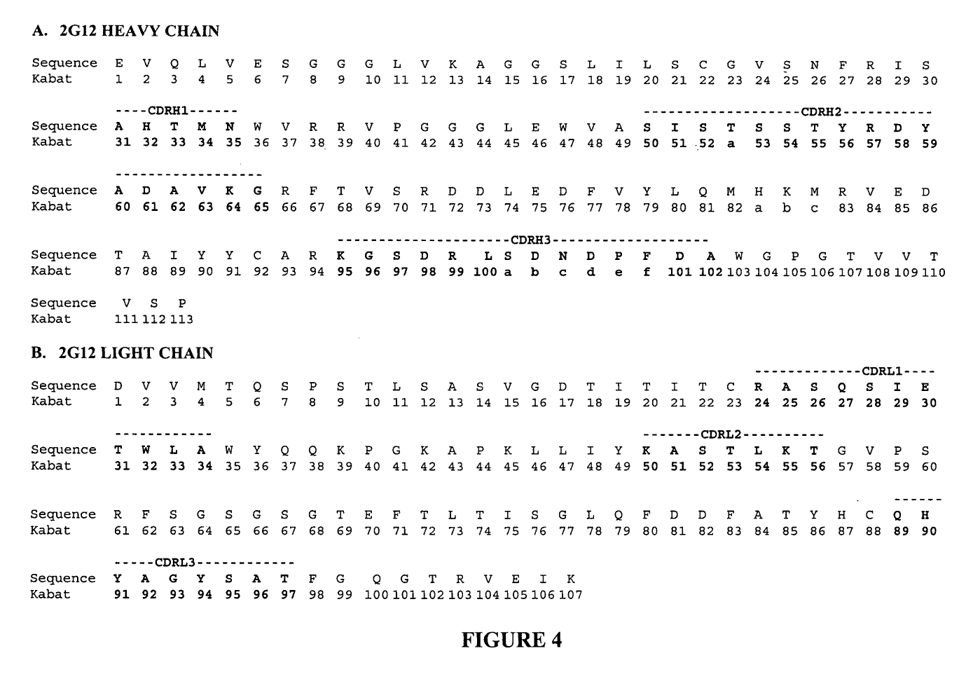

The Modified Fragment Assembly and Ligation (mFAL-SPA) method, as described in U.S. Application Publication No. 2010-0081575 also was employed to generate nucleic acid libraries which are diversified at the same four amino acid positions (A, G, Y, S), in the light chain CDR3 of 2G12 Fab. The details of this method are as follows and shown in FIG. 3.

1. Generation of Pools of Randomized Duplexes

Six pools of randomized oligonucleotides (AGYS, SYGA, AGYS+1, SYGA+1, AGYS+2, and SGYA+2) were designed and generated for use in forming three pools of randomized duplexes (DO, DO+1, and DO+2) (see FIG. 3A). The sequences of these randomized oligonucleotides are set forth in Table 14, below. Each oligonucleotide in each of these randomized pools was synthesized based on a reference sequence (which contained part of the wild-type 2G 12 light chain CDR3 nucleotide sequence), but contained randomized portions, represented in underlined type in Table 14 for oligonucleotides AGYS, SYGA, AGYS+1, SYGA+1, AGYS+2, and SGYA+2. The region encoding the light chain CDR3 region in these oligonucleotides is represented in bold type. The randomized portions were synthesized using the NNK or NNT doping strategy as described above for the overlap PCR mutagenesis. The reference wild-type 2G12 sequence used to design the AGYS, SYGA, AGYS+1, SYGA+1, AGYS+2, and SGYA+2 pools of randomized oligonucleotides also is listed in Table 14. The region encoding the light chain CDR3 is indicated in bold.

The randomized oligonucleotides were designed such that each oligonucleotide in each of the pools contained a region complementary to an oligonucleotide in another pool. For example, oligonucleotides in pool AGYS were complementary to oligonucleotides in pool SYGA, oligonucleotides in pool AGYS+1 were complementary to oligonucleotides in pool SYGA+1, and oligonucleotides in pool AGYS+2 were complementary to oligonucleotides in pool SYGA+2. The oligonucleotides in each pool further were designed, whereby, following hybridization of the pairs of oligonucleotides through these complementary regions, two nucleotide 5′-end overhangs would be generated, to facilitate ligation in subsequent steps. The nucleotides that become the 5′-end overhangs are indicated in italics in Table 14 for oligonucleotides AGYS, SYGA, AGYS+1, SYGA+1, AGYS+2, and SGYA+2. The nucleotides in the randomized pools were labeled with 5′ phosphate groups.

TABLE 14
Primers for mFAL-SPA

| | | | SEQ |
|---|---|---|---|
| | | | ID |
| Name | nt | Sequences | NO |
| 2G12LCF | 41 | GCCGCTGTGCCATCGCTCAGTAACaattgaattaa | 13 |
| | | ggagga | |
| L1R | 34 | gggcggcgctcttcG [symbol] CGAAGTCGTCGAACTG | 24 |
| 2G12 | 55 | CTACCTACCACTGCCAGCACTACGCTGGTTACTCT | 15 |
| reference | | GCTACCTTCGGTCAGGGTAC | |
| sequence | | | |
| AGYS | 55 | [symbol] CCTACCACTGCCAGCACTATNNKNNKNNKNNK | 16 |
| | | GCTACCTTCGGTCAGGGTAC | |
| SYGA | 55 | [symbol] GTACCCTGACCGAAGGTAGCMNNMNNMNNMNN | 25 |
| | | ATAGTGCTGGCAGTGGTAGG | |
| AGYS+1 | 58 | [symbol] CCTACCACTGCCAGCACTATNNKNNKNNKNNK | 17 |
| | | NNKGCTACCTTCGGTCAGGGTAC | |
| SYGA+1 | 58 | [symbol] GTACCCTGACCGAAGGTAGCMNNMNNMNNMNN | 26 |
| | | MNNATAGTGCTGGCAGTGGTAGG | |
| AGYS+2 | 61 | [symbol] CCTACCACTGCCAGCACTATNNKNNKNNKNNK | 18 |
| | | NNKNNKGCTACCTTCGGTCAGGGTAC | |
| SYGA+2 | 61 | [symbol] GTACCCTGACCGAAGGTAGCMNNMNNMNNMNN | 27 |
| | | MNNMNNATAGTGCTGGCAGTGGTAGG | |
| L2F | 34 | gggcggcgctcttcC [symbol] TGTTGAAATCAAACGT | 28 |
| 2G12LCR | 48 | GCCGCTGTGCCATCGCTCAGTAACTTAATTAATTA | 19 |
| | | GCATTCACCACGG | |

The 2G12LCF, L1R and 2G12LCR primers were purified by HPLC. The AGYS, SYGA, AGYS+1, SYGA+1, AGYS+2, and SYGA+2 primers contain a 5′ phosphate.

In order to form the DO, DO+1, and DO+2 randomized duplexes, 50 μL oligonucleotide 1 (at 100 μM) and 50 μL oligonucleotide 2 (see Table 14) (100 μM) as set forth in Table 15 for each reaction were mixed with 1 μL of 5M NaCl. The mixture was denatured at 95° C. for 5 min and slowly cooled to ambient temperature (25° C.) on a heat block covered with a Styrofoam® box to allow duplex oligonucleotide (DO) formation.

TABLE 15
Oligonucleotide pairings for generation of randomized duplexes

| | DO | DO + 1 | DO + 2 |
|---|---|---|---|
| Oligonucleotide 1 | AGYS | AGYS + 1 | AGYS + 2 |
| Oligonucleotide 2 | SGYA | SGYA + 1 | SGYA + 2 |
| Size (bp) | 55 | 58 | 61 |

2. Generation of Reference Sequence Duplexes by PCR

PCR amplification was carried out to generate two reference sequence duplexes (LC1 and LC2) (see FIG. 3B). Duplexes in pool 1 (LC1) were 385 nucleotides in length, and duplexes in pool 2 (LC2) were 387 nucleotides in length. For this process, two pools of forward oligonucleotide primers (2G12LCF and L2F) and two pools of reverse oligonucleotide primers (L1R and 2G12LCR) were synthesized. The sequences of the primers in each pool are set forth in Table 14, above.

Two of the primers, L1R and L2F, used to generate the reference sequence duplexes contained a 5′ sequence of nucleotides corresponding to a SapI restriction endonuclease cleavage site (GCTCTTC) (SEQ ID NO: 29). This enzyme cuts duplex polynucleotides to leave a 3-nucleotide overhang of any sequence at its 5′ end, beginning at one nucleotide in the 3′ direction from this recognition sequence. The restriction endonuclease recognition site is indicated in italics in Table 14, above, while the three-nucleotide overhang in each primer pool is indicated in bold. The oligonucleotides were designed such that the potential three nucleotide overhang of each primer pool was complementary to one of the three nucleotide overhangs generated in the randomized duplexes. The oligonucleotides were designed in this manner to facilitate ligation in a subsequent step.

Primers in the 2G12LCF pool contained a sequence of nucleotides corresponding to a MfeI restriction endonuclease recognition site. Primers in the 2G12LCR pool contained a sequence of nucleotides corresponding to a Pad restriction endonuclease site (the MfeI and Pad restriction sites are indicated in bold in Table 14). These restriction endonuclease recognition sites facilitated ligation of the assembled duplexes into vectors in subsequent steps.

Further, the forward primer pool 2G12LCF and the reverse primer pool 2G12LCR contained a non gene-specific sequence region that is identical to the CALX24 primer (SEQ ID NO:21) at the 5′ ends of the primers. Thus, the reference sequence duplexes LC1 and LC2, generated by PCR with these primers/oligonucleotides, contained a duplex of these regions at each end of the reference sequence duplex. These regions served as templates for the primer CALX24, which was used in the subsequent single primer amplification (SPA) step, described below.

To form duplexes using these primers, the 2G12 3Ala LC pCAL IT* vector was used as a template in three separate PCR amplifications. For these reactions, primer pair pools, 2G12LCF/L1R and L2F/2G12LCR, were used to amplify duplex pool LC1 and duplex pool LC2 (Table 16). For each reaction, 200 picomoles (pmol) of each primer (10 μL), 1 microgram (μg) of the 2G12 3Ala LC pCAL IT* vector template (10 μL of 100 ng/μL stock) were incubated in the presence of 10 μL Advantage HF2 Polymerase Mix (Clontech), 50 μL of 10c HF2 reaction buffer, 50 μL of 10×dNTP mixture, 360 μL PCR grade water in a 500 μL reaction volume. The PCR was carried out using the following reaction conditions: 1 minute denaturation at 95° C., followed by 20 cycles of 5 seconds of denaturation at 95° C., 10 seconds of annealing at 50° C., and 30 seconds of extension at 68° C., then finishing with a 1 minute incubation at 68° C. The amplified fragments were gel-purified using a Gel Extraction Kit (Qiagen) according to the manufacturer's instruction. The purified products were run on 1% agarose gel and each fragment was gel-purified with Gel Extraction Kit (Qiagen) according to the manufacturer's instruction.

TABLE 16
Primer pairs for duplex pools

| Fragment | LC1 | LC2 |
|---|---|---|
| 5′ primer | 2G12LCF | L2F |
| 3′ primer | L1R | 2G12LCR |
| Size (bp) | 385 | 387 |

After amplification by PCR, 20 pmoles of LC1 (385 bp) and LC2 (387 bp) were digested with SapI (New England Biolabs). The digested fragments were purified with PCR purification column (Qiagen) according to the manufacturer's instruction.

3. Ligation of Digested Reference Sequence Duplexes and Randomized Duplexes to Form Intermediate Duplexes

The digested reference sequence duplexes and the randomized duplexes were hybridized and ligated to form intermediate duplexes (see FIG. 3D). This process was carried out as follows. Three ligation reactions, one for each randomized duplex (DO, DO+1, and DO+2), were prepared. Each randomized duplex (DO, DO+1, or DO+2) was mixed in equimolar amounts (5.19 picomoles) with both reference duplexes, LC1 and LC2, in the presence of 80 μL 5×T4 DNA ligase buffer and ligated with 20 units of T4 DNA Ligase in a 400 μL volume, at room temperature (˜25° C.) overnight. The reaction was purified with PCR purification column and run on 1% agarose gel and each fragment was gel purified (Qiagen) according to the manufacturer's instruction.

4. Formation of Duplex Cassettes by Single Primer Amplification

Following the formation of the intermediate duplexes, a single primer amplification (SPA) reaction was used to generate amplified randomized assembled duplexes (see FIG. 3D). Amplification was carried out using 140 μL of the intermediate duplex (LC1/DO/LC2, LC1/DO+1/LC2, or LC1/DO+2/LC2) and 6 μL CALX24 primer (100 μmol), in the presence of 10 μL Advantage HF2 Polymerase Mix, 50 μl, 10×HF2 buffer, 50 μL 10×dNTP, 244 μI, of PCR grade water in a 500 μL reaction volume. The PCR was carried out using the following reaction conditions: denaturation at 95° C. for 1 min, followed by 20 cycles of denaturation at 95° C. for 5 seconds, annealing and extension at 68° C. for 1 min, then finished with an incubation at 68° C. for 3 min.

The resulting collections of amplified assembled duplexes were column purified with a PCR purification column (Qiagen) and run on 1% agarose gel and purified with Gel Extraction Kit (Qiagen) according to the manufacturer's instruction. Each duplex cassette LC 1/DO/LC2, LC1/DO+1/LC2, and LC1/DO+2/LC2 represents the AGYS, AGYS+1 and AGYS+2 libraries, respectively.

5. Formation of the Variant 2G12 Nucleic Acid Libraries

Five μg of each library (AGYS, AGYS+1 or AGYS+2) was digested with MfeI and PacI restriction enzymes and purified over a PCR purification column (Qiagen), according to the manufacturer's instruction. The vector DNA, 2G12 3Ala LC pCAL IT* (60 μg), also was digested with MfeI and Pad, run on a 0.7% agarose gel, and the 5139 bp vector fragment was purified using Gel Extraction Kit (Qiagen). Each vector was ligated with the assembled duplex cassettes described above, to generate three libraries, each containing randomized 2G12 Fab encoding nucleic acid members.

The MfeI/PacI digested vector and library fragments were ligated in the presence of 10 μL T4 DNA ligase (10 units) (Invitrogen) and 5× ligation reaction buffer (Invitrogen) in a 200 μL reaction volume at ambient temperature (22-25° C.) overnight. The ng and pmol amounts of the vector and library fragments used in the ligation reactions is shown in Table 17.

TABLE 17
Amounts of vector and library fragments used in ligation reactions

| Library | Amount | AGYS | AGYS + 1 | AGYS + 2 |
|---|---|---|---|---|
| Vector | ng | 1066.77 | 1066.77 | 8139.06 |
| | pmol | 0.316 | 0.315 | 2.405 |
| Fragment | ng | 385.58 | 387.142 | 2965.63 |
| | pmol | 0.789 | 0.790 | 6.026 |

6. Transformation

The ligation reactions were purified over PCR purification column (Qiagen) and electroporated into NEB 10-beta cells (New England Biolabs) at 2000 V in cuvettes with 0.1 cm gap. The cells were resuspended in SOC medium and incubated at 37° C. for 1 hr. Thirty mL of SuperBroth medium containing 20 μg/mL of carbenicillin and 20 mM of glucose were added to the culture and titrated on to LB agar plates containing 100 μg/mL of carbenicillin and 20 mM of glucose. The cells were incubated at 37° C. for 1 hr and added to 200 mL of SuperBroth medium with 50 μg/mL of carbenicillin and 20 mM of glucose. The culture was incubated overnight at 37° C. Maxiprep DNA was prepared from the overnight culture using HiSpeed Maxiprep Kit (Qiagen) according to the manufacturer's protocol.

The size of each library was 3.15×10⁸ for AGYS, 3.98×10⁸ for AGYS+1, and 1.59×10⁹ for AGYS+2.

### Example 3

### Preparation of Formalin-Fixed Candida albicans Cells

Formalin fixed C. albicans cells were prepared for use as the C. albicans target antigen for phage selection. A starter culture was first prepared by inoculation of 10 mL of YPD medium with a single colony of C. albicans (Cat. No. 10231, ATCC, stored at −20° C.). The cells were cultured at 37° C. with shaking at 170 rpm for 24 hours, before 500 μL of culture was removed and transferred into 10 mL of fresh YPD medium. This was repeated to generate 10 individual cultures, which were incubated at 37° C. with agitation at 170 rpm for 24 hours. The C. albicans cells were centrifuged at 4000 rpm for 10 minutes and the cell pellet was resuspended in 1×PBS. This washing step was repeated twice before the cell pellet was fixed in 1% formalin (diluted in 1×PBS). The cells were incubated with shaking for 30 minutes at room temperature before being centrifuged at 4000 rpm for 10 minutes. The cell pellet was resuspended in 1×PBS. The cells were washed twice more with PBS before being counted using a hemocytometer. The C. albicans cell density was adjusted to 1×10⁸ cells per mL, and the cells were aliquotted into 1 mL stocks and stored at −20° C. or −80° C. The fixed C. albicans cells were thawed on ice prior to use before each round of selection.

### Example 4

### Selection of Domain-Exchanged Antibodies Specific for Candida albicans

Diversified 2G12-derived domain-exchanged antibodies having specificity for Candida albicans were selected using phage display techniques. Each 2G12 library generated as described in Example 2 was introduced into electrocompetent DH5α VCSM13 dsDNA CL F-cells for expression on the surface of the cells in phagemids. The phage were then screened for specificity for C. albicans using formalin-fixed C. albicans cells as the target antigen. The selection protocol is described in general below.

A. Preparation of Electrocompetent DH5α VCSM13 dsDNA CL F-Cells

To generate the electrocompetent DH5α VCSM13 dsDNA CL F-cells for subsequent use in the display of phage, double-stranded DNA from VCSM 13 helper phage was purified before being transformed into DH5α cells. These cells were then treated to become electrocompetent.

1. Purification of VCSM13 Helper Phage dsDNA

Double-stranded DNA from VCSM13 helper phage was purified using the Qiafilter Midiprep or Maxiprep Kit (Qiagen), per the manufacturer's instructions. Briefly, a colony of XL1-Blue MRF′ cells (Stratagene) was transferred into 10 ml of Superbroth (SB) media (30 g Bacto tryptone, 20 g Yeast extract, 10 g MOPS, in 1 liter water, pH 7.0) in a 50-ml conical tube. Tetracycline was added to a final concentration of 10 μg/mL, and the culture was incubated with shaking at 37° C. until an OD600 of 0.3 was reached (corresponding approximately to 2.5×10⁸ cells/mL). The culture was scaled up to between 50 and 100 mL, tetracycline was added to a final concentration of 10 μg/mL. For culture volumes of approximately 50-100 mL, the Qiagen Qiafilter Midiprep was used for purification. For culture volumes of approximately 200 mL, the Qiagen Qiafilter Maxiprep was used for purification.

VCSM13 helper phage (Stratagene) were added to the culture at a multiplicity of infection (MOI) of 10:1 (phage-to-cells ratio). The culture was incubates at 37° C. (without agitation) for 15 minutes to allow the phage to attach to the cells, before being incubated for a further hour with shaking at 37° C. Kanamycin was added to the culture at a final concentration of 25 μg/mL, and the culture was incubated with shaking at 37° C. for a further 8 hours. The cell debris was pelleted by centrifugation and the supernatant was transferred to a fresh conical tube. The pellet was stored at either −20° C. or −80° C. until required. The titer of the supernatant was determined and typically found to be between 7.5×10¹⁰ and 1×10¹² pfu/mL.

The cell pellet was resuspended in 4 mL of Buffer P1 if a Midiprep was being used for purification, or 10 mL of Buffer P1 if a Maxiprep was being used for purification. The DNA was purified as per the manufacturer's instructions. Following elution from the Qiagen-tip 100 (if a Midiprep kit was used) or Qiagen-tip 500 (if a Maxiprep kit was used), the VCSM13 DNA was precipitated by the addition of 0.7 volumes of room temperature isopropanol and centrifugation at >15,000×g for 60 minutes at 4° C. The DNA pellets were washed with 2 mL or 5 mL (for Midiprep or Maxiprep purifications, respectively) of 70% ethanol and centrifuged at >15,000×g for 10 minutes at 4° C. The VCSM13 DNA pellet was air dried for 5-10 minutes and dissolved in a suitable volume of TE buffer, pH 8.0, or 10 nM Tris-Cl, pH 8.5. The concentration of VCSM13 DNA was then measured.

2. Preparation of Electrocompetent VCSM13 DH5α Cell Line

To prepare the electrocompetent VCSM13 DH5α cell line, sterile SOC was first pre-heated to 37° C. and the electroporator settings were adjusted to: 2000V [20 kV/cm field strength], resistance to 200Ω, capacitance to 25 μF. Electroporation cuvettes (0.1 centimeter gap) were pre-chilled at −20° C. and transferred to an ice bucket prior to use. Electrocompetent ElectroMax DH5α-E cells (Invitrogen) were thawed on ice before 100 ng of the purified VCSM13 DNA was added to the cells. The cells were then incubated for 5 minutes on ice and transferred from the 1.5 mL tube into each pre-chilled electroporation cuvette. To avoid arcing and to ensure optimal DNA entry, a 2-5% volume of DNA to cell ratio typically was used. The electroporation cuvettes were tapped gently until the mixture of cells and DNA settled flush with the bottom of the cuvette, and any external water or condensation on the cuvette was wiped away. The sample was pulsed once and the cuvette was quickly removed and 1000 μl of pre-warmed SOC media was added to the cells.

The cells were then transferred to a sterile 50 mL conical polypropylene tube, and the remaining cells in the cuvette were flushed twice more with 1 mL, so that the cells were resuspended in a final volume of 3 mL SOC media. Superbroth media was added to the cells for a final volume of 10 mL, and the cells were incubated at 37° C. with shaking at 250 rpm, for 1 hour. (To calculate the transformation efficiency, 90 μl of SOC was aliquotted into an ELISA dilution plate, and 10 μL of the cells (DH5α cells with VCSM13 DNA) was added to the top well and a 6 step, 10-fold dilution series was prepared. Seventy-five μL of the diluted cells were plated on LB agar/kanamycin plates (LB agar with 25 μg/mL kanamycin and 20 mM D-glucose), and the liquid was allowed to dry for a minimum of 15 minutes before being incubated at 37° C. overnight).

After the 1 hour incubation, 0.5 mL of the DH5α VCSM13 dsDNA CL F-cells were inoculated into a 500 mL flask containing 50 mL of SB media, and kanamycin was added to a final concentration of 25 μg/mL. The flask was incubated at 37° C. overnight with shaking at 250 rpm. Ten mL of the overnight culture was added to 1 L of SB in a 2 L flask, and kanamycin was added to a final concentration of 25 μg/mL. The cells were grown at 37° C. with shaking at 250 rpm until the culture reached an OD 600 of approximately 0.6-0.7, so that the cells were harvested at early to mid-log phase (cell density of approximately 4-5×10⁷ cells/mL). The cells were chilled on ice for approximately 20 minutes, and kept in an ice/water bath for the subsequent steps. All containers used in the subsequent steps also were chilled before adding any cells.

The DH5α VCSM13 dsDNA CL F-cells were transferred to three large centrifuge bottles and centrifuged at 4000×g for 20 min at 4° C. The supernatant was decanted and the cells remaining in the bottle were placed on ice. The cell pellets were then resuspended in 10 mL of ice cold 10% glycerol, and the bottles were then filled with approximately 400 mL of ice cold 10% glycerol. The cells were again centrifuged at 4000×g for 20 min at 4° C., the supernatant was decanted, and the cells remaining in the bottle were placed on ice. The cell pellets were resuspended in 10 mL of ice cold 10% glycerol, and another approximately 400 mL 10% glycerol was added to fill the bottle before the cells were again centrifuged at 4000×g for 20 min at 4° C. The supernatant was removed and the cells were resuspended in approximately 25 mL ice cold 10% glycerol and transferred to a pre-chilled 50 mL falcon tube. The cells were pelleted by centrifugation at 4000 rpm for 30 minutes and the supernatant was carefully removed. The final cell pellet was resuspended in 4-5 mL ice cold 10% glycerol, having a concentration of about 1−3×10¹° cells/mL. The resulting electrocompetent DH5α VCSM13 dsDNA CL F-cells were aliquotted in 100 μL volumes into several pre-chilled sterile 1.5 mL tubes, on ice, before being frozen in a dry ice/ethanol bath or in liquid nitrogen and stored at −80° C.

B. Phage Display and Selection of Domain-Exchanged Antibodies Specific for C. albicans.

1. Electroporation of 2G12 Library DNA into DH5α VCSM13 dsDNA CL F-Cells and Library Expansion.

The six libraries generated in Example 2 were individually electroporated and screened. For electroporation of 2G12 library DNA into electrocompetent DH5α VCSM13 dsDNA CL F-cells, the electroporator settings were adjusted as follows: 2000V (20 kV/cm field strength), resistance to 200Ω, and capacitance to 25 The electroporation cuvettes (0.1 centimeter gap) were pre-chilled at −20° C. and transferred to an ice bucket until use. Electrocompetent DH5α VCSM13 dsDNA CL F-cells (prepared as described in Example 9.A.1, above) were thawed on ice. Pre-chilled 2G12 library DNA was then added to the cells and incubated on ice for 5 minutes. Typically, 100 ng of library DNA in 2-5 μL was added to 100 μL of cells. The volume of cells and amount of DNA added was dependent upon the scale of the electroporation. For a mini electroporation, 100-500 ng DNA was added to 100-500 μl, cells, which resulting in approximately 1×10⁸ to 1×10⁹ cfu. For a midi electroporation, 500-1000 ng DNA was added to 500-1000 μL cells, resulting in approximately 1×10⁹ to 1×10¹⁰ cfu. For a maxi electroporation, 1500-3000 ng DNA was added to 1500-3000 μL cells, resulting in greater that 1×10¹⁰ cfu. One hundred μL of the cells, premixed with the library DNA, was then added to each electroporation cuvette, which was tapped gently until the cell mixture settled flush with the bottom of the cuvette. Thus, for a mini electroporation, there were 1-5 cuvettes; for a midi electroporation, there were 5-10 cuvettes; and for a maxi electroporation, there were 15-30 cuvettes. Any external water or condensation on the cuvette was removed before the samples were pulsed once.

The cuvettes were removed and 1000 μl of prewarmed (37° C.) SOC media was added to resuspend and quench the cells. The cells were transferred to a sterile 50 mL conical polypropylene tube, and the SOC flush process was repeated two more times, resulting in 3 mL of cells from each electroporation cuvette. 2YT medium (containing 16 g Bacto tryptone, 10 g Yeast extract and 5 g NaCl per liter) was added to the cells in each tube to a final volume of 10 mL per tube. Sterile glucose was then added to a final concentration of 20 mM. The cells were incubated at 37° C. with shaking at 250 rpm for 1 hour. (To calculate the transformation efficiency, 90 al of SOC was aliquoted into an ELISA dilution plate, and 10 μl, of the cells (DH5α VCSM13 dsDNA CL F-cells with library DNA) was added to the top well and a 6 step, 10-fold dilution series was prepared. Seventy-five μL of the diluted cells were plated on LB agar/carbenicillin plates (LB agar with 100 μg/mL carbenicillin and 20 mM D-glucose), and the liquid was allowed to dry for a minimum of 15 minutes before being incubated at 37° C. overnight).

Following the 1 hour incubation, the cells were transferred to a 100 mL bottle and 2YT media was added to a final volume of 50 mL before kanamycin (final concentration of 25 μg/mL) and carbenicillin (final concentration of 50 μg/mL) also were added for library expansion. For every 100 nanograms of library DNA electroporated (i.e. for every electroporation cuvette), a separate culture bottle with 50 mL 2YT final volume was used (i.e. for a mini electroporation, there was 1−5×50 mL 2YT; for a midi electroporation, there was 5−10×50 mL 2YT; and for a maxi electroporation, there was 15−30×50 mL 2YT). The library was then expanded by incubation of the cells at 37° C. with shaking at 250 rpm for 2 hours.

2. Phagemid Expression

Following the library expansion, the cell suspension was centrifuged at room temperature for 25 minutes at 4000 rpm and the cell pellet was resuspended in 2YT media to a final volume of such that the OD595 of the bacterial culture was 0.3. Kanamycin was added to a final concentration of 25 μg/mL, carbenicillin was added to a final concentration of 50 μg/mL, and IPTG was added to a final concentration of 1 mM (for variations of the protocol in which pCAL libraries rather than pCAL IT* libraries are used, IPTG is not added). The cells were incubated at 30° C., 300 rpm for 9 hours, then incubated at 4° C. with shaking at 200 rpm until needed.

3. Phage Precipitation and Preparation for Capture

To precipitate the phage, the cultures bottles containing the expressed phage were removed from the 4° C. incubator and centrifuged at 4000 rpm for 30 minutes. Thirty-two mL of the supernatant was transferred to a 50 mL Nalgene centrifuge tube and 8 mL of 20% PEG with 2.5M NaCl was added (a ratio of 4:1 supernatant: 20% PEG with 2.5M NaCl). The tube was inverted 10 times before being incubated on ice for 30 minutes. The centrifuge tube was spun at 13,000 rpm for 30 minutes at 4° C., and the supernatant was pour off. The tube was inverted on a paper towel for 5-10 minutes to remove any excess media. The phage pellet on the bottom of the tube was carefully resuspended (without any bubbles forming) in 1000 μL 1×PBS if a mini electroporation was originally performed, 3750 μL 1×PBS if a midi electroporation was originally performed, or 10000 μL 1×PBS if a maxi electroporation was originally performed. The resuspended phage were transferred to an appropriate number of sterile 1.5 mL microcentrifuge tubes, which were centrifuged at 13,500 rpm, at 25° C. for 5 minutes to pellet cell debris. Finally, supernatant containing the resuspended phage was mixed at a 1:1 ratio with 8% nonfat dry milk (NFDM; reconstituted in 1×PBS) for a final concentration of 4% NFDM. Any unused supernatant was transferred to a sterile 1.5 mL microcentrifuge tube.

4. Phage Capture

An appropriate amount of phage (1000 μL for a mini scale selection; 5000 μL for a midi scale selection; 15000 μL for a maxi scale selection), was added to an 1.5 mL tube or 50 mL conical tube (depending on the scale of selection). The phage were then mixed with Tween20 to a final concentration of 0.05% Tween20, and 1×10⁸ formalin-fixed C. albicans cells. The mixture was then incubated with rocking for 2 hours at 37° C. The C. albicans cells were washed by centrifugation at 4000 rpm for 5 minutes, removal of the supernatant, and resuspension in 1500 μL, 5000 μL or 15000 μL PBS/0.05% Tween20 (for mini, midi and maxi scale selections, respectively). The washing procedure was repeated four times for a total of 5 washes.

5. Phage Elution

To elute the phage, 150 μL, 500 μL or 1000 μL of 0.1M glycine, pH 2.2 (for a mini, midi or maxi scale selection, respectively), was added to the cells and incubated for 10 minutes at room temperature. The tube was vortexed repeatedly to ensure complete elution of all of the phage. After centrifugation to pellet the cells, the glycine containing the eluted phage was transferred to a sterile 1.5 mL tube and was neutralized with the addition of 15 μL, 50 μL or 100 μL of 2M Tris base, pH 9.0 (for a mini, midi or maxi scale selection, respectively).

The phage were then used to infect 2.5 mL, 7.5 mL or 15 mL (for a mini, midi or maxi scale selection, respectively) of XL1-Blue MRF′ cells (OD600 of 0.6-1.5).

The cells were incubated for 30 minutes at room temperature. The cells were spread on a Corning bioassay tray (LB agar containing 100 μg/mL carbenicillin, 100 mM D-glucose), with 2.5 mL cells per tray. The tray was incubated at room temperature for 30 minutes before being incubated at 37° C. for 12 hours.

6. DNA Purification and Further Rounds of Selection

After the 12 hour incubation, the cells were scraped from the tray and DNA was purified using a Qiagen DNA purification kit according to the manufacturers instructions. Additional rounds of selection were then performed by electroporating the purified DNA into the electrocompetent DH5α VCSM13 dsDNA CL F-cells, and proceeding with the phage expression, precipitation, capture and elution, as described above. To wash the phage-bound cells (from Example 4.B.4, above) in the subsequent selection rounds, the following wash conditions were used: Round 2: 5 washes as described for the first round; Round 3; 10 washes with vigorous vortexing and pipetting the cells up and down; Rounds 4-8; 10 washes with vigorous vortexing and pipetting the cells up and down, including a 5 minute incubation at room temperature with rocking between each wash.

### Summary of Library Screening

Table 18 below summarizes the screening for the various CDRL3 libraries generated in Example 2A above. The AGYS and QHYA libraries, which were generated separately, were screened together, with a total of 5 rounds of panning performed. The AGYS+1 and AGYS+2 libraries were screened separately, with a total of 6 rounds of panning performed. 400 clones each from the last two rounds of panning for each screened library were tested for binding to C. albicans using ELISA as described in Example 5 below.

TABLE 18
Library Screening Summary

| | Overlap PCR Libraries | | |
|---|---|---|---|
| Library | AGYS/QHYA | AGYS + 1 | AGYS + 2 |
| # Rounds | 5 | 6 | 6 |
| Clones Tested | 400 from | 400 from | 400 from |
| by ELISA | round 4 | round 5 | round 5 |
| Clones Tested | 400 from | 400 from | 400 from |
| by ELISA | round 5 | round 6 | round 6 |

### Example 5

### Preparation of Candida albicans and Control Antigen for ELISA Screening of Fabs

C. albicans cells were prepared for use as the C. albicans target antigen for ELSA screening of the Fab polyclonal pre-selected library isolated in the phage display screening described in Example 4. A starter culture was first prepared by inoculation of 10 mL of YPD medium with a single colony of C. albicans (Cat. No. 10231, ATCC). The cells were cultured at 37° C. with shaking at 170 rpm for 24 hours, before 500 μl, of culture was removed and transferred into 10 mL of fresh YPD medium. The culture was then diluted 1:3 in YPD medium and plated at 100 μL per well of the ELISA microplate (Reacti-Bind White Opaque 96-well plate). The plate was sealed with Qiagen tape pad and incubated at 37° C. for 8-16 hours. Following incubation, the plate was washed 5 times with PBS with 0.05% Tween20. Finally, the ELISA plate was blocked with 250 μl, of 4% NFDM-PBS at 37° C. for 2 hours and then used in the ELISA assay described in Example 6.

ELISA plates containing chicken albumin or goat anti-human Fab were also prepared for negative controls. 100 ng of chicken albumin or goat anti-human Fab (100 μL, diluted in PBS) was added to each well of an ELISA microplate (Reacti-Bind White Opaque 96-well plate). The ELISA plate was incubated overnight with rocking at 4° C. Following incubation, the plate was washed 5 times with PBS with 0.05% Tween20. Finally, the ELISA plate was blocked with 250 μL of 4% NFDM-PBS at 37° C. for 2 hours and then used in the ELISA assay described in Example 6.

### Example 6

### ELISA Screening of Fab Candidates for Binding to Candida albicans

The polyclonal library DNA that was pre-selected by phage display in Example 4 was then further screened for identification of single Fab clones that bind to C. albicans. A summary of the number of clones and the round from which they were selected for the various libraries that were screened in Example 4 is shown in Table 18 above. One nanogram of library DNA prepared using a Qiagen Qiafilter according to the manufacturer's instructions was transformed into electrocompetent DH5 Alpha E (F-) cells (Invitrogen). The transformed cells were plated onto LB agar plates containing 100 μg/mL carbenicillin and 100 mM glucose to obtain single colonies. The culture plates were inverted and incubated at 37° C. for 14-16 hours.

Individual colonies were then inoculated into a 96 deep well (1 mL volume) parental microplate containing 1.2 mL SB media containing 50 μg/ml carbenicillin and 20 mM glucose. The parental plate was incubated at 37° C. with shaking at 300 rpm for 12-14 hours.

Following incubation of the parental microplate cultures, a 96 deep well daughter microplate was prepared with 1.0 mL SB culture containing 50 μg/ml carbenicillin and 1 mM IPTG. 200 μA of supernatant from the parental plate was transferred to the daughter plate. The parental plate was centrifuged at 4000 rpm for 20 minutes, and the supernatant was discarded. The parental plate was stored at −80° C. The parental plate was saved for later preparation of DNA and sequence analysis after clonal target antigen recognition to C. albicans was determined. For induction of antibody expression, the daughter plate was incubated at 30° C. with shaking at 300 rpm for 8 hours. The daughter plate was were then stored at −80° C., overnight.

The following day, the daughter plate was removed from −80° C. storage and subjected to three freeze/thaw cycles of 37° C. water bath for 5 minutes, followed by incubation in a dry ice ethanol bath for 5 minutes to lyse the cells. The microplate then was spun at 4000 rpm in the tabletop centrifuge for 30 minutes to clear the lysate. The soluble, freeze thawed antibodies (supernatants) from the daughter plate were then diluted 1:1 with 8% NFDM-1×PBS+0.1% Tween20 buffer into a 96 well dilution plate.

ELISA plates were coated with C. albicans, chicken albumin and goat anti-human Fab as described above in Example 4. The 4% NFDM-PBS blocking solution was discarded and the ELISA plates were washed two times with 1×PBS+0.05% Tween20 wash buffer. 100 μl of the diluted antibodies from the daughter plate was transferred from the 96 well dilution plate to the ELISA plates containing the C. albicans, chicken albumin and goat anti-human Fab. Dilutions of the 2G12 IgG antibody were employed as a positive control. For the negative control, several wells received no primary antibody. The ELISA plates were then incubated at 37° C. for 1 hour with rocking. Following antibody incubation, the media from the ELISA plate wells was discarded to remove unbound antibody and the plates were washed 10 times with 1×PBS+0.05% Tween20 wash buffer.

For detection of Fab antibody binding, an anti-human Fab secondary antibody (Goat Anti Human Fab Min X (Pierce, 31414)) was employed. 100 μl of the diluted secondary antibody (1:50,000, diluted according to manufacturers instructions, using 4% NFDM-1×PBS+0.05% Tween20 as dilution buffer) was added to each well of the ELISA plates. The ELISA plates were then incubated at 37° C. for 1 hour with rocking. Following incubation, the secondary antibody solution was discarded to remove unbound antibody and the ELISA plates were washed 5 times with 1×PBS+0.05% Tween20 wash buffer. 50 μl of Supersignal ELISA Femtomax Sensitivity Substrate solution was added to each ELISA plate well. The ELISA plates were then read by measuring luminescence (relative light units (RLU)) using a Biotek Synergy2 luminometer. Positive hits were identified as wells that had greater than 10 times the relative light units (RLU) over background. Clones with RLU values less than 10 times over background and were not selected for follow up. Background was calculated by averaging the values from control wells without primary antibody.

Antibody 2G12 was diluted from concentrations of 0.0001 to 25 p.g/mL and tested for binding to goat anti-Human Fab to generate a standard curve. Using linear regression analysis obtained from the standard curve, estimated working concentrations of the antibody lysates were calculated and values were expressed in nanograms per mL. Specific binding of clones to C. albicans was normalized for antibody expression (RLU) per nanogram of antibody.

DNA from positive clones identified in the screen was prepared using the stored parental plates and sequence analysis was performed. Sequencing was performed for both the heavy and light chains of positive clones. A summary of the clones identified in the screen are shown in Tables 19a and 19b. The approximate affinities (in μg/mL) for selected Fabs are set forth in Table 20a below. Affinity can be converted to molar affinity based on the average molecular weight of a Fab (−50 kDa). Therefore, the affinity is approximately 20 nM for antibody A4F10, 30 nM for antibody A1G7, 40 nM for antibody P2H 12, and 180 nM for antibodies 1H12 and 4F8. The relative light unit value of selected antibodies evaluated in the luminescent C. albicans ELISA binding assay and the mean FITC fluorescence value of selected antibodies evaluated in the C. albicans FACS binding assay are set forth in Table 20b. Of these clones, 10 were selected for further study (see Table 22 below).

TABLE 19a
AGYS CDRL3 Mutants Identified by ELISA as binding to C. Albicans

| | K--- | R--- | M--- | Q--- | V/S--- | I/L--- | E/H--- |
|---|---|---|---|---|---|---|---|
| -EWR | QHYKEWRAT | QHYREWRAT | | | | | |
| | (SEQ ID | (SEQ ID | | | | | |
| | NO: 30) | NO: 218) | | | | | |
| -EWS | QHYKEWSAT | QHYREWRAT | | | | | |
| | (SEQ ID | (SEQ ID | | | | | |
| | NO: 31) | NO: 32) | | | | | |
| -EWW | QHYKEWWAT | QHYREWWAT | | | | | |
| | (SEQ ID | (SEQ ID | | | | | |
| | NO: 34) | NO: 35) | | | | | |
| -EWH | | QHYREWHAT | | | | | |
| | | (SEQ ID | | | | | |
| | | NO: 680) | | | | | |
| -EFN | | QHYREFNAT | | | | | |
| | | (SEQ ID | | | | | |
| | | NO: 219) | | | | | |
| -EFH | | QHYREFHAT | | | | | |
| | | (SEQ ID | | | | | |
| | | NO: 220) | | | | | |
| -SYN | | | | | | QHYLSYNAT | |
| | | | | | | (SEQ ID | |
| | | | | | | NO: 681) | |
| -SWS | | | | | | QHYLSWSAT | |
| | | | | | | (SEQ ID | |
| | | | | | | NO: 36) | |
| -AWS | | | | | | QHYLAWSAT | |
| | | | | | | (SEQ ID | |
| | | | | | | NO: 33) | |
| -PFN | QHYKPFNAT | QHYRPFNAT | QHYMPFNAT | QHYQPFNAT | | QHYLPFNAT | QHYEPFNAT |
| | (SEQ ID | (SEQ ID | (SEQ ID | (SEQ ID | | (SEQ ID | (SEQ ID |
| | NO: 37) | NO: 38) | NO: 39) | NO: 40) | | NO: 41) | NO: 42) |
| | | | QHYMPFNAS | | | | |
| | | | (SEQ ID | | | | |
| | | | NO: 222) | | | | |
| -PFE | QHYKPFEAT | QHYRPFEAT | | | | QHYIPFEAT | |
| | (SEQ ID | (SEQ ID | | | | (SEQ ID | |
| | NO: 43) | NO: 44) | | | | NO: 223) | |
| -PFQ | QHYKPFQAT | QHYRPFQAT | | QHYQPFQAT | | QHYIPFQAT | |
| | (SEQ ID | (SEQ ID | | (SEQ ID | | (SEQ ID | |
| | NO: 45) | NO: 46) | | NO: 47) | | NO: 48) | |
| | QHYKPFSAS | | | | | QHYLPFQAT | |
| | (SEQ ID | | | | | (SEQ ID | |
| | NO: 49) | | | | | NO: 224) | |
| -PFS | QHYKPFSAT | QHYRPFSAT | | QHYQPFSAT | QHYVPFSAT | QHYIPFSAT | QHYHPFSAT |
| | (SEQ ID | (SEQ ID | | (SEQ ID | (SEQ ID | (SEQ ID | (SEQ ID |
| | NO: 280) | NO: 50) | | NO: 51) | NO: 52) | NO: 225) | NO: 53) |
| -PFH | QHYKPFHAT | QHYRPFHAT | QHYMPFHAT | QHYQPFHAT | | | QHYEPFHAT |
| | (SEQ ID | (SEQ ID | (SEQ ID | (SEQ ID | | | (SEQ ID |
| | NO: 54) | NO: 55) | NO: 56) | NO: 226) | | | NO: 57) |
| -PFR | QHYKPFRAT | | QHYMPFRAT | QHYQPFRAT | QHYVPFRAT | | QHYEPFRAT |
| | (SEQ ID | | (SEQ ID | (SEQ ID | (SEQ ID | | (SEQ ID |
| | NO: 58) | | NO: 281) | NO: 227) | NO: 59) | | NO: 60) |
| -PFA | QHYKPFAAT | | | | QHYVPFAAT | QHYIPFAAT | |
| | (SEQ ID | | | | (SEQ ID | (SEQ ID | |
| | NO: 61) | | | | NO: 62) | NO: 63) | |
| -PFD | QHYKPFDAT | | QHYMPFDAT | QHYQPFDAT | | | |
| | (SEQ ID | | (SEQ ID | (SEQ ID | | | |
| | NO: 64) | | NO: 65) | NO: 679) | | | |
| -PFK | | | QHYMPFKAT | QHYQPFKAT | QHYVPFKAT | QHYLPFKAT | |
| | | | (SEQ ID | (SEQ ID | (SEQ ID | (SEQ ID | |
| | | | NO: 66) | NO: 682) | NO: 228) | NO: 229) | |
| -PFT | | | QHYMPFTAT | | | | |
| | | | (SEQ ID | | | | |
| | | | NO: 67) | | | | |
| -PFP | | | QHYMPFPAT | | | | |
| | | | (SEQ ID | | | | |
| | | | NO: 68) | | | | |
| -PFW | QHYKPFWAT | | | QHYQPFWAT | QHYSPFWAT | | |
| | (SEQ ID | | | (SEQ ID | (SEQ ID | | |
| | NO: 230) | | | NO: 69) | NO: 70) | | |
| -PFV | | | | QHYQPFVAT | | | |
| | | | | (SEQ ID | | | |
| | | | | NO: 231) | | | |
| -PFG | | | | | | QHYLPFGAS | |
| | | | | | | (SEQ ID | |
| | | | | | | NO: 244) | |
| -PFM | QHYKPFMAT | | | | | | |
| | (SEQ ID | | | | | | |
| | NO: 687) | | | | | | |
| -PWS | | QHYRPWSAT | | | | | |
| | | (SEQ ID | | | | | |
| | | NO: 221) | | | | | |
| -PWW | | QHYRPWWAT | | | | QHYLPWWAT | |
| | | (SEQ ID | | | | (SEQ ID | |
| | | NO: 232) | | | | NO: 233) | |
| -PYR | QHYKPYRAT | | QHYMPYRAT | QHYQPYRAT | | QHYLPYRAT | QHYEPYRAT |
| | (SEQ ID | | (SEQ ID | (SEQ ID | | (SEQ ID | (SEQ ID |
| | NO: 72) | | NO: 73) | NO: 74) | | NO: 75) | NO: 76) |
| | | | QHYMPYRAS | | | | |
| | | | (SEQ ID | | | | |
| | | | NO: 71) | | | | |
| -PYD | QHYKPYDAT | | | | | | |
| | (SEQ ID | | | | | | |
| | NO: 77) | | | | | | |
| -PYS | QHYKPYSAT | | QHYMPYSAT | QHYQPYSAT | | | |
| | (SEQ ID | | (SEQ ID | (SEQ ID | | | |
| | NO: 78) | | NO: 234) | NO: 235) | | | |
| -PYV | | | | QHYQPYVAT | | | QHYEPYVAT |
| | | | | (SEQ ID | | | (SEQ ID |
| | | | | NO: 79) | | | NO: 236) |
| -PYK | | | QHYMPYKAT | | | | QHYEPYKAT |
| | | | (SEQ ID | | | | (SEQ ID |
| | | | NO: 237) | | | | NO: 80) |
| -PYQ | | | | | | QHYLPYQAS | |
| | | | | | | (SEQ ID | |
| | | | | | | NO: 81) | |
| -PYN | | | QHYMPYNAT | QHYQPYNAT | | QHYLPYNAS | |
| | | | (SEQ ID | (SEQ ID | | (SEQ ID | |
| | | | NO: 238) | NO: 239) | | NO: 240) | |
| -PYW | | | | | QHYSPYWAT | | |
| | | | | | (SEQ ID | | |
| | | | | | NO: 241) | | |
| -PYE | | | | | | QHYLPYEAS | |
| | | | | | | (SEQ ID | |
| | | | | | | NO: 242) | |
| -PYL | | | | | | | QHYEPYLAT |
| | | | | | | | (SEQ ID |
| | | | | | | | NO: 243) |
| -DFS | QHYKDFSAT | | | | | | |
| | (SEQ ID | | | | | | |
| | NO: 245) | | | | | | |
| -AYQ | | | QHYMAYQAT | | | | |
| | | | (SEQ ID | | | | |
| | | | NO: 246) | | | | |
| -AYD | | | QHYMAYDAT | | | | |
| | | | (SEQ ID | | | | |
| | | | NO: 683) | | | | |
| -AFN | | | | QHYQAFNAT | | | |
| | | | | (SEQ ID | | | |
| | | | | NO: 247) | | | |

TABLE 19b
AGYS+1 and QHYA CDRL3 Mutants Identified by ELISA
as binding to C. Albicans

| AGYS+1 | | QHYA | |
|---|---|---|---|
| QHYRPHTGAT | (SEQ ID NO: 82) | NPLSGYSAT | (SEQ ID NO: |
| | | | 248) |
| QHYTAHDGAT | (SEQ ID NO: 83) | | |
| QHYTAHRGAT | (SEQ ID NO: 84) | | |
| QHYRAHTGAT | (SEQ ID NO: 85) | | |
| QHYTAHTGAT | (SEQ ID NO: 86) | | |
| QHYTDHHGAT | (SEQ ID NO: 87) | | |
| QHYTDHKGAT | (SEQ ID NO: 88) | | |
| QHYTDHRGAT | (SEQ ID NO: 89) | | |
| QHYTDHYGAT | (SEQ ID NO: 90) | | |
| QHYTAHNGAT | (SEQ ID NO: 684) | | |
| QHYTPHFGAT | (SEQ ID NO: 685) | | |
| QHYRAHSGAT | (SEQ ID NO: 686) | | |

TABLE 20a
Affinities of selected Fabs

| | | Approximate Affinity (method of |
|---|---|---|
| Fab | CDRL3 | determination) |
| 4F8 | QHYKEWRAT | 9 μg/mL (FACS) |
| 1F8 | QHYLPFNAT | unknown |
| 1H12 | QHYMPYRAS | 9 μg/mL (luminescent ELISA) |
| A4F10 | QHYTDHYGAT | 1 μg/mL (luminescent ELISA) |
| A1G7 | QHYTDHRGAT | 1.5 μg/mL (luminescent ELISA) |
| P2H12 | QHYTDHHGAT | 2 μg/mL (luminescent ELISA) |

TABLE 20b
Binding data of Fabs as measure by ELISA and FACS

| Fab | CDRL3 | ELISA (RFU) | FACS (MF) |
|---|---|---|---|
| ID-1-RD4-P2C10 | QHYKPFMAT | 7900 | 10635 |
| ID-1-RD4-P2G9 | QHYQPFDAT | 4000 | 2350 |
| ID-1-RD5-P2H8 | QHYREWHAT | 3500 | 4930 |
| ID-2-RD4-P1A5 | QHYLSYNAT | 8137 | 4003 |
| ID-2-RD6-P1H10 | QHYQPFKAT | 5000 | 2190 |
| ID-2-RD6-P1H11 | QHYMAYDAT | 13000 | 3756 |
| ID-3-RD6-P1H10 | QHYTAHNGAT | 50000 | 3379 |
| ID-3-RD5-P2E9 | QHYTPHFGAT | 8000 | 8912 |
| ID-3-RD5-P2H7 | QHYRAHSGAT | 27000 | 10571 |
| 1H12 | QHYMPYRAS | 1709 | 2076 |
| Assay Background | | 950 | 100 |

### Example 7

### Generation of IgG

In this example, Fab antibodies identified in Example 6 above were converted into IgGs by cloning into either the 2G12 pCALM 8 His mammalian expression vector (SEQ ID NO:211) or the 2G12 pDR12 mammalian expression vector (SEQ ID NO:212), both of which contained the 2G12 heavy chain set forth in SEQ ID NOS:209 (identical to SEQ ID NO:160) and 210, respectively. Primers specific to the 5′ and 3′ end of the light chain of 2G12 were generated. The primers additionally contained sequences for restriction sites to allow cloning into each respective vector.

A. Cloning into pCALM Mammalian Expression Vector

Primers 2G12IgGLC-F and 2G12IgGLC-R (set forth in Table 21 below) were used to amplify the light chains of Fabs 1H12, 4F8 and 1F8. XhoI (2G12IgGLC-F) and EcoRI (2G12IgGLC-R) restriction sites are shown in bold in Table 21 below. For each reaction, each variant DNA (100 ng) was mixed with 20 pmoles of 2G12IgGLC-F and 20 pmoles of 2G12IgGLC-R and incubated in the presence of 1 μL Advantage HF2 Polymerase Mix (Clontech), 5 μL of 10c HF2 reaction buffer, 5 μL of 10×dNTP mixture and PCR grade water to a final reaction volume of 50 μL. The PCR was carried out using the following reaction conditions: 1 minute denaturation at 95° C., followed by 30 cycles of 5 seconds of denaturation at 95° C., 10 seconds of annealing at 60° C., and 30 seconds of extension at 68° C., then finishing with a 3 minute incubation at 68° C. The amplified fragments (735 bp) were gel-purified using a Gel Extraction Kit (Qiagen) according to the manufacturer's instruction. The purified products were run on 1% agarose gel and each fragment was gel-purified with Gel Extraction Kit (Qiagen) according to the manufacturer's instruction.

The gel-purified fragments were digested with XhoI and EcoRI and subsequently ligated into the similarly digested 2G12 pCALM 8 His mammalian expression vector in the presence of T4 DNA ligase.

B. Cloning into pDR12 Mammalian Expression Vector

Primers 2G12HindIIILC-F1, 2G12HindIIILC-F2 and 2G12EcoRILC-R (set forth in Table 21 below) were used to amplify the light chains of Fabs 1H12, A2A12, P2H12, A1E8, A1G7, A4F10, A5G10, P4H12, and P1F9. HindIII and EcoRI restriction sites are shown in bold in Table 21 below. For each reaction, each variant DNA (diluted 1:100 in Buffer EB) was mixed with 20 pmoles of 2G12HindIIILC-F1, 2 pmoles of 2G12HindIIILC-F2 and 20 pmoles of 2G12EcoRILC-R and incubated in the presence of 1 μL Advantage HF2 Polymerase Mix (Clontech), 5 μL of 10c HF2 reaction buffer, 5 μL of 10×dNTP mixture and PCR grade water to a final reaction volume of 50 μL. The PCR was carried out using the following reaction conditions: 1 minute denaturation at 95° C., followed by 30 cycles of 5 seconds of denaturation at 95° C., 10 seconds of annealing at 60° C., and 30 seconds of extension at 68° C., then finishing with a 3 minute incubation at 68° C. The amplified fragments (735 bp) were gel-purified using a Gel Extraction Kit (Qiagen) according to the manufacturer's instruction. The purified products were run on 1% agarose gel and each fragment was gel-purified with Gel Extraction Kit (Qiagen) according to the manufacturer's instruction.

The gel-purified fragments were digested with HindIII and EcoRI and subsequently ligated into the similarly digested 2G12 pDR12 mammalian expression vector in the presence of T4 DNA ligase.

TABLE 21
2G12 IgG Light Chain Primers

| | | | SEQ |
|---|---|---|---|
| | | | ID |
| Name | nt | Sequences | NO |
| 2G12IgGLC-F | 42 | GGTCCCTGGCTCGAGTGAGGTTGTTAT | 204 |
| | | GACCCAGTCTCCGTC | |
| 2G12IgGLC-R | 44 | CCTGGTACCGAATTCTTAGCATTCACC | 205 |
| | | ACGGTTGAAAGATTTGG | |
| 2G12HindIIILC- | 63 | GTAAGCAAGCTTATGGACATGAGAGTG | 206 |
| F1 | | CCTGCACAGCTGCTGGGACTGCTGCTG | |
| | | CTGTGGCTG | |
| 2G12HindIIILC- | 62 | GGACTGCTGCTGCTGTGGCTGCCAGGC | 207 |
| F2 | | GCCAAGTGCGACGTTGTTATGACCCAG | |
| | | TCTCCGTC | |
| 2G12EcoRILC-R | 46 | CGCTACGAATTCTCAGCATTCACCACG | 208 |
| | | GTTGAAAGATTTGGTAACC | |

TABLE 22
CDRL3s and library screened selected for
conversion to IgGs

| | | SEQ ID NO | SEQ ID NO | Library |
|---|---|---|---|---|
| IgG | CDRL3 | (CDRL3) | (VL) | Screened |
| 1H12 | QHYMPYRAS | 71 | 132 | AGYS |
| 1F8 | QHYLPFNAT | 41 | 102 | AGYS |
| 4F8 | QHYKEWRAT | 30 | 91 | AGYS |
| A1E8 | QHYTDHKGAT | 88 | 149 | AGYS+1 |
| A1G7 | QHYTDHRGAT | 89 | 150 | AGYS+1 |
| P1F9 | QHYRAHTGAT | 85 | 146 | AGYS+1 |
| A2A12 | QHYTAHTGAT | 86 | 147 | AGYS+1 |
| P2H12 | QHYTDHHGAT | 87 | 148 | AGYS+1 |
| A4F10 | QHYTDHYGAT | 90 | 151 | AGYS+1 |
| P4H12 | QHYTAHRGAT | 84 | 145 | AGYS+1 |
| A5G10 | QHYRPHTGAT | 82 | 143 | AGYS+1 |

### Example 8

### Characterization of 2G12 Variants with Improved Affinity for C. albicans

In this example the IgGs generated in Example 7 were assayed for their ability to bind to C. albicans and various other Candida species, namely C. krusei, C. tropicalis, and C. glabrata by both FACS assay and ELISA.

A. C. albicans Binding by FACS Assay

Selected IgG antibodies generated in Example 7 were tested for their ability to bind C. albicans by FACS assay. The C. albicans cells were prepared as follows. A starter culture was first prepared by inoculation of 10 mL of YPD medium with a single colony of C. albicans (Cat. No. 10231, ATCC). The cells were cultured at 37° C. with shaking at 170 rpm for 24 hours and subsequently washed 2× with PBS. The cells were fixed by incubating in 1% formaldehyde in PBS for 30 min at room temperature. Following fixation, the cells were washed 2× in PBS, resuspended in fresh PBS and counted (cells/mL).

Approximately 1×10⁶ C. albicans cells in PBS were transferred to each well of a 96-well deep well plate. The plate was subsequently centrifuged to pellet the cells and the supernatant was removed. The cells were then resuspended in 125 μL of 2% BSA in PBS (a 1:5 dilution of a 1 0% stock solution). The IgG antibodies were serially diluted in PBS (from a concentration of 0.1 to 200 nM). 125 pt each dilution was added to each well (final concentration of 1% BSA). 125 μL of PBS was added to control wells. The plate was then centrifuged for 30 seconds to pool the liquid at the bottom of the wells followed by incubation for 1 hour at room temperature with shaking.

Following incubation, the plate was centrifuged for 5 minutes at 5000 rpm to pellet the cells. The supernatant was removed by inverting the plate and the cells were washed 2× with 1 mL PBS. The cells were resuspended in 250 μL1% BSA in PBS containing 5 μg/mL secondary antibody (anti human IgG, Alexa fluor 488, Invitrogen). The plate was then centrifuged for 30 seconds to pool the liquid at the bottom of the wells followed by incubation for 1 hour at room temperature with shaking while shielded from all light. Following incubation, the plate was centrifuged for 5 minutes at 5000 rpm to pellet the cells. The supernatant was removed by inverting the plate and the cells were washed 2× with 1 mL PBS. The cells were resuspended in 200 μl, PBS. FACS was performed in a FL-1 channel, using the sample that contained only PBS as a control.

The data is shown in Table 23 below, which sets forth the antibody and concentration at 50% maximum binding. 2G12 LC 3ALA (SEQ ID NO:159) which contains three alanine mutations in light chain CDRL3 does not show appreciable binding to C. albicans. Wildtype 2G12 binds at about 150 nM while CDRL3 mutants 1H12 (QHYMPYRAS, SEQ ID NO:71), 1F8 (QHYLPFNAT, SEQ ID NO:41) and 4F8 (QHYKEWRAT, SEQ ID NO:30) all have from 10- to 30-fold increased binding affinity to C. albicans. 2G12 Polymun (Cat. No. AB002, Polymun Scientific) binds at approximately 500 nM. The difference in affinity between 2G12 and 2G12 Polymun is due to the fact the 2G12 contains IgG aggregates (approximately 8-10% aggregates) which increase the affinity for binding to C. albicans.

TABLE 23
Binding to C. albicans by FACS

| Antibody (IgG) | [50% Max] | |
|---|---|---|
| 2G12 LC 3ALA | N/D | |
| 2G12 Polymun | 500 | nM |
| 2G12 | 150 | nM |
| 1H12 | 5.2 | nM |
| 1F8 | 15.1 | nM |
| 4F8 | 9.4 | nM |

B. C. krusei, C. tropicalis, and C. glabrata binding by FACS Assay Selected IgGs were analyzed for their ability to bind to C. albicans, C. krusei, C. tropicalis, and C. glabrata by FACS assay. The C. krusei, C. tropicalis, and C. glabrata used in the assay were clinical isolates. The assay was performed as described in Example 8.A. above. The antibodies were tested at concentrations between 0.1 and 1000 nM. 2G12 Polymun (Cat. No. AB002, Polymun Scientific) was used as a control. The antibodies that were tested are set forth in Table 24. Antibodies A1E8, A1G7, A2A12, P2H12, A4F10, and A5G10 bind C. albicans and C. krusei with an affinity of approximately 50 nM. Antibodies A1E8, A1G7, A2A12, P2H12, A4F10, and A5G10 bind C. tropicalis with an affinity between approximately 50-100 nM. Antibodies A1E8, A1 G7, A2A12, P2H12, A4F10, and A5G10 bind C. glabrata but do not show appreciable binding at the tested antibody concentrations. 2G12 Polymun does not show appreciable binding to any of the isolates. Selected affinities for the various Candida isolates are set forth in Table 25 below. CDRL3 mutants 1H12 and P1F9 bind to all 4 isolates with low nanomolar affinity.

TABLE 24
IgGs screened for binding to C. albicans, C.
krusei, C. tropicalis, and C. glabrata by FACS

| | | SEQ ID NO | SEQ ID NO | Library |
|---|---|---|---|---|
| IgG | CDRL3 | (CDRL3) | (VL) | Screened |
| 2G12 | QHYAGYSAT | 2 | — | N/A |
| Polymun | | | | |
| 1H12 | QHYMPYRAS | 30 | 91 | AGYS |
| A1E8 | QHYTDHKGAT | 88 | 149 | AGYS+1 |
| A1G7 | QHYTDHRGAT | 89 | 150 | AGYS+1 |
| P1F9 | QHYRAHTGAT | 85 | 146 | AGYS+1 |
| A2A12 | QHYTAHTGAT | 86 | 147 | AGYS+1 |
| P2H12 | QHYTDHHGAT | 87 | 148 | AGYS+1 |
| A4F10 | QHYTDHYGAT | 90 | 151 | AGYS+1 |
| P4H12 | QHYTAHRGAT | 84 | 145 | AGYS+1 |
| A5G10 | QHYRPHTGAT | 82 | 143 | AGYS+1 |

TABLE 25
Selected affinities for C. albicans, C. krusei,
C. tropicalis, and C. glabrata

| | C. albicans | C. krusei | C. tropicalis | C. glabrata |
|---|---|---|---|---|
| 2G12 | ~500 nM | ~1000 nM | n/a | n/a |
| Polymun | | | | |
| 1H12 | 10 nM | 17 nM | 23 nM | 12 nM |
| P1F9 | 23 nM | 27 nM | 51 nM | 102 nM |
| P4H12 | N/D | N/D | 21 nM | N/D |
| A5G10 | N/D | N/D | N/D | 72 nM |

C. C. albicans ELISA Binding Assay

Select IgG antibodies generated in Example 7 were tested for their ability to bind C. albicans by ELISA assay. Binding was detected by detecting a colorimetric change (absorbance at 450 nm) or by detecting bioluminescence.

General Procedure

The C. albicans cells were prepared as follows. A starter culture was first prepared by inoculation of 10 mL of YPD medium with a single colony of C. albicans (Cat. No. 10231, ATCC). The cells were cultured at 37° C. with shaking at 170 rpm for 24 hours. A coating culture was prepared by transferring 500 μL of starter culture into 10 mL of YPD medium. The cells were cultured at 37° C. with shaking at 170 rpm for 24 hours.

Following incubation, the coating culture was diluted 1:3 in YPD medium and plated in a 96-well plate (see Table 26 below). A negative control plate was prepared by coating with chicken albumin (Sigma) at a concentration of 2 μg/mL in PBS. The plates were sealed with Qiagen tape pad and incubated at 37° C. overnight. Following overnight incubation, the plates were washed 5× with PBS containing 0.05% Tween20. The plates were then blocked with 4% NFDM in PBS (see Table 26 below) and incubated at 37° C. for 2 hours. Following blocking, the plates were washed 2× with PBS containing 0.05% Tween20.

The IgGs to be tested were serially diluted in 4% NFDM in PBS with 0.05% Tween20 and each dilution series was transferred to a C. albicans coated plate and an chicken albumin coated plate. 4% NFDM in PBS with 0.05% Tween20 was added to one well of each plate for a “secondary only” control. The plates were sealed with Qiagen tape pad and incubated at 37° C. for 2 hours. Following incubation, the plates were washed 5× with PBS containing 0.05% Tween20. Goat anti-Human Fab Min X secondary antibody (Cat. No. 31414, Pierce) was added to each well according to the dilutions and amounts listed in Table 26 below. The plates were sealed with Qiagen tape pad and incubated at 37° C. for 1 hour. Following incubation, the plates were washed 5× with PBS containing 0.05% Tween20.

TABLE 26
Summary of volume of reagents used in ELISA

| | Coating C. | Block (4% | | Goat anti-Human Fab |
|---|---|---|---|---|
| | Albicans | NFDM in | | Min X Secondary |
| Assay | Cells | PBS) | IgG | Antibody |
| Colorimetric | 50 μL | 130 μL | 50 μL | 1:1000 dilution |
| | | | | 50 μL |
| Luminescent | 100 μL | 250 μL | 100 μL | 1:50000 dilution |
| | | | | 100 μL |

Detection

Colorimetric: Add 50 μL TMB Substrate (Cat. No. 34021, Pierce) to each well and incubate for 5-10 minutes. Stop the reaction by adding 50 μL 1.0 N. H₂S0₄ and read the absorbance at 450 nm using an ELISA plate reader.

Luminescence: Add 50 μl, Supersignal ELISA Femtomax Sensitivity Substrate (Pierce) to each well. Measure the luminescence (RLU, relative light units) using a Biotek Synergy2 luminometer.

Results

Selected IgGs were analyzed for their ability to bind to C. albicans using colorimetric detection. The antibodies were tested at concentrations between 0.0001 and 500 nM. 2G12 Polymun (Cat. No. AB002, Polymun Scientific) and 2F5 Polymun (Cat. No. AB0001, Polymun Scientific) were used as controls. The data is set forth in Table 27 below. Antibody 2F5 Polymun, a monoclonal antibody that binds HIV gp120, did not bind to C. albicans. 2G12 Polymun bound with a 50% Max concentration of 76.3 nM while 2G12 had a 8-fold higher affinity. The difference in affinity between 2G12 and 2G12 Polymun is due to the fact the 2G12 contains IgG aggregates which increase the affinity for binding to C. albicans. CDRL3 mutants 1H12, 1F8 and 4F8 all bind with a 50% Max concentration of approximately 1 nM.

TABLE 27
Binding to C. albicans by ELISA

| Antibody (IgG) | CDRL3 | [50% Max] |
|---|---|---|
| 2F5 Polymun | — | N/D |
| 2G12 Polymun | QHYAGYSAT | 76.3 nM |
| 2G12 | QHYAGYSAT | 9.7 nM |
| 1H12 | QHYMPYRAS | 0.4 nM |
| 1F8 | QHYLPFNAT | 0.9 nM |
| 4F8 | QHYKEWRAT | 1.3 nM |

The antibodies listed in Table 22 above were tested for their ability to bind C. albicans by ELISA using both colorimetric and luminescent detection. The antibodies were tested at concentrations between 0.05 and 700 nM. 2G12 Polymun (Cat. No. AB002, Polymun Scientific) and Fab AC8 were used as controls. Selected data is set forth in Table 27 below. Negative control Fab AC8 did not bind to C. albicans. 2G12 Polymun did not show appreciable binding by luminescence and bound with an EC50 of approximately 500 nM using colorimetric detection. CDRL3 mutant 1H12 had the highest affinity of all the antibodies tested. Antibodies A1E8, A1G7, P1F9, A2A12, P2H12, A4F10, P4H12 and A5G10 all bind C. albicans with the same affinity, between 3.2 and 19 nM.

TABLE 27
Binding to C. albicans by ELISA

| | | Colorimetric | Luminescent |
|---|---|---|---|
| IgG | CDRL3 | ELISA | ELISA |
| 2G12 | QHYAGYSAT | ~500 nM | N/A |
| Polymun | | | |
| 1H12 | QHYMPYRAS | 0.82 nM | 6.1 nM |
| P1F9 | QHYRAHTGAT | 3.2 nM | 19 nM |

Since modifications will be apparent to those of skill in this art, it is intended that this invention be limited only by the scope of the appended claims.

1. An anti-Candida antibody, wherein:
the antibody is a domain-exchanged antibody that binds to an epitope presented on Candida with an affinity of equal to or less than or about 100 nM;
the antibody is not 2G12; and 2G12 comprises a variable heavy chain comprising a sequence of amino acids set forth as amino acids 4-120 of SEQ ID NO:154 and a variable light chain comprising a sequence of amino acids set forth as amino acids 4-105 of SEQ ID NO:155.

2. The anti-Candida antibody of claim 1 that is a full length domain-exchanged antibody or is an antibody fragment thereof selected from a domain-exchanged Fab fragment, a domain-exchanged scFv fragment, a domain-exchanged Fab hinge fragment, domain-exchanged scFv tandem fragment, domain-exchanged scFv tandem fragment or a domain-exchanged single chain Fab fragment.

3. The anti-Candida antibody of claim 1 that is a modified 2G12 antibody, wherein the modification(s) comprises replacement, addition or deletion of an amino acid.

4. The anti-Candida antibody of claim 3, wherein the modification(s) is/are in a complementarity determining region (CDR).

5. The anti-Candida antibody of claim 4, wherein amino acid residue 57 in the heavy chain, based on Kabat numbering, is not modified.

6. The anti-Candida antibody of claim 3 that comprises a variable light chain (V_{L}) complementarity determining region 3 (CDR3) having a sequence of amino acids that contains one or more modifications compared to the V_{L} CDR3 of 2G12 set forth in SEQ ID NO:2.

7. The anti-Candida antibody of claim 6, wherein a modification(s) is selected from among:
an amino acid replacement at one or more positions selected from among L89, L90, L91, L92, L93, L94 and L95, based on Kabat numbering; and
an amino acid addition immediately before or immediately following one or more positions selected from among L89, L90, L91, L92, L93, L94 and L95, based on Kabat numbering.

8. The anti-Candida antibody of claim 7, wherein the one or more modifications are at one or more positions selected from among L92, L93, L94, and L95 of the V_{L} CDR3 of 2G12, based on Kabat numbering.

9. The anti-Candida antibody of claim 7, further comprising an amino acid addition after amino acid residue L95, based on Kabat numbering.

10. The anti-Candida antibody of claim 7, wherein the one or more modifications are at one or more positions selected from among L89, L90, L91, and L92 of the V_{L} CDR3 of 2G12, based on Kabat numbering.

11. The anti-Candida antibody of claim 3, wherein the V_{L} CDR3 of the anti-Candida antibody comprises a V_{L} CDR3 having an amino acid sequence set forth in any of SEQ ID NOS: 30-90, 218-248, 280 or 281 or a sequence having 70%, 75%, 80%, 85%, 90%, 91%, 92%, 93%, 94%, 95%, 96%, 97%, 98%, or 99% sequence identity to any of SEQ ID NOS: 30-90, 218-248, 280 or 281.

12. The anti-Candida antibody of claim 11, wherein the V_{L} CDR3 is selected from among QHYMPYRAS (SEQ ID NO:71), QHYLPFNAT (SEQ ID NO:41), QHYKEWRAT (SEQ ID NO:30), QHYTDHKGAT (SEQ ID NO:88), QHYTDHRGAT (SEQ ID NO:89), QHYRAHTGAT (SEQ ID NO:85), QHYTAHTGAT (SEQ ID NO:86), QHYTDHHGAT (SEQ ID NO:87), QHYTDHYGAT (SEQ ID NO:90), QHYTAHRGAT (SEQ ID NO:84), and QHYRPHTGAT (SEQ ID NO:82), or a sequence having 70%, 75%, 80%, 85%, 90%, 91%, 92%, 93%, 94%, 95%, 96%, 97%, 98%, or 99% sequence identity to any of SEQ ID NOS: 30, 41, 71, 82 or 84-90.

13. The anti-Candida antibody of claim 3, wherein:
the heavy chain comprises a 2G12 variable heavy chain (V_{H}) CDR1 set forth in SEQ ID NO: 163, a 2G12 V_{H} CDR2 set forth in SEQ ID NO:164, and a 2G12 V_{H} CDR3 set forth in SEQ ID NO:152; and
the light chain comprises a 2G12 V_{L} CDR1 set forth in SEQ ID NO:165, a 2G12 V_{L} CDR2 set forth in SEQ ID NO:166, and a modified 2G12 V_{L} CDR3 set forth in any of SEQ ID NOS: 30-90, 218-248, 280 or 281.

14. The anti-Candida antibody of claim 3, comprising:
a variable light chain comprising a sequence of amino acids set forth as amino acids 4-105 of any of SEQ ID NO: 91-151, 249-279 or 282-283, or a sequence having at least 70% sequence identity therewith; and
a variable heavy chain comprising a sequence of amino acids set forth as amino acids 4-120 of SEQ ID NO:154.

15. The anti-Candida antibody of claim 14 comprising:
a variable light chain having a sequence of amino acids set forth as amino acids 1-107 of SEQ ID NOS: 457-508 or 518-550; amino acids 1-108 of SEQ ID NOS: 91-142, 249-279, 282-283 or 509-517; or amino acids 1-109 of SEQ ID NOS: 143-151, or a sequence having at least 70% sequence identity therewith; and
a variable heavy chain having a sequence of amino acids set forth in SEQ ID NO:154.

16. The anti-Candida antibody of claim 3, wherein the V_{L} CDR3 of the anti-Candida antibody comprises a V_{L} CDR3 having an amino acid sequence set forth in any of SEQ ID NOS: 678-686 or a sequence having 70%, 75%, 80%, 85%, 90%, 91%, 92%, 93%, 94%, 95%, 96%, 97%, 98%, or 99% sequence identity to any of SEQ ID NOS: 678-686.

17. The anti-Candida antibody of claim 16, wherein:
the heavy chain comprises a 2G12 variable heavy chain (V_{H}) CDR1 set forth in SEQ ID NO: 163, a 2G12 V_{H} CDR2 set forth in SEQ ID NO:164, and a 2G12 V_{H} CDR3 set forth in SEQ ID NO:152; and
the light chain comprises a 2G12 V_{L} CDR1 set forth in SEQ ID NO:165, a 2G12 V_{L} CDR2 set forth in SEQ ID NO:166, and a modified 2G12 V_{L} CDR3 set forth in any of SEQ ID NOS: 678-686.

18. The anti-Candida antibody of claim 3, comprising:
a variable light chain comprising a sequence of amino acids set forth as amino acids 4-105 of any of SEQ ID NO: 687-695, or a sequence having at least 70% sequence identity therewith; and
a variable heavy chain comprising a sequence of amino acids set forth as amino acids 4-120 of SEQ ID NO:154.

19. The anti-Candida antibody of claim 18, comprising:
a variable light chain having a sequence of amino acids set forth as amino acids 1-107 of SEQ ID NOS: 696-701; amino acids 1-108 of SEQ ID NOS: 687-692 or 702-704; or amino acids 1-109 of SEQ ID NOS: 693-695, or a sequence having at least 70% sequence identity therewith; and
a variable heavy chain having a sequence of amino acids set forth in SEQ ID NO:154.

20. The anti-Candida antibody of claim 3 that is a Fab, a Fab dimer, or a full-length antibody.

21. The anti-Candida antibody of claim 1 that has greater affinity for Candida compared to the affinity of the corresponding form of the domain-exchanged antibody 2G12.

22. The anti-Candida antibody of claim 1, wherein the Candida species is selected from among C. albicans, C. tropicalis, C. parapsilosis, C. krusei, C. glabrata, C. lusitaniae, C. dubliniensis and C. guilliermondii.

23. The anti-Candida antibody of claim 1, comprising:
a light chain having a sequence of amino acids set forth in SEQ ID NO:91, 102, 132, 143, 145-151, 457, 468, 498, 509, 511-517; and
a heavy chain having a sequence of amino acids set forth in SEQ ID NO:10; or having a sequence of amino acids set forth in SEQ ID NO:209 or 210.

24. The anti-Candida antibody of claim 1, comprising:
(a) a light chain having a sequence of amino acids set forth in SEQ ID NO:91, 102, 132, 143, 145, 146, 457, 468, 498, 509, 511 or 512; and a heavy chain having a sequence of amino acids set forth in SEQ ID NO:10; or having a sequence of amino acids set forth in SEQ ID NO: 209 or 210; or:
(b) a light chain having a sequence of amino acids set forth in SEQ ID NOS:687-704; and a heavy chain having a sequence of amino acids set forth in SEQ ID NO:10; or having a sequence of amino acids set forth in SEQ ID NO:209 or 210.

25. The anti-Candida antibody of claim 1 that is a dimer.

26. The anti-Candida antibody of claim 3, wherein the antibody or antibody fragment is conjugated to polyethylene glycol (PEG) and/or a multimerization domain.

27. A domain exchanged antibody that binds to the same epitope as an antibody of claim 1, wherein:
the antibody is not 2G12; and
2G12 comprises a variable heavy chain comprising a sequence of amino acids set forth as amino acids 4-120 of SEQ ID NO:154 and a variable light chain comprising a sequence of amino acids set forth as amino acids 4-105 of SEQ ID NO:155.

28. A combination, comprising:
an anti-Candida antibody of claim 1; and
an antifungal agent or one or more additional antifungal antibodies that differ from the first antibody.

29. The combination of claim 28, that comprises an additional antifungal antibody, wherein the additional antifungal antibody is an anti-Candida antibody, whereby the combination comprises two or more different anti-Candida antibodies.

30. The combination of claim 29, wherein the additional antibody is a full-length antibody, a single-chain Fv (scFv), Fab, Fab′, F(ab′)₂, Fv, dsFv, diabody, Fd, Fd′ fragment, or a domain-exchanged antibody or antibody fragment thereof.

31. The combination of claim 28, wherein the antibody and the antifungal agent or additional antifungal antibody are formulated as a single composition or separate compositions.

32. A pharmaceutical composition, comprising:
an anti-Candida antibody of claim 1 in a pharmaceutically acceptable carrier or excipient.

33. The pharmaceutical composition of claim 32 that comprises a plurality of anti-Candida antibodies.

34. The pharmaceutical composition of claim 32 that is formulated for systemic, parenteral, topical, oral, mucosal, intranasal, subcutaneous, aerosolized, intravenous, bronchial, pulmonary, vaginal, vulvovaginal, esophageal, or oesophageal administration.

35. A method of treating or preventing a fungal infection in a subject, comprising administering to the subject a therapeutically effective amount of the pharmaceutical composition of claim 32.

36. The method of claim 35, wherein the human subject is a human infant, a human infant born prematurely or at risk of hospitalization for a fungal infection, an elderly human, a human subject who has congenital immunodeficiency, acquired immunodeficiency, HIV, leukemia, or non-Hodgkin lymphoma, is receiving or has received high dosage antibiotic therapy, or a human subject having an organ or tissue transplant or a blood transfusion.

37. A polynucleotide(s) encoding an antibody of claim 1.

38. A polynucleotide encoding the light chain of an antibody of claim 1.

39. A vector(s), comprising the a polynucleotide(s) encoding an antibody of claim 1 or encoding the light chain of an antibody of claim 1.

40. A host cell, comprising a vector of claim 39.

41. The anti-Candida antibody of claim 1 that is selected from among 1H12, 1F8, 4F8, A1E8, A1G7, P1F9, A2A12, P2H12, A4F10, P4H12, A5G10, ID-1-RD4-P2C10, ID-1-RD4-P2G9, ID-1-RD5-P2H8, ID-2-RD4-P1A5, ID-2-RD6-P1H10, ID-2-RD6-P1H11, ID-3-RD6-P1H10, ID-3-RD5-P2E9 or ID-3-RD5-P2H and an antibody fragment thereof.

42. The anti-Candida antibody of claim 41 that is a Fab dimer.
